(19) United States
(12) Patent Application Publication
(10) Pub. No.: US 2021/0292840 A1
(43) Pub. Date: Sep. 23, 2021

(54) SMALL RNA PREDICTORS FOR ALZHEIMER'S DISEASE

(71) Applicant: SRNALYTICS, INC., Needham, MA (US)

(72) Inventors: David W. SALZMAN, Needham, MA (US); Alan P. SALZMAN, Needham, MA (US); Neal C. FOSTER, Needham, MA (US); Nathan S. RAY, Needham, MA (US)

(21) Appl. No.: 17/262,045

(22) PCT Filed: Jul. 25, 2019
(86) PCT No.: PCT/US19/43508

§ 371 Date: Jan. 21, 2021

Related U.S. Application Data
(60) Provisional application No. 62/703,172, filed on Jul. 25, 2018.

(51) Int. Cl. C12Q 1/6883

(52) U.S. Cl. CPC C12Q 1/6883; C12Q 2600/178; C12Q 2600/158; C12Q 2600/112

(57) ABSTRACT

The present disclosure provides methods and kits for evaluating Alzheimer's disease (AD) activity, including in patients undergoing treatment for AD or a candidate treatment for AD, as well as in animal and cell models. Specifically, the present disclosure provides biomarkers (sRNA predictors) that are binary predictors of disease activity, and are useful for detecting and/or evaluating AD disease stage, grade and progression, prognosis, and response to therapy or candidate therapy. The biomarkers are further useful in the context of drug discovery and clinical trials, to identify candidate pharmaceutical interventions (or other therapies) that are useful for the treatment of disease.

### PRIORITY

This application claims the benefit of, and priority to, U.S. Provisional Application No. 62/703,172, filed Jul. 25, 2018, the contents of which are hereby incorporated by reference in its entirety.

### BACKGROUND

Alzheimer's disease (AD) is the most common neurodegenerative disease, as it accounts for nearly 70% of all cases of dementia and affects up to 20% of individuals older than 80 years. Various morphological and histological changes in the brain serve as hallmarks of modern day AD neuropathology. Specifically, two neurological phenomena have been observed: amyloid plaques and neurofibrillary tangles. Disease progression can be categorized as Braak stages, with six stages of disease propagation having been distinguished with respect to the location of the tangle-bearing neurons and the severity of changes in the brain: Braak stages I/II: transentorhinal (temporal lobe) stages, clinically silent cases; Braak stages III/IV: limbic stages, incipient Alzheimer's disease; and Braak stages V/VI: neocortical stages, fully developed Alzheimer's disease.

Alzheimer's patients begin presenting early symptoms, such as difficulties with memory like remembering recent events and also forming new memories. Visuospatial and language problems often follow or accompany the onset of early symptoms involving memory. As the disease progresses, individuals slowly lose the ability to perform the activities of daily living, and eventually, attention, verbal ability, problem solving, reasoning, and all forms of memory become seriously impaired. Indeed, progression of AD is often accompanied by changes in personality, such as increased apathy, anger, dependency, aggressiveness, paranoia and occasionally inappropriate sexual behavior. In the latter stages of AD, individuals may be incapable of communication, show signs of complete confusion, and bedridden.

There are two types of Alzheimer's: early-onset and late-onset, and both types have a genetic component. Early-onset AD patients begin to present symptoms between their 30s and mid-60s and is very rare, while late-onset AD, the most common type, see patients presenting signs and symptoms in the patients' mid-60s. Late-onset AD is known to involve a genetic risk factor, a form of apolipoprotein E (APOE), APOE e4, on chromosome 19, that increases a person's risk.

At this time, there is no cure for AD, and available treatments usually offer, at most, a temporary slowing of the symptomatic deterioration. In addition, Alzheimer's can only be absolutely diagnosed after death, by examination of brain tissue and pathology in an autopsy.

Thus, the identification of disease-modifying therapies is the main objective for pharmaceutical intervention and drug discovery. However, these efforts are hampered by the fact that there are no clinically meaningful biomarkers to aid in drug discovery and development. Such biomarkers need to be accessible, prognostic, and/or disease-specific. Discovery and investigation of therapeutic interventions, including pharmaceutical interventions, would benefit from the availability of biomarkers correlative of underlying disease processes.

Diagnostic tests to evaluate Alzheimer's disease activity are needed, for example, to aid treatment and decision making in affected individuals, as well as for use as biomarkers in drug discovery and clinical trials, including for patient enrollment, stratification, and disease monitoring.

### SUMMARY OF THE INVENTION

The present disclosure provides methods and kits for evaluating Alzheimer's disease (AD) activity, including in patients undergoing treatment for AD or a candidate treatment for AD, as well as in animal and cell models. Specifically, the present disclosure provides biomarkers (sRNA predictors) that are binary predictors of disease activity, and are useful for detecting and/or evaluating AD disease stage, grade, progression, prognosis, and response to therapy or candidate therapy. The biomarkers are further useful in the context of drug discovery and clinical trials, to identify candidate pharmaceutical interventions (or other therapies) that are useful for the treatment or management of disease (e.g., treatment or progression monitoring).

In various aspects and embodiments, the invention involves detecting binary small RNA (sRNA) predictors of Alzheimer's disease or Alzheimer's disease activity, in cells or in a biological sample from a subject or patient. The sRNA sequences are identified as being present in samples of an AD experimental cohort, while not being present in any samples of a comparator cohort (“positive sRNA predictors”). The invention thereby detects sRNAs that are binary predictors, exhibiting 100% Specificity for Alzheimer's disease.

In some embodiments, the invention provides a method for evaluating AD activity in a subject or patient. The method comprises providing a biological sample from a subject or patient exhibiting symptoms and signs of AD, and determining the presence, absence, or level of one or more sRNA predictors in the sample. The presence or level of sRNA predictors is correlative with disease activity.

The positive sRNA predictors include one or more sRNA predictors from Table 2A, Table 4A, and Table 7A (SEQ ID NOS: 1-403). For example, the positive sRNA predictors may include one or more sRNA predictors from Table 2A (SEQ ID NOS: 1 to 46), which were identified in sRNA sequence data of brain tissue samples of AD patients, but were absent from non-disease controls, and various other non-Alzheimer's neurodegenerative disease controls (e.g., Parkinson's disease). In some embodiments, the relative or absolute amount of the one or more predictors is correlative with disease stage or severity. In some embodiments, the positive sRNA predictors include one or more sRNA predictors from Table 4A (SEQ ID NOS: 47-254), which were identified in sRNA sequence data of cerebrospinal fluid (CSF) samples of AD patients, but were absent from healthy controls, and various other non-Alzheimer's neurodegenerative disease controls (e.g., Parkinson's disease). In some embodiments, the positive sRNA predictors include one or more sRNA predictors from Table 4A (SEQ ID NOS: 255-403), which were identified in sRNA sequence data of serum samples of AD patients, but were absent from healthy controls, and various other non-Alzheimer's neurodegenerative disease controls (e.g., Parkinson's disease).

In some embodiments, the number of predictors that is present in a sample, or the accumulation of one or more of the predictors, directly correlates with the progression of AD or underlying severity of disease or active symptoms. In some embodiments, the positive sRNA predictors include one or more sRNA predictors from Table 5 (SEQ ID NOS: 58, 189, 78, 172, 193, 97, 122, 215, 248, 164, 120, 93, 126, 253, 112, 144, 213, 244, 123, 222, 150, 240, 52, 220, 221, 169, 165, and 212), which correlate with Braak stages of AD progression (e.g., in CSF samples). In some embodiments, the positive sRNA predictors include one or more from Table 8 (SEQ ID NOS: 257, 270, 272, 273, 279, 286, 288, 314, 319, 325, 332, 341, 374, 391, and 393), which correlate with Braak stages of AD progression (e.g., in serum samples).

In some embodiments, the presence, absence, or level of at least 1, 2, 3, 4, or 5 sRNAs, or at least 10 sRNAs, or at least 40 sRNAs from one or more of Table 2A, Table 4A, and/or Table 7A are determined (SEQ ID NOS: 1-403). In some embodiments, the presence or absence of at least one negative sRNA predictor is also determined, which are identified uniquely in non-AD samples, such as healthy controls. In some embodiments, a panel of sRNAs comprising positive predictors from Table 2A, Table 4A, and/or Table 7A is tested against the sample. In some embodiments, the panel may comprise at least 2, or at least 5, or at least 10, or at least 20, or at least 25 sRNAs from Table 2A, Table 4A, and/or Table 7A. In some embodiments, the panel comprises all sRNAs from Table 2A, Table 4A, and/or Table 7A. For example, a sample may be positive for at least about 2, 3, 4, or 5 sRNA predictors in Table 2A, Table 4A, and/or Table 7A, indicating active disease, with more severe or advanced disease being correlative with about 10, 15 or about 20 sRNA predictors. In some embodiments, the relative or absolute amount of the sRNA predictors in Table 2A, Table 4A, and/or Table 7A are directly correlative with disease grade or severity (e.g., Braak stage).

Generally, the presence of at least 1, 2, 3, 4, or 5 positive predictors is predictive of AD activity. In some embodiments, a panel of 5 to about 100, or about 5 to about 60, sRNA predictors are tested against the sample. While not each experimental sample will be positive for each positive predictor, the panel is large enough to provide 100% Sensitivity against the training cohorts (e.g., the experimental cohort). That is, each sample in the experimental cohort has the presence of one or more positive sRNA predictors. In such embodiments, the presence or absence of the sRNA predictors in the panel provides (by definition) 100% Specificity and 100% Sensitivity against the training set (i.e., the experimental cohort). In still other embodiments, the sRNA predictors are employed in computational classifier algorithms, including non-bootstrapped and/or bootstrapped classification algorithms. Examples including supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naive Bayes Classifier, Perceptron, Quadratic classifiers, Kernel Estimation, k-Nearest Neighbor, Learning Vector Quantization, and Principal Components Analysis. These classification algorithms may rely on the presence and absence of other sRNAs, other than sRNA predictors. For example, the classifier may rely on the presence of absence of a panel of isoforms (including, but not limited to microRNA isoforms known as ‘isomiRs’), which can optionally include one or more sRNA predictors (i.e., which were identified in sRNA sequence data as unique to a disease condition).

sRNAs can be identified or detected in any biological samples, including solid tissues and/or biological fluids. sRNAs can be identified or detected in animals (e.g., vertebrates and invertebrates), or in some embodiments, cultured cells or the media of cultured cells. For example, the sample may be a biological fluid sample from a human or animal subject (e.g., a mammalian subject), such as blood, serum, plasma, urine, saliva, or cerebrospinal fluid. In some embodiments, the sample is a solid tissue such as brain tissue.

In various embodiments, detection of the sRNAs involves one of various detection platforms, which can employ reverse-transcription, amplification, and/or hybridization of a probe, including quantitative or qualitative PCR, or Real-Time PCR. PCR detection formats can employ stem-loop primers for RT-PCR in some embodiments, and optionally in connection with fluorescently-labeled probes. In some embodiments, sRNAs are detected by a hybridization assay or RNA sequencing (e.g., NextGen sequencing). In some embodiments, RNA sequencing is used in connection with specific primers amplifying the sRNA predictors or other sRNAs in a panel.

The invention involves detection of sRNAs (such as isomiRs) in cells or animals (or samples derived therefrom) that display symptoms and signs of AD. In some embodiments, the invention involves detection of sRNA predictors in cells or animals (or samples derived therefrom) that contain a form of apolipoprotein E (APOE), APOE e4. In various embodiments, the number and/or identity of the sRNA predictors, or the relative amount thereof, is correlative with disease activity for patients, subjects, or cells having a APOE e4 allele. In some embodiments, the sRNA predictor is indicative of AD biological processes in patients or subjects that are otherwise considered Asymptomatic.

In some embodiments, the invention provides a kit comprising a panel of from 2 to about 100 sRNA predictor assays, or from about 5 to about 75 sRNA predictor assays, or from 5 to about 20 sRNA predictor assays. In these embodiments, the kit may comprise sRNA predictor assays (e.g., reagents for such assays) to determine the presence or absence of sRNA predictors from Table 2A, Table 4A, and/or Table 7A. Such assays may comprise reverse transcription (RT) primers, amplification primers and probes (such as fluorescent probes or dual labeled probes) specific for the sRNA predictors over other non-predictive sequences. In some embodiments, the kit is in the form of an array or other substrate containing probes for detection of sRNA predictors by hybridization.

In some aspects, the invention provides kits for evaluating samples for Alzheimer's disease activity. In various embodiments, the kits comprise sRNA-specific probes and/or primers configured for detecting a plurality of sRNAs listed in Table 2A, Table 4A, and/or Table 7A (SEQ ID NOS: 1-403). In some embodiments, the kit comprises sRNA-specific probes and/or primers configured for detecting at least 5, or at least 10, or at least 20, or at least 40 sRNAs listed in Table 2A, Table 4A, and/or Table 7A (SEQ ID NOS: 1-403).

In still other embodiments, the invention involves constructing disease classifiers based on the presence or absence of particular sRNA molecules (e.g., isomiRs or other types of sRNAs). These disease classifiers are powerful tools for discriminating disease conditions that present with similar symptoms, as well as determining disease subtypes, including predicting the course of the disease, predicting response to treatment, and disease monitoring. Generally, sRNA panels (e.g., panels of distinct sRNA variants) will be determined from sequence data in one or more training sets representing one or more disease conditions of interest. sRNA panels and the classifier algorithm can be constructed using, for example, supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naive Bayes Classifier, Perceptron, Quadratic classifiers, Kernel Estimation, k-Nearest Neighbor, Learning Vector Quantization, and Principal Components Analysis. Once the classifier is trained, independent subjects can be evaluated for the disease conditions by detecting the presence or absence, in a biological sample from the subject, of the sRNA markers in the panel, and applying the classification algorithm. Classifiers can be binary classifiers (i.e., classify among two conditions), or may classify among three, four, five, or more disease conditions. The classifiers rely on the presence and absence of sRNAs in the panel, rather than discriminating normal and abnormal levels of sRNAs.

For example, in some embodiments, the invention provides a method for evaluating a subject for one or more disease conditions. The method comprises providing a biological sample of the subject, and determining the presence or absence of a plurality of sRNAs in the sRNA panel. This profile of “present and absent” sRNAs (binary markers) is used to classify the condition of the subject among two or more disease conditions using the disease classifier. The disease classifier will have been trained based on the presence and absence of the sRNAs in the sRNA panel in a set of training samples. For example, the training samples are annotated as positive or negative for the one or more disease conditions (and may be annotated for disease subtype, grade, or treatment regimen), as well as the presence or absence (and in some embodiment, level) of the sRNAs in the panel.

The presence or absence of the sRNAs in the panel is determined in the training set from sRNA sequence data. That is, individual sRNA sequences are identified in the sRNA sequence data by trimming 3′ sequencing adaptors and without consolidating sRNA sequence variants to a reference sequence or genetic locus. For example, after trimming, the unique sequence reads within each disease condition or comparator condition are compiled (i.e., a read count for each unique sequence is prepared). Thus, the presence or absence of specific sRNA sequences, such as isomiRs, are determined in each disease condition, and these variants are not consolidated to reference sequences. These sequences can be used as “binary” markers, that is, evaluated based on their presence or absence in samples, as opposed to discriminating normal and abnormal levels.

Once identified in the sequence data, and selected for inclusion in the computational classifier, molecular detection reagents for the sRNAs in the panel can be prepared. Such detection platforms include quantitative RT-PCR assays, including those employing stem loop primers and fluorescent probes.

Other aspects and embodiments of the invention will be apparent from the following detailed description.

### BRIEF DESCRIPTION OF THE DRAWINGS

FIG. 1A-D depicts ROC/AUC curves for the various IBD classes and controls: Control (1A), Crohn's disease (1B), Ulcerative colitis (1C), and Diverticular disease (1D).

FIG. 2 depicts a heat map showing the proportion of accurate multi-class disease predictions against their true reference identies.

### DESCRIPTION OF THE TABLES

Tables 1A to 1B characterize brain tissue sample cohorts, including Alzheimer's disease (AD) cohort (Table 1A), and control cohort including healthy control and various other non-Alzheimer's neurological disorder controls (Table 1B).

Tables 2A shows sRNA positive predictors in brain tissue samples for AD (SEQ ID NOs: 1-46) with read count, specificity, and sensitivity (e.g., frequency). Table 2B shows positive predictors for AD across brain tissue samples, with number of biomarkers per sample and percent coverage.

Tables 3A to 3B characterize cerebrospinal fluid (CSF) sample cohorts, including Alzheimer's disease (AD) cohort (Table 3A), and control cohort including healthy control and various other non-Alzheimer's neurological disorder controls (Table 3B).

Table 4A shows sRNA positive predictors in CSF for AD (SEQ ID NOs: 47-254) with read count, specificity, and sensitivity (e.g., frequency). Table 4B shows positive predictors for AD across CSF samples, with number of biomarkers per sample and percent coverage.

Table 5 shows a panel of 28 identified sRNA biomarkers from CSF that show correlation to Braak Stage that can be used in the monitoring of AD.

Tables 6A to 6B characterize serum sample cohorts, including Alzheimer's disease (AD) cohort (Table 6A), and control cohort including healthy control and various other non-Alzheimer's neurological disorder controls (Table 6B).

Table 7A shows sRNA positive predictors in serum for AD (SEQ ID NOs: 255-403) with read count, specificity, and sensitivity (e.g., frequency). Table 7B shows positive predictors for AD across serum samples, with number of biomarkers per sample and percent coverage.

Table 8 shows a panel of 15 identified sRNA biomarkers from serum that show correlation to Braak Stage that can be used in the monitoring of AD.

Table 9 depicts a panel of sRNA biomarkers from colon epithelium tissue for Controls (“Normal” individuals) of Inflammatory Bowel Disease.

Table 10 shows a panel of sRNA biomarkers from colon epithelium tissue for Crohn's disease.

Table 11 shows a panel of sRNA biomarkers from colon epithelium tissue for Ulcerative colitis.

Table 12 depicts a panel of sRNA biomarkers from colon epithelium tissue for Diverticular disease.

### DETAILED DESCRIPTION OF THE INVENTION

The present disclosure provides methods and kits for evaluating Alzheimer's disease (AD) activity, including in patients undergoing treatment for AD or a candidate treatment for AD, as well as in animal and cell models. Specifically, the present disclosure provides biomarkers (sRNA predictors) that are binary predictors of disease activity, and are useful for detecting and/or evaluating underlying disease processes, disease grade, progression, and response to therapy or candidate therapy. The biomarkers are further useful in the context of drug discovery and clinical trials, to identify candidate therapies that are useful for treatment of AD or AD symptoms, as well as to select or stratify patients, and monitor disease progression or treatment.

In various aspects and embodiments, the invention involves detecting binary small RNA (sRNA) predictors of Alzheimer's disease or Alzheimer's disease activity, in a cell or biological sample. The sRNA sequences are identified as being present in samples of an AD experimental cohort, while not being present in any samples in a comparator cohort. These sRNA markers are termed “positive sRNA predictors”, and by definition provide 100% Specificity. In some embodiments, the method further comprises detecting one or more sRNA sequences that are present in one or more samples of the comparator cohort, and which are not present in any of the samples of the experimental cohort. These predictors are termed “negative sRNA predictors”, and provide additional level of confidence to the predictions. In contrast to detecting dysregulated sRNAs (such as miRNAs that are up- or down-regulated), the invention provides sRNAs that are binary predictors for Alzheimer's disease activity.

small RNA species (“sRNAs”) are non-coding RNAs less than 200 nucleotides in length, and include microRNAs (miRNAs) (including iso-miRs), Piwi-interacting RNAs (piRNAs), small interfering RNAs (siRNAs), vault RNAs (vtRNAs), small nucleolar RNAs (snoRNAs), transfer RNA-derived small RNAs (tsRNAs), ribosomal RNA-derived small RNA fragments (rsRNAs), small rRNA-derived RNAs (srRNA), and small nuclear RNAs (U-RNAs), as well as novel uncharacterized RNA species. Generally, “iso-miR” refers to those sequences that have variations with respect to a reference miRNA sequence (e.g., as used by miRBase). In miRBase, each miRNA is associated with a miRNA precursor and with one or two mature miRNA (-5p and -3p). Deep sequencing has detected a large amount of variability in miRNA biogenesis, meaning that from the same miRNA precursor many different sequences can be generated. There are four main variations of iso-miRs: (1) 5′ trimming, where the 5′ cleavage site is upstream or downstream from the referenced miRNA sequence; (2) 3′ trimming, where the 3′ cleavage site is upstream or downstream from the reference miRNA sequence; (3) 3′ nucleotide addition, where nucleotides are added to the 3′ end of the reference miRNA; and (4) nucleotide substitution, where nucleotides are changed from the miRNA precursor.

U.S. 2018/0258486, filed on Jan. 23, 2018, and PCT/US2018/014856 filed Jan. 23, 2018 (the full contents of which are hereby incorporated by reference), disclose processes for identifying sRNA predictors. The process includes computational trimming of 3′ adapters from RNA sequencing data, and sorting data according to unique sequence reads.

In some embodiments, the invention provides a method for evaluating Alzheimer's disease (AD) activity. The method comprises providing a cell or biological sample from a subject or patient presenting symptoms and signs of AD, or providing RNA extracted therefrom, and determining the presence or absence of one or more sRNA predictors in the cell or sample. The presence of the one or more sRNA predictors is indicative of Alzheimer's disease activity.

The term “Alzheimer's disease activity” refers to active disease processes that result (directly or indirectly) in AD symptoms and overall decline in cognition, behavior, and/or motor skills and coordination. The term Alzheimer's disease activity can further refer to the relative health of affected cells. In some embodiments, the AD activity is indicative of neuron viability.

The positive sRNA predictors include one or more sRNA predictors from Tables 2A, 4A, or 7A (SEQ ID NOS: 1-403). Sequences disclosed herein are shown as the reverse transcribed DNA sequence. For example, the positive sRNA predictors may include one or more sRNA predictors from Table 2A (SEQ ID NOS: 1-46), which are indicative of AD and/or AD stage, as identified in sequence data of brain tissue samples. In some embodiments, the positive sRNA predictors include one or more sRNA predictors from Table 4A (SEQ ID NOS: 47 to 154), which are indicative of AD and/or AD stage, as identified in sequence data of CSF samples. In some embodiments, the positive sRNA predictors include one or more from Table 7A (SEQ ID NOS: 155-403), which are indicative of AD and/or AD stage, as identified in sequence data of serum samples.

Specifically, Tables 2A and 2B show sRNA positive predictors for AD, as identified in brain tissue samples. These sRNA predictors were present in a cohort of AD brain tissue samples (as the Experimental Group), but were not present in any of the Comparator Group samples, which were comprised of non-disease samples, as well as various other non-Alzheimer's neurological disease samples. Table 2A shows positive predictors for AD regardless of Braak stage. The positive predictors each provides 100% Specificity for the presence of AD in the cohort. Tables 2A and 2B shows the average read count across AD brain tissue samples for the positive predictors. In some embodiments, the number of predictors that is present in a sample directly correlates with the Braak stage of AD.

Tables 4A and 4B show sRNA positive predictors for AD, as identified in cerebrospinal fluid (CSF) samples. These sRNA predictors were present in a cohort of AD CSF samples (as the Experimental Group), but were not present in any of the Comparator Group samples, which were comprised of Healthy samples, as well as various other non-Alzheimer's neurological disease samples. Table 4A shows positive predictors for AD regardless of Braak stage. The positive predictors each provides 100% Specificity for the presence of AD in the cohort. Tables 4A and 4B shows the average read count across AD CSF samples for the positive predictors. In some embodiments, the number of predictors that is present in a sample directly correlates with the Braak stage of AD.

Tables 7A and 7B show sRNA positive predictors for AD, as identified in serum samples. These sRNA predictors were present in a cohort of AD serum samples (as the Experimental Group), but were not present in any of the Comparator Group samples, which were comprised of Healthy samples, as well as various other non-Alzheimer's neurological disease samples. Table 7A shows positive predictors for AD regardless of Braak stage. The positive predictors each provides 100% Specificity for the presence of AD in the cohort. Tables 7A and 7B shows the average read count across AD serum samples for the positive predictors. In some embodiments, the number of predictors that is present in a sample directly correlates with the Braak stage of AD.

In various embodiments, the presence, absence, or level of at least five sRNAs are determined, including positive and negative predictors and other potential controls. In some embodiments, the presence or absence of at least 8 sRNAs, or at least 10 sRNAs, or at least about 50 sRNAs are determined. The total number of sRNAs determined, in some embodiments, is less than about 1000 or less than about 500, or less than about 200, or less than about 100, or less than about 50. Therefore, the presence, absence, or level of sRNAs can be determined using any number of specific molecular detection assays.

In some embodiments, the presence, absence, or level of at least 2, or at least 5, or at least 10 sRNAs from Table 2A, Table 4A, and/or Table 7A are determined (SEQ ID NOS: 1-403). In some embodiments, the presence, absence, or level of at least one negative sRNA predictor is also determined. In some embodiments, a panel of sRNAs comprising positive predictors from Table 2A are determined, and the panel may comprise at least 2, at least 5, at least 10, or at least 20 sRNAs from Table 2A. In some embodiments, the panel comprises all sRNAs from Table 2A. In some embodiments, a panel of sRNAs comprising positive predictors from Table 4A are determined, and the panel may comprise at least 2, at least 5, at least 10, or at least 20 sRNAs from Table 4A. In some embodiments, the panel comprises all sRNAs from Table 4A. In some embodiments, a panel of sRNAs comprising positive predictors from Table 7A are determined, and the panel may comprise at least 2, at least 5, at least 10, or at least 20 sRNAs from Table 7A. In some embodiments, the panel comprises all sRNAs from Table 7A.

In some embodiments, the one or more (or all) positive sRNA predictors are each present in at least about 10% of AD samples in the experimental cohort, or at least about 20% of AD samples in the experimental cohort, or at least about 30% of AD samples in the experimental cohort, or at least about 40% of AD samples in the experimental cohort. In some embodiments, the identity and/or number of predictors identified correlates with active disease processes (e.g., Braak stage). For example, a sample may be positive for at least 1, 2, 3, 4, or 5 sRNA predictors in Tables 2A, 4A, and/or 7A, indicating disease from brain tissue, CSF, and/or serum samples, with more severe or advanced disease processes being correlative with about 10, or at least about 15, or at least about 20 sRNA predictors in Table 4A or 7A. In some embodiments, the absolute level (e.g., sequencing read count) or relative level (e.g., using a qualitative assay such as Real Time PCR) is determined for the sRNA predictors in Table 4A or Table 7A, which can be correlative with Braak stage.

In some embodiments, samples that test negative for the presence of the positive sRNA predictors, test positive for at least 1, or at least about 5, or at least about 10, or at least about 20, or at least about 30, or at least about 40, or at least about 50, or at least about 100 negative sRNA predictors. Negative predictors can be specific for healthy individuals or other disease states (such as PD or dementia). Individuals testing positive for AD, will typically not test positive for the presence of any negative predictors.

Generally, the presence of at least 1, 2, 3, 4, or 5 positive predictors, and the absence of all of the negative predictors is predictive of AD activity. In some embodiments, a panel of from 5 to about 100, or from about 5 to about 60 sRNA predictors are detected in the sample. While not each experimental sample will be positive for each positive predictor, the panel is large enough to provide at least about 75%, at least about 80%, at least about 85%, at least about 90%, at least about 95%, or about 100% coverage for the condition in an AD cohort. By selecting a panel in which a plurality of sRNA predictors are present in each sample of the experimental cohort, the panel will be tuned to provide for 100 Sensitivity and 100 Specificity for the training samples (the experimental cohort and the comparator cohort).

In various embodiments, detection of the sRNA predictors involves one of various detection platforms, which can employ reverse-transcription, amplification, and/or hybridization of a probe, including quantitative or qualitative PCR, or RealTime PCR. PCR detection formats can employ stem-loop primers for RT-PCR in some embodiments, and optionally in connection with fluorescently-labeled probes. In some embodiments, sRNAs are detected by RNA sequencing, with computational trimming of the 3′ sequencing adaptor. Sequencing can employ reverse-transcription and/or amplification using at most one specific primer for the binary predictor.

Generally, a real-time polymerase chain reaction (qPCR) monitors the amplification of a targeted DNA molecule during the PCR, i.e. in real-time. Real-time PCR can be used quantitatively, and semi-quantitatively. Two common methods for the detection of PCR products in real-time PCR are: (1) non-specific fluorescent dyes that intercalate with any double-stranded DNA (e.g., SYBR Green (I or II), or ethidium bromide), and (2) sequence-specific DNA probes consisting of oligonucleotides that are labelled with a fluorescent reporter which permits detection only after hybridization of the probe with its complementary sequence (e.g. TAQMAN).

In some embodiments, the assay format is TAQMAN real-time PCR. TAQMAN probes are hydrolysis probes that are designed to increase the Specificity of quantitative PCR. The TAQMAN probe principle relies on the 5′ to 3′ exonuclease activity of Taq polymerase to cleave a dual-labeled probe during hybridization to the complementary target sequence, with fluorophore-based detection. TAQMAN probes are dual labeled with a fluorophore and a quencher, and when the fluorophore is cleaved from the oligonucleotide probe by the Taq exonuclease activity, the fluorophore signal is detected (e.g., the signal is no longer quenched by the proximity of the labels). As in other quantitative PCR methods, the resulting fluorescence signal permits quantitative measurements of the accumulation of the product during the exponential stages of the PCR. The TAQMAN probe format provides high Sensitivity and Specificity of the detection.

In some embodiments, sRNA predictors present in the sample are converted to cDNA using specific primers, e.g., stem-loop primers to interrogate one or both ends of the sRNA. Amplification of the cDNA may then be quantified in real time, for example, by detecting the signal from a fluorescent reporting molecule, where the signal intensity correlates with the level of DNA at each amplification cycle.

Alternatively, sRNA predictors in the panel, or their amplicons, are detected by hybridization. Exemplary platforms include surface plasmon resonance (SPR) and microarray technology. Detection platforms can use microfluidics in some embodiments, for convenient sample processing and sRNA detection.

Generally, any method for determining the presence of sRNAs in samples can be employed. Such methods further include nucleic acid sequence based amplification (NASBA), flap endonuclease-based assays, as well as direct RNA capture with branched DNA (QuantiGene™), Hybrid Capture™ (Digene), or nCounter™ miRNA detection (nanostring). The assay format, in addition to determining the presence of miRNAs and other sRNAs may also provide for the control of, inter alia, intrinsic signal intensity variation. Such controls may include, for example, controls for background signal intensity and/or sample processing, and/or hybridization efficiency, as well as other desirable controls for detecting sRNAs in patient samples (e.g., collectively referred to as “normalization controls”).

In some embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In some embodiments, RNA is extracted from the sample prior to sRNA processing for detection. RNA may be purified using a variety of standard procedures as described, for example, in RNA Methodologies, A laboratory guide for isolation and characterization, 2nd edition, 1998, Robert E. Farrell, Jr., Ed., Academic Press. In addition, there are various processes as well as products commercially available for isolation of small molecular weight RNAs, including mirVANA™ Paris miRNA Isolation Kit (Ambion), miRNeasy™ kits (Qiagen), MagMAX™ kits (Life Technologies), and Pure Link™ kits (Life Technologies). For example, small molecular weight RNAs may be isolated by organic extraction followed by purification on a glass fiber filter. Alternative methods for isolating miRNAs include hybridization to magnetic beads. Alternatively, miRNA processing for detection (e.g., cDNA synthesis) may be conducted in the biofluid sample, that is, without an RNA extraction step.

In some embodiments, the presence or absence of the sRNAs are determined in a subject sample by nucleic acid sequencing, and individual sRNAs are identified by a process that comprises computational trimming a 3′ sequencing adaptor from individual sRNA sequences. See U.S. 2018/0258486, filed on Jan. 23, 2018, and PCT/US2018/014856, filed on Jan. 23, 2018, which are hereby incorporated by reference in their entireties. In some embodiments, the sequencing process can reverse-transcribe and/or amplify the sRNA predictors using primers specific for the biomarker.

Generally, assays can be constructed such that each assay is at least 80%, or at least 85%, or at least 90%, or at least 95%, or at least 98% specific for the sRNA (e.g., iso-miR) over an annotated sequence and/or other non-predictive iso-miRs and sRNAs. Annotated sequences can be determined with reference to miRBase. For example, in preparing sRNA predictor-specific real-time PCR assays, PCR primers and fluorescent probes can be prepared and tested for their level of Specificity. Bicyclic nucleotides or other modifications involving the 2′ position (e.g., LNA, cET, and MOE), or other nucleotide modifications (including base modifications) can be employed in probes to increase the Sensitivity or Specificity of detection. Specific detection of isomiRs and sRNAs is disclosed in US 2018/0258486, which is hereby incorporated by reference in its entirety.

sRNA predictors can be identified in any biological samples, including solid tissues and/or biological fluids. sRNA predictors can be identified in animals (e.g., vertebrate and invertebrate subjects), or in some embodiments, cultured cells or media from cultured cells. For example, the sample is a biological fluid sample from human or animal subjects (e.g., a mammalian subject), such as blood, serum, plasma, urine, saliva, or cerebrospinal fluid. miRNAs can be found in biological fluid, as a result of a secretory mechanism that may play an important role in cell-to-cell signaling. See, Kosaka N, et al., Circulating microRNA in body fluid: a new potential biomarker for cancer diagnosis and prognosis, Cancer Sci. 2010; 101: 2087-2092). miRs from cerebrospinal fluid and serum have been profiled according to conventional methods with the goal of stratifying patients for disease status and pathology features. Burgos K, et al., Profiles of Extracellular miRNA in Cerebrospinal Fluid and Serum from Patients with Alzheimer's and Parkinson's Diseases Correlate with Disease Status and Features of Pathology, PLOS ONE Vol. 9, Issue 5 (2014). In some embodiments, the sample is a solid tissue sample, which may comprise neurons. In some embodiments, the tissue sample is a brain tissue sample, such as from the frontal cortex region. In some embodiments, sRNA predictors are identified in at least two different types of samples, including brain tissue and a biological fluid such as blood. In some embodiments, sRNA predictors are identified in at least three different types of samples, including brain tissue, cerebrospinal fluid (CSF), and blood.

The invention involves detection of sRNA predictors in cells or animals that exhibit an Alzheimer's disease genotype or phenotype. In some embodiments, the sRNA predictor is indicative of AD biological processes in patients or subjects that are otherwise considered non-Alzheimer's patients or subjects. In some embodiments, the sRNA predictor is indicative of specific Braak stage of AD.

In some embodiments, the sRNA predictors are indicative of Braak Stage I and/or II of Alzheimer's disease processes. Braak Stage I/II refers to the transentorhinal (temporal lobe) area of the brain that develops argyrophilic neurofibrillary tangles and neurophil threads over the course of AD progression. Braak Stage I/II is known to be clinically silent at this point in the AD processes.

In some embodiments, the sRNA predictors are indicative of Braak Stage III and/or IV of Alzheimer's disease processes. Braak Stage III/IV refers to the limbic area of the brain that develops argyrophilic neurofibrillary tangles and neurophil threads over the course of AD progression. Braak Stage III/IV is known to be incipient Alzheimer's disease at this point in the AD processes.

In some embodiments, the sRNA predictors are indicative of Braak Stage V and/or VI of Alzheimer's disease processes. Braak Stage V/VI refers to the neocortical area of the brain that develops argyrophilic neurofibrillary tangles and neurophil threads over the course of AD progression. Braak Stage V/VI is known to be full developed Alzheimer's disease at this point in the AD processes.

In some embodiments, the method is repeated to determine the sRNA predictor profile over time, for example, to determine the impact of a therapeutic regimen, or a candidate therapeutic regimen. For example, a subject or patient may be evaluated at a frequency of at least about once per year, or at least about once every six months, or at least once per month, or at least once per week. In some embodiments, a decline in the number of predictors present over time, or a slower increase in the number of predictors detected over time, is indicative of slower disease progression or milder disease symptoms. Embodiments of the invention are useful for constructing animal models for AD treatment, as well as useful as biomarkers in human clinical trials.

In some aspects, the invention provides kits for evaluating samples for Alzheimer's disease activity. In various embodiments, the kits comprise sRNA-specific probes and/or primers configured for detecting a plurality of sRNAs listed in Tables 2A, 4A, and or 7A (SEQ ID NOS: 1-403). In some embodiments, the kit comprises sRNA-specific probes and/or primers configured for detecting at least 2, at least 5, or at least 10, or at least 20, or at least 40 sRNAs listed in Tables 2A, 4A, and or 7A (SEQ ID NOS: 1-403). In some embodiments, the kit comprises sRNA-specific probes and/or primers configured for detecting at least 2, 3, 4, 5, or at least 10, or at least 20 sRNAs listed in Table 2A (SEQ ID NOS: 1-46). In some embodiments, the kit comprises sRNA-specific probes and/or primers configured for detecting at least 2, 3, 4, 5, or at least 10, or at least 20, or at least 40 sRNAs listed in Table 4A (SEQ ID NOS: 47-254). In some embodiments, the kit comprises sRNA-specific probes and/or primers configured for detecting at least 2, 3, 4, 5, or at least 10, or at least 20 sRNAs listed in Table 7A (SEQ ID NOS: 255-403).

The kits may comprise probes and/or primers suitable for a quantitative or qualitative PCR assay, that is, for specific sRNA predictors. In some embodiments, the kits comprise a fluorescent dye or fluorescent-labeled probe, which may optionally comprise a quencher moiety. In some embodiments, the kit comprises a stem-loop RT primer, and in some embodiments may include a stem-loop primer to interrogate each of the sRNA ends. In some embodiments, the kit may comprise an array of sRNA-specific hybridization probes.

In some embodiments, the invention provides a kit comprising reagents for detecting a panel of from 5 to about 100 sRNA predictors, or from about 5 to about 50 sRNA predictors, or from 5 to about 20 sRNAs. In these embodiments, the kit may comprise at least 5, at least 10, at least 20 sRNA predictor assays (e.g., reagents for such assays). In various embodiments, the kit comprises at least 10 positive predictors and at least 5 negative predictors. In some embodiments, the kit comprises a panel of at least 5, or at least 10, or at least 20, or at least 40 sRNA predictor assays, the sRNA predictors being selected from Table 2A, Table 4A, and/or Table 7A. In some embodiments, at least 1 sRNA predictor is selected from Table 4B or Table 7B. Such assays may comprise reverse transcription (RT) primers, amplification primers and probes (such as fluorescent probes or dual labeled probes) specific for the sRNA predictors over annotated sequences as well as other (non-predictive) variations. In some embodiments, the kit is in the form of an array or other substrate containing probes for detection of sRNA predictors by hybridization.

In still other embodiments, the invention involves constructing disease classifiers that classify samples based on the presence or absence of particular sRNA molecules. These disease classifiers are powerful tools for discriminating disease conditions that present with similar symptoms, as well as determining disease subtypes, including predicting the course of the disease, predicting response to treatment, and disease monitoring. Generally, sRNA panels (e.g., panels of distinct sRNA variants) will be determined from sequence data in one or more training sets representing one or more disease conditions of interest. sRNA panels and the classifier algorithm can be constructed using, for example, one or more of supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naive Bayes Classifier, Perceptron, Quadratic classifiers, Kernel Estimation, k-Nearest Neighbor, Learning Vector Quantization, and Principal Components Analysis. Once the classifier is trained, independent subjects can be evaluated for the disease conditions by detecting the presence or absence, in a biological sample from the subject, of the sRNA markers in the panel, and applying the classification algorithm. Classifiers can be binary classifiers (i.e., classify among two conditions), or may classify among three, four, five, or more disease conditions. In some embodiments, the classifier can classify among at least ten disease conditions.

For example, in some embodiments, the invention provides a method for evaluating a subject for one or more disease conditions. The method comprises providing a biological sample of the subject, and determining the presence or absence of a plurality of sRNAs in the sRNA panel. This profile of “present and absent” sRNAs (binary markers) is used to classify the condition of the subject among two or more disease conditions using the disease classifier. The disease classifier will have been trained based on the presence and absence of the sRNAs in the sRNA panel in a set of training samples. For example, the training samples are annotated as positive or negative for the one or more disease conditions, as well as the presence or absence (or level) of the sRNAs in the panel. In some embodiments, samples are annotated for one or more of disease grade or stage, disease subtype, therapeutic regimen, and drug sensitivity or resistance.

The presence or absence of the sRNAs in the panel is determined in the training set from sRNA sequence data. That is, individual sRNA sequences are identified in the sRNA sequence data by trimming the 5′ and/or 3′ sequencing adaptors and without consolidating sRNA sequence variants to a reference sequence or genetic locus. For example, after trimming, the unique sequence reads within each sample and disease condition or comparator condition are each compiled. Thus, the presence or absence of specific sRNA sequences, such as isoforms, are determined in each sample and for each disease condition, and these variants are not consolidated to reference sequences. These sequences can be used as “binary” markers, that is, evaluated based on their presence or absence in samples, as opposed to discriminating normal and abnormal levels.

In some embodiments, during construction of the classifier, sRNAs are preselected for training. For example, sRNA families can be identified in which variation increases in a disease condition and/or increases with severity of a disease condition, and/or which variation may normalize or be ameliorated in response to a therapeutic regimen. For example, sRNA pre-selection can involve grouping sRNA isoforms (such as isomiRs) into ‘families’ based on biologically relevant sequence hyper-features (e.g. ‘seed sequence’ nucleotides 2-8 from the 5′ end of the sRNA isoform, and/or single nucleotide polymorphisms) outside of a lower and upper bound threshold where the lower bound threshold is 0 to 100 trimmed reads per million reads, and the upper bound threshold is 0 to 100 trimmed reads per million reads. These families are evaluated for variation that is correlative with disease activity, and these entire families, or variations with a read count above or below the threshold are selected as candidates for inclusion in the classifier. In some embodiments, these families include at least one sRNA predictor that is unique in at least one of the disease conditions.

Once identified in the sequence data, and selected for inclusion in the computational classifier, molecular detection reagents for the sRNAs in the panel can be prepared. Such detection platforms include quantitative RT-PCR assays, including those employing stem loop primers and fluorescent probes, as described herein. In some embodiments, independent samples are evaluated by sRNA sequencing, rather than migrating to a molecular detection platform.

sRNA panels (e.g., binary sRNA markers used for classification) may contain from about 4 to about 200 sRNAs, or in some embodiments, from about 4 to about 100 sRNAs. In some embodiments, the sRNA panel contains from about 10 to about 100 sRNAs, or from about 10 to about 50 sRNAs.

Classifiers can be trained on various types of samples, including solid tissue samples, biological fluid samples, or cultured cells in some embodiments. When evaluating the subject, biological samples from which sRNAs are evaluated can include biological fluids such as blood, serum, plasma, urine, saliva, or cerebrospinal fluid. Alternatively, the biological sample of the subject is a solid tissue biopsy.

In various embodiments, the training set has at least 50 samples, or at least 100 samples, or at least 200 samples. In some embodiments, the training set includes at least 10 samples for each disease condition or at least 20 or at least 50 samples for each disease condition. A higher number of samples can provide for better statistical powering.

Disease classifiers in accordance with this disclosure can be constructed for various types of disease conditions. For example, in some embodiments, the disease conditions are diseases of the central nervous system. Such diseases can include at least two neurodegenerative diseases involving symptoms of dementia. In some embodiments, at least two disease conditions are selected from Alzheimer's Disease, Parkinson's Disease, Huntington's Disease, Mild Cognitive Impairment, Progressive Supranuclear Palsy, Frontotemporal Dementia, Lewy Body Dementia, and Vascular Dementia. Alternatively, at least two disease conditions are neurodegenerative diseases involving symptoms of loss of movement control, such as Parkinson's Disease, Amyotrophic Lateral Sclerosis, Huntington's Disease, Multiple Sclerosis, and Spinal Muscular Atrophy. In still other embodiments, at least two disease conditions are demyelinating diseases, optionally including multiple sclerosis, optic neuritis, transverse myelitis, and neuromyelitis optica.

Accordingly, in some embodiments, at least one disease condition is selected from Alzheimer's Disease, Parkinson's Disease, Huntington's Disease, Multiple Sclerosis, Amyotrophic Lateral Sclerosis, and Spinal Muscular Atrophy; and training samples are annotated for disease stage, disease severity, drug responsiveness, or course of disease progression.

In still other embodiments, the disease conditions are cancers of different tissue or cell origin. In some embodiments, the disease conditions are drug sensitive versus drug resistant cancer, or sensitivity across two or more therapeutic agents. In such embodiments, the biological sample from the subject can be a tumor or cancer cell biopsy.

In some embodiments, the disease conditions are inflammatory or immunological diseases, and optionally including one or more of Systemic Lupus Erythematosus (SLE), scleroderma, autoimmune vasculitis, diabetes mellitus (type 1 or type 2), Grave's disease, Addison's disease, Sjogren's syndrome, thyroiditis, rheumatoid arthritis, myasthenia gravis, multiple sclerosis, fibromyalgia, psoriasis, Crohn's disease, ulcerative colitis, diverticular disease and celiac disease. For example, the classifier can distinguish gastrointestinal inflammatory conditions such as, but not limited to, Crohn's disease, ulcerative colitis, and diverticular disease. In such embodiments, the biological samples from the subject to be tested can be biological fluid samples such as blood, serum, or plasma, or can be biopsy tissue such as colon epithelial tissue.

In some embodiments, the disease conditions are cardiovascular diseases, optionally including stratification for risk of acute event. In some embodiments, the cardiovascular diseases include one or more of coronary artery disease (CAD), myocardial infarction, stroke, congestive heart failure, hypertensive heart disease, cardiomyopathy, heart arrhythmia, congenital heart disease, valvular heart disease, carditis, aortic aneurysms, peripheral artery disease, and venous thrombosis.

In various embodiments, at least one, or at least two, or at least five, or at least ten sRNAs in the panel are positive sRNA predictors. That is, the positive sRNA predictors were identified as present in a plurality of samples annotated as positive for a disease condition in the training set, and absent in all samples annotated as negative for the disease condition in the training set. In some embodiments, with respect to a disease classifier including Alzheimer's Disease as a disease condition, the sRNA panel may include one or more, or two or more, or five or more, or ten or more, sRNAs from Table 2A, Table 4A, and/or Table 7A (SEQ ID NOS: 1-403).

In some embodiments, the sRNA panel includes one or more sRNA predictors from Table 2A (SEQ ID NOS: 1 to 46). In some embodiments, the sRNA panel includes one or more sRNA predictors from Table 4A (SEQ ID NOS: 47-254). In some embodiments, the sRNA panel includes one or more sRNA predictors from Table 4A (SEQ ID NOS: 255-403). In some embodiments, the sRNA panel includes one or more sRNA predictors from Table 5 (SEQ ID NOS: 58, 189, 78, 172, 193, 97, 122, 215, 248, 164, 120, 93, 126, 253, 112, 144, 213, 244, 123, 222, 150, 240, 52, 220, 221, 169, 165, and 212), which correlate with Braak stages of AD progression in CSF. In some embodiments, the sRNA panel include one or more sRNAs from Table 8 (SEQ ID NOS: 257, 270, 272, 273, 279, 286, 288, 314, 319, 325, 332, 341, 374, 391, and 393), which correlate with Braak stages of AD progression in serum.

Other aspects and embodiments of the invention will be apparent from the following examples.

### EXAMPLES

### Example 1: Binary Classifiers for Alzheimer's Disease were Identified in Either an Experimental or Comparator Group of Brain Tissue, Cerebrospinal Fluid, or Serum

To identify binary small RNA predictors for Alzheimer's Disease, small RNA sequencing data was downloaded from the GEO and dbGaP Databases and used as a Discovery Set (Table 1A-1B: Brain Samples, Table 3A-3B CSF Samples, and Table 6A-6B SER Samples). All samples, regardless of material, were derived from postmortem-verified Alzheimer's or non-Alzheimer's samples (healthy controls or other non-Alzheimer's related neurological diseases such as Parkinson's, Parkinson's with Dementia, Huntington's, etc.).

The overall process is described below:

| | Sample | Number of |
|---|---|---|
| Diagnosis | Material | Samples (N) |
| Alzheimer's Disease | brain tissue | 17 |
| Controls | brain tissue | 123 |
| Healthy | | 51 |
| other non-Alzheimer's Neurological Disease | | 72 |
| Alzheimer's Disease | CSF | 64 |
| Controls | CSF | 109 |
| Healthy | | 68 |
| other non-Alzheimer's Neurological Disease | | 41 |
| Alzheimer's Disease | SER | 51 |
| Controls | SER | 130 |
| Healthy | | 70 |
| other non-Alzheimer's Neurological Disease | | 60 |

CSF = cerebrospinal fluid, SER = serum.

Files were converted from a .sra to .fastq format using the SRA Tool Kit v2.8.0 for Centos, and .fastq formatted files were processed as described in U.S. 2018/0258486 and International Application No. PCT/US2018/014856, filed on Jan. 23, 2018 (which are hereby incorporated by reference in their entireties). Specifically, all .fastq data files were processed by trimming adapter sequences using the (Regex) regular expression-based search and trim algorithm, where 5′ TGGAATTCTCGGGTGCCAAGGAA 3′ (SEQ ID NO: 404) (containing up to a 15 nucleotide 3′-end truncation) was input to identify the 3′ adapter sequence, and a Levenshtein Distance of 2 or a Hamming Distance of 5. Parameters for Regex searching requires that the 1^{st} nucleotide of the user-specified search term to be unaltered with respect to nucleotide insertions, deletions, and/or swaps.

Samples are compiled in 1 of 2 groups, either an Experimental Group or a Comparator Group. sRNA-Split identifies small RNAs that are unique to either the Experimental Group or Comparator Group, as well as small RNAs that are present in both the Experimental Group and Comparator Group. Small RNAs that are unique to either the Experimental Group or Comparator Group have 100% Specificity (by definition). Unique (binary) small RNAs serve as classifiers for the Group in which they were identified. Binary small RNA classifiers can be used in non-bootstrapped and/or bootstrapped computational classification algorithms (e.g. supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naive Bayes Classifier, Perceptron, Quadratic classifiers, Kernel Estimation, k-Nearest Neighbor, Learning Vector Quantization, and Principal Components Analysis, etc.), and they can also be used as targets for Quantitative Reverse-Transcription Polymerase Chain Reaction (RT-qPCR).

Binary small RNA classifiers were identified by analyzing trimmed, small RNA reads with sRNA-Split. Trimmed reads were converted to trimmed-reads per million reads. Biomarkers were filtered such that each sample needed to have a minimum of 1 marker providing coverage. To identify biomarkers correlated with Braak Stage, small RNAs had to be present in a minimum of 3 consecutive Braak Stages and have a Pearson Correlation Coefficient of ≥0.75.

Specific biomarker panels containing binary small RNA predictors (present in samples of the Experimental Group, but not present in any samples of the Comparator Group) were identified as follows:

(1) AD vs non-AD

(A) Brain Tissue (Table 2)

(B) CSF (Table 4)

(C) Serum (Table 7)

### (2) Alzheimer's Disease Monitoring

(A) CSF (Table 5)

(B) Serum (Table 8)

Probability scores (p-values) were calculated for each individual binary small RNA predictor using a Chi-Square 2×2 Contingency Table and one-tailed Fisher's Exact Probability Test.

Probability scores (p-values) were calculated for panels of binary small RNA predictor for each Experimental Group using a Chi-Square 2×2 Contingency Table and one-tailed Fisher's Exact Probability Test (all giving 100% Specificity and 100% Sensitivity).

### Example 2: Construction of Multi-Class Disease Classifiers of Inflammatory Bowel Disease (IBD)

To construct disease classifiers that classify IBD samples based on the presence or absence of particular sRNA molecules, sRNA panels were determined from sequence data in various training sets representing different disease conditions of interest, such as Crohn's disease, ulcerative colitis, and diverticular disease.

### Samples

All samples were collected according to their respective Institutional Review Board (IRB) approval and have patient consent for unrestricted use. Data was collected from electronic medical records and chart review. Clinical Data includes information such as: age, gender, race, ethnicity, weight, body mass index, smoking history, alcohol use history, family history of disease. Disease-related data includes information such as: diagnosis, age at Inflammatory Bowel Disease (IBD) diagnosis, current and prior medications, comorbidities, age at proctocolectomy and Ileal Pouch Anal Anastomosis (IPAA), as well as pouch age, time from closure of ileostomy, or from pouch surgery (where applicable from patients undergoing these procedures).

Biopsies were taken from the colon epithelium. Inoperable Ulcerative Colitis (IUC), Operable Ulcerative Colitis (OUC), Crohn's Disease (CD), Diverticular Disease (DD), Polyps/Polyposis (PP), Serrated Polyps/Polyposis (SPP), colon cancer, (CC), rectal cancer (RC) were defined according to clinical, endoscopic, histologic, and imaging studies. Further inclusion criteria were the presence of ileitis for CD patients and having a normal terminal ileum as seen by endoscopy and confirmed by histology for IUC patients. Individuals who required a colonoscopy for routine screening and were verified as having non-diseased bowel tissue by endoscopy and/or histology were labeled as normal controls.

All biopsies were assessed by a minimum of two (2) institutional IBD-trained pathologists and consensus scores and diagnoses were provided according to clinical and industry standard diagnostic protocols. Briefly, active inflammatory characteristics were scored according to neutrophil infiltration (0-3) and area of ulceration (0-3), each sample was classified into inactive, cryptitis, crypt abscess, numerous crypt abscesses (>3/high power field) and ulceration. Original Geboes Score (OGS) or Simplified Geboes Score (SGS) was used to classify UC. Chron's Disease Activity Index (CDAI) and Crohn's Disease Endoscopic Index of Severity (CDEIS) was used to classify CD. Hinchey Classification was used to characterize DD. Colorectal cancers, polyps and serrated polyps were classified according to the most recent recommendations of the Multi-Society Task Force on Colorectal Cancer (CRC).

An overview of the IBD samples used is displayed below:

| | Diagnosis | | | |
|---|---|---|---|---|
| | | Crohn's | Ulcerative | Diverticular |
| | Normal | disease | Colitis | Disease |
| Tissue Type | Colon | Colon | Colon | Colon |
| | Epithelium | Epithelium | Epithelium | Epithelium |
| N | 64 | 35 | 139 | 20 |
| Gender (F:M) | 26:38 | 14:21 | 50:89 | 6:14 |
| Age at sampling, years, | 56.4 ± 13.5 | 36.6 ± 15.8 | 45.5 ± 14.1 | 44.9 ± 10.6 |
| mean ± SD (range) | (26-82) | (15-76) | 32-57) | (31-69) |
| Age at IBD diagnosis, years, | NA | 30.4 ± 12.1 | 32.1 ± 11.6 | 26.2 ± 8.7 |
| mean ± SD (range) | | (18-48) | (16-51) | (21-55) |
| IBD duration, years, | NA | 13.3 (3-53) | 10.5 (3-28) | 12.6 (25-53) |
| mean ± SD (range) | | | | |
| Ashkenazi origin | 5 | 2 | 9 | 1 |
| Non-Ashkenazi origin | 53 | 31 | 120 | 17 |
| Mixed origin | 6 | 2 | 10 | 2 |
| Never smoker | 56 | 28 | 122 | 19 |
| Past smokers | 5 | 2 | 10 | 1 |
| Current smokers | 3 | 5 | 7 | 0 |
| Body mass index, | 25.5 ± 2.9 | 27 ± 5.3 | 25.8 ± 6.1 | 23.3 ± 5.2 |
| mean ± SD (range) | (17-30) | (18-31) | (15-41) | (18-40) |
| Family history of IBD | 2 | 3 | 8 | 1 |
| Steroid exposure | NA | NA | 110 | NA |
| Severity Score (B1:B2:B3) | NA | 7:6:8 | NA | NA |

To identify small RNA predictors for disease classes associated with IBD, small RNA sequencing data was downloaded from the GEO Database and used as a Discovery Set. small RNA sequencing data was downloaded from the Geodatabase studies for Crohn's disease (GSE66208), Ulcerative colitis (GSE114591), Diverticular disease (GSE89667), and Normal/Control (GSE118504).

Files were converted from a .sra to .fastq format using the SRA Tool Kit v2.8.0 for Centos, and .fastq formatted files were processed as described in U.S. 2018/0258486 and International Application No. PCT/US2018/014856, filed on Jan. 23, 2018 (which are hereby incorporated by reference in their entireties). Specifically, all .fastq data files were processed by trimming adapter sequences using the (Regex) regular expression-based search and trim algorithm, where 5′ TGGAATTCTCGGGTGCCAAGGAA 3′ (SEQ ID NO: 404) (containing up to a 15 nucleotide 3′-end truncation) was input to identify the 3′ adapter sequence, and a Levenshtein Distance of 2 or a Hamming Distance of 5. Parameters for Regex searching requires that the 1^{st} nucleotide of the user-specified search term to be unaltered with respect to nucleotide insertions, deletions, and/or swaps.

Samples are compiled in 1 of 2 groups, either an Experimental Group or a Comparator Group. sRNA-Split identifies small RNAs that are unique to either the Experimental Group or Comparator Group, as well as small RNAs that are present in both the Experimental Group and Comparator Group. Small RNAs that are unique to either the Experimental Group or Comparator Group have 100% Specificity (by definition). Unique (binary) small RNAs serve as classifiers for the Group in which they were identified. Binary small RNA classifiers can be used in non-bootstrapped and/or bootstrapped computational classification algorithms (e.g. supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naive Bayes Classifier, Perceptron, Quadratic classifiers, Kernel Estimation, k-Nearest Neighbor, Learning Vector Quantization, and Principal Components Analysis, etc.), and they can also be used as targets for Quantitative Reverse-Transcription Polymerase Chain Reaction (RT-qPCR).

Binary small RNA classifiers were identified by analyzing trimmed, small RNA reads with sRNA-Split. Trimmed reads were converted to trimmed-reads per million reads. Biomarkers were filtered such that each sample needed to have a minimum of 1 marker providing coverage.

### Per-Class Metrics

Per-class metrics were determined for each class in order to identify markers that are most important for identifying the disease class. sRNA panels were determined from sequence data in various training sets representing different disease conditions of interest. Specific biomarker panels containing small RNA predictors of disease class were identified as follows:
Controls (Healthy individuals/“Normal” individuals): Table 9;
Crohn's disease: Table 10;
Ulcerative colitis: Table 11; and
Diverticular disease: Table 12.

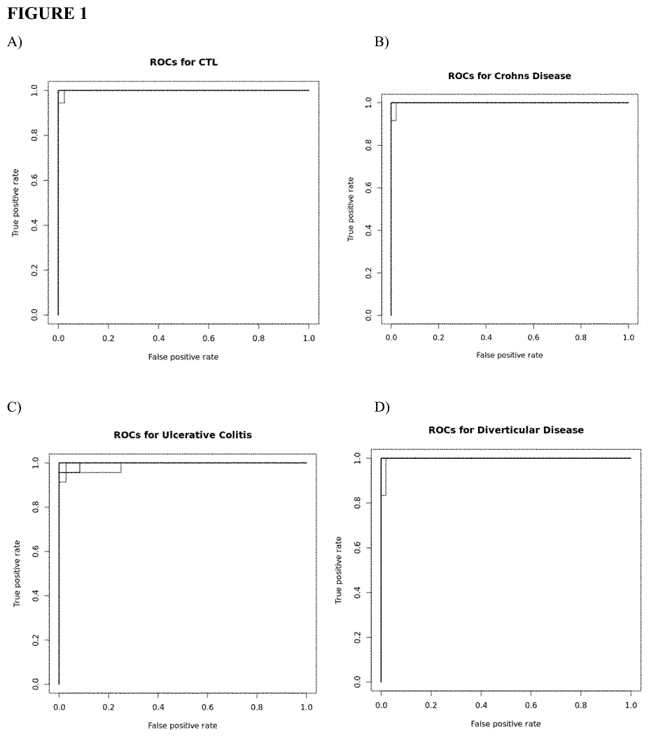

By using a supervised, non-parametric, logistical regression machine learning model, the final selection marker count was reduced from 128 to 100 maximum. In order to assess the classification model's performance, ROC/AUC curves were obtained for each set of markers identified per class, where ROC is a probability curve and AUC represents the degree or measure of separability. The ROC curve is plotted with true positive rate against the false positive rate. ROC/AUC curves were established for the various IBD classes and controls, as discussed above, and these are depicted in FIG. 1.

### Multi-Class Disease Classification

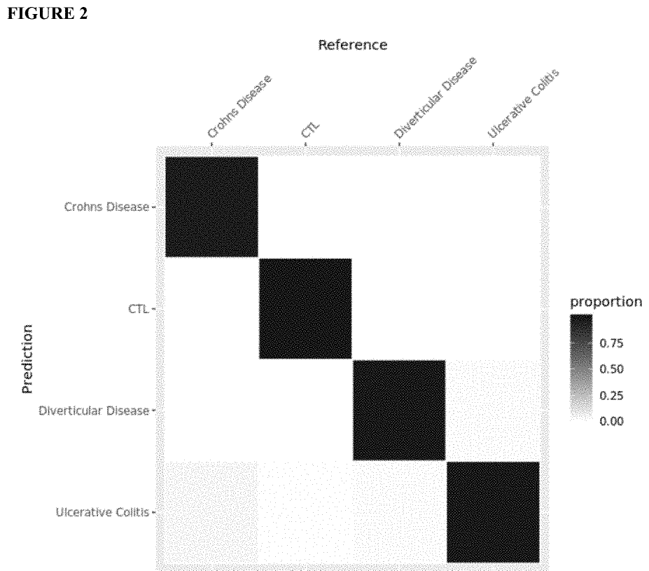

The disease classifier was trained based on the positive or negative markers of the sRNA panels, as well as the presence or absence of the sRNAs in the panels identified above for Controls, Crohn's disease, ulcerative colitis, and diverticular disease. In order to assess the accuracy of the computational model when the class metrics were all combined, a test was run to evaluate the model's identification predictive power against reference samples of each class. It was found that the model had an accuracy rate of 98%. FIG. 2 depicts a heat map showing the proportion of accurate predictions of disease class against their true reference identies. These results are also shown in the matrix below:

| | Reference | | | |
|---|---|---|---|---|
| | Crohn's | | Diverticular | Ulcerative |
| Prediction | Disease | Control | Disease | Colitis |
| Crohn's | 116 | 0 | 0 | 0 |
| Disease | | | | |
| Control | 0 | 179 | 0 | 0 |
| Diverticular | 0 | 0 | 59 | 4 |
| Disease | | | | |
| Ulcerative | 4 | 1 | 1 | 226 |
| Colitis | | | | |

### REFERENCES

1. Santa-Maria I, Alaniz M E, Renwick N, Cela C et al. Dysregulation of microRNA-219 promotes neurodegeneration through post-transcriptional regulation of tau. J Clin Invest 2015 February; 125(2):681-6. PMID: 25574843
2. Lau P, Bossers K, Janky R, Salta E et al. Alteration of the microRNA network during the progression of Alzheimer's disease. EMBO Mol Med 2013 October; 5(10):1613-34. PMID: 24014289
3. Hebert S S, Wang W X, Zhu Q, Nelson P T. A study of small RNAs from cerebral neocortex of pathology-verified Alzheimer's disease, dementia with lewy bodies, hippocampal sclerosis, frontotemporal lobar dementia, and non-demented human controls. J Alzheimers Dis 2013; 35(2):335-48. PMID: 23403535
4. Hoss A G, Labadorf A, Beach T G, Latourelle J C et al. microRNA Profiles in Parkinson's Disease Prefrontal Cortex. Front Aging Neurosci 2016; 8:36. PMID: 26973511
5. Hoss A G, Labadorf A, Latourelle J C, Kartha V K et al. miR-10b-5p expression in Huntington's disease brain relates to age of onset and the extent of striatal involvement. BMC Med Genomics Mar. 1, 2015; 8:10. PMID: 25889241
6. Burgos K, Malenica I, Metpally R, Courtright A, et al. Profiles of extracellular miRNA in cerebrospinal fluid and serum from patients with Alzheimer's and Parkinson's diseases correlate with disease status and features of pathology. PLoS One. 2014; 9(5):e94839. PMID: 24797360

TABLE 1A
Experimental Alzheimer's disease cohort for
biomarker discovery, taken from brain samples.

| | | Study | | | Age at | Braak |
|---|---|---|---|---|---|---|
| Group | Sample ID | Number | Disease Type | Gender | Death | score |
| Experimental | SRR1658350 | GSE63501 | Alzheimer's | F | 90 | III-IV |
| Experimental | SRR1658353 | GSE63501 | Alzheimer's | F | 90 | III-IV |
| Experimental | SRR1103943 | GSE48552 | Alzheimer's | M | 79 | V |
| Experimental | SRR828723 | GSE46131 | Alzheimer's | F | 83 | V |
| Experimental | SRR1658347 | GSE63501 | Alzheimer's | F | 92 | V-VI |
| Experimental | SRR1658348 | GSE63501 | Alzheimer's | F | 91 | V-VI |
| Experimental | SRR1658349 | GSE63501 | Alzheimer's | M | 86 | V-VI |
| Experimental | SRR1658351 | GSE63501 | Alzheimer's | M | 98 | V-VI |
| Experimental | SRR1103944 | GSE48552 | Alzheimer's | F | 80 | VI |
| Experimental | SRR1103945 | GSE48552 | Alzheimer's | M | 67 | VI |
| Experimental | SRR1103946 | GSE48552 | Alzheimer's | F | 67 | VI |
| Experimental | SRR1103947 | GSE48552 | Alzheimer's | F | 68 | VI |
| Experimental | SRR1103948 | GSE48552 | Alzheimer's | F | 72 | VI |
| Experimental | SRR828724 | GSE46131 | Alzheimer's | F | 86 | VI |
| Experimental | SRR828725 | GSE46131 | Alzheimer's | F | 67 | VI |
| Experimental | SRR828726 | GSE46131 | Alzheimer's | F | 75 | VI |
| Experimental | SRR828727 | GSE46131 | Alzheimer's | F | 86 | VI |
| AVERAGE | NA | NA | NA | NA | 81.00 ± | NA |
| | | | | | 10.1 | |

TABLE 1B
Comparator cohort for AD biomarker discovery, taken from brain samples, including
healthy controls and various other non-Alzheimer's neurological disorders.

| | | Study | | | Age at | Braak |
|---|---|---|---|---|---|---|
| Group | Sample ID | Number | DiseaseType | Gender | Death | score |
| Comparator | SRR828715 | GSE46131 | Bilateral hippocampal | F | 84 | 0 |
| | | | sclerosis | | | |
| Comparator | SRR828716 | GSE46131 | Bilateral hippocampal | F | 84 | 0 |
| | | | sclerosis | | | |
| Comparator | SRR828718 | GSE46131 | Bilateral hippocampal | F | 101 | 0 |
| | | | sclerosis | | | |
| Comparator | SRR1658356 | GSE72962 | Control | M | 93 | 0 |
| Comparator | SRR1658357 | GSE72962 | Control | M | 92 | 0 |
| Comparator | SRR1658359 | GSE72962 | Control | F | 84 | 0 |
| Comparator | SRR1658360 | GSE72962 | Control | F | 85 | 0 |
| Comparator | SRR1103937 | GSE48552 | Control | M | 80 | 0 |
| Comparator | SRR1103938 | GSE48552 | Control | M | 78 | 0 |
| Comparator | SRR1103939 | GSE48552 | Control | F | 52 | 0 |
| Comparator | SRR1103940 | GSE48552 | Control | F | 74 | 0 |
| Comparator | SRR828708 | GSE46131 | Control | F | 75 | 0 |
| Comparator | SRR828709 | GSE46131 | Control | F | 84 | 0 |
| Comparator | SRR828719 | GSE46131 | Dementia with Lewy | M | 78 | 0 |
| | | | bodies | | | |
| Comparator | SRR828720 | GSE46131 | Dementia with Lewy | M | 78 | 0 |
| | | | bodies | | | |
| Comparator | SRR828721 | GSE46131 | Dementia with Lewy | F | 85 | 0 |
| | | | bodies | | | |
| Comparator | SRR828722 | GSE46131 | Dementia with Lewy | M | 68 | 0 |
| | | | bodies | | | |
| Comparator | SRR828710 | GSE46131 | FTLD (TDP43 negative) | F | 37 | 0 |
| Comparator | SRR828711 | GSE46131 | FTLD (TDP43 positive) | F | 53 | 0 |
| Comparator | SRR828712 | GSE46131 | FTLD (TDP43 positive) | M | 48 | 0 |
| Comparator | SRR828713 | GSE46131 | FTLD (TDP43 positive) | F | 87 | 0 |
| Comparator | SRR828714 | GSE46131 | Progressive supranuclear | M | 70 | 0 |
| | | | palsy | | | |
| Comparator | SRR1103941 | GSE48552 | Control | M | 83 | I |
| Comparator | SRR1103942 | GSE48552 | Control | F | 78 | I |
| Comparator | SRR1658345 | GSE63501 | Control | F | 82 | I-II |
| Comparator | SRR1658355 | GSE63501 | Control | M | 90 | I-II |
| Comparator | SRR1658346 | GSE63501 | Control | M | 94 | III-IV |
| Comparator | SRR1658352 | GSE63501 | TPD | F | 93 | III-IV |
| Comparator | SRR1658354 | GSE63501 | TPD | F | 88 | III-IV |
| Comparator | SRR1658358 | GSE63501 | TPD | F | 96 | III-IV |
| Comparator | SRR1759212 | GSE72962 | Control | M | 73 | NA |
| Comparator | SRR1759213 | GSE72962 | Control | M | 91 | NA |
| Comparator | SRR1759214 | GSE72962 | Control | M | 82 | NA |
| Comparator | SRR1759215 | GSE72962 | Control | M | 97 | NA |
| Comparator | SRR1759216 | GSE72962 | Control | M | 86 | NA |
| Comparator | SRR1759217 | GSE72962 | Control | M | 91 | NA |
| Comparator | SRR1759218 | GSE72962 | Control | M | 81 | NA |
| Comparator | SRR1759219 | GSE72962 | Control | M | 79 | NA |
| Comparator | SRR1759220 | GSE72962 | Control | M | 63 | NA |
| Comparator | SRR1759221 | GSE72962 | Control | M | 66 | NA |
| Comparator | SRR1759222 | GSE72962 | Control | M | 69 | NA |
| Comparator | SRR1759223 | GSE72962 | Control | M | 79 | NA |
| Comparator | SRR1759224 | GSE72962 | Control | M | 61 | NA |
| Comparator | SRR1759225 | GSE72962 | Control | M | 58 | NA |
| Comparator | SRR1759226 | GSE72962 | Control | M | 70 | NA |
| Comparator | SRR1759227 | GSE72962 | Control | M | 66 | NA |
| Comparator | SRR1759228 | GSE72962 | Control | M | 60 | NA |
| Comparator | SRR1759229 | GSE72962 | Control | M | 76 | NA |
| Comparator | SRR1759230 | GSE72962 | Control | M | 61 | NA |
| Comparator | SRR1759231 | GSE72962 | Control | M | 62 | NA |
| Comparator | SRR1759232 | GSE72962 | Control | M | 69 | NA |
| Comparator | SRR1759233 | GSE72962 | Control | M | 61 | NA |
| Comparator | SRR1759234 | GSE72962 | Control | M | 93 | NA |
| Comparator | SRR1759235 | GSE72962 | Control | M | 53 | NA |
| Comparator | SRR1759236 | GSE72962 | Control | M | 57 | NA |
| Comparator | SRR1759237 | GSE72962 | Control | M | 43 | NA |
| Comparator | SRR1759238 | GSE72962 | Control | F | 71 | NA |
| Comparator | SRR1759239 | GSE72962 | Control | M | 46 | NA |
| Comparator | SRR1759240 | GSE72962 | Control | M | 40 | NA |
| Comparator | SRR1759241 | GSE72962 | Control | M | 44 | NA |
| Comparator | SRR1759242 | GSE72962 | Control | M | 57 | NA |
| Comparator | SRR1759243 | GSE72962 | Control | M | 80 | NA |
| Comparator | SRR1759244 | GSE72962 | Control | F | 75 | NA |
| Comparator | SRR1759245 | GSE72962 | Control | F | 76 | NA |
| Comparator | SRR1759246 | GSE72962 | Control | M | 68 | NA |
| Comparator | SRR1759247 | GSE72962 | Control | M | 64 | NA |
| Comparator | SRR1759248 | GSE64977 | Huntington's Disease | M | 55 | NA |
| Comparator | SRR1759249 | GSE64977 | Huntington's Disease | M | 69 | NA |
| Comparator | SRR1759250 | GSE64977 | Huntington's Disease | M | 71 | NA |
| Comparator | SRR1759251 | GSE64977 | Huntington's Disease | M | 48 | NA |
| Comparator | SRR1759252 | GSE64977 | Huntington's Disease | M | 40 | NA |
| Comparator | SRR1759253 | GSE64977 | Huntington's Disease | M | 72 | NA |
| Comparator | SRR1759254 | GSE64977 | Huntington's Disease | M | 43 | NA |
| Comparator | SRR1759255 | GSE64977 | Huntington's Disease | M | 68 | NA |
| Comparator | SRR1759256 | GSE64977 | Huntington's Disease | M | 59 | NA |
| Comparator | SRR1759257 | GSE64977 | Huntington's Disease | M | 68 | NA |
| Comparator | SRR1759258 | GSE64977 | Huntington's Disease | M | 57 | NA |
| Comparator | SRR1759259 | GSE64977 | Huntington's Disease | M | 48 | NA |
| Comparator | SRR1759260 | GSE64977 | Huntington's Disease | M | 68 | NA |
| Comparator | SRR1759261 | GSE64977 | Huntington's Disease | M | 54 | NA |
| Comparator | SRR1759262 | GSE64977 | Huntington's Disease | M | 68 | NA |
| Comparator | SRR1759263 | GSE64977 | Huntington's Disease | M | 61 | NA |
| Comparator | SRR1759264 | GSE64977 | Huntington's Disease | M | 48 | NA |
| Comparator | SRR1759265 | GSE64977 | Huntington's Disease | M | 69 | NA |
| Comparator | SRR1759266 | GSE64977 | Huntington's Disease | F | 68 | NA |
| Comparator | SRR1759267 | GSE64977 | Huntington's Disease | M | 55 | NA |
| Comparator | SRR1759268 | GSE64977 | Huntington's Disease | M | 50 | NA |
| Comparator | SRR1759269 | GSE64977 | Huntington's Disease | M | 51 | NA |
| Comparator | SRR1759270 | GSE64977 | Huntington's Disease | M | 79 | NA |
| Comparator | SRR1759271 | GSE64977 | Huntington's Disease | M | 50 | NA |
| Comparator | SRR1759272 | GSE64977 | Huntington's Disease | M | 75 | NA |
| Comparator | SRR1759273 | GSE64977 | Huntington's Disease | M | 53 | NA |
| Comparator | SRR2353419 | GSE72962 | Parkinson's Disease | M | 80 | NA |
| Comparator | SRR2353421 | GSE72962 | Parkinson's Disease | M | 80 | NA |
| Comparator | SRR2353424 | GSE72962 | Parkinson's Disease | M | 81 | NA |
| Comparator | SRR2353425 | GSE72962 | Parkinson's Disease | M | 77 | NA |
| Comparator | SRR2353426 | GSE72962 | Parkinson's Disease | M | 64 | NA |
| Comparator | SRR2353428 | GSE72962 | Parkinson's Disease | M | 94 | NA |
| Comparator | SRR2353430 | GSE72962 | Parkinson's Disease | M | 85 | NA |
| Comparator | SRR2353431 | GSE72962 | Parkinson's Disease | M | 75 | NA |
| Comparator | SRR2353432 | GSE72962 | Parkinson's Disease | M | 74 | NA |
| Comparator | SRR2353433 | GSE72962 | Parkinson's Disease | M | 89 | NA |
| Comparator | SRR2353434 | GSE72962 | Parkinson's Disease | M | 66 | NA |
| Comparator | SRR2353435 | GSE72962 | Parkinson's Disease | M | 65 | NA |
| Comparator | SRR2353436 | GSE72962 | Parkinson's Disease | M | 85 | NA |
| Comparator | SRR2353438 | GSE72962 | Parkinson's Disease | M | 64 | NA |
| Comparator | SRR2353442 | GSE72962 | Parkinson's Disease | M | 74 | NA |
| Comparator | SRR2353443 | GSE72962 | Parkinson's Disease | M | 68 | NA |
| Comparator | SRR2353444 | GSE72962 | Parkinson's Disease | M | 79 | NA |
| Comparator | SRR2353445 | GSE72962 | Parkinson's Disease | M | 70 | NA |
| Comparator | SRR2353417 | GSE72962 | Parkinson's Disease with | M | 74 | NA |
| | | | Dementia | | | |
| Comparator | SRR2353418 | GSE72962 | Parkinson's Disease with | M | 83 | NA |
| | | | Dementia | | | |
| Comparator | SRR2353420 | GSE72962 | Parkinson's Disease with | M | 83 | NA |
| | | | Dementia | | | |
| Comparator | SRR2353422 | GSE72962 | Parkinson's Disease with | M | 84 | NA |
| | | | Dementia | | | |
| Comparator | SRR2353423 | GSE72962 | Parkinson's Disease with | M | 88 | NA |
| | | | Dementia | | | |
| Comparator | SRR2353427 | GSE72962 | Parkinson's Disease with | M | 85 | NA |
| | | | Dementia | | | |
| Comparator | SRR2353429 | GSE72962 | Parkinson's Disease with | M | 80 | NA |
| | | | Dementia | | | |
| Comparator | SRR2353437 | GSE72962 | Parkinson's Disease with | M | 64 | NA |
| | | | Dementia | | | |
| Comparator | SRR2353439 | GSE72962 | Parkinson's Disease with | M | 75 | NA |
| | | | Dementia | | | |
| Comparator | SRR2353440 | GSE72962 | Parkinson's Disease with | M | 68 | NA |
| | | | Dementia | | | |
| Comparator | SRR2353441 | GSE72962 | Parkinson's Disease with | M | 95 | NA |
| | | | Dementia | | | |
| Comparator | SRR1759274 | GSE64977 | Pre-AD | F | 86 | NA |
| Comparator | SRR1759275 | GSE64977 | Pre-AD | M | 49 | NA |
| AVERAGE | NA | NA | NA | NA | 71.32 ± | NA |
| | | | | | 14.7 | |

TABLE 2A
Disease Specific Biomarkers for Alzheimer's Disease Identified in Brain Tissue

| | | | Frequency | | p-value in |
|---|---|---|---|---|---|
| Seq. ID | Sequence | Total Reads | (Sensitivity) | Specificity | Discovery set |
| 1 | CAGGCAGTTACAGATCGAACTCC | 45 | 47.06% | 100% | 8.142E−09 |
| 2 | GGTCAGTTACAGATCGAAC | 31 | 47.06% | 100% | 8.142E−09 |
| 3 | CTGGCTGGGTTGTTCGAGACCCGC | 38 | 41.18% | 100% | 1.083E−07 |
| 4 | TTATGTGATGACTTACA | 78 | 35.29% | 100% | 1.319E−06 |
| 5 | TTCTGTGATGACTTACA | 48 | 35.29% | 100% | 1.319E−06 |
| 6 | AGGTTATGGGTTCGTGTCCCACC | 40 | 35.29% | 100% | 1.319E−06 |
| 7 | TCTTGCTCCGTCCACTCC | 38 | 35.29% | 100% | 1.319E−06 |
| 8 | GGTAGAGCATGGGACTCTTAATCGC | 35 | 35.29% | 100% | 1.319E−06 |
| 9 | TCGTGCTGGGCCCATAACC | 28 | 35.29% | 100% | 1.319E−06 |
| 10 | GGGTTGTGGGTTCGGGTCCCACC | 24 | 35.29% | 100% | 1.319E−06 |
| 11 | TTTATCACGTTCGCCTC | 23 | 35.29% | 100% | 1.319E−06 |
| 12 | AGGTTCCGGGCTCGGGACCCGGC | 23 | 35.29% | 100% | 1.319E−06 |
| 13 | CATATGTGGTGAATACGTGTT | 22 | 35.29% | 100% | 1.319E−06 |
| 14 | GCGGTAGAGCATGGGACTCTTAATCCC | 22 | 35.29% | 100% | 1.319E−06 |
| 15 | GATCCATTGGGGTTTCCCCGCGCAGGT | 21 | 35.29% | 100% | 1.319E−06 |
| 16 | CCATGGGACTCTTAATCC | 20 | 35.29% | 100% | 1.319E−06 |
| 17 | GGTAAACATCTCCGACTGGAA | 20 | 35.29% | 100% | 1.319E−06 |
| 18 | AGGGTGTGGGTTCGAATCCCACC | 73 | 29.41% | 100% | 1.484E−05 |
| 19 | AAGGTTCCGGGTTCGTGTCGCGGC | 62 | 29.41% | 100% | 1.484E−05 |
| 20 | AAGTTTCCGGGTTCGGGCCCCGGC | 62 | 29.41% | 100% | 1.484E−05 |
| 21 | AGGTTGTGGATTCGTGTCCCACC | 55 | 29.41% | 100% | 1.484E−05 |
| 22 | GAAGTTCCGGGTTCGGGTCCCGGC | 52 | 29.41% | 100% | 1.484E−05 |
| 23 | AGGCTGTGGGTTCGAATCCCACC | 39 | 29.41% | 100% | 1.484E−05 |
| 24 | GGGTGTGATGACTTACA | 37 | 29.41% | 100% | 1.484E−05 |
| 25 | AAGTTTCCGGGTTCGGGACCCGGC | 35 | 29.41% | 100% | 1.484E−05 |
| 26 | AAGGTTCCGGGTTCGGTTCCCGGC | 34 | 29.41% | 100% | 1.484E−05 |
| 27 | ACTGTGGACTCTGAATCCA | 31 | 29.41% | 100% | 1.484E−05 |
| 28 | AAGGTTCCGGGTTCGGGTACCGGC | 31 | 29.41% | 100% | 1.484E−05 |
| 29 | GCACGGGACTCTTAATCCC | 30 | 29.41% | 100% | 1.484E−05 |
| 30 | AAGTTTGTGGGTTCGTATCCCACC | 28 | 29.41% | 100% | 1.484E−05 |
| 31 | GGAGTGTGGGTTCGTGTCCCATC | 27 | 29.41% | 100% | 1.484E−05 |
| 32 | AGGTTGTGGGTTCGAGGCCCACC | 26 | 29.41% | 100% | 1.484E−05 |
| 33 | AGAGTTTCCGGGTTCGTGTCCCGGC | 25 | 29.41% | 100% | 1.484E−05 |
| 34 | TTGAGGGTGCGTGTCCCT | 24 | 29.41% | 100% | 1.484E−05 |
| 35 | AGAGGTTCCGGGGTCGGGTCCCGGC | 24 | 29.41% | 100% | 1.484E−05 |
| 36 | AGTGTGAGGGTTCGTGTCCCT | 23 | 29.41% | 100% | 1.484E−05 |
| 37 | CACCCGTAGTACCGACCTCGCG | 23 | 29.41% | 100% | 1.484E−05 |
| 38 | AGAGGTTCCGAGTTCGGGTCCCGGC | 23 | 29.41% | 100% | 1.484E−05 |
| 39 | TCCCCGGTGGTCTAGTGGTTAGGATTCCGCGCT | 23 | 29.41% | 100% | 1.484E−05 |
| 40 | GACGTCGGATCAGAAGA | 22 | 29.41% | 100% | 1.484E−05 |
| 41 | TTTTGGGATGACTTACA | 22 | 29.41% | 100% | 1.484E−05 |
| 42 | TTCACGTAATCCAGGAAAAGCT | 22 | 29.41% | 100% | 1.484E−05 |
| 43 | GAGGTTACGGGTTCGTGTCCCGGC | 22 | 29.41% | 100% | 1.484E−05 |
| 44 | ATGTGACTCTTAATCTC | 21 | 29.41% | 100% | 1.484E−05 |
| 45 | AGGGTGTGGGTTCGTCCCACC | 21 | 29.41% | 100% | 1.484E−05 |
| 46 | TATAGCACTCTGGACTCTGAATCCAGC | 20 | 29.41% | 100% | 1.484E−05 |

TABLE 2B
Disease Specific Biomarkers for Alzheimer's Disease Identified in Brain Tissue

Stage

| | NA | NA | NA | NA | NA | NA | Braak V |
|---|---|---|---|---|---|---|---|
| Seq. ID | SRR1658347 | SRR1658348 | SRR1658349 | SRR1658350 | SRR1658351 | SRR1658353 | SRR828723 |
| 1 | | 0.549 | 0.225 | 2.012 | | | |
| 2 | | 0.549 | | 0.063 | | | |
| 3 | | | 0.674 | 0.44 | | | |
| 4 | | | | | | | |
| 5 | | | | | | | |
| 6 | 0.092 | 2.563 | 0.075 | 1.383 | 0.085 | 0.146 | |
| 7 | | | | | | | 0.181 |
| 8 | | | | | | | |
| 9 | | | | | | | |
| 10 | | 1.464 | | 0.754 | 0.085 | | |
| 11 | | 0.183 | | | | | |
| 12 | 0.092 | 0.732 | 0.15 | 0.88 | 0.085 | 0.146 | |
| 13 | | | | | | | |
| 14 | | | | | | | |
| 15 | | | | | | | |
| 16 | | | | | | | |
| 17 | | | | | | | |
| 18 | 0.277 | 2.014 | 0.075 | 3.583 | 0.085 | | |
| 19 | 0.277 | 6.407 | 0.449 | 1.006 | 0.17 | | |
| 20 | 0.277 | 3.844 | 0.15 | 2.2 | 0.085 | | |
| 21 | | 3.295 | 0.075 | 2.075 | 0.17 | | |
| 22 | 0.185 | 5.858 | 0.075 | 0.943 | 0.17 | | |
| 23 | 0.092 | 1.098 | 0.15 | 1.823 | 0.085 | | |
| 24 | | | | | | | |
| 25 | 0.092 | 3.478 | 0.3 | 0.503 | 0.255 | | |
| 26 | 0.185 | 2.929 | 0.075 | 0.88 | 0.085 | | |
| 27 | | | 0.075 | 1.634 | 0.17 | | |
| 28 | 0.277 | 2.014 | 0.524 | 0.566 | 0.085 | | |
| 29 | 0.185 | 0.366 | 0.15 | 1.257 | 0.34 | | |
| 30 | | 0.732 | 0.15 | 1.194 | 0.17 | | |
| 31 | 0.092 | 2.929 | 0.075 | 0.377 | 0.255 | | |
| 32 | | 1.098 | 0.075 | 1.006 | 0.17 | | |
| 33 | 0.092 | 3.112 | 0.3 | 0.126 | | | |
| 34 | 0.831 | 0.366 | 0.075 | 0.629 | 0.17 | | |
| 35 | 0.554 | 2.197 | 0.075 | 0.126 | 0.255 | | |
| 36 | 0.554 | 0.915 | 0.075 | 0.44 | 0.34 | | |
| 37 | | | | | | | 1.268 |
| 38 | 0.092 | 2.929 | 0.15 | 0.189 | 0.085 | | |
| 39 | | | | | | | 0.906 |
| 40 | 0.554 | 2.197 | 0.15 | 0.063 | | | |
| 41 | | | | | | | |
| 42 | | | 0.15 | | | | 1.087 |
| 43 | 0.092 | 2.929 | 0.075 | 0.189 | 0.085 | | |
| 44 | 0.092 | 0.549 | | 0.943 | | | |
| 45 | | 1.647 | 0.075 | 0.566 | 0.085 | | |
| 46 | | | | | | | |
| # Biomarkers Per | 20 | 28 | 27 | 29 | 23 | 2 | 4 |
| Sample | | | | | | | |
| % Coverage | 43% | 61% | 59% | 63% | 50% | 4% | 9% |

Stage

| | Braak VI | Braak VI | Braak VI | Braak VI | Braak VI | Braak VI | Braak VI |
|---|---|---|---|---|---|---|---|
| Seq. ID | SRR1103943 | SRR1103944 | SRR1103945 | SRR1103946 | SRR1103947 | SRR1103948 | SRR828724 |
| 1 | 0.074 | 0.199 | 0.111 | 0.139 | 0.108 | | |
| 2 | 0.074 | 0.598 | 0.445 | 0.278 | 0.867 | 0.378 | |
| 3 | | 0.299 | 0.445 | 0.417 | 0.65 | 0.284 | |
| 4 | 0.222 | 0.498 | 0.668 | 1.252 | 0.867 | 3.595 | |
| 5 | 0.296 | 0.299 | 0.223 | 0.626 | 0.433 | 2.46 | |
| 6 | | | | | | | |
| 7 | 0.37 | 0.598 | | 1.183 | 0.433 | 0.473 | |
| 8 | 0.296 | 0.498 | 0.223 | 0.765 | 0.542 | 0.757 | |
| 9 | 0.37 | 0.199 | 0.223 | 0.835 | 0.433 | 0.284 | |
| 10 | 0.074 | | 0.111 | 0.07 | | | |
| 11 | | 0.199 | 0.445 | 0.905 | 0.217 | 0.095 | |
| 12 | | | | | | | |
| 13 | 0.074 | 0.299 | 0.334 | 0.348 | 0.65 | 0.378 | |
| 14 | 0.074 | | 0.111 | 0.557 | 0.758 | 0.378 | 0.211 |
| 15 | 0.148 | 0.199 | 0.334 | 0.278 | 0.325 | 0.662 | |
| 16 | 0.222 | 0.299 | 0.111 | 0.626 | 0.217 | 0.189 | |
| 17 | 0.222 | 0.199 | 0.668 | 0.209 | 0.108 | 0.473 | |
| 18 | | | | | | | |
| 19 | | | | | | | |
| 20 | | | | | | | |
| 21 | | | | | | | 0.211 |
| 22 | | | | | | | |
| 23 | | | | | | | |
| 24 | 0.296 | 0.1 | | 0.835 | 0.325 | 1.608 | |
| 25 | | | | | | | |
| 26 | | | | | | | |
| 27 | | | 0.111 | 0.07 | | | |
| 28 | | | | | | | |
| 29 | | | | | | | |
| 30 | | 0.1 | | | | | |
| 31 | | | | | | | |
| 32 | | | 0.111 | | | | |
| 33 | | | | | | | 0.211 |
| 34 | | | | | | | |
| 35 | | | | | | | |
| 36 | | | | | | | |
| 37 | | | | | | | 0.634 |
| 38 | | | | | | | |
| 39 | | | | | | | 2.747 |
| 40 | | | | | 0.108 | | |
| 41 | | 0.199 | 0.111 | 0.696 | 0.758 | 0.189 | |
| 42 | | | | | | | 2.536 |
| 43 | | | | | | | |
| 44 | | | | 0.07 | 0.108 | | |
| 45 | | | | 0.07 | | | |
| 46 | | 0.199 | 0.78 | 0.278 | 0.217 | 0.473 | |
| # Biomarkers Per | 14 | 17 | 18 | 21 | 19 | 16 | 6 |
| Sample | | | | | | | |
| % Coverage | 30% | 37% | 39% | 46% | 41% | 35% | 13% |

| | Stage | | |
|---|---|---|---|
| | Braak VI | Braak VI | Braak VI |
| Seq. ID | SRR828725 | SRR828726 | SRR828727 |
| 1 | | | |
| 2 | | | |
| 3 | | | |
| 4 | | | |
| 5 | | | |
| 6 | | | |
| 7 | | | |
| 8 | | | |
| 9 | | | |
| 10 | | | |
| 11 | | | |
| 12 | | | |
| 13 | | | |
| 14 | | | |
| 15 | | | |
| 16 | | | |
| 17 | | | |
| 18 | | | |
| 19 | | | |
| 20 | | | |
| 21 | | | |
| 22 | | | |
| 23 | | | |
| 24 | | | |
| 25 | | | |
| 26 | | | |
| 27 | | | |
| 28 | | | |
| 29 | | | |
| 30 | | | |
| 31 | | | |
| 32 | | | |
| 33 | | | |
| 34 | | | |
| 35 | | | |
| 36 | | | |
| 37 | 4.334 | 6.641 | 30.067 |
| 38 | | | |
| 39 | 4.334 | 1.811 | 30.067 |
| 40 | | | |
| 41 | | | |
| 42 | 4.334 | 0.604 | |
| 43 | | | |
| 44 | | | |
| 45 | | | |
| 46 | | | |
| # Biomarkers Per | 3 | 3 | 2 |
| Sample | | | |
| % Coverage | 7% | 7% | 4% |

TABLE 3A
Experimental Alzheimer's disease cohort for
biomarker discovery, taken from CSF samples

| | | | | Age at | Disease | Braak |
|---|---|---|---|---|---|---|
| | Sample ID | Disease Type | Gender | Death | Duration | Score |
| Experimental | SRR1568546 | Alzheimer's | F | 91 | 19 | II |
| Experimental | SRR1568552 | Alzheimer's | M | 79 | 5 | II |
| Experimental | SRR1568556 | Alzheimer's | M | 90 | 1 | III |
| Experimental | SRR1568685 | Alzheimer's | M | 85 | 1 | III |
| Experimental | SRR1568693 | Alzheimer's | F | 91 | 4 | III |
| Experimental | SRR1568751 | Alzheimer's | M | 83 | 3 | III |
| Experimental | SRR1568420 | Alzheimer's | F | 77 | 3 | IV |
| Experimental | SRR1568436 | Alzheimer's | F | 88 | 3 | IV |
| Experimental | SRR1568488 | Alzheimer's | M | 82 | 9 | IV |
| Experimental | SRR1568533 | Alzheimer's | F | 86 | NA | IV |
| Experimental | SRR1568540 | Alzheimer's | F | 91 | 10 | IV |
| Experimental | SRR1568585 | Alzheimer's | F | 89 | 9 | IV |
| Experimental | SRR1568644 | Alzheimer's | F | 79 | 14 | IV |
| Experimental | SRR1568651 | Alzheimer's | M | 88 | 5 | IV |
| Experimental | SRR1568655 | Alzheimer's | M | 87 | 9 | IV |
| Experimental | SRR1568733 | Alzheimer's | M | 80 | 3 | IV |
| Experimental | SRR1568743 | Alzheimer's | F | 85 | 5 | IV |
| Experimental | SRR1568368 | Alzheimer's | M | 87 | 12 | V |
| Experimental | SRR1568370 | Alzheimer's | M | 86 | 21 | V |
| Experimental | SRR1568397 | Alzheimer's | M | 83 | 8 | V |
| Experimental | SRR1568406 | Alzheimer's | M | 75 | 10 | V |
| Experimental | SRR1568408 | Alzheimer's | M | 76 | 2 | V |
| Experimental | SRR1568445 | Alzheimer's | M | 76 | 4 | V |
| Experimental | SRR1568454 | Alzheimer's | M | 80 | 8 | V |
| Experimental | SRR1568467 | Alzheimer's | M | 75 | 7 | V |
| Experimental | SRR1568474 | Alzheimer's | F | 86 | 9 | V |
| Experimental | SRR1568480 | Alzheimer's | F | 75 | 5 | V |
| Experimental | SRR1568514 | Alzheimer's | F | 78 | 8 | V |
| Experimental | SRR1568522 | Alzheimer's | F | 87 | 5 | V |
| Experimental | SRR1568573 | Alzheimer's | F | 86 | 17 | V |
| Experimental | SRR1568638 | Alzheimer's | M | 75 | 6 | V |
| Experimental | SRR1568642 | Alzheimer's | F | 86 | 10 | V |
| Experimental | SRR1568665 | Alzheimer's | F | 81 | 7 | V |
| Experimental | SRR1568667 | Alzheimer's | F | 85 | 1 | V |
| Experimental | SRR1568673 | Alzheimer's | M | 75 | 8 | V |
| Experimental | SRR1568687 | Alzheimer's | M | 82 | 7 | V |
| Experimental | SRR1568704 | Alzheimer's | F | 86 | 5 | V |
| Experimental | SRR1568718 | Alzheimer's | F | 74 | 7 | V |
| Experimental | SRR1568388 | Alzheimer's | F | 97 | 5 | VI |
| Experimental | SRR1568422 | Alzheimer's | F | 84 | 15 | VI |
| Experimental | SRR1568432 | Alzheimer's | F | 60 | 5 | VI |
| Experimental | SRR1568434 | Alzheimer's | F | 74 | 12 | VI |
| Experimental | SRR1568440 | Alzheimer's | F | 84 | 14 | VI |
| Experimental | SRR1568456 | Alzheimer's | M | 78 | 8 | VI |
| Experimental | SRR1568489 | Alzheimer's | F | 70 | 4 | VI |
| Experimental | SRR1568495 | Alzheimer's | F | 74 | 8 | VI |
| Experimental | SRR1568524 | Alzheimer's | F | 70 | 5 | VI |
| Experimental | SRR1568529 | Alzheimer's | F | 57 | 10 | VI |
| Experimental | SRR1568537 | Alzheimer's | F | 65 | 3 | VI |
| Experimental | SRR1568539 | Alzheimer's | F | 82 | 11 | VI |
| Experimental | SRR1568561 | Alzheimer's | M | 87 | 6 | VI |
| Experimental | SRR1568565 | Alzheimer's | M | 78 | 5 | VI |
| Experimental | SRR1568599 | Alzheimer's | M | 85 | 5 | VI |
| Experimental | SRR1568610 | Alzheimer's | F | 68 | 8 | VI |
| Experimental | SRR1568640 | Alzheimer's | M | 83 | 6 | VI |
| Experimental | SRR1568647 | Alzheimer's | M | 77 | 1 | VI |
| Experimental | SRR1568661 | Alzheimer's | F | 93 | 3 | VI |
| Experimental | SRR1568663 | Alzheimer's | M | 81 | 7 | VI |
| Experimental | SRR1568672 | Alzheimer's | F | 78 | 7 | VI |
| Experimental | SRR1568677 | Alzheimer's | F | 90 | 12 | VI |
| Experimental | SRR1568722 | Alzheimer's | M | 83 | 8 | VI |
| Experimental | SRR1568740 | Alzheimer's | M | 80 | 10 | VI |
| Experimental | SRR1568747 | Alzheimer's | F | 89 | 9 | VI |
| Experimental | SRR1568755 | Alzheimer's | F | 79 | 10 | VI |
| AVERGAGE | NA | NA | NA | 81.00 ± | NA | NA |
| | | | | 10.1 | | |

TABLE 3B
Comparator cohort for AD biomarker discovery, taken from CSF samples, including
healthy controls and various other non-Alzheimer's neurological disorders

| | | | | Age at | Braak |
|---|---|---|---|---|---|
| Group | Sample ID | Disease Type | Gender | Death | Score |
| Comparator | SRR1568380 | Control | F | 88 | II |
| Comparator | SRR1568384 | Control | F | 78 | III |
| Comparator | SRR1568386 | Control | F | 90 | III |
| Comparator | SRR1568393 | Control | F | 80 | III |
| Comparator | SRR1568404 | Control | M | 85 | III |
| Comparator | SRR1568413 | Control | M | 89 | IV |
| Comparator | SRR1568415 | Control | F | 88 | III |
| Comparator | SRR1568417 | Control | M | 80 | II |
| Comparator | SRR1568428 | Control | M | 80 | I |
| Comparator | SRR1568441 | Control | M | 86 | II |
| Comparator | SRR1568447 | Control | F | 85 | III |
| Comparator | SRR1568459 | Control | F | 78 | IV |
| Comparator | SRR1568461 | Control | M | 82 | IV |
| Comparator | SRR1568463 | Control | F | 83 | II |
| Comparator | SRR1568469 | Control | F | 86 | IV |
| Comparator | SRR1568476 | Control | M | 82 | III |
| Comparator | SRR1568482 | Control | M | 75 | IV |
| Comparator | SRR1568484 | Control | M | 91 | IV |
| Comparator | SRR1568491 | Control | F | 88 | III |
| Comparator | SRR1568493 | Control | M | 84 | II |
| Comparator | SRR1568497 | Control | F | 87 | III |
| Comparator | SRR1568499 | Control | M | 84 | II |
| Comparator | SRR1568501 | Control | M | 73 | II |
| Comparator | SRR1568505 | Control | M | 78 | II |
| Comparator | SRR1568508 | Control | M | 89 | III |
| Comparator | SRR1568520 | Control | F | 84 | III |
| Comparator | SRR1568526 | Control | F | 90 | III |
| Comparator | SRR1568527 | Control | F | 75 | III |
| Comparator | SRR1568542 | Control | F | 88 | III |
| Comparator | SRR1568544 | Control | F | 87 | IV |
| Comparator | SRR1568550 | Control | F | 76 | I |
| Comparator | SRR1568559 | Control | M | 87 | IV |
| Comparator | SRR1568563 | Control | M | 76 | I |
| Comparator | SRR1568567 | Control | M | 94 | IV |
| Comparator | SRR1568569 | Control | M | 71 | I |
| Comparator | SRR1568578 | Control | F | 91 | IV |
| Comparator | SRR1568581 | Control | M | 82 | III |
| Comparator | SRR1568583 | Control | M | 65 | I |
| Comparator | SRR1568589 | Control | F | 99 | III |
| Comparator | SRR1568591 | Control | M | 92 | IV |
| Comparator | SRR1568593 | Control | M | 38 | 0 |
| Comparator | SRR1568601 | Control | M | 97 | III |
| Comparator | SRR1568602 | Control | M | 53 | I |
| Comparator | SRR1568605 | Control | M | 80 | III |
| Comparator | SRR1568608 | Control | M | 85 | III |
| Comparator | SRR1568612 | Control | F | 59 | I |
| Comparator | SRR1568614 | Control | F | 95 | III |
| Comparator | SRR1568620 | Control | F | 84 | IV |
| Comparator | SRR1568626 | Control | M | 93 | I |
| Comparator | SRR1568632 | Control | F | 92 | III |
| Comparator | SRR1568635 | Control | M | 74 | II |
| Comparator | SRR1568649 | Control | M | 90 | III |
| Comparator | SRR1568653 | Control | M | 84 | III |
| Comparator | SRR1568659 | Control | M | 78 | II |
| Comparator | SRR1568670 | Control | M | 83 | I |
| Comparator | SRR1568675 | Control | M | 79 | I |
| Comparator | SRR1568681 | Control | M | 84 | III |
| Comparator | SRR1568695 | Control | F | 87 | III |
| Comparator | SRR1568697 | Control | M | 90 | III |
| Comparator | SRR1568706 | Control | F | 73 | I |
| Comparator | SRR1568708 | Control | M | 78 | III |
| Comparator | SRR1568712 | Control | F | 70 | I |
| Comparator | SRR1568720 | Control | M | 86 | II |
| Comparator | SRR1568727 | Control | F | 76 | I |
| Comparator | SRR1568731 | Control | F | 88 | III |
| Comparator | SRR1568735 | Control | M | 81 | IV |
| Comparator | SRR1568741 | Control | M | 69 | I |
| Comparator | SRR1568749 | Control | F | 91 | III |
| Comparator | SRR1568366 | Parkinson's Disease | M | 70 | III |
| Comparator | SRR1568382 | Parkinson's Disease | M | 85 | II |
| Comparator | SRR1568424 | Parkinson's Disease | F | 86 | IV |
| Comparator | SRR1568450 | Parkinson's Disease | M | 89 | III |
| Comparator | SRR1568457 | Parkinson's Disease | F | 79 | IV |
| Comparator | SRR1568486 | Parkinson's Disease | M | 73 | I |
| Comparator | SRR1568512 | Parkinson's Disease | F | 87 | I |
| Comparator | SRR1568531 | Parkinson's Disease | F | 81 | III |
| Comparator | SRR1568554 | Parkinson's Disease | M | 86 | III |
| Comparator | SRR1568576 | Parkinson's Disease | F | 79 | II |
| Comparator | SRR1568630 | Parkinson's Disease | M | 80 | II |
| Comparator | SRR1568700 | Parkinson's Disease | M | 81 | I |
| Comparator | SRR1568702 | Parkinson's Disease | M | 77 | III |
| Comparator | SRR1568716 | Parkinson's Disease | F | 77 | II |
| Comparator | SRR1568724 | Parkinson's Disease | F | 83 | III |
| Comparator | SRR1568726 | Parkinson's Disease | F | 89 | IV |
| Comparator | SRR1568738 | Parkinson's Disease | F | 78 | III |
| Comparator | SRR1568364 | Parkinson's Disease | F | 73 | III |
| | | with Dementia | | | |
| Comparator | SRR1568372 | Parkinson's Disease | F | 87 | IV |
| | | with Dementia | | | |
| Comparator | SRR1568400 | Parkinson's Disease | F | 78 | III |
| | | with Dementia | | | |
| Comparator | SRR1568402 | Parkinson's Disease | F | 82 | III |
| | | with Dementia | | | |
| Comparator | SRR1568412 | Parkinson's Disease | M | 74 | I |
| | | with Dementia | | | |
| Comparator | SRR1568426 | Parkinson's Disease | M | 78 | III |
| | | with Dementia | | | |
| Comparator | SRR1568430 | Parkinson's Disease | M | 79 | II |
| | | with Dementia | | | |
| Comparator | SRR1568443 | Parkinson's Disease | M | 70 | II |
| | | with Dementia | | | |
| Comparator | SRR1568452 | Parkinson's Disease | M | 83 | III |
| | | with Dementia | | | |
| Comparator | SRR1568478 | Parkinson's Disease | F | 84 | II |
| | | with Dementia | | | |
| Comparator | SRR1568516 | Parkinson's Disease | M | 83 | 0 |
| | | with Dementia | | | |
| Comparator | SRR1568518 | Parkinson's Disease | F | 82 | III |
| | | with Dementia | | | |
| Comparator | SRR1568548 | Parkinson's Disease | M | 75 | III |
| | | with Dementia | | | |
| Comparator | SRR1568571 | Parkinson's Disease | M | 74 | III |
| | | with Dementia | | | |
| Comparator | SRR1568575 | Parkinson's Disease | M | 75 | IV |
| | | with Dementia | | | |
| Comparator | SRR1568616 | Parkinson's Disease | F | 85 | III |
| | | with Dementia | | | |
| Comparator | SRR1568624 | Parkinson's Disease | F | 84 | IV |
| | | with Dementia | | | |
| Comparator | SRR1568628 | Parkinson's Disease | M | 83 | III |
| | | with Dementia | | | |
| Comparator | SRR1568657 | Parkinson's Disease | F | 87 | II |
| | | with Dementia | | | |
| Comparator | SRR1568683 | Parkinson's Disease | M | 72 | I |
| | | with Dementia | | | |
| Comparator | SRR1568689 | Parkinson's Disease | M | 76 | III |
| | | with Dementia | | | |
| Comparator | SRR1568710 | Parkinson's Disease | M | 83 | III |
| | | with Dementia | | | |
| Comparator | SRR1568729 | Parkinson's Disease | F | 79 | II |
| | | with Dementia | | | |
| Comparator | SRR1568753 | Parkinson's Disease | M | 85 | III |
| | | with Dementia | | | |
| AVERGAGE | NA | NA | NA | 81.41 ± 8.5 | NA |

TABLE 4A
Disease Specific Biomarkers for Alzheimer's Disease Identified in CSF

| | | | Frequency | | p-value in |
|---|---|---|---|---|---|
| Seq. ID | Sequence | Total Reads | (Sensitivity) | Specificity | Discovery set |
| 47 | CCACGGACTCCCAAAAGCAGCTT | 16 | 9.38% | 100% | 2.20E−03 |
| 48 | ACCCCGTAGATCCGACCTTGTGA | 14 | 9.38% | 100% | 2.20E−03 |
| 49 | TCACCGGGTGTACATCAAGC | 9 | 9.38% | 100% | 2.20E−03 |
| 50 | CAACGGAATCTCCAAAGCAGCT | 9 | 9.38% | 100% | 2.20E−03 |
| 51 | TCTTGCACTCGTCCCGGCCTCAT | 9 | 9.38% | 100% | 2.20E−03 |
| 52 | TTTCGGCACTGAGGCCT | 8 | 9.38% | 100% | 2.20E−03 |
| 53 | TCACCCGGGTGTCAATCAGCTG | 8 | 9.38% | 100% | 2.20E−03 |
| 54 | CCCCCGTCGAACCGCCCTTGCGA | 8 | 9.38% | 100% | 2.20E−03 |
| 55 | GTTAAAATTCCTGAACCGGGACGCGGC | 33 | 9.38% | 100% | 2.20E−03 |
| 56 | GGTTCGTGCTGACGGCCTGTATCCTAGGCTACA | 31 | 9.38% | 100% | 2.20E−03 |
| | CCCTGAGGACT | | | | |
| 57 | CCCCCGTCGAACCGACCTTG | 27 | 9.38% | 100% | 2.20E−03 |
| 58 | TTCACAGTGGCTCAGTTCTGCC | 21 | 9.38% | 100% | 2.20E−03 |
| 59 | TTAAACTCTGTCGTGCTGG | 19 | 9.38% | 100% | 2.20E−03 |
| 60 | GCTAATACCGGATAAGAAAGC | 18 | 9.38% | 100% | 2.20E−03 |
| 61 | TCCCTGGTGGTCTGGTGGTTAGGAGTCGGCGC | 18 | 9.38% | 100% | 2.20E−03 |
| 62 | TAAAGTGCTGACCGTGCAGAT | 16 | 9.38% | 100% | 2.20E−03 |
| 63 | TCCTCTGTAGTTCAGTCGGTAGAAC | 13 | 9.38% | 100% | 2.20E−03 |
| 64 | TCCCTGTGGTCTAATGGTTAGGATCCGGCGCT | 13 | 9.38% | 100% | 2.20E−03 |
| 65 | CCTTGGCTGGGAGAACGCCTGGGAATACCGGG | 12 | 9.38% | 100% | 2.20E−03 |
| | TGCTGTAGGCTT | | | | |
| 66 | CAACATAGCGAGCCCCCGTCTCT | 11 | 9.38% | 100% | 2.20E−03 |
| 67 | CAGTTGCCACGTTCCCGTGG | 10 | 9.38% | 100% | 2.20E−03 |
| 68 | TGTAAACCTCCTGGCCTGGAAGCT | 10 | 9.38% | 100% | 2.20E−03 |
| 69 | CGCATTGCCGAGTAGCTATGTTCGGATG | 10 | 9.38% | 100% | 2.20E−03 |
| 70 | GACGGAAAGACCCCATGAACCTTTACTGTAGCT | 10 | 9.38% | 100% | 2.20E−03 |
| | TTGTATTGGAC | | | | |
| 71 | GGCTAATACCTGGGACTC | 9 | 9.38% | 100% | 2.20E−03 |
| 72 | CGCGGGGTGGAGCAGCCTGGTAGCT | 9 | 9.38% | 100% | 2.20E−03 |
| 73 | CGGGTCGTGGGTTCGCCCCACGTTGGGCGC | 9 | 9.38% | 100% | 2.20E−03 |
| 74 | TCTACAGTCCGACGATACGACTCTTAGCGG | 9 | 9.38% | 100% | 2.20E−03 |
| 75 | GGGCCCCTACCCGGCCGTCGCCGGCAGTCGAG | 9 | 9.38% | 100% | 2.20E−03 |
| 76 | TCTTCCGTAGTGTAGTGGTTATGACGTTCGCCT | 9 | 9.38% | 100% | 2.20E−03 |
| 77 | TCAAGGCTAAAACTCAAA | 8 | 9.38% | 100% | 2.20E−03 |
| 78 | TACAGTACTGTGCTAACTGAAAA | 8 | 9.38% | 100% | 2.20E−03 |
| 79 | GCCACGGTGGCCGAGTGGTTAAGGC | 8 | 9.38% | 100% | 2.20E−03 |
| 80 | CCCCCACTGCTACATTTGACTGTCTT | 8 | 9.38% | 100% | 2.20E−03 |
| 81 | ACGGATAAAAGGTACCTCGGGGATAAC | 8 | 9.38% | 100% | 2.20E−03 |
| 82 | CTTCTAGAAATTTCTGAAAATGCTCTG | 8 | 9.38% | 100% | 2.20E−03 |
| 83 | CCCCCCACTGCTAAATTTGACTGGCTACT | 8 | 9.38% | 100% | 2.20E−03 |
| 84 | GGCCGCGTGCCTAATGGATAAGGCGTCTGAT | 8 | 9.38% | 100% | 2.20E−03 |
| 85 | CTGTGAGGGTGAGCGAATCGCTGAAAGCCGGC | 8 | 9.38% | 100% | 2.20E−03 |
| | C | | | | |
| 86 | GCTTGCGGAGTGTAGTGGTTATCACGTTCGCCT | 8 | 9.38% | 100% | 2.20E−03 |
| 87 | CAACGGATAAAAGGTACTCTAGGGATAACAGG | 8 | 9.38% | 100% | 2.20E−03 |
| | CT | | | | |
| 88 | CATTGGTGGTTCCGTGGTAGAATTCTCGCCTGC | 8 | 9.38% | 100% | 2.20E−03 |
| | C | | | | |
| 89 | GGCTGGTCCGATGGTAGTGGGGTATCAGAACT | 8 | 9.38% | 100% | 2.20E−03 |
| | TG | | | | |
| 90 | TTGACCTTACCGGATGGCACAAAGAGAAGTGG | 8 | 9.38% | 100% | 2.20E−03 |
| | GCAAGTTC | | | | |
| 91 | TCCCTAGTTCGTTTCTGGGAGCGGAGACCA | 49 | 9.38% | 100% | 2.20E−03 |
| 92 | TCCCATGTGGTCTAGCGGTTAGGATTCCT | 29 | 9.38% | 100% | 2.20E−03 |
| 93 | CGGGCCTTTCGGGGCCTCTTCCCCGGGC | 22 | 9.38% | 100% | 2.20E−03 |
| 94 | GTGGTTCCGGCTTTGGAC | 18 | 9.38% | 100% | 2.20E−03 |
| 95 | GTGCTAATCTGCGATAAGCGTCGGT | 16 | 9.38% | 100% | 2.20E−03 |
| 96 | TCAGTGCATCACCGACCTTTGTT | 15 | 9.38% | 100% | 2.20E−03 |
| 97 | TCCCTGAGACCCTTTAAACCTGT | 15 | 9.38% | 100% | 2.20E−03 |
| 98 | CTAGTACGAGAGGACCGGAGTGGACGCATC | 15 | 9.38% | 100% | 2.20E−03 |
| 99 | GAGGCAGCAGTAGGGAATAT | 14 | 9.38% | 100% | 2.20E−03 |
| 100 | TAGCACCATTTGCAATCGGTTG | 14 | 9.38% | 100% | 2.20E−03 |
| 101 | TTAGACAGTTCGGTCCCTATCTGCC | 14 | 9.38% | 100% | 2.20E−03 |
| 102 | TGATGTCGGCTCATCTCATCCTGGGGCT | 14 | 9.38% | 100% | 2.20E−03 |
| 103 | AATCCTGGTCGGACATCA | 13 | 9.38% | 100% | 2.20E−03 |
| 104 | TGCACCATGGTTCTCTGAGCATG | 13 | 9.38% | 100% | 2.20E−03 |
| 105 | TGGGGAGTTCGAGTCTCTCCGCCCCTGCCA | 13 | 9.38% | 100% | 2.20E−03 |
| 106 | CCAAGGGGTCGTGGGTTCGAATCCTGCCAGCC | 13 | 9.38% | 100% | 2.20E−03 |
| | GCACCA | | | | |
| 107 | TCGTGATACAGTTCGGTC | 12 | 9.38% | 100% | 2.20E−03 |
| 108 | TCCGGGGAGCACGCCTGTTCGAGTATCGT | 12 | 9.38% | 100% | 2.20E−03 |
| 109 | GCCCCGTTCGTCTAGCGGCCTAGGACGCCGGCC | 12 | 9.38% | 100% | 2.20E−03 |
| | TCT | | | | |
| 110 | CTTCCACAACGTTCCCG | 11 | 9.38% | 100% | 2.20E−03 |
| 111 | TTCGATCCCGTCATCACC | 11 | 9.38% | 100% | 2.20E−03 |
| 112 | AAAGAGGAGGAGAGGAGAAC | 11 | 9.38% | 100% | 2.20E−03 |
| 113 | TCCACCACGTTCCCGTGGTAAATCAGCTTG | 11 | 9.38% | 100% | 2.20E−03 |
| 114 | GCAAGCAGGGGTCGTCGGTTCGATCCCGTCATC | 11 | 9.38% | 100% | 2.20E−03 |
| | CTCCACCA | | | | |
| 115 | CCCCCACGTTCCCGTTGG | 10 | 9.38% | 100% | 2.20E−03 |
| 116 | TTTGGTATCTGCGCTCTGC | 10 | 9.38% | 100% | 2.20E−03 |
| 117 | CACCTTGCGCAATCAGGACTGA | 10 | 9.38% | 100% | 2.20E−03 |
| 118 | GGGATAGTAGGTCGTTGCCAACC | 10 | 9.38% | 100% | 2.20E−03 |
| 119 | GGAAGAACGGGTGCTGTAGGCTTT | 10 | 9.38% | 100% | 2.20E−03 |
| 120 | CGAGACCAGGACTTTGATAGGCTGGGTG | 10 | 9.38% | 100% | 2.20E−03 |
| 121 | AAGCAGCAATGCGACGTATAGGGTCTGACGCC | 10 | 9.38% | 100% | 2.20E−03 |
| | T | | | | |
| 122 | TCAAATGGTAGAGCGCTCGCTTGGCTTGCGAG | 10 | 9.38% | 100% | 2.20E−03 |
| | A | | | | |
| 123 | GACCCAGTTGCCTAATTGGATAAGGCATCAGCC | 10 | 9.38% | 100% | 2.20E−03 |
| | T | | | | |
| 124 | TCCCTGGTGGTCTGGTGGTTAGGAGTCGGCGCT | 10 | 9.38% | 100% | 2.20E−03 |
| | CT | | | | |
| 125 | ATAGATCCTGAAACCGC | 9 | 9.38% | 100% | 2.20E−03 |
| 126 | CTCTTCGAGGCCCTGTAAT | 9 | 9.38% | 100% | 2.20E−03 |
| 127 | AGGTCCTCAATACGTATTTG | 9 | 9.38% | 100% | 2.20E−03 |
| 128 | CAAGGCAAAGACGCGTAGCT | 9 | 9.38% | 100% | 2.20E−03 |
| 129 | AACTGGAGAGTTTGATTCTGGCT | 9 | 9.38% | 100% | 2.20E−03 |
| 130 | CGGTGAATACGTTCCCGGGCCTT | 9 | 9.38% | 100% | 2.20E−03 |
| 131 | TTCCCTTTTTAATCCTATGCCTG | 9 | 9.38% | 100% | 2.20E−03 |
| 132 | AGCACGCGCGCACGTGTTAGGACC | 9 | 9.38% | 100% | 2.20E−03 |
| 133 | CAGATGGCGGAATTGGTAGACGCGCT | 9 | 9.38% | 100% | 2.20E−03 |
| 134 | CGTGGTTCATTTCCCCCTTTCGGGCG | 9 | 9.38% | 100% | 2.20E−03 |
| 135 | GGTCGATGATGATTGGTAAAAGGTCTG | 9 | 9.38% | 100% | 2.20E−03 |
| 136 | GTCGCCGGTTCAAGTCCGGCAGTCGGCTCCA | 9 | 9.38% | 100% | 2.20E−03 |
| 137 | AACACCGTGGAAGTTCGAGTCTTCTCCTGGGCA | 9 | 9.38% | 100% | 2.20E−03 |
| | CCA | | | | |
| 138 | AGGGATGTCGCTCAACG | 8 | 9.38% | 100% | 2.20E−03 |
| 139 | GCCTGTAGTCGTGCCCG | 8 | 9.38% | 100% | 2.20E−03 |
| 140 | AATCGATCGAGGGCTTAAC | 8 | 9.38% | 100% | 2.20E−03 |
| 141 | GCAACCATCCTCTGCTACC | 8 | 9.38% | 100% | 2.20E−03 |
| 142 | TCAACTTCGGAACTGCCTT | 8 | 9.38% | 100% | 2.20E−03 |
| 143 | ACATTGGGACTGAGCCACGGC | 8 | 9.38% | 100% | 2.20E−03 |
| 144 | GGAGGGGAGTGAAATAGAACC | 8 | 9.38% | 100% | 2.20E−03 |
| 145 | TGAATACCGTGCTGTAGGCTT | 8 | 9.38% | 100% | 2.20E−03 |
| 146 | CTAATCGATCGAGGGCTTAACC | 8 | 9.38% | 100% | 2.20E−03 |
| 147 | TGACCGGGAGTCAATCAGCTTG | 8 | 9.38% | 100% | 2.20E−03 |
| 148 | TGAGGGGCAGAGCGCGAGACTA | 8 | 9.38% | 100% | 2.20E−03 |
| 149 | TGCGGACAAGGGGAATCTGACT | 8 | 9.38% | 100% | 2.20E−03 |
| 150 | TTATGTAGTAGATTGTTATAGT | 8 | 9.38% | 100% | 2.20E−03 |
| 151 | CCCCGTCCGCCCCCCGTTCCCCC | 8 | 9.38% | 100% | 2.20E−03 |
| 152 | GGAGGGGCAGAGAGCGAGCCTTT | 8 | 9.38% | 100% | 2.20E−03 |
| 153 | TAGGGGTGAAAGGCTAAACAAAC | 8 | 9.38% | 100% | 2.20E−03 |
| 154 | TGTCTGAACATGGGGGGACCACC | 8 | 9.38% | 100% | 2.20E−03 |
| 155 | TTCATTCGGCTGTCCGAGATGTA | 8 | 9.38% | 100% | 2.20E−03 |
| 156 | AGCTAGACAGCAGGACGGTGGCCA | 8 | 9.38% | 100% | 2.20E−03 |
| 157 | TTATGGCCAGGCTGTCTCCACCCGA | 8 | 9.38% | 100% | 2.20E−03 |
| 158 | AATAGAACCTGAAACCGGATGCCTAC | 8 | 9.38% | 100% | 2.20E−03 |
| 159 | CGCGCTCGCCGGCCGAGGTGGGATCCC | 8 | 9.38% | 100% | 2.20E−03 |
| 160 | GCGGATGTGGCTCAGCTGGTAGAGCATC | 8 | 9.38% | 100% | 2.20E−03 |
| 161 | CTCGTACCAAACGAGAACTTTGAAGGCCGAAG | 8 | 9.38% | 100% | 2.20E−03 |
| 162 | GCGGCTGTAGTGTAGTGGTGATCACGTTCGCCC | 8 | 9.38% | 100% | 2.20E−03 |
| 163 | ACGTAGAGGCCGGAGGTTCGAATCCTCTCACCC | 8 | 9.38% | 100% | 2.20E−03 |
| | C | | | | |
| 164 | TCATTGGTGGTTCAGTGGTAGACTTCTCGCCTG | 8 | 9.38% | 100% | 2.20E−03 |
| | CC | | | | |
| 165 | ACGATGTGGGATTGCATTGACAATCAGGAGGT | 8 | 9.38% | 100% | 2.20E−03 |
| | TGGCT | | | | |
| 166 | AACCTATCTGTGTAGGATAGGTGGGAGGCTTT | 8 | 9.38% | 100% | 2.20E−03 |
| | GAAGTC | | | | |
| 167 | CTAAATACTCGTACATGACC | 16 | 10.94% | 100% | 7.63E−04 |
| 168 | CCCTAGCTTGTGCGCTCCTGGA | 15 | 10.94% | 100% | 7.63E−04 |
| 169 | TGCAACTCGACTCCATGAAGTC | 10 | 10.94% | 100% | 7.63E−04 |
| 170 | TCCCCGTAATCTTCATAATCCGGAG | 8 | 10.94% | 100% | 7.63E−04 |
| 171 | GCATTGGTGGTTCGGTGGTAGAATGCTCGCCTG | 17 | 10.94% | 100% | 7.63E−04 |
| 172 | TTCGAGCCCCGCGGGTGCTTACTGACCCTTT | 15 | 10.94% | 100% | 7.63E−04 |
| 173 | ACTTGGCTGGGAGACCGCCTGGGAATACCGGG | 14 | 10.94% | 100% | 7.63E−04 |
| | TGCTGTATGCT | | | | |
| 174 | CCCCATGAAGTCGGAGTCGCTAGTAATCGCAG | 13 | 10.94% | 100% | 7.63E−04 |
| | AT | | | | |
| 175 | AATTGGCATGAGTCCACTTTAAATCCTTTAACG | 12 | 10.94% | 100% | 7.63E−04 |
| | AGGATCCAT | | | | |
| 176 | CAAAACTCCCGTGCTGATC | 10 | 10.94% | 100% | 7.63E−04 |
| 177 | TGCCCGTTGGTCTAGGGGGATGATTCTCGCTT | 10 | 10.94% | 100% | 7.63E−04 |
| 178 | TCCTCGATAGCTCAGTTGGTAGAGCGCCGGACT | 10 | 10.94% | 100% | 7.63E−04 |
| 179 | CGAGCCCAGGTTGGAGAGCCA | 9 | 10.94% | 100% | 7.63E−04 |
| 180 | GATCAGCTACCGTCGTAGTTC | 9 | 10.94% | 100% | 7.63E−04 |
| 181 | GTCTTTTTGTCCTCCTATGCCTG | 9 | 10.94% | 100% | 7.63E−04 |
| 182 | ATGGTTCGCACTCTGGACTCTGAAT | 9 | 10.94% | 100% | 7.63E−04 |
| 183 | CCACGTTCCCGTGGATTCCACCACGTTCCCGGG | 9 | 10.94% | 100% | 7.63E−04 |
| | G | | | | |
| 184 | CCTAAAAAGACGGATGTTGCTGAGTGTGGACC | 9 | 10.94% | 100% | 7.63E−04 |
| | TGG | | | | |
| 185 | TAGAAACCGGGCGGAAACA | 8 | 10.94% | 100% | 7.63E−04 |
| 186 | CTGGAGACCGGGGTTCGATTTCCCGACGGGGA | 8 | 10.94% | 100% | 7.63E−04 |
| | GCC | | | | |
| 187 | TCTGCTGAGGCTAAGCCCGTGTTCTAAAGATTT | 8 | 10.94% | 100% | 7.63E−04 |
| | GT | | | | |
| 188 | CCATGTGTCGTAGGTTCGAATCCTATCGGGGCC | 8 | 10.94% | 100% | 7.63E−04 |
| | GCCA | | | | |
| 189 | TCAGTGCATGACCGAACTTGT | 26 | 10.94% | 100% | 7.63E−04 |
| 190 | TAGTTGGTTTTCGGAACTGAGGCCA | 20 | 10.94% | 100% | 7.63E−04 |
| 191 | GGACAGTGTCTGGTGGGTAGTTTGACTGGGGC | 16 | 10.94% | 100% | 7.63E−04 |
| | GGTCTCCT | | | | |
| 192 | TGCCCTTTGTCATCCTCTTCCTG | 14 | 10.94% | 100% | 7.63E−04 |
| 193 | CGCTACCTCAGATCAGGACGTGGCGACCCGCT | 14 | 10.94% | 100% | 7.63E−04 |
| | GAAT | | | | |
| 194 | GTTGTCGTGGGTTCGAGCCCCATCAGCCACCCC | 13 | 10.94% | 100% | 7.63E−04 |
| | A | | | | |
| 195 | GCGGAAGTAGTTCAGTGGTAGAACATCA | 12 | 10.94% | 100% | 7.63E−04 |
| 196 | CGCGACCTCAGATCAGACGTGGCGACCCGCTG | 12 | 10.94% | 100% | 7.63E−04 |
| | AGTGTAAGC | | | | |
| 197 | GCAGGTTCAGTCCTGCCGCGGTCGC | 11 | 10.94% | 100% | 7.E3E−04 |
| 198 | GTGATATAGACAGCAGGACGGTGGCCA | 11 | 10.94% | 100% | 7.E3E−04 |
| 199 | CCAGTGTGAAAGTAGGTTATCTTCAGGCT | 11 | 10.94% | 100% | 7.E3E−04 |
| 200 | GTACCGGGTGTAAATCAGCTG | 10 | 10.94% | 100% | 7.E3E−04 |
| 201 | CACCGAAATCGCGGATATGAGCGTTCCT | 10 | 10.94% | 100% | 7.E3E−04 |
| 202 | AGTCTGGCACGGTGAAGAGACATGAGAGGGG | 10 | 10.94% | 100% | 7.E3E−04 |
| 203 | GTAACCGGGGTTCGAATCCCCGTAGGGACGCC | 10 | 10.94% | 100% | 7.63E04 |
| | A | | | | |
| 204 | GCTGCATGGCCGTCGTC | 9 | 10.94% | 100% | 7.E3E−04 |
| 205 | CGGGCGCTGTAGGCTTTT | 9 | 10.94% | 100% | 7.E3E−04 |
| 206 | GTCCTCTCGGCCGCACCA | 9 | 10.94% | 100% | 7.E3E−04 |
| 207 | CGCAGAGTCGCGCAGCGGAAG | 9 | 10.94% | 100% | 7.E3E−04 |
| 208 | CGGGGIGTAGCTTAGCCTGGTA | 9 | 10.94% | 100% | 7.E3E−04 |
| 209 | GCCGGCTAGCTCAGTCGGTAGAG | 9 | 10.94% | 100% | 7.E3E−04 |
| 210 | TTCCGTTTGTCATCCTATGGCTG | 9 | 10.94% | 100% | 7.E3E−04 |
| 211 | ATCCTGTCTGAATATGGGGGGACC | 9 | 10.94% | 100% | 7.E3E−04 |
| 212 | GGCTCATAACCCGAAGGTCGTCGGT | 9 | 10.94% | 100% | 7.63E−04 |
| 213 | TCCAGGGTTCAGTTCCCTGTTCGGGCG | 9 | 10.94% | 100% | 7.E3E−04 |
| 214 | ACGGATAAAAGGTACCTCGGGGATAACAG | 9 | 10.94% | 100% | 7.63E−04 |
| 215 | GCATTTGTGGTGCAGTGGTAGAATTCTAGCCT | 9 | 10.94% | 100% | 7.63E−04 |
| 216 | CACAACGAGATCACCTCTGGGTCGTCTGCCGGT | 9 | 10.94% | 100% | 7.63E−04 |
| | CTCCACC | | | | |
| 217 | CTGCACTACAGCCTGGGCAACATAGCGAGACCC | 9 | 10.94% | 100% | 7.E3E−04 |
| | CGTCTCTA | | | | |
| 218 | ATTGACCGATTGAGAGCT | 8 | 10.94% | 100% | 7.E3E−04 |
| 219 | CCGGGGCCACGTGCCCGTGG | 8 | 10.94% | 100% | 7.63E04 |
| 220 | GTTCAGATCCCGGACGAGCCA | 8 | 10.94% | 100% | 7.E3E−04 |
| 221 | TCAAACAGAACTTTGAAGGCCGAAG | 8 | 10.94% | 100% | 7.63E−04 |
| 222 | CGTGTTCAGGTGACGTCGGGGTCACC | 8 | 10.94% | 100% | 7.63E−04 |
| 223 | TGTCGGGCTGGGGCGCGAAGCGGGGC | 8 | 10.94% | 100% | 7.63E−04 |
| 224 | GCCCGGCTAGCTCAGTCGGTAGATCATGAGAC | 8 | 10.94% | 100% | 7.63E−04 |
| | A | | | | |
| 225 | TCCCACATCGTCCAGCGGTTAGGATTCCTGGTT | 8 | 10.94% | 100% | 7.63E−04 |
| 226 | TCCCTGGTGGTCTAGTGACTAGGATTCGGCGCT | 8 | 10.94% | 100% | 7.63E−04 |
| | T | | | | |
| 227 | ACAAACCGGAGGAAGGT | 9 | 12.50% | 100% | 2.62E−04 |
| 228 | CTCGACCCTTCGAACGCACTTGCGGCCCCGGGT | 26 | 12.50% | 100% | 2.62E−04 |
| | T | | | | |
| 229 | GTAGTACCGCCATGTCTGT | 9 | 12.50% | 100% | 2.62E−04 |
| 230 | CGGTGGCACCACGTTCCCGGGG | 9 | 12.50% | 100% | 2.62E−04 |
| 231 | GCCACGATCGACTGAGATTCAGCCTTTGTTCTG | 9 | 12.50% | 100% | 2.62E−04 |
| | TAGATTTGT | | | | |
| 232 | TAGAGGTTATCACGTCTGCTT | 8 | 12.50% | 100% | 2.62E−04 |
| 233 | CAGATGGTAGTGGGTTATCAGAACTT | 8 | 12.50% | 100% | 2.62E−04 |
| 234 | GCTTGCGTAGGGTAGTGGTTATCACGTTCGCCT | 8 | 12.50% | 100% | 2.62E−04 |
| 235 | TAGACCGCCTGGGAATACCGGTTGCTGTAGGCT | 24 | 12.50% | 100% | 2.62E−04 |
| | T | | | | |
| 236 | GGGAGGCTTTGAAGTGTGGACGCCAGTCTGC | 16 | 12.50% | 100% | 2.62E−04 |
| 237 | GGGATGAACCGACCGCCGGGTT | 15 | 12.50% | 100% | 2.62E−04 |
| 238 | GTCGGCAGTTCAATCCTGCCCATGGGCACCA | 13 | 12.50% | 100% | 2.62E−04 |
| 239 | ATAGTGCGTGTTCCCGTGTGAAAGTAGGTCATC | 10 | 12.50% | 100% | 2.62E−04 |
| | GTCAGGCT | | | | |
| 240 | GGTCATCTCGGGGGAACCT | 9 | 12.50% | 100% | 2.62E−04 |
| 241 | CACTCCAGCCTGGGCAACATAGCGCGACCCCGT | 9 | 12.50% | 100% | 2.62E−04 |
| | CTCTTA | | | | |
| 242 | TACGCCTGTCTGGGCGTCGC | 8 | 12.50% | 100% | 2.62E−04 |
| 243 | TGACCGGGGTAAATAAGCTTG | 8 | 12.50% | 100% | 2.62E−04 |
| 244 | CAGCGATCCGAGGTCAAATCTCGGTGGAACCTC | 8 | 12.50% | 100% | 2.62E−04 |
| | C | | | | |
| 245 | GGCTGGTCCGATGGGAGGGGGTTATCAGAACT | 10 | 14.06% | 100% | 8.90E−05 |
| | TAT | | | | |
| 246 | CAGTTCGGTCCCTATCTGCCGTGG | 17 | 14.06% | 100% | 8.90E−05 |
| 247 | TCAGTGCACTAAAGCACTTTGT | 10 | 14.06% | 100% | 8.90E−05 |
| 248 | GACGGATTGCGTAACTTGTTCAGACT | 15 | 14.06% | 100% | 8.90E−05 |
| 249 | TGGGAGAGTAGGTCGCCGCCGGACA | 14 | 14.06% | 100% | 8.90E−05 |
| 250 | GACGAAGACTGACGCTCAGGTGCGAAAGC | 14 | 14.06% | 100% | 8.90E−05 |
| 251 | GGGGTAGAGCACTGTTTAG | 10 | 14.06% | 100% | 8.90E−05 |
| 252 | GAAGTAGAAAAGAGCACATGGTGGATG | 13 | 15.62% | 100% | 2.98E−05 |
| 253 | TATTACACTCGTCCCGGCCTC | 13 | 17.19% | 100% | 9.88E−06 |
| 254 | TACCTGGTGGTATAGTGGTTAGGATTCGGCGCT | 22 | 18.75% | 100% | 3.23E−06 |
| | CT | | | | |

TABLE 4B
Disease Specific Biomarkers for Alzheimer's Disease Identified in CSF

| | Stage | | | | | | |
|---|---|---|---|---|---|---|---|
| | Braak II | Braak II | Braak III | Braak III | Braak III | Braak III | Braak IV |
| Seq. ID | SRR1568546 | SRR1568552 | SRR1568556 | SRR1568685 | SRR1568693 | SRR1568751 | SRR1568420 |
| 47 | 1.126 | | 0.9 | | | | |
| 48 | 1.126 | | 0.257 | | | | |
| 49 | 1.126 | | 0.257 | | | | |
| 50 | 1.126 | | 0.386 | | | | |
| 51 | 1.126 | 0.16 | 0.386 | | | | |
| 52 | 1.126 | | | | 1.74 | | |
| 53 | 2.252 | | 0.257 | | | | |
| 54 | 1.126 | | 0.129 | | | | |
| 55 | | | 0.129 | | | | |
| 56 | 2.252 | | 2.058 | | | | |
| 57 | | | 1.544 | | | | |
| 58 | | | 0.386 | | | | |
| 59 | | 0.16 | | | | 0.114 | |
| 60 | | 0.16 | | | | | |
| 61 | | | | 0.454 | | | |
| 62 | | | 1.286 | | | | |
| 63 | | | | | 0.58 | | |
| 64 | | | | 0.303 | | | |
| 65 | | | | | 2.899 | | |
| 66 | | | 0.129 | | | | |
| 67 | | | | 0.151 | | | |
| 68 | | | 0.129 | | | | |
| 69 | | 0.32 | 0.257 | | | | |
| 70 | | 0.16 | | | | | |
| 71 | | | 0.129 | | | | |
| 72 | | 0.16 | | | | | |
| 73 | | | | | 0.58 | | |
| 74 | | | | 0.151 | | 0.114 | |
| 75 | | | | | 2.319 | | |
| 76 | | | | 0.151 | | | |
| 77 | | 0.32 | | 0.151 | | | |
| 78 | | | 0.129 | | | | |
| 79 | | 0.32 | | | | | |
| 80 | 1.126 | | 0.386 | | | | |
| 81 | | 0.48 | | | | | |
| 82 | | | | 0.151 | | | |
| 83 | | | 0.257 | | | | |
| 84 | | | | | 0.58 | | |
| 85 | | 0.48 | | 0.151 | | | |
| 86 | | | | 0.151 | | | |
| 87 | | 0.16 | | 0.454 | | | |
| 88 | 1.126 | | 0.257 | | | | |
| 89 | | 0.32 | | | | | |
| 90 | | | | 0.151 | | | |
| 91 | | | | 0.151 | | | |
| 92 | | | | | | | |
| 93 | | | | | | | |
| 94 | | | | | | | |
| 95 | | | 0.129 | | | | |
| 96 | | | | | | | |
| 97 | | | | | | | |
| 98 | | | | | | 0.228 | |
| 99 | | | | | | | |
| 100 | | | 0.515 | | | | |
| 101 | | | | | | | |
| 102 | | | | | | | |
| 103 | | | | | | 0.228 | |
| 104 | | | | | | | |
| 105 | | | | | | | |
| 106 | | | | 0.303 | | | |
| 107 | | | | | | | |
| 108 | | | | | | | |
| 109 | | 0.16 | | | | | |
| 110 | | | | | | | |
| 111 | | | | | | | |
| 112 | | | | | | | 3.673 |
| 113 | | | | | | | |
| 114 | | | | | | | |
| 115 | | | | | | 0.114 | |
| 116 | | | 0.386 | | | | |
| 117 | | | | | | | |
| 118 | | | | | | | |
| 119 | | | | | | | |
| 120 | | | | | | | |
| 121 | | | | | | | |
| 122 | | | | | | | |
| 123 | | | | | | | |
| 124 | | | | | | | |
| 125 | | | | | | | |
| 126 | | | | | | | |
| 127 | | | | | | | |
| 128 | | 0.48 | | | | | |
| 129 | | | 0.129 | 0.151 | | | |
| 130 | | 0.32 | | | | | |
| 131 | | | | | | | |
| 132 | | | | | | | |
| 133 | | | | | | | |
| 134 | | | | | | | |
| 135 | 2.252 | | | | | | |
| 136 | | | | | | | |
| 137 | | | | | | | |
| 138 | | | | | | 0.228 | |
| 139 | | | 0.129 | | | | |
| 140 | | | | | | | |
| 141 | | | | | | | |
| 142 | | | 0.129 | | | | |
| 143 | | | | | | | |
| 144 | | | | | | | |
| 145 | | | | | | | |
| 146 | | | | | | | |
| 147 | | 0.16 | | | | | |
| 148 | | | 0.257 | | | | |
| 149 | | | | | | | |
| 150 | | | | | | | |
| 151 | | | | | | | |
| 152 | | | | | | | |
| 153 | | 0.32 | | | | | |
| 154 | | | | | | | |
| 155 | | | | | | | |
| 156 | | | | | | 0.114 | |
| 157 | | | | | | | |
| 158 | | | | | | | |
| 159 | | | | | | | |
| 160 | | | | | | | |
| 161 | | | | | | | |
| 162 | | | | | | | |
| 163 | | | | | | | |
| 164 | | | | | | | |
| 165 | | | | | | | |
| 166 | | | | | | | |
| 167 | | 1.12 | | 0.303 | | | |
| 168 | 2.252 | | 0.9 | | | | |
| 169 | 1.126 | | | | 0.58 | | |
| 170 | 1.126 | | 0.257 | | | | |
| 171 | | | | 0.303 | | | |
| 172 | | | | | 2.319 | | |
| 173 | | | | | 3.479 | | |
| 174 | | 0.48 | | | | | |
| 175 | | | | | 1.16 | | |
| 176 | | | | | 1.16 | | |
| 177 | | | | 0.151 | | | |
| 178 | | 0.16 | | | | | |
| 179 | | 0.16 | | | | | |
| 180 | | | | | 1.16 | | |
| 181 | | | | 0.303 | | 0.114 | |
| 182 | | | | 0.151 | | | |
| 183 | | 0.16 | | | 0.58 | | |
| 184 | | | 0.129 | | | 0.114 | |
| 185 | | | | 0.303 | | | |
| 186 | | | | | | 0.114 | 1.469 |
| 187 | | 0.16 | | | | | |
| 188 | | | | | 0.58 | | |
| 189 | | | | | | | |
| 190 | | | | | | | |
| 191 | | 0.16 | | | | | |
| 192 | | | | | | | |
| 193 | | | | | | | |
| 194 | | | | 0.303 | | | |
| 195 | | | | | | 0.114 | |
| 196 | | | | | | | |
| 197 | | | | | | | |
| 198 | | | | | | | |
| 199 | | | | | | | |
| 200 | | | | | | | |
| 201 | | | | 0.151 | | | |
| 202 | | | | | | | |
| 203 | | 0.16 | | | | | |
| 204 | | | | | | | |
| 205 | | | | 0.151 | | | |
| 206 | | | | 0.151 | | | |
| 207 | | | | | | | |
| 208 | | | | | | | |
| 209 | | | | | | | |
| 210 | | | | 0.151 | | | |
| 211 | | | | | | | |
| 212 | | | | | | | |
| 213 | | | | | | | |
| 214 | | 0.16 | | | | | |
| 215 | | | | | | | 0.735 |
| 216 | | | | 0.303 | | | |
| 217 | | | | | | | |
| 218 | 1.126 | | | | | | |
| 219 | | | | | | | |
| 220 | | | | | | | |
| 221 | | | | | | | |
| 222 | | | | | | | |
| 223 | | 0.32 | | | | | |
| 224 | | | | | | | |
| 225 | | | | | | | |
| 226 | | | | | | | |
| 227 | | 0.16 | | | | 0.114 | |
| 228 | | | | | 3.479 | | |
| 229 | | | | | | 0.114 | |
| 230 | | | | | | 0.114 | |
| 231 | | | | | | 0.114 | |
| 232 | | | | | | 0.114 | |
| 233 | | | | 0.151 | | | |
| 234 | | | | 0.151 | | 0.114 | |
| 235 | | | | | | | |
| 236 | | | | | | | |
| 237 | | | | | | | |
| 238 | | | | | | | |
| 239 | | | | | | | |
| 240 | | | | | | | 0.735 |
| 241 | | | | | | | |
| 242 | | | | | | | |
| 243 | | | | | | 0.114 | |
| 244 | | | | | | | |
| 245 | | 0.32 | | | | 0.114 | |
| 246 | | 0.16 | | | | | |
| 247 | | | | 0.151 | | | |
| 248 | | | | | | | |
| 249 | | | 0.129 | | 0.58 | | |
| 250 | | | | | | | |
| 251 | | | | | | | |
| 252 | | 0.16 | | | | | |
| 253 | | | | | | | |
| 254 | | | | 0.303 | | 0.114 | |
| # Biomarkers Per | 16 | 31 | 31 | 30 | 16 | 20 | 4 |
| Sample | | | | | | | |
| % Coverage | 5% | 10% | 10% | 10% | 5% | 6% | 1% |
| | Stage | | | | | | |
| | Braak IV | Braak IV | Braak IV | Braak IV | Braak IV | Braak IV | Braak IV |
| Seq. ID | SRR1568436 | SRR1568488 | SRR1568533 | SRR1568540 | SRR1568585 | SRR1568644 | SRR1568651 |
| 47 | | | | 0.298 | | | |
| 48 | | | | | | | |
| 49 | | 0.489 | | | | | |
| 50 | | 0.245 | | 0.298 | | | |
| 51 | | | | 0.595 | | | |
| 52 | | | | | 0.584 | | |
| 53 | | 0.245 | | 0.298 | | | |
| 54 | | | | 0.298 | | | |
| 55 | | | | 0.298 | | | |
| 56 | | | | 1.191 | | | |
| 57 | | | | 0.298 | | | |
| 58 | | 0.489 | | 0.595 | | | |
| 59 | | | | | | | |
| 60 | | | | | | | 0.646 |
| 61 | 0.391 | | | | | | |
| 62 | | | | 0.298 | | | |
| 63 | | | 0.377 | | | | |
| 64 | 0.391 | | 0.377 | | | | |
| 65 | | 0.489 | | | | | |
| 66 | | 0.489 | | | | | |
| 67 | | | 0.377 | | | | |
| 68 | | | | 0.595 | | | |
| 69 | | | | | | | |
| 70 | | | | | | 0.286 | |
| 71 | | | | | | | 0.646 |
| 72 | | | | | | | 0.215 |
| 73 | | 0.245 | | | | | |
| 74 | 0.391 | | | | | | |
| 75 | | | | 0.298 | | | |
| 76 | 0.391 | | | | | | |
| 77 | | | | | | | |
| 78 | | 0.489 | | 0.298 | | | |
| 79 | | | | | | | 0.215 |
| 80 | | | | 0.298 | | | |
| 81 | 0.195 | | | | | | |
| 82 | 0.195 | | | | | | |
| 83 | | | | | | | |
| 84 | | | | | | | 0.43 |
| 85 | | | | | | | |
| 86 | 0.391 | | | | | | |
| 87 | | | | | | | |
| 88 | | 0.245 | | 0.595 | | | |
| 89 | | | | | | | |
| 90 | 0.195 | | | | | 0.143 | |
| 91 | | | | | | | |
| 92 | 0.195 | | 0.377 | | | | |
| 93 | | 3.913 | | | | | |
| 94 | | 0.978 | | | | | |
| 95 | | | | | | | |
| 96 | | 2.201 | | | | | |
| 97 | | | | | | 0.143 | |
| 98 | | | | | | | |
| 99 | | | | | | 0.143 | |
| 100 | | 0.978 | | 0.298 | | 0.286 | |
| 101 | 0.195 | | | | | | |
| 102 | | | | | | | |
| 103 | | | | | | | |
| 104 | | | | | | 0.429 | |
| 105 | | | | | | | 0.215 |
| 106 | | | | | | | |
| 107 | | | | | | | 0.215 |
| 108 | 0.195 | | 0.377 | | | 0.286 | 0.646 |
| 109 | | | | | | | |
| 110 | | 0.245 | | | | | |
| 111 | | | | | | | 0.215 |
| 112 | | | | | | | |
| 113 | 0.391 | | | | | | |
| 114 | | | | | | | |
| 115 | | | | | | | |
| 116 | | | | | | | |
| 117 | | | 0.377 | | | | |
| 118 | | | 0.377 | | | | |
| 119 | | | | | | 0.143 | |
| 120 | | | | | | | |
| 121 | | | | | 0.584 | 0.143 | |
| 122 | 0.195 | | | | | | |
| 123 | | | | | | 0.572 | |
| 124 | 0.391 | | 0.377 | | | | |
| 125 | | 0.245 | | | | | |
| 126 | | | | | | | |
| 127 | | | | 0.595 | | | |
| 128 | | | | | | | |
| 129 | | | | | | | |
| 130 | | | | | | | |
| 131 | | 0.489 | | | | | |
| 132 | | | | | 0.584 | | |
| 133 | | | | | | | 0.43 |
| 134 | | | | | 0.584 | | |
| 135 | | | | | | | |
| 136 | | | | | | | |
| 137 | | | | | | 0.143 | |
| 138 | | | | | | | |
| 139 | | | | | | | |
| 140 | | | | | | | 0.215 |
| 141 | 0.195 | | | | | | |
| 142 | | | | | | | |
| 143 | | | | | | 0.286 | |
| 144 | | | | 0.298 | | | |
| 145 | | | | | | 0.143 | |
| 146 | | | | | | 0.286 | |
| 147 | | | | | | | |
| 148 | | 0.489 | | 0.298 | | | |
| 149 | | | | | | 0.143 | |
| 150 | | | | | | 0.286 | |
| 151 | 0.195 | | | | | | |
| 152 | | | | | | | 0.215 |
| 153 | | | | | | | |
| 154 | | | | | | | |
| 155 | | | | | | 0.286 | |
| 156 | | | | | | | |
| 157 | | | | | | | |
| 158 | 0.195 | | | | | | |
| 159 | | | 0.377 | | 0.584 | | |
| 160 | | | | | | 0.143 | |
| 161 | | | | | | | 0.215 |
| 162 | | | | | | 0.143 | |
| 163 | | | | | | 0.286 | |
| 164 | | | 0.377 | | | | |
| 165 | | | 1.132 | | | | |
| 166 | 0.195 | | | | | 0.286 | |
| 167 | | | | | | | |
| 168 | | | | 0.298 | | | |
| 169 | | | | | | | 0.43 |
| 170 | | | | | | | |
| 171 | 0.781 | | 0.377 | | | | |
| 172 | | | | | 1.752 | | |
| 173 | | | | | 0.584 | | |
| 174 | | | | | | | 0.646 |
| 175 | 0.195 | | | | 2.336 | | |
| 176 | 0.391 | | | | | | |
| 177 | 0.391 | | | | | | |
| 178 | 0.195 | | | 0.298 | | | |
| 179 | | | | | | | |
| 180 | | 0.245 | | | 1.168 | | |
| 181 | 0.195 | | | | | | |
| 182 | | 0.245 | | | | | |
| 183 | | | | | | | |
| 184 | 0.391 | | | | | | |
| 185 | | | | | | | |
| 186 | 0.195 | | | | | | |
| 187 | | | | | | | |
| 188 | | | | | | | 0.43 |
| 189 | | 0.978 | | | | | |
| 190 | 0.195 | | | | | | |
| 191 | | | | | | | |
| 192 | | | | | | 0.143 | |
| 193 | | | | | | 0.143 | |
| 194 | | | | | | | |
| 195 | | | | | | | |
| 196 | | | | | | 0.143 | |
| 197 | | | | | | 0.143 | |
| 198 | | | | | | | |
| 199 | | | 0.377 | | | | 0.215 |
| 200 | | | | | | | 0.215 |
| 201 | | | | | | | |
| 202 | | | | | | | |
| 203 | | | | | | | |
| 204 | | | | | | | 0.215 |
| 205 | | | | | | | |
| 206 | | | | | | | |
| 207 | | 0.245 | | | | | |
| 208 | | 0.245 | | | | | |
| 209 | | | | | 0.584 | | |
| 210 | | | | | | 0.143 | |
| 211 | | | | | | | |
| 212 | 0.195 | | | | | | |
| 213 | | | | | | 0.286 | |
| 214 | | | | | | | |
| 215 | | | | | | | 0.215 |
| 216 | | | | | | | |
| 217 | | | | | 1.752 | | |
| 218 | | | | 0.298 | | | |
| 219 | | | | | | 0.143 | |
| 220 | 0.391 | | | | | 0.143 | |
| 221 | | | | | 0.584 | | |
| 222 | 0.195 | | | | | | |
| 223 | | | | | | | |
| 224 | | | | | | | |
| 225 | 0.195 | | | | | | |
| 226 | 0.195 | | | | | | |
| 227 | | | | | | 0.143 | |
| 228 | | | | | 2.336 | | |
| 229 | | | | | | 0.143 | |
| 230 | | | | | | | 0.215 |
| 231 | | | | | | | |
| 232 | | | | | | | 0.215 |
| 233 | 0.195 | | | | | | |
| 234 | 0.195 | | | | | | |
| 235 | | | | | | | |
| 236 | | | | | | | 1.076 |
| 237 | | 0.978 | | 0.298 | | 0.143 | |
| 238 | | | | | | | |
| 239 | | | | | | | |
| 240 | | | | | | | |
| 241 | | 0.489 | | | | | |
| 242 | | | | | | 0.143 | |
| 243 | | | | | | | |
| 244 | 0.195 | | | | | | |
| 245 | | | | | | 0.143 | |
| 246 | | | | | | | |
| 247 | | | | | | | |
| 248 | | | | | | 0.143 | |
| 249 | | | | | | | |
| 250 | 0.195 | | | | | | |
| 251 | | | 0.377 | | | | |
| 252 | | | | | | | 0.215 |
| 253 | | | 0.377 | | | | |
| 254 | 0.976 | | 0.377 | | | | |
| # Biomarkers Per | 37 | 24 | 16 | 23 | 13 | 35 | 24 |
| Sample | | | | | | | |
| % Coverage | 12% | 8% | 5% | 7% | 4% | 11% | 8% |
| | Stage | | | | | | |
| | Braak IV | Braak IV | Braak IV | Braak V | Braak V | Braak V | Braak V |
| Seq. ID | SRR1568655 | SRR1568733 | SRR1568743 | SRR1568368 | SRR1568370 | SRR1568397 | SRR1568406 |
| 47 | | 0.614 | | | | | |
| 48 | | 0.614 | | | | | |
| 49 | | | | | | 0.928 | |
| 50 | | | | | | | |
| 51 | | | | | | | |
| 52 | 0.503 | | | | | | |
| 53 | | | | | | 0.464 | |
| 54 | | 0.307 | | | | | |
| 55 | | | | 0.093 | | | |
| 56 | | 0.614 | | | | | |
| 57 | | 0.921 | | | | | |
| 58 | | | | | | 0.928 | |
| 59 | | | | | 1.549 | | |
| 60 | | | | | | | |
| 61 | | | | | | | |
| 62 | | 0.614 | | | | | |
| 63 | | | | 0.186 | | | |
| 64 | | | | | | | |
| 65 | | | | | | | |
| 66 | 0.503 | | | | | 1.391 | |
| 67 | | | | | | | |
| 68 | | | | | | | |
| 69 | | | | | | | |
| 70 | | | | | | | |
| 71 | | | | | | | |
| 72 | | | | | | | |
| 73 | | | | | | | |
| 74 | | | | | 0.282 | | |
| 75 | | | | | 0.141 | 0.464 | 0.075 |
| 76 | | | | | | | |
| 77 | | | | | | | |
| 78 | | 0.307 | | | | | |
| 79 | | | | 0.093 | | | |
| 80 | | 0.307 | | | | | |
| 81 | | | | | | | |
| 82 | | | | | | | |
| 83 | | | 0.145 | | | | |
| 84 | | | | | | | |
| 85 | | | | | | | |
| 86 | | | | | | | |
| 87 | | | | | 0.141 | | |
| 88 | | | | | | | |
| 89 | | 0.307 | | | | | |
| 90 | | | | | | | |
| 91 | | | | | | | |
| 92 | | | | | 0.141 | | |
| 93 | 0.503 | | | | | | |
| 94 | 2.517 | | | 0.279 | 0.563 | | |
| 95 | | | | | 0.422 | | |
| 96 | | 0.307 | | | | 0.464 | |
| 97 | | | | | | | |
| 98 | | | | 0.093 | | | |
| 99 | | | | 0.372 | 0.422 | | |
| 100 | | 0.307 | | | | | |
| 101 | | | | | 0.141 | | |
| 102 | 0.503 | | | | | | |
| 103 | | | | | | | |
| 104 | | 0.307 | | | | | |
| 105 | | | | | 0.141 | | |
| 106 | | | | | | | |
| 107 | | | | | 0.845 | | |
| 108 | | | | | | | |
| 109 | | | | | | | 0.075 |
| 110 | 0.503 | | | | 0.282 | | |
| 111 | | | | | 0.141 | | |
| 112 | | | | | | 0.464 | |
| 113 | | | | | | | |
| 114 | | | 0.145 | | | | |
| 115 | | | | | | | |
| 116 | | | | | 0.282 | | |
| 117 | | | | | 0.563 | | |
| 118 | | | | | | | |
| 119 | | 1.535 | | | | | |
| 120 | | | 0.145 | | | | |
| 121 | | | | | | | |
| 122 | | | | | | | |
| 123 | | | | | | | |
| 124 | | | | | | | |
| 125 | 0.503 | | | | | | |
| 126 | | 0.614 | | | | | |
| 127 | | | | | | 0.928 | |
| 128 | | | | | | | |
| 129 | | | | | 0.282 | | |
| 130 | | | | | | | 0.075 |
| 131 | | | | | | | |
| 132 | 0.503 | | | | | | |
| 133 | | | | | | | |
| 134 | | | | 0.186 | 0.141 | | |
| 135 | | | | | | | |
| 136 | | | 0.145 | | | | 0.075 |
| 137 | | | | | | | |
| 138 | | | | | | | |
| 139 | | | | | | | |
| 140 | | | | 0.186 | | | |
| 141 | | | | | | | |
| 142 | | | | | | | 0.075 |
| 143 | | | | 0.186 | | | |
| 144 | | | | | | | |
| 145 | | | | | | | |
| 146 | | | | | | | |
| 147 | | | | 0.093 | | | |
| 148 | | | | | | | |
| 149 | | | | | 0.422 | | 0.075 |
| 150 | | | | | | | |
| 151 | | | | | | | |
| 152 | | | | | | | |
| 153 | | | | | | | 0.075 |
| 154 | | 0.307 | | | | | 0.075 |
| 155 | | | | | | | |
| 156 | | | | 0.093 | 0.141 | | |
| 157 | | | 0.145 | | 0.282 | | |
| 158 | | | | | | | 0.075 |
| 159 | | | | | | | |
| 160 | | 0.614 | | | | | |
| 161 | | | | | | | |
| 162 | | | | | | | |
| 163 | | | | | | | |
| 164 | | | | | | | |
| 165 | | | | | | | |
| 166 | | | | 0.186 | | | |
| 167 | | 0.307 | | | | | |
| 168 | | 0.307 | | | | | |
| 169 | | | | | 0.282 | | |
| 170 | | 0.307 | | | | | |
| 171 | | | | | | | |
| 172 | | | | | | 0.464 | |
| 173 | | | | | | | 0.075 |
| 174 | | | | | | | |
| 175 | | 0.307 | | | | | |
| 176 | | | | | | | |
| 177 | | | | | | | |
| 178 | | | | | | | 0.075 |
| 179 | | | 0.145 | | | 0.928 | |
| 180 | 0.503 | | | | 0.141 | | |
| 181 | | | | | | | |
| 182 | | | | | | | |
| 183 | | | | | | 0.464 | |
| 184 | | | | | | | |
| 185 | | 0.307 | | | | | |
| 186 | | | | | | | |
| 187 | 0.503 | | | | 0.282 | 0.464 | |
| 188 | | | | | | 0.464 | |
| 189 | | 0.307 | | | | | |
| 190 | | | | | | | |
| 191 | | | | | 0.422 | | |
| 192 | | | | | | | |
| 193 | | | | | | | |
| 194 | | | | | 0.282 | | |
| 195 | | | | | 0.141 | | |
| 196 | | | | | | | |
| 197 | | | | | 0.282 | | |
| 198 | | 0.921 | | | | | |
| 199 | | | | | | | |
| 200 | | | | 0.093 | | | |
| 201 | | | | | | | 0.149 |
| 202 | | 0.307 | | | 0.141 | | |
| 203 | | | | | 0.282 | | |
| 204 | | | | | 0.282 | | |
| 205 | | | | | | | |
| 206 | | | | | | | |
| 207 | | | | 0.186 | 0.282 | | |
| 208 | 1.007 | | | | | | |
| 209 | | | | 0.279 | | | |
| 210 | | | | | | | |
| 211 | | | 0.145 | | 0.141 | | 0.075 |
| 212 | | | | 0.093 | 0.422 | | |
| 213 | | | | | 0.141 | | |
| 214 | | | | | | | |
| 215 | | | | | | | |
| 216 | | | | | | | |
| 217 | | 0.307 | | | | | |
| 218 | 0.503 | | | 0.093 | 0.282 | | |
| 219 | | | | 0.093 | | | |
| 220 | | | | | | | |
| 221 | | | | | | | |
| 222 | | | | | | | |
| 223 | | | | | | | |
| 224 | | | 0.145 | | | | |
| 225 | | | | | 0.141 | | |
| 226 | | | | | | | |
| 227 | | | | | | | |
| 228 | | | | 0.093 | | | |
| 229 | | | | | | | |
| 230 | | | | | | | 0.075 |
| 231 | | | 0.145 | | 0.282 | | |
| 232 | | | | | | | |
| 233 | | 0.307 | | | | | |
| 234 | | | | | | | |
| 235 | | 3.991 | | | | | |
| 236 | | | | 0.279 | 0.141 | | |
| 237 | | | | | | 0.464 | |
| 238 | | | 0.145 | | 0.141 | | 0.149 |
| 239 | | | 0.145 | | | | |
| 240 | | | | | 0.141 | | |
| 241 | | | 0.145 | | | | |
| 242 | | | | | | | |
| 243 | | | | | 0.141 | | 0.075 |
| 244 | | | | | | | |
| 245 | | | | | | | |
| 246 | | | 0.289 | | 0.563 | | |
| 247 | | | 0.145 | | | | 0.075 |
| 248 | | | | | | | |
| 249 | | | | | 0.141 | 0.464 | |
| 250 | | | | | 0.141 | | |
| 251 | | | | | | | |
| 252 | | | 0.145 | 0.279 | | | |
| 253 | | | | | | | |
| 254 | | | | | | | |
| # Biomarkers Per | 12 | 27 | 15 | 21 | 44 | 15 | 17 |
| Sample | | | | | | | |
| % Coverage | 4% | 9% | 5% | 7% | 14% | 5% | 5% |
| | Stage | | | | | | |
| | Braak V | Braak V | Braak V | Braak V | Braak V | Braak V | Braak V |
| Seq. ID | SRR1568408 | SRR1568445 | SRR1568454 | SRR1568467 | SRR1568474 | SRR1568480 | SRR1568514 |
| 47 | | | | | | | |
| 48 | | | | | 0.366 | | |
| 49 | | | | | | | |
| 50 | | | | | 0.731 | | |
| 51 | | | | | 0.366 | | |
| 52 | | | 0.277 | | | | |
| 53 | | | | | | | |
| 54 | | | | | | | |
| 55 | | | | | | 0.188 | |
| 56 | | | | | 1.097 | | |
| 57 | | | | | 1.462 | | |
| 58 | | | | | | | |
| 59 | | | | | | | |
| 60 | | | | | | 0.188 | |
| 61 | | 0.227 | | | | | |
| 62 | | | | | 0.366 | | |
| 63 | | | 0.832 | | | | |
| 64 | | | | | | | |
| 65 | | | | | | | |
| 66 | | | | | 0.731 | | |
| 67 | | 0.227 | | | | | |
| 68 | | | | | | | 0.128 |
| 69 | | | | | | | |
| 70 | | | | | | | |
| 71 | | | | | 0.366 | | |
| 72 | | | | | | | |
| 73 | 0.46 | | | | | | |
| 74 | | | | 0.391 | | | |
| 75 | | | | | | | |
| 76 | | 0.34 | | | | | |
| 77 | | | | | | | |
| 78 | | | | | 0.366 | | |
| 79 | | | | | | | |
| 80 | | | | | 0.366 | | |
| 81 | | | | | 0.366 | | |
| 82 | | | | | 0.366 | | |
| 83 | | | | | | | |
| 84 | | | | 0.195 | | | |
| 85 | | | | | | | |
| 86 | | 0.113 | | | | | |
| 87 | | | | 0.195 | | | |
| 88 | | | | | | | |
| 89 | | | | | | | |
| 90 | | | | | | | |
| 91 | | | | | | | |
| 92 | 0.115 | | | | | | |
| 93 | | | | | 0.366 | | |
| 94 | | | | | | | |
| 95 | | | | | | | |
| 96 | | | | | 0.366 | | |
| 97 | | | | | | | 0.064 |
| 98 | | | | | | | |
| 99 | | | | | | | |
| 100 | | | | | | | |
| 101 | | | | | | | |
| 102 | | | | | | | |
| 103 | | | | | | | |
| 104 | | | | | | | 0.191 |
| 105 | | | | | | | |
| 106 | | | | | | | |
| 107 | | | | | | | |
| 108 | | 0.113 | | | | | |
| 109 | | | | | | | |
| 110 | | | | | | | |
| 111 | | | | | | | |
| 112 | | 0.113 | | 0.391 | | | |
| 113 | | | | 0.195 | | | |
| 114 | | | | 0.195 | | | |
| 115 | | | | 0.195 | | | |
| 116 | 0.23 | | | | | | |
| 117 | | | | | | | |
| 118 | | | | | | | |
| 119 | 0.115 | | | | | | |
| 120 | | | | | | | |
| 121 | | | | | | | |
| 122 | 0.46 | | | | | | 0.064 |
| 123 | | | | | | | 0.128 |
| 124 | | 0.227 | | | | | |
| 125 | | | | | | | |
| 126 | | | | | | | |
| 127 | | | | | 0.731 | | |
| 128 | | | | | | | |
| 129 | | | | | | | |
| 130 | | | | 0.391 | | | |
| 131 | | | | | | | 0.064 |
| 132 | | | | | | | |
| 133 | | 0.113 | | | | | |
| 134 | 0.345 | | | | | | |
| 135 | | | | | | | 0.064 |
| 136 | | | | | | | |
| 137 | | | | | 0.366 | | |
| 138 | | | | | | | |
| 139 | 0.115 | | | | | | |
| 140 | | | | | | | |
| 141 | | | | | | | 0.064 |
| 142 | | | | | | | |
| 143 | 0.115 | | | | | | |
| 144 | | | | | | | |
| 145 | 0.115 | | | | | | |
| 146 | | | | | | | |
| 147 | | | | | | | |
| 148 | | | | | | | |
| 149 | 0.115 | | | | | | |
| 150 | | | | | | | |
| 151 | 0.115 | | | | | | |
| 152 | | | | 0.195 | | | |
| 153 | | | | | | | |
| 154 | | | | | | | |
| 155 | | | | | | | 0.064 |
| 156 | | | | | | | |
| 157 | | | | | | | |
| 158 | | | | | | | |
| 159 | | | | | | | 0.064 |
| 160 | | | | | | | |
| 161 | | | | | | | |
| 162 | | | | | | | 0.191 |
| 163 | | | | | | | |
| 164 | | 0.113 | | | | | |
| 165 | | | | | | 0.188 | |
| 166 | | | | | | | |
| 167 | | | | | 0.731 | | |
| 168 | | | | | 0.731 | | |
| 169 | | | | | | | |
| 170 | | | | | | | |
| 171 | | 0.227 | | | | 0.188 | |
| 172 | | 0.113 | | | | | |
| 173 | | | | | | | |
| 174 | | 0.113 | | | | | |
| 175 | | | | | | | |
| 176 | | | | | | | |
| 177 | | 0.113 | | | | | |
| 178 | | | | 0.195 | | | |
| 179 | | | | | | 0.188 | |
| 180 | | | | | | | |
| 181 | | 0.113 | | | | | |
| 182 | | | | | | | |
| 183 | | | | | | | |
| 184 | | | | | | | |
| 185 | | | | | | | |
| 186 | | | | | | | |
| 187 | | | | | | | |
| 188 | | | | | | | |
| 189 | | | | | 0.731 | | |
| 190 | | | | | | | |
| 191 | | 0.113 | | | | | |
| 192 | | | | | | | 0.064 |
| 193 | | | | | | | 0.191 |
| 194 | | | | | | | |
| 195 | 0.23 | | | | | | |
| 196 | | | | | | | 0.064 |
| 197 | 0.345 | | | | | | |
| 198 | 0.115 | 0.113 | | | | | |
| 199 | | | | | | | |
| 200 | | | | | | | |
| 201 | | | | | | | |
| 202 | | | | | | | |
| 203 | | | | | 1.097 | 0.188 | |
| 204 | | | | | | | |
| 205 | | | | 0.195 | | | |
| 206 | | | | | | | |
| 207 | 0.115 | | | | | | |
| 208 | | | | | | | |
| 209 | | | | | | | |
| 210 | | | | | | | |
| 211 | | | | | | | |
| 212 | | | | | | | |
| 213 | | | | | | | 0.064 |
| 214 | | 0.113 | | | | | |
| 215 | | | | | | 0.188 | |
| 216 | | | | | | | |
| 217 | 0.115 | | | | | | |
| 218 | | | | | | | |
| 219 | | | | | | | 0.064 |
| 220 | | 0.113 | | | | | |
| 221 | 0.115 | | | | | | |
| 222 | 0.23 | 0.113 | | | | | |
| 223 | 0.115 | | | | 0.366 | | |
| 224 | | | | | | | 0.128 |
| 225 | | | | | | | |
| 226 | 0.115 | | | | | | |
| 227 | | | | 0.195 | | | |
| 228 | 0.115 | | | | | | |
| 229 | | | | | | | |
| 230 | | | | | | | |
| 231 | | | | | | | |
| 232 | 0.115 | | | | | | |
| 233 | | | | 0.195 | | 0.188 | |
| 234 | | 0.113 | | | | | |
| 235 | | | | | | | |
| 236 | | | | | | 0.188 | |
| 237 | | | | | | | |
| 238 | | | | | | | |
| 239 | | | | | | | |
| 240 | 0.23 | | | | | | 0.064 |
| 241 | | | | | | | |
| 242 | | | | | | | 0.064 |
| 243 | | | | | | | 0.064 |
| 244 | 0.115 | | | 0.195 | | | |
| 245 | | | | | | | |
| 246 | | | | | | | |
| 247 | | | | | | | |
| 248 | | | | | | | |
| 249 | | | | | 1.097 | | |
| 250 | | | | | | | |
| 251 | | | | | | | |
| 252 | | | | | | | |
| 253 | | 0.227 | | | 0.366 | | 0.064 |
| 254 | | 0.227 | | | | | |
| # Biomarkers Per | 24 | 22 | 2 | 14 | 23 | 9 | 21 |
| Sample | | | | | | | |
| % Coverage | 8% | 7% | 1% | 4% | 7% | 3% | 7% |
| | Stage | | | | | | |
| | Braak V | Braak V | Braak V | Braak V | Braak V | Braak V | Braak V |
| Seq. ID | SRR1568522 | SRR1568573 | SRR1568638 | SRR1568642 | SRR1568665 | SRR1568667 | SRR1568673 |
| 47 | | | | 0.391 | | | |
| 48 | | | | 0.391 | | | |
| 49 | | | | 0.391 | | | |
| 50 | | | | | | | |
| 51 | | | | | | | |
| 52 | | | | | | | |
| 53 | | | | | | | |
| 54 | | | | 0.783 | | | |
| 55 | | | | | | | |
| 56 | | | | 1.566 | | | |
| 57 | | | | 0.783 | | | |
| 58 | | | | 0.391 | | | |
| 59 | | | 0.335 | | | | |
| 60 | | | | | | | |
| 61 | | | | | | | |
| 62 | | | 0.112 | | | | |
| 63 | | | | | | 0.418 | |
| 64 | | | | | | | |
| 65 | | | | | | | |
| 66 | | | | | | | |
| 67 | | | | | | | |
| 68 | 0.26 | | | | | | |
| 69 | 0.26 | | | | | | |
| 70 | | 0.081 | | | | | |
| 71 | | | | | | | |
| 72 | | 0.162 | | | | 0.084 | |
| 73 | | | 0.112 | | | 0.084 | |
| 74 | | | | | | | |
| 75 | | | | | | | |
| 76 | | | | | | | |
| 77 | | | 0.112 | | | | |
| 78 | | | | | | | |
| 79 | 0.13 | | | | | | |
| 80 | | | | 0.391 | | | |
| 81 | | | | | 0.074 | | |
| 82 | | | | 0.391 | | | |
| 83 | | | | | 0.148 | | |
| 84 | | | | | | | |
| 85 | | | | 0.391 | | | |
| 86 | | | | | | | |
| 87 | | | | | | | |
| 88 | | | | | | | |
| 89 | | | | | | | 0.127 |
| 90 | | | | 0.783 | 0.074 | | |
| 91 | 0.13 | | 4.798 | | | | |
| 92 | | | | | | | |
| 93 | | | | | | | |
| 94 | | | | | | | |
| 95 | | 0.081 | | | | 0.251 | |
| 96 | | | | | | | |
| 97 | 0.52 | | | | | | |
| 98 | | 0.081 | | | | 0.585 | |
| 99 | | 0.162 | | | | | |
| 100 | | | | | | | |
| 101 | | | | | | 0.418 | |
| 102 | | | | | | 0.251 | 0.127 |
| 103 | | 0.081 | | | | 0.251 | |
| 104 | 0.26 | | | | | | |
| 105 | | | | | | 0.335 | |
| 106 | | 0.162 | | | | 0.502 | |
| 107 | | 0.081 | | | | 0.167 | 0.127 |
| 108 | | | | | | | |
| 109 | 0.26 | | 0.335 | | | 0.167 | |
| 110 | | | 0.335 | | | | |
| 111 | | 0.081 | | | | 0.418 | |
| 112 | | | | | | | |
| 113 | | 0.081 | | | | 0.167 | |
| 114 | | | | | | 0.167 | |
| 115 | | | | | | | 0.381 |
| 116 | | | 0.112 | | | | |
| 117 | | | | | | | |
| 118 | | 0.162 | | | | | 0.381 |
| 119 | | | | | | | |
| 120 | | | | | | 0.251 | 0.127 |
| 121 | | | | | 0.148 | 0.084 | 0.127 |
| 122 | | | | | | | |
| 123 | 0.13 | | | | | | |
| 124 | | | | | | | |
| 125 | | 0.081 | | | | 0.167 | |
| 126 | | | | | | | 0.254 |
| 127 | | | | 0.391 | | | |
| 128 | 0.13 | | | | | | 0.254 |
| 129 | | | | | | 0.084 | 0.381 |
| 130 | | | | | | 0.167 | |
| 131 | 0.13 | | | | | | |
| 132 | | | | | | | |
| 133 | | | | | | | 0.127 |
| 134 | | | | | | 0.084 | |
| 135 | | | | | | | |
| 136 | | 0.081 | | | | 0.167 | |
| 137 | | | | | | 0.335 | 0.127 |
| 138 | | 0.081 | | | | 0.084 | |
| 139 | | | | | | | 0.254 |
| 140 | | | | | | | 0.127 |
| 141 | | | | | | | |
| 142 | | | | | | | |
| 143 | | | | | | 0.084 | |
| 144 | | 0.081 | | | | | 0.254 |
| 145 | | | | | | | |
| 146 | | | | | 0.074 | | |
| 147 | | | | | | 0.084 | |
| 148 | | | | | | | |
| 149 | | | 0.112 | | | | |
| 150 | 0.26 | | | | | | |
| 151 | | | | | | | |
| 152 | | | | | | | |
| 153 | | 0.081 | | | | | |
| 154 | | | | | | | |
| 155 | 0.13 | | | | | | |
| 156 | | | 0.335 | | | | |
| 157 | | 0.081 | | | | 0.167 | |
| 158 | | | | | | | |
| 159 | | | | | | | |
| 160 | | | | | | | |
| 161 | | 0.081 | | | | | |
| 162 | 0.13 | | | | | | |
| 163 | | 0.162 | | | 0.074 | | |
| 164 | | | | 0.391 | | | |
| 165 | | 0.081 | | | | | |
| 166 | 0.13 | 0.081 | | | | | |
| 167 | | | | | | | |
| 168 | | | | 0.391 | | | |
| 169 | | 0.081 | | | | 0.167 | |
| 170 | | | | 0.391 | | | |
| 171 | | | | | | | |
| 172 | | | | | | | |
| 173 | | | | | | | |
| 174 | | | | | | | |
| 175 | | | | | | | |
| 176 | 0.26 | | | 0.391 | | | |
| 177 | | | | | | 0.084 | |
| 178 | | | | | | 0.335 | |
| 179 | | | | | | | |
| 180 | | | | | | | |
| 181 | | | | | | | |
| 182 | | | | | | | |
| 183 | | | 0.112 | | | | |
| 184 | | | | | | 0.167 | |
| 185 | | | | | | | |
| 186 | | | | | | 0.084 | |
| 187 | | | 0.112 | | | | |
| 188 | | | | | | | 0.127 |
| 189 | | | | 1.566 | | | |
| 190 | | | | 0.391 | | | 0.127 |
| 191 | | 0.243 | | | | 0.418 | |
| 192 | 0.13 | | | | | | |
| 193 | 0.13 | | | | | | |
| 194 | | | | | | 0.167 | 0.127 |
| 195 | | | | | 0.074 | | |
| 196 | 0.13 | | | | | | |
| 197 | | | | | | | |
| 198 | | | | | | | |
| 199 | | | 0.112 | | | 0.084 | |
| 200 | | | | | | | 0.127 |
| 201 | | | | | | 0.084 | |
| 202 | | 0.243 | | | | | |
| 203 | | | | | | | |
| 204 | | | | | | 0.167 | |
| 205 | | | | | | 0.084 | |
| 206 | | | | | | | |
| 207 | | | 0.112 | | | 0.084 | |
| 208 | | | | | 0.074 | 0.084 | |
| 209 | | 0.081 | | | | | |
| 210 | | | | | | | |
| 211 | | | | | | | |
| 212 | | 0.081 | | | | 0.084 | |
| 213 | 0.26 | | | | | | |
| 214 | | | | | 0.074 | | 0.254 |
| 215 | | | | 0.783 | | 0.084 | |
| 216 | | | | | | 0.084 | |
| 217 | | | | | | | |
| 218 | | | | | | 0.084 | |
| 219 | 0.13 | | | | | | |
| 220 | | | | | | | |
| 221 | | 0.081 | | | | | 0.254 |
| 222 | | | | | | | |
| 223 | | 0.081 | | | | | |
| 224 | | | | | | | |
| 225 | | | | | | | |
| 226 | | 0.081 | | | | 0.084 | |
| 227 | | 0.081 | | | | | |
| 228 | | | | | | | |
| 229 | 0.13 | | | | | | 0.254 |
| 230 | 0.13 | | | | | | |
| 231 | | | | | | | |
| 232 | | | | | | | |
| 233 | | | | | | | |
| 234 | | | | | | | 0.127 |
| 235 | | 0.081 | | | | | |
| 236 | | 0.081 | | | | 0.084 | |
| 237 | | | 0.223 | | | | |
| 238 | | 0.081 | | | | | |
| 239 | | | | | | 0.084 | |
| 240 | 0.13 | 0.081 | | | | | |
| 241 | | | | | | | |
| 242 | 0.13 | 0.081 | | | | 0.084 | |
| 243 | | | | | | 0.084 | 0.127 |
| 244 | | | | | | | |
| 245 | | 0.081 | | | 0.074 | 0.084 | |
| 246 | | 0.162 | | | | 0.167 | |
| 247 | | | | | | | |
| 248 | 0.26 | | | | | 0.167 | |
| 249 | | | | | | | |
| 250 | | 0.081 | | | | 0.084 | |
| 251 | | 0.081 | | | | 0.084 | 0.127 |
| 252 | | | | | | 0.084 | 0.127 |
| 253 | 0.13 | | | | | | |
| 254 | | | | | | | |
| # of biomarkers | 26 | 39 | 15 | 19 | 10 | 55 | 26 |
| per sample | | | | | | | |
| % Coverage | 8% | 12% | 5% | 6% | 3% | 18% | 8% |
| | Stage | | | | | | |
| | Braak V | Braak V | Braak V | Braak VI | Braak VI | Braak VI | Braak VI |
| Seq. ID | SRR1568687 | SRR1568704 | SRR1568718 | SRR1568388 | SRR1568422 | SRR1568432 | SRR1568434 |
| 47 | | | | | | | |
| 48 | | | | | | | |
| 49 | | | | | | | |
| 50 | | | | | | | |
| 51 | | | | | | | |
| 52 | | | | | | | |
| 53 | | | | | 0.197 | | |
| 54 | | | | | | | |
| 55 | | | | | | | |
| 56 | | | | | | | |
| 57 | | | | | | | |
| 58 | | | | | | | |
| 59 | | | | | | | |
| 60 | | | | | | | |
| 61 | | 0.64 | | | | | |
| 62 | | | | | | | |
| 63 | | | | | | | |
| 64 | | 0.512 | | | | | |
| 65 | 0.311 | | | 0.597 | | | |
| 66 | | | | | 0.395 | | |
| 67 | | | | | | | |
| 68 | | | | | | | 0.098 |
| 69 | | | | | | | |
| 70 | | | | | | 0.274 | |
| 71 | | | 0.494 | | 0.395 | | |
| 72 | | | | | | | |
| 73 | | | | | | | |
| 74 | | | | | | | |
| 75 | | | | | | | |
| 76 | | | | | | | |
| 77 | | | | | | | |
| 78 | | | | | | | |
| 79 | | | | | | | |
| 80 | | | | | | | |
| 81 | | | | | | | |
| 82 | | | | | | | |
| 83 | | 0.128 | | | | | |
| 84 | | | | | | | |
| 85 | | | | | | | |
| 86 | | | | | | | |
| 87 | | | | | | | |
| 88 | | | | | | | |
| 89 | | | | | | | 0.098 |
| 90 | | | | | | | |
| 91 | | | | | | 0.137 | |
| 92 | | | | | | | |
| 93 | | | | | | | |
| 94 | 0.311 | | | | | | |
| 95 | | | | | | | |
| 96 | | | | 0.597 | | | |
| 97 | | | | | | 0.411 | 0.295 |
| 98 | | | | | | | |
| 99 | | | | | | | |
| 100 | | | | | | | |
| 101 | | | | | | | |
| 102 | | | 1.975 | | | | |
| 103 | | | | | | | |
| 104 | | | | | | 0.274 | 0.196 |
| 105 | | | | | | | |
| 106 | | | | | | | |
| 107 | | | | | | | |
| 108 | | | | | | | 0.393 |
| 109 | | | | | | | 0.295 |
| 110 | | | | 1.194 | | | |
| 111 | | | | | | | |
| 112 | | | | | 0.197 | | 0.098 |
| 113 | | | | | | | |
| 114 | | | | | | | |
| 115 | | | | | 0.197 | | |
| 116 | | | | | | | |
| 117 | | | | | | 0.137 | |
| 118 | | | | | | 0.137 | |
| 119 | | | | | | | |
| 120 | | | | | | | |
| 121 | | | | | | | |
| 122 | | | | | | | |
| 123 | | | | | | 0.137 | 0.098 |
| 124 | | 0.128 | | | | | |
| 125 | | | | | | | |
| 126 | | | | | 0.395 | 0.137 | |
| 127 | | | | 0.597 | | | |
| 128 | | | | | | | |
| 129 | | | | | | | |
| 130 | | | | | | | |
| 131 | | | | | | 0.274 | 0.098 |
| 132 | 0.311 | | | | | | |
| 133 | | | | | | | |
| 134 | | | | | | | |
| 135 | | 0.128 | | | | | |
| 136 | | | | | | | 0.196 |
| 137 | | | | | | | |
| 138 | | | | | | | |
| 139 | | | | | 0.197 | | 0.098 |
| 140 | | | | | | | 0.098 |
| 141 | | | | | 0.592 | | |
| 142 | | | | | | 0.137 | |
| 143 | | | | | | | |
| 144 | | | | | | | |
| 145 | | | | | | | |
| 146 | | | | | | | |
| 147 | | | | | | | |
| 148 | | | | | | | |
| 149 | | | | | | | |
| 150 | | | | | | 0.137 | 0.098 |
| 151 | | | | | 0.395 | 0.274 | 0.098 |
| 152 | | | | | | | |
| 153 | | | | | | | |
| 154 | | | | | | | |
| 155 | | | | | | 0.274 | 0.098 |
| 156 | | | | | | | 0.098 |
| 157 | | | | | | | |
| 158 | | | | | | 0.137 | |
| 159 | | | | | 0.395 | | |
| 160 | | 0.128 | | | | | |
| 161 | | 0.256 | | | | | |
| 162 | | | | | | 0.137 | |
| 163 | | | | | | | |
| 164 | | 0.256 | | | | | |
| 165 | | | | | | | 0.098 |
| 166 | | | | | | | |
| 167 | | | | | | | |
| 168 | | | | | | | |
| 169 | | | | | | | |
| 170 | | | | | | | |
| 171 | | | | | | | |
| 172 | 1.243 | | | | | | |
| 173 | 0.311 | | | | | | |
| 174 | | | | | | | |
| 175 | 0.311 | | | | | | |
| 176 | | | | 0.597 | | | |
| 177 | | | | | | | |
| 178 | | | | | | 0.137 | |
| 179 | | | | | | | |
| 180 | 0.311 | | | | | | |
| 181 | | 0.128 | | | | | 0.098 |
| 182 | 0.622 | | | | | | |
| 183 | | | | 0.597 | | | |
| 184 | 0.311 | | | | | | |
| 185 | | 0.128 | | | | 0.137 | |
| 186 | | 0.128 | | | | | |
| 187 | | | | | | | |
| 188 | | | | 0.597 | | | |
| 189 | | | | 0.597 | | | |
| 190 | | | | | | | |
| 191 | | | | | | | |
| 192 | | | | | | 0.411 | 0.491 |
| 193 | | | | | 0.986 | 0.137 | 0.196 |
| 194 | | | | | | | |
| 195 | | | | | | | |
| 196 | | | | | 0.197 | 0.274 | |
| 197 | | | | | 0.395 | | |
| 198 | | | | | | | |
| 199 | | | | | | | |
| 200 | | | | | | | |
| 201 | | | | | | | |
| 202 | | | | | | 0.137 | |
| 203 | | | | | | | |
| 204 | | | | | | | 0.098 |
| 205 | | 0.256 | | | | | 0.098 |
| 206 | 0.311 | | | | 0.592 | | |
| 207 | | | | 0.597 | | | |
| 208 | | 0.128 | | | | | |
| 209 | 0.311 | | | | | | |
| 210 | | | | | | 0.411 | 0.098 |
| 211 | | | | | | | |
| 212 | | | | | | | |
| 213 | | | | | | | |
| 214 | | | | 0.597 | | | |
| 215 | | | | | | | |
| 216 | | | | | | | 0.098 |
| 217 | 0.311 | | | | | | |
| 218 | 0.311 | | | | | | |
| 219 | | | | | | | |
| 220 | | 0.128 | | | | | |
| 221 | | 0.128 | | | | | |
| 222 | | | | | | | |
| 223 | | | | | | 0.137 | |
| 224 | | | | | 0.197 | 0.137 | 0.098 |
| 225 | | | | | 0.197 | 0.137 | 0.098 |
| 226 | | | | | | | |
| 227 | | | 0.494 | | | | |
| 228 | 1.554 | | | | | | |
| 229 | | | | | | 0.137 | 0.098 |
| 230 | | | 0.988 | | 0.197 | | |
| 231 | | 0.128 | | | | | 0.098 |
| 232 | | | | | | | 0.098 |
| 233 | | | | | | | |
| 234 | | | | | | | |
| 235 | | | | | 0.197 | | |
| 236 | | | | | | | |
| 237 | | | | 1.791 | | 0.137 | |
| 238 | | | | | | | |
| 239 | | | | | | | |
| 240 | | | | | | 0.137 | |
| 241 | | 0.128 | | 0.597 | | | |
| 242 | | | | | | | 0.098 |
| 243 | 0.311 | | | | | | |
| 244 | | 0.128 | | | 0.197 | | |
| 245 | | | | | | | |
| 246 | | | | | | 0.274 | |
| 247 | | 0.128 | | | | | |
| 248 | | | | | | 0.137 | 0.098 |
| 249 | | | 0.988 | | | | |
| 250 | | | 0.494 | | | | |
| 251 | | | | | | | 0.098 |
| 252 | | | | | | | |
| 253 | | 0.128 | | | | | |
| 254 | | 0.256 | | | | | |
| # Biomarkers Per | 15 | 21 | 6 | 12 | 19 | 30 | 32 |
| Sample | | | | | | | |
| % Coverage | 5% | 7% | 2% | 4% | 6% | 10% | 10% |
| | Stage | | | | | | |
| | Braak VI | Braak VI | Braak VI | Braak VI | Braak VI | Braak VI | Braak VI |
| Seq. ID | SRR1568440 | SRR1568456 | SRR1568489 | SRR1568495 | SRR1568524 | SRR1568529 | SRR1568537 |
| 47 | | | | | | | 1.539 |
| 48 | | | | | | | 2.694 |
| 49 | | | | | | | |
| 50 | | | | | | | 0.385 |
| 51 | | | | | | | 0.385 |
| 52 | | | | | | | |
| 53 | | | | | | | |
| 54 | | | | | | | 0.77 |
| 55 | | | | 0.189 | | | |
| 56 | | | | | | | |
| 57 | | | | | | | 1.924 |
| 58 | | | | | | | 4.233 |
| 59 | | | | | | | |
| 60 | | | | | | | |
| 61 | 0.177 | | | | 0.665 | | |
| 62 | | | | | | | 0.385 |
| 63 | | | | | | | |
| 64 | 0.53 | | | | 0.133 | | |
| 65 | | | | | | | |
| 66 | | | | | | | |
| 67 | 0.177 | | | | | | |
| 68 | | | | | | | |
| 69 | | | | | | | |
| 70 | | | | | | | |
| 71 | | 0.051 | | | | | |
| 72 | | 0.101 | | | | | |
| 73 | | | | | | | |
| 74 | | | | | | | |
| 75 | | | | | | | |
| 76 | | | | | | | |
| 77 | | 0.101 | | | | | |
| 78 | | | | | | | 0.77 |
| 79 | | 0.101 | | | | | |
| 80 | | | | | | | |
| 81 | | | | | 0.133 | | |
| 82 | | | | | | | 0.385 |
| 83 | | | | | 0.133 | | |
| 84 | | | | | | | |
| 85 | | | | | | | |
| 86 | 0.353 | | | | 0.133 | 0.205 | |
| 87 | | | | | | | |
| 88 | | | | | | | |
| 89 | | | | | | | |
| 90 | | | | | | | |
| 91 | 0.177 | | | | | | |
| 92 | | 1.216 | | | | | |
| 93 | | | | | | | |
| 94 | | | | | | | |
| 95 | | 0.355 | | | 0.133 | | |
| 96 | | | | | | | 0.77 |
| 97 | | | | | | | |
| 98 | | 0.152 | | | | | |
| 99 | | 0.152 | | | | | |
| 100 | | | | | | | 0.77 |
| 101 | 0.353 | 0.101 | | 0.284 | | | |
| 102 | | | | | | | |
| 103 | 0.177 | 0.253 | | | | | |
| 104 | | | | | | | |
| 105 | 0.177 | 0.203 | | 0.189 | | | |
| 106 | | 0.051 | | | | | |
| 107 | | 0.051 | | | | | |
| 108 | | | | | | | |
| 109 | | | | | | | |
| 110 | | | | | | | |
| 111 | | 0.051 | | 0.189 | | | |
| 112 | | | | | | | |
| 113 | | 0.203 | | | | | |
| 114 | | 0.203 | | | | | |
| 115 | | | | | | | |
| 116 | | | | | | | |
| 117 | | | | | | | |
| 118 | | 0.101 | | | | | |
| 119 | | | | 0.095 | | | |
| 120 | | 0.051 | | | 0.266 | | |
| 121 | | | | | | | |
| 122 | | | | | | | |
| 123 | | | | | | | |
| 124 | | | | | 0.399 | 0.205 | |
| 125 | | | | | | | |
| 126 | | 0.051 | | | | | |
| 127 | | | | | | | 0.385 |
| 128 | | | | | 0.133 | | |
| 129 | | | | 0.095 | | | |
| 130 | | | | | | | |
| 131 | | | | | | | |
| 132 | | 0.051 | | | | | |
| 133 | | | | | | | |
| 134 | | | | | | | |
| 135 | | | 0.423 | | | | |
| 136 | | | | | | | |
| 137 | | | | | | | |
| 138 | 0.353 | 0.051 | | 0.095 | | | |
| 139 | | | | | | | |
| 140 | | 0.101 | | | | | |
| 141 | | | | | 0.133 | | |
| 142 | | 0.101 | | | | | |
| 143 | | | | | | | |
| 144 | | 0.051 | | | | | |
| 145 | | | | | | 0.205 | |
| 146 | 0.177 | | | 0.189 | | | |
| 147 | | 0.152 | | | 0.133 | | |
| 148 | | | | 0.095 | | | 0.385 |
| 149 | | | | | | | |
| 150 | | | | | | | |
| 151 | 0.177 | | | | | | |
| 152 | | | | | | | |
| 153 | | | | | | | |
| 154 | | | | | | | |
| 155 | | | | | | | |
| 156 | | | | | | | |
| 157 | 0.177 | | | 0.095 | | | |
| 158 | | 0.101 | | | | | |
| 159 | | | | | | | |
| 160 | | | | | | | |
| 161 | | 0.051 | | | | | |
| 162 | | | | | | | |
| 163 | | | | | | | |
| 164 | | | | | 0.266 | | |
| 165 | | 0.051 | | | | | |
| 166 | | | | | | 0.205 | |
| 167 | | | | | | | |
| 168 | | | | | | | 0.385 |
| 169 | | | | 0.095 | | | |
| 170 | | | | 0.095 | | | 0.385 |
| 171 | 0.353 | | | | 0.665 | | |
| 172 | | | | | | | |
| 173 | | | | | | | |
| 174 | | 0.051 | | | | | |
| 175 | 0.177 | | | | | | |
| 176 | | | | | | | 0.385 |
| 177 | 0.177 | | | | 0.133 | | |
| 178 | | | | | | | |
| 179 | 0.177 | | | 0.095 | | | |
| 180 | | | | | | | |
| 181 | | | | | | | |
| 182 | | | | | 0.133 | | |
| 183 | | | | | | | |
| 184 | 0.177 | | | | | | |
| 185 | | 0.051 | | | 0.133 | | |
| 186 | | | | | | | |
| 187 | | | | | | | |
| 188 | | | | 0.095 | | | |
| 189 | | | | | | | 5.002 |
| 190 | | 0.709 | | 0.095 | | | |
| 191 | 0.177 | 0.101 | | | | | |
| 192 | | | | | | | |
| 193 | | | | | | | |
| 194 | | | | | | | |
| 195 | | | | 0.095 | 0.133 | | |
| 196 | | | | | | | |
| 197 | | | | 0.095 | | 0.205 | |
| 198 | 0.177 | 0.051 | | | | 0.616 | |
| 199 | | | | | | | |
| 200 | | 0.203 | | | | | |
| 201 | | 0.051 | | | | | |
| 202 | | | | | | 0.205 | |
| 203 | | 0.051 | | | | | |
| 204 | | 0.051 | | | | | |
| 205 | | | | 0.095 | | 0.411 | |
| 206 | | 0.051 | | 0.095 | 0.133 | | |
| 207 | | | | | | | |
| 208 | | | | | 0.266 | | |
| 209 | | 0.051 | | | | | |
| 210 | | | | | | | |
| 211 | | 0.101 | | 0.095 | | | |
| 212 | | 0.051 | | 0.095 | | | |
| 213 | | | | | 0.133 | 0.205 | |
| 214 | | | | | | | |
| 215 | | | | | | | |
| 216 | | | | | | | |
| 217 | 0.177 | | | | | | |
| 218 | | | | | | | |
| 219 | | | | 0.189 | | | |
| 220 | | | | 0.095 | 0.133 | | |
| 221 | | | | 0.095 | | | |
| 222 | | | | 0.095 | | 0.205 | |
| 223 | | | | | | | |
| 224 | | | 0.141 | | | | |
| 225 | | | | | | | |
| 226 | 0.177 | | | | 0.266 | 0.205 | |
| 227 | | 0.051 | | 0.189 | | | |
| 228 | | | | | | | |
| 229 | | | | | | | |
| 230 | | | | 0.095 | | | |
| 231 | | | | | | | |
| 232 | | 0.051 | | | | | |
| 233 | 0.177 | | | | | | |
| 234 | 0.177 | | | | | | |
| 235 | | 0.051 | | | | 0.205 | |
| 236 | | 0.152 | | | | | |
| 237 | | | | | | | 0.77 |
| 238 | | | | | | | |
| 239 | | 0.051 | | | | | |
| 240 | | | | 0.095 | | | |
| 241 | 0.177 | | | 0.095 | | 0.205 | |
| 242 | | | | | | | |
| 243 | | 0.051 | | | | | |
| 244 | 0.177 | | | | | | |
| 245 | | | | | | | |
| 246 | | | | 0.095 | | | |
| 247 | | | | | 0.266 | | |
| 248 | | | | | | | |
| 249 | | | | 0.095 | | | |
| 250 | | 0.101 | | 0.473 | | | |
| 251 | | 0.051 | | | | | |
| 252 | | | | | 0.266 | | |
| 253 | | 0.051 | | | 0.133 | | |
| 254 | 0.177 | | | | 0.266 | | |
| # Biomarkers Per | 26 | 50 | 2 | 32 | 26 | 13 | 19 |
| Sample | | | | | | | |
| % Coverage | 8% | 16% | 1% | 10% | 8% | 4% | 6% |
| | Stage | | | | | | |
| | Braak VI | Braak VI | Braak VI | Braak VI | Braak VI | Braak VI | Braak VI |
| Seq. ID | SRR1568539 | SRR1568561 | SRR1568565 | SRR1568599 | SRR1568610 | SRR1568640 | SRR1568647 |
| 47 | | | | | | | |
| 48 | | | | | | | |
| 49 | | | | | | | |
| 50 | | | | | | | |
| 51 | | | | | | | |
| 52 | | | | 0.111 | | | |
| 53 | | | | | | | |
| 54 | | | | | | | |
| 55 | | | | | | | |
| 56 | | | | | | | |
| 57 | | | | | | | |
| 58 | | | | | | | |
| 59 | | | | 0.223 | | | 0.273 |
| 60 | | 0.453 | | | 0.547 | 0.96 | |
| 61 | | | | | | | |
| 62 | | | | | | | |
| 63 | | | | | | | 0.273 |
| 64 | | | | | | | |
| 65 | | | | | | | |
| 66 | | | | | | | |
| 67 | | | 0.654 | | 0.274 | | |
| 68 | | | | | | | |
| 69 | | 0.091 | | | | | 0.547 |
| 70 | | | | | | | |
| 71 | | | | | | | |
| 72 | | 0.181 | | | | | |
| 73 | | | | | | | |
| 74 | | | | | | | 0.273 |
| 75 | | | | | | | |
| 76 | | | 0.164 | | | | |
| 77 | | | | 0.111 | | | |
| 78 | | | | | | | |
| 79 | | | | | | | |
| 80 | | | | | | | |
| 81 | | | | | | 0.16 | |
| 82 | | | | | | | |
| 83 | | | | | | | 0.273 |
| 84 | | | | | | 0.16 | 0.547 |
| 85 | | | | 0.111 | 0.274 | | |
| 86 | | | | | | | |
| 87 | | | | 0.111 | | | |
| 88 | | | | | | | |
| 89 | | | | 0.223 | | | |
| 90 | | | | | | | |
| 91 | | | | | | | |
| 92 | | | | | | | 0.273 |
| 93 | | 0.181 | 0.164 | | 0.274 | | |
| 94 | | | | | | | |
| 95 | | | | | | | |
| 96 | | | | | | | |
| 97 | | | | | | | |
| 98 | | | | | | | |
| 99 | | | | | | | |
| 100 | | | | | | | |
| 101 | | | | | | | |
| 102 | | | | | | 0.64 | |
| 103 | | 0.091 | | | | | |
| 104 | | | | | | | |
| 105 | | | | | | | |
| 106 | | | | 0.111 | | 0.16 | |
| 107 | | | | | | | |
| 108 | | | | | | | |
| 109 | | | | | | | |
| 110 | | | | | | | |
| 111 | | | | | | | |
| 112 | | | | | | | |
| 113 | | 0.091 | | | | | |
| 114 | 0.166 | | | | | | |
| 115 | 0.166 | | | 0.334 | | | |
| 116 | | 0.091 | | | | | 0.273 |
| 117 | | | | 0.223 | | 0.16 | |
| 118 | | | | | | | 0.273 |
| 119 | | | 0.164 | | | 0.16 | |
| 120 | | | | | | 0.32 | |
| 121 | | 0.363 | | | | | |
| 122 | | 0.181 | | 0.111 | | | |
| 123 | | | | | | 0.16 | |
| 124 | | | | | | | |
| 125 | | | | | 0.274 | 0.48 | |
| 126 | | | | | | | |
| 127 | | | | | | | |
| 128 | | 0.091 | | | | 0.16 | |
| 129 | | | | | | | |
| 130 | | 0.091 | | 0.111 | | | |
| 131 | | | 0.327 | | | | |
| 132 | | | | | | | |
| 133 | | 0.091 | | 0.334 | | | 0.273 |
| 134 | | | | | | | |
| 135 | | 0.091 | | | | | |
| 136 | | | | | | 0.32 | |
| 137 | | 0.091 | | 0.111 | | | |
| 138 | | | | | | | |
| 139 | | 0.181 | | | | | |
| 140 | | | | | | 0.16 | |
| 141 | | | | | | | |
| 142 | 0.166 | | | 0.223 | | | |
| 143 | | | | | 0.274 | 0.16 | |
| 144 | | | | | | | |
| 145 | | | | 0.334 | | 0.16 | |
| 146 | | | | 0.111 | | 0.16 | |
| 147 | | 0.091 | | | | | |
| 148 | | | | | | 0.16 | |
| 149 | | | | | | | |
| 150 | | | | | | 0.16 | |
| 151 | | | | | | | |
| 152 | | | 0.327 | 0.111 | 0.547 | | |
| 153 | | 0.091 | | | | | 0.273 |
| 154 | | | | 0.223 | 0.547 | | |
| 155 | | | | | | | |
| 156 | | | | | | 0.16 | |
| 157 | | | | | | | |
| 158 | | | | 0.111 | | 0.32 | |
| 159 | | | | 0.223 | | | |
| 160 | | 0.091 | | | | | 0.273 |
| 161 | | | | | | | |
| 162 | | | | | | 0.16 | |
| 163 | | 0.091 | | | | | |
| 164 | | | 0.164 | | | | |
| 165 | | | | | | | |
| 166 | | | | | | | |
| 167 | | 0.091 | | | 0.547 | | |
| 168 | | | | | | | |
| 169 | | | | | | | |
| 170 | | | | 0.111 | | | |
| 171 | | | | | | | |
| 172 | | | | | | | |
| 173 | | | | | | | |
| 174 | | 0.272 | | | 0.274 | | 0.273 |
| 175 | | | | | | | |
| 176 | | 0.091 | | | | | |
| 177 | | | | 0.334 | | | |
| 178 | | | | | | | |
| 179 | | 0.181 | | | | | |
| 180 | | | | | | | |
| 181 | | | | 0.223 | | | |
| 182 | | | | 0.111 | | | |
| 183 | | 0.272 | | 0.111 | | | |
| 184 | | | | | | | |
| 185 | | | | | | | |
| 186 | | | | | | | |
| 187 | | 0.091 | 0.164 | | | | |
| 188 | | 0.091 | | | | | |
| 189 | | | | | | | |
| 190 | | | | | | | |
| 191 | | | | | | | |
| 192 | | | | | | 0.32 | |
| 193 | | | | | | | |
| 194 | | | | | | | |
| 195 | | | | | | 0.8 | |
| 196 | | | | | | 0.16 | |
| 197 | | | | 0.111 | | | |
| 198 | | | | | 0.274 | | |
| 199 | | | | 0.334 | | | |
| 200 | | | | | 0.274 | | |
| 201 | | | 0.164 | | | 0.48 | 0.273 |
| 202 | | | | | | | |
| 203 | | | 0.164 | | 0.274 | | |
| 204 | 0.166 | | | | | 0.16 | |
| 205 | | | | | | | |
| 206 | | | | | | 0.16 | |
| 207 | | | | | | | |
| 208 | | | | | | | |
| 209 | | | | | | | |
| 210 | 0.166 | | 0.164 | | | | |
| 211 | | | | | | | |
| 212 | | | | | | | |
| 213 | | | | | | | |
| 214 | | | | | 0.274 | | 0.547 |
| 215 | | | 0.327 | | | | |
| 216 | | | | | | 0.32 | 0.273 |
| 217 | | | | | | | |
| 218 | | | | | | | |
| 219 | | | 0.164 | | | 0.16 | |
| 220 | | 0.091 | | | | | |
| 221 | | | | | | | |
| 222 | | 0.091 | | 0.111 | | | |
| 223 | | | | | | | |
| 224 | | | | | | | |
| 225 | | | | | | 0.32 | |
| 226 | | | | | | | |
| 227 | | | | | | | |
| 228 | | | | | | | |
| 229 | | | | | | 0.16 | |
| 230 | | | | 0.111 | | | |
| 231 | 0.166 | | | | | 0.16 | 0.273 |
| 232 | 0.166 | 0.091 | | 0.111 | | | |
| 233 | | | | 0.111 | | | |
| 234 | 0.166 | | | 0.111 | | | |
| 235 | | | 0.491 | | 0.821 | | |
| 236 | | 0.091 | | | | | |
| 237 | | | | | | | |
| 238 | | | | 0.111 | | 0.16 | |
| 239 | | | | 0.111 | | 0.48 | 0.273 |
| 240 | | | | | | | |
| 241 | | | | | | | |
| 242 | | | | | | 0.16 | |
| 243 | | | | | | | |
| 244 | | 0.091 | | | | | |
| 245 | | 0.091 | | 0.111 | | | |
| 246 | | | | | 0.274 | | |
| 247 | | | 0.164 | | | 0.16 | |
| 248 | | 0.091 | | 0.223 | | 0.16 | |
| 249 | | | | 0.223 | | | 0.547 |
| 250 | | 0.091 | | | | | |
| 251 | | | | | | | 0.273 |
| 252 | | | | 0.111 | 0.274 | | |
| 253 | 0.333 | 0.091 | 0.164 | | | | |
| 254 | 0.166 | | 0.164 | 0.334 | | | |
| # Biomarkers Per | 10 | 35 | 17 | 39 | 17 | 37 | 20 |
| Sample | | | | | | | |
| % Coverage | 3% | 11% | 5% | 12% | 5% | 12% | 6% |

| | Stage | | | | | | | |
|---|---|---|---|---|---|---|---|---|
| | Braak VI | Braak VI | Braak VI | Braak VI | Braak VI | Braak VI | Braak VI | Braak VI |
| Seq. ID | SRR1568661 | SRR1568663 | SRR1568672 | SRR1568677 | SRR1568722 | SRR1568740 | SRR1568747 | SRR1568755 |
| 47 | | | | | | | | |
| 48 | | | | | | | | |
| 49 | | | | 0.475 | | | | |
| 50 | | | | | | | | |
| 51 | | | | | | | | |
| 52 | | | | | | | | |
| 53 | | | | | | | | |
| 54 | | | | | | | | |
| 55 | | | | | | | 6.415 | |
| 56 | | | | | | | | |
| 57 | | | | | | | | |
| 58 | | | | | | | | |
| 59 | | | | | | | | |
| 60 | | | | | | | | |
| 61 | | | | | | | | |
| 62 | | | | | | | | |
| 63 | | | | | | | | |
| 64 | | | | | | | | |
| 65 | | | | 0.95 | 0.562 | | | |
| 66 | | | | | | | | |
| 67 | | | | | | | | |
| 68 | | | | | | | | 0.173 |
| 69 | | | | | | | | 0.086 |
| 70 | | | | | 0.562 | | | 0.259 |
| 71 | | | | | | | | |
| 72 | | | | | | | | |
| 73 | | | | 0.475 | | | | |
| 74 | | | | | | | | |
| 75 | 0.672 | | | | | | | |
| 76 | | | 0.11 | | | 0.233 | | |
| 77 | | | | | | | | 0.086 |
| 78 | | | | | | | | |
| 79 | | | 0.11 | | | | | |
| 80 | | | | | | | | |
| 81 | | | | | | | | |
| 82 | | | | | | | | 0.259 |
| 83 | | | | | | | | |
| 84 | | | 0.11 | | | | | |
| 85 | | | | | | | 0.238 | |
| 86 | | | | | | | | |
| 87 | | | | | 0.562 | | | |
| 88 | 0.672 | | | | 0.562 | | | |
| 89 | | | 0.11 | | | | | |
| 90 | | | 0.22 | | | | | |
| 91 | | | | | | | | 0.173 |
| 92 | | | | | | | | |
| 93 | | | | | | | | |
| 94 | | | | | 0.562 | | | |
| 95 | | | | | | | | |
| 96 | | | | | | | | |
| 97 | | | | | | | | 0.259 |
| 98 | | | | | | | | 0.086 |
| 99 | | | | | | | | 0.086 |
| 100 | | | | | | | | |
| 101 | | | | | | | | |
| 102 | | | 0.11 | | | | | |
| 103 | | | | | | | | |
| 104 | | | | | | | | |
| 105 | | | | | | | | |
| 106 | | | | | | | | |
| 107 | | | | | | | | |
| 108 | | | | | | | | |
| 109 | | | | | | | | |
| 110 | | | | 0.95 | | | | |
| 111 | | | | | | | | |
| 112 | | | | | | | | |
| 113 | | | | | | | | |
| 114 | | | | | | | | 0.173 |
| 115 | | | | | | | | |
| 116 | | | | | | | | |
| 117 | | | 0.11 | | | | | |
| 118 | | | | | | | | |
| 119 | | | | | | | | |
| 120 | | | | | | | | |
| 121 | | | | | | | | |
| 122 | 0.672 | | | | | | | |
| 123 | | | | | | | | |
| 124 | | | | | | | | |
| 125 | | | | | | | | |
| 126 | | 0.2 | | | | | | |
| 127 | | | | | | | | |
| 128 | | | | | | | | |
| 129 | | | | | | | | |
| 130 | | | | | | | | |
| 131 | | | | | | | | |
| 132 | 0.672 | | | | 2.248 | | | |
| 133 | | | | | | | | |
| 134 | | | | 0.475 | | | | |
| 135 | 0.672 | | | | | | | |
| 136 | | | | | | | | |
| 137 | | | | | | | | |
| 138 | | | | | | | | |
| 139 | | | | | | | | |
| 140 | | | | | | | | |
| 141 | | | 0.11 | 0.475 | | | | |
| 142 | | | | | | | | |
| 143 | | | | | | | | |
| 144 | | 0.2 | | | | | | 0.173 |
| 145 | | | 0.11 | | | | | |
| 146 | | | | | | | | |
| 147 | | | | | | | | |
| 148 | | | | | | | | |
| 149 | | | | | | | | 0.086 |
| 150 | | | | | | | | 0.086 |
| 151 | | | | | | | | |
| 152 | | | | | | | 0.238 | |
| 153 | | | | | | | 0.475 | |
| 154 | | | 0.11 | | | | | 0.086 |
| 155 | | | | | | | | 0.086 |
| 156 | | | | | | | | |
| 157 | | | | | | | | |
| 158 | | | | | | | | |
| 159 | | | | | 0.562 | | | |
| 160 | | | | | | | | 0.173 |
| 161 | | | 0.22 | | | | | 0.086 |
| 162 | | | | | | | | 0.086 |
| 163 | | | 0.11 | 0.475 | | | | |
| 164 | | | | | | | | |
| 165 | | | 0.11 | | | | | |
| 166 | | | | | | | | |
| 167 | | | | | | 0.233 | | |
| 168 | | | | | | | | |
| 169 | | | | | | | | |
| 170 | | | | | | | | |
| 171 | | | | | | | | |
| 172 | 0.672 | | | 0.475 | | | | |
| 173 | 0.672 | | | 0.95 | 1.124 | | | |
| 174 | | | | | | | | |
| 175 | 1.345 | | | | | | | |
| 176 | | | | | | | | |
| 177 | | | | | | | | |
| 178 | | | | | | | | |
| 179 | | | | | | | | |
| 180 | 0.672 | | | | | | | |
| 181 | | | | | | | | |
| 182 | 1.345 | | | 0.475 | | | | |
| 183 | | | | | | | | |
| 184 | | | | | | | | 0.086 |
| 185 | | | 0.11 | | | | | |
| 186 | | 0.2 | 0.11 | | | | | |
| 187 | | | | | | | | |
| 188 | | | | | | | | |
| 189 | | | | | | | | 0.086 |
| 190 | | | 0.11 | | | | | 0.086 |
| 191 | | | | | | | | |
| 192 | | | | | | | | 0.086 |
| 193 | | | | | | | | 0.086 |
| 194 | | 0.599 | | 0.475 | | | | 0.173 |
| 195 | | | | | | | | |
| 196 | | | | | | | | 0.431 |
| 197 | | | | | | | | |
| 198 | | | | | | | | |
| 199 | | | | | 1.124 | | | 0.173 |
| 200 | | | 0.11 | | | | | 0.086 |
| 201 | | | | | | | | |
| 202 | | | 0.22 | | | | 0.238 | |
| 203 | | | | | | | | |
| 204 | | | | | | | | |
| 205 | | | | | | | | |
| 206 | | | | | | | | |
| 207 | | | | | | | | |
| 208 | | | | | | | | 0.086 |
| 209 | | | | | 0.562 | | 0.238 | |
| 210 | | | | | | | 0.238 | |
| 211 | | | 0.22 | | | | | 0.086 |
| 212 | | | | | | | | |
| 213 | | | | | | | | 0.086 |
| 214 | | | | | | | | |
| 215 | | | | | | 0.233 | | |
| 216 | | 0.2 | | | | | | 0.086 |
| 217 | 0.672 | | | | 0.562 | | | |
| 218 | | | | | | | | |
| 219 | | | | | | | | |
| 220 | | | | | | | | |
| 221 | | | 0.11 | | | | | |
| 222 | | | | | | | | |
| 223 | | | | | 0.562 | | 0.238 | |
| 224 | | | 0.11 | | | | | |
| 225 | | | | | | | | 0.086 |
| 226 | | | | | | | | |
| 227 | | | | | | | | |
| 228 | 4.035 | | | 0.95 | 0.562 | | | |
| 229 | | | | | | 0.233 | | |
| 230 | | | | | | | | |
| 231 | | | | | | | | |
| 232 | | | | | | | | |
| 233 | | | 0.11 | | | | | |
| 234 | | | | | | | | |
| 235 | | | | | | | | 0.086 |
| 236 | | | | | | | | |
| 237 | | | | | | | | |
| 238 | | 0.2 | | | | | | 0.431 |
| 239 | | 0.2 | | | | | | 0.086 |
| 240 | | | | | | | | |
| 241 | | 0.2 | | | | | | |
| 242 | | | | | | 0.233 | | |
| 243 | | | | | | | | |
| 244 | | | | | | | | 0.086 |
| 245 | | | | | | | 0.238 | |
| 246 | | 0.399 | | | | | | |
| 247 | | 0.2 | | | | 0.233 | | |
| 248 | | 0.798 | | | | | | |
| 249 | | | | | | | | |
| 250 | | | | | | | | 0.086 |
| 251 | | | | | | | 0.475 | 0.086 |
| 252 | | | | | | | | 0.086 |
| 253 | | | | | | | | |
| 254 | | | 0.11 | | | | | |
| # Biomarkers | 12 | 11 | 23 | 12 | 13 | 6 | 10 | 39 |
| Per Sample | | | | | | | | |
| % Coverage | 4% | 4% | 7% | 4% | 4% | 2% | 3% | 12% |

TABLE 5
Identified sRNA biomarkers in cerebrospinal fluid that have a positive
correlation with Braak Stage in order to monitor Alzheimer's Disease

| | Total | Braak | Braak | Braak | Braak | Braak | | Frequency |
|---|---|---|---|---|---|---|---|---|
| Seq. ID | Reads | II Avg | III Avg | IV Avg | V Avg | VI Avg | Hits | (Sensitivity) |
| 58 | 21 | 0.000 | 0.386 | 0.542 | 0.660 | 4.233 | 4 | 9.38% |
| 189 | 26 | 0.000 | 0.000 | 0.643 | 1.149 | 1.895 | 3 | 10.94% |
| 78 | 8 | 0.000 | 0.129 | 0.365 | 0.366 | 0.770 | 4 | 9.38% |
| 172 | 15 | 0.000 | 2.319 | 1.752 | 0.607 | 0.574 | 4 | 10.94% |
| 193 | 14 | 0.000 | 0.000 | 0.143 | 0.161 | 0.351 | 3 | 10.94% |
| 97 | 15 | 0.000 | 0.000 | 0.143 | 0.292 | 0.322 | 3 | 9.38% |
| 122 | 10 | 0.000 | 0.000 | 0.195 | 0.262 | 0.321 | 3 | 9.38% |
| 215 | 9 | 0.000 | 0.000 | 0.475 | 0.352 | 0.280 | 3 | 10.94% |
| 248 | 15 | 0.000 | 0.000 | 0.143 | 0.214 | 0.251 | 3 | 14.06% |
| 164 | 8 | 0.000 | 0.000 | 0.377 | 0.253 | 0.215 | 3 | 9.38% |
| 120 | 10 | 0.000 | 0.000 | 0.145 | 0.189 | 0.212 | 3 | 9.38% |
| 93 | 22 | 0.000 | 0.000 | 2.208 | 0.366 | 0.206 | 3 | 9.38% |
| 126 | 9 | 0.000 | 0.000 | 0.614 | 0.254 | 0.196 | 3 | 9.38% |
| 253 | 13 | 0.000 | 0.000 | 0.377 | 0.183 | 0.154 | 3 | 17.19% |
| 112 | 11 | 0.000 | 0.000 | 3.673 | 0.323 | 0.148 | 3 | 9.38% |
| 144 | 8 | 0.000 | 0.000 | 0.298 | 0.168 | 0.141 | 3 | 9.38% |
| 213 | 9 | 0.000 | 0.000 | 0.286 | 0.155 | 0.141 | 3 | 10.94% |
| 244 | 8 | 0.000 | 0.000 | 0.195 | 0.146 | 0.138 | 3 | 12.50% |
| 123 | 10 | 0.000 | 0.000 | 0.572 | 0.129 | 0.132 | 3 | 9.38% |
| 222 | 8 | 0.000 | 0.000 | 0.195 | 0.172 | 0.126 | 3 | 10.94% |
| 150 | 8 | 0.000 | 0.000 | 0.286 | 0.260 | 0.120 | 3 | 9.38% |
| 240 | 9 | 0.000 | 0.000 | 0.735 | 0.129 | 0.116 | 3 | 12.50% |
| 52 | 8 | 1.126 | 1.740 | 0.544 | 0.277 | 0.111 | 5 | 9.38% |
| 220 | 8 | 0.000 | 0.000 | 0.267 | 0.121 | 0.106 | 3 | 10.94% |
| 221 | 8 | 0.000 | 0.000 | 0.584 | 0.145 | 0.103 | 3 | 10.94% |
| 169 | 10 | 1.126 | 0.580 | 0.430 | 0.177 | 0.095 | 5 | 10.94% |
| 165 | 8 | 0.000 | 0.000 | 1.132 | 0.135 | 0.086 | 3 | 9.38% |
| 212 | 9 | 0.000 | 0.000 | 0.195 | 0.170 | 0.073 | 3 | 10.94% |

TABLE 6A
Experimental Alzheimer's disease cohort for
biomarker discovery, taken from serum samples.

| | | | | Age at | Disease | Braak |
|---|---|---|---|---|---|---|
| Group | SRR ID | DiseaseType | Gender | Death | Durration | score |
| Experimental | SRR1568369 | Alzheimer's | 1 | 87 | 12 | V |
| Experimental | SRR1568371 | Alzheimer's | 1 | 86 | 21 | V |
| Experimental | SRR1568407 | Alzheimer's | 1 | 75 | 10 | V |
| Experimental | SRR1568409 | Alzheimer's | 1 | 76 | 2 | V |
| Experimental | SRR1568411 | Alzheimer's | 1 | 67 | 9 | V |
| Experimental | SRR1568421 | Alzheimer's | 2 | 77 | 3 | IV |
| Experimental | SRR1568433 | Alzheimer's | 2 | 60 | 5 | VI |
| Experimental | SRR1568435 | Alzheimer's | 2 | 74 | 12 | VI |
| Experimental | SRR1568437 | Alzheimer's | 2 | 88 | 3 | IV |
| Experimental | SRR1568446 | Alzheimer's | 1 | 76 | 4 | V |
| Experimental | SRR1568455 | Alzheimer's | 1 | 80 | 8 | V |
| Experimental | SRR1568468 | Alzheimer's | 1 | 75 | 7 | V |
| Experimental | SRR1568475 | Alzheimer's | 2 | 86 | 9 | V |
| Experimental | SRR1568481 | Alzheimer's | 2 | 75 | 5 | V |
| Experimental | SRR1568490 | Alzheimer's | 2 | 70 | 4 | VI |
| Experimental | SRR1568496 | Alzheimer's | 2 | 74 | 8 | VI |
| Experimental | SRR1568515 | Alzheimer's | 2 | 78 | 8 | V |
| Experimental | SRR1568523 | Alzheimer's | 2 | 87 | 5 | V |
| Experimental | SRR1568525 | Alzheimer's | 2 | 70 | 5 | VI |
| Experimental | SRR1568530 | Alzheimer's | 2 | 57 | 10 | VI |
| Experimental | SRR1568534 | Alzheimer's | 2 | 86 | NA | IV |
| Experimental | SRR1568538 | Alzheimer's | 2 | 65 | 3 | VI |
| Experimental | SRR1568541 | Alzheimer's | 2 | 91 | 10 | IV |
| Experimental | SRR1568547 | Alzheimer's | 2 | 91 | 19 | II |
| Experimental | SRR1568553 | Alzheimer's | 1 | 79 | 5 | II |
| Experimental | SRR1568557 | Alzheimer's | 1 | 90 | 1 | III |
| Experimental | SRR1568562 | Alzheimer's | 1 | 87 | 6 | VI |
| Experimental | SRR1568566 | Alzheimer's | 1 | 78 | 5 | VI |
| Experimental | SRR1568580 | Alzheimer's | 1 | 86 | 4 | II |
| Experimental | SRR1568586 | Alzheimer's | 2 | 89 | 9 | IV |
| Experimental | SRR1568598 | Alzheimer's | 1 | 82 | 12 | VI |
| Experimental | SRR1568600 | Alzheimer's | 1 | 85 | 5 | VI |
| Experimental | SRR1568611 | Alzheimer's | 2 | 68 | 8 | VI |
| Experimental | SRR1568623 | Alzheimer's | 1 | 90 | NA | V |
| Experimental | SRR1568639 | Alzheimer's | 1 | 75 | 6 | V |
| Experimental | SRR1568641 | Alzheimer's | 1 | 83 | 6 | VI |
| Experimental | SRR1568643 | Alzheimer's | 2 | 86 | 10 | V |
| Experimental | SRR1568645 | Alzheimer's | 2 | 79 | 14 | IV |
| Experimental | SRR1568648 | Alzheimer's | 1 | 77 | 1 | VI |
| Experimental | SRR1568652 | Alzheimer's | 1 | 88 | 5 | IV |
| Experimental | SRR1568666 | Alzheimer's | 2 | 81 | 7 | V |
| Experimental | SRR1568669 | Alzheimer's | 2 | 84 | 5 | V |
| Experimental | SRR1568674 | Alzheimer's | 1 | 75 | 8 | V |
| Experimental | SRR1568678 | Alzheimer's | 2 | 90 | 12 | VI |
| Experimental | SRR1568686 | Alzheimer's | 1 | 85 | 1 | III |
| Experimental | SRR1568705 | Alzheimer's | 2 | 86 | 5 | V |
| Experimental | SRR1568719 | Alzheimer's | 2 | 74 | 7 | V |
| Experimental | SRR1568734 | Alzheimer's | 1 | 80 | 3 | IV |
| Experimental | SRR1568744 | Alzheimer's | 2 | 85 | 5 | IV |
| Experimental | SRR1568748 | Alzheimer's | 2 | 89 | 9 | VI |
| Experimental | SRR1568756 | Alzheimer's | 2 | 79 | 10 | VI |
| NA | NA | NA | NA | 80.02 ± 8.1 | 7.16 ± 4.1 | NA |

TABLE 6B
Comparator cohort for AD biomarker discovery, taken from serum samples, including
healthy controls and various other non-Alzheimer's neurological disorders.

| | | | | Age at | Disease | Braak |
|---|---|---|---|---|---|---|
| Group | SRR ID | DiseaseType | Gender | Death | Durration | score |
| Comparator | SRR1568594 | Control | 1 | 38 | NA | 0 |
| Comparator | SRR1568429 | Control | 1 | 80 | NA | I |
| Comparator | SRR1568551 | Control | 2 | 76 | NA | I |
| Comparator | SRR1568564 | Control | 1 | 76 | NA | I |
| Comparator | SRR1568570 | Control | 1 | 71 | NA | I |
| Comparator | SRR1568584 | Control | 1 | 65 | NA | I |
| Comparator | SRR1568603 | Control | 1 | 53 | NA | I |
| Comparator | SRR1568613 | Control | 2 | 59 | NA | I |
| Comparator | SRR1568627 | Control | 1 | 93 | NA | I |
| Comparator | SRR1568671 | Control | 1 | 83 | NA | I |
| Comparator | SRR1568676 | Control | 1 | 79 | NA | I |
| Comparator | SRR1568699 | Control | 1 | 68 | NA | I |
| Comparator | SRR1568707 | Control | 2 | 73 | NA | I |
| Comparator | SRR1568713 | Control | 2 | 70 | NA | I |
| Comparator | SRR1568728 | Control | 2 | 76 | NA | I |
| Comparator | SRR1568742 | Control | 1 | 69 | NA | I |
| Comparator | SRR1568381 | Control | 2 | 88 | NA | II |
| Comparator | SRR1568442 | Control | 1 | 86 | NA | II |
| Comparator | SRR1568449 | Control | 2 | 82 | NA | II |
| Comparator | SRR1568464 | Control | 2 | 83 | NA | II |
| Comparator | SRR1568473 | Control | 1 | 91 | NA | II |
| Comparator | SRR1568494 | Control | 1 | 84 | NA | II |
| Comparator | SRR1568500 | Control | 1 | 84 | NA | II |
| Comparator | SRR1568502 | Control | 1 | 73 | NA | II |
| Comparator | SRR1568506 | Control | 1 | 78 | NA | II |
| Comparator | SRR1568507 | Control | 2 | 77 | NA | II |
| Comparator | SRR1568636 | Control | 1 | 74 | NA | II |
| Comparator | SRR1568646 | Control | 1 | 94 | NA | II |
| Comparator | SRR1568660 | Control | 1 | 78 | NA | II |
| Comparator | SRR1568721 | Control | 1 | 86 | NA | II |
| Comparator | SRR1568385 | Control | 2 | 78 | NA | III |
| Comparator | SRR1568387 | Control | 2 | 90 | NA | III |
| Comparator | SRR1568394 | Control | 2 | 80 | NA | III |
| Comparator | SRR1568405 | Control | 1 | 85 | NA | III |
| Comparator | SRR1568416 | Control | 2 | 88 | NA | III |
| Comparator | SRR1568448 | Control | 2 | 85 | NA | III |
| Comparator | SRR1568477 | Control | 1 | 82 | NA | III |
| Comparator | SRR1568492 | Control | 2 | 88 | NA | III |
| Comparator | SRR1568498 | Control | 2 | 87 | NA | III |
| Comparator | SRR1568509 | Control | 1 | 89 | NA | III |
| Comparator | SRR1568521 | Control | 2 | 84 | NA | III |
| Comparator | SRR1568528 | Control | 2 | 75 | NA | III |
| Comparator | SRR1568543 | Control | 2 | 88 | NA | III |
| Comparator | SRR1568582 | Control | 1 | 82 | NA | III |
| Comparator | SRR1568590 | Control | 2 | 99 | NA | III |
| Comparator | SRR1568606 | Control | 1 | 80 | NA | III |
| Comparator | SRR1568609 | Control | 1 | 85 | NA | III |
| Comparator | SRR1568615 | Control | 2 | 95 | 2 | III |
| Comparator | SRR1568633 | Control | 2 | 92 | NA | III |
| Comparator | SRR1568634 | Control | 1 | 68 | NA | III |
| Comparator | SRR1568650 | Control | 1 | 90 | NA | III |
| Comparator | SRR1568654 | Control | 1 | 84 | NA | III |
| Comparator | SRR1568682 | Control | 1 | 84 | NA | III |
| Comparator | SRR1568696 | Control | 2 | 87 | NA | III |
| Comparator | SRR1568698 | Control | 1 | 90 | NA | III |
| Comparator | SRR1568709 | Control | 1 | 78 | NA | III |
| Comparator | SRR1568732 | Control | 2 | 88 | NA | III |
| Comparator | SRR1568750 | Control | 2 | 91 | NA | III |
| Comparator | SRR1568414 | Control | 1 | 89 | 5 | IV |
| Comparator | SRR1568460 | Control | 2 | 78 | NA | IV |
| Comparator | SRR1568462 | Control | 1 | 82 | NA | IV |
| Comparator | SRR1568470 | Control | 2 | 86 | NA | IV |
| Comparator | SRR1568483 | Control | 1 | 75 | 3 | IV |
| Comparator | SRR1568485 | Control | 1 | 91 | 7 | IV |
| Comparator | SRR1568545 | Control | 2 | 87 | NA | IV |
| Comparator | SRR1568560 | Control | 1 | 87 | NA | IV |
| Comparator | SRR1568568 | Control | 1 | 94 | 8 | IV |
| Comparator | SRR1568579 | Control | 2 | 91 | NA | IV |
| Comparator | SRR1568592 | Control | 1 | 92 | NA | IV |
| Comparator | SRR1568621 | Control | 2 | 84 | NA | IV |
| Comparator | SRR1568377 | Parkinson's | 1 | 72 | 9 | I |
| | | Disease | | | | |
| Comparator | SRR1568487 | Parkinson's | 1 | 73 | 18 | I |
| | | Disease | | | | |
| Comparator | SRR1568513 | Parkinson's | 2 | 87 | 9 | I |
| | | Disease | | | | |
| Comparator | SRR1568680 | Parkinson's | 1 | 88 | 0 | I |
| | | Disease | | | | |
| Comparator | SRR1568701 | Parkinson's | 1 | 81 | 8 | I |
| | | Disease | | | | |
| Comparator | SRR1568375 | Parkinson's | 1 | 75 | 8 | II |
| | | Disease | | | | |
| Comparator | SRR1568383 | Parkinson's | 1 | 85 | 15 | II |
| | | Disease | | | | |
| Comparator | SRR1568419 | Parkinson's | 1 | 82 | 13 | II |
| | | Disease | | | | |
| Comparator | SRR1568466 | Parkinson's | 1 | 73 | 13 | II |
| | | Disease | | | | |
| Comparator | SRR1568511 | Parkinson's | 1 | 79 | 4 | II |
| | | Disease | | | | |
| Comparator | SRR1568577 | Parkinson's | 2 | 79 | NA | II |
| | | Disease | | | | |
| Comparator | SRR1568631 | Parkinson's | 1 | 80 | 25 | II |
| | | Disease | | | | |
| Comparator | SRR1568717 | Parkinson's | 2 | 77 | 21 | II |
| | | Disease | | | | |
| Comparator | SRR1568746 | Parkinson's | 1 | 73 | 17 | II |
| | | Disease | | | | |
| Comparator | SRR1568367 | Parkinson's | 1 | 70 | 12 | III |
| | | Disease | | | | |
| Comparator | SRR1568379 | Parkinson's | 1 | 80 | 10 | III |
| | | Disease | | | | |
| Comparator | SRR1568396 | Parkinson's | 1 | 86 | 7 | III |
| | | Disease | | | | |
| Comparator | SRR1568399 | Parkinson's | 1 | 71 | 12 | III |
| | | Disease | | | | |
| Comparator | SRR1568451 | Parkinson's | 1 | 89 | NA | III |
| | | Disease | | | | |
| Comparator | SRR1568532 | Parkinson's | 2 | 81 | 6 | III |
| | | Disease | | | | |
| Comparator | SRR1568555 | Parkinson's | 1 | 86 | 4 | III |
| | | Disease | | | | |
| Comparator | SRR1568692 | Parkinson's | 1 | 88 | 1 | III |
| | | Disease | | | | |
| Comparator | SRR1568703 | Parkinson's | 1 | 77 | 4 | III |
| | | Disease | | | | |
| Comparator | SRR1568725 | Parkinson's | 2 | 83 | 21 | III |
| | | Disease | | | | |
| Comparator | SRR1568739 | Parkinson's | 2 | 78 | 23 | III |
| | | Disease | | | | |
| Comparator | SRR1568363 | Parkinson's | 2 | 82 | 10 | IV |
| | | Disease | | | | |
| Comparator | SRR1568390 | Parkinson's | 2 | 79 | 6 | IV |
| | | Disease | | | | |
| Comparator | SRR1568425 | Parkinson's | 2 | 86 | 11 | IV |
| | | Disease | | | | |
| Comparator | SRR1568439 | Parkinson's | 2 | 85 | 18 | IV |
| | | Disease | | | | |
| Comparator | SRR1568458 | Parkinson's | 2 | 79 | 20 | IV |
| | | Disease | | | | |
| Comparator | SRR1568472 | Parkinson's | 2 | 81 | 4 | IV |
| | | Disease | | | | |
| Comparator | SRR1568504 | Parkinson's | 2 | 77 | 23 | IV |
| | | Disease | | | | |
| Comparator | SRR1568536 | Parkinson's | 1 | 76 | 9 | IV |
| | | Disease | | | | |
| Comparator | SRR1568588 | Parkinson's | 1 | 84 | 17 | IV |
| | | Disease | | | | |
| Comparator | SRR1568596 | Parkinson's | 1 | 80 | 9 | IV |
| | | Disease | | | | |
| Comparator | SRR1568619 | Parkinson's | 1 | 73 | 11 | IV |
| | | Disease | | | | |
| Comparator | SRR1568715 | Parkinson's | 2 | 83 | 1 | IV |
| | | Disease | | | | |
| Comparator | SRR1568737 | Parkinson's | 1 | 76 | 2 | IV |
| | | Disease | | | | |
| Comparator | SRR1568517 | Parkinson's | 1 | 83 | 15 | 0 |
| | | Disease with | | | | |
| | | Dementia | | | | |
| Comparator | SRR1568684 | Parkinson's | 1 | 72 | 27 | I |
| | | Disease with | | | | |
| | | Dementia | | | | |
| Comparator | SRR1568431 | Parkinson's | 1 | 79 | 23 | II |
| | | Disease with | | | | |
| | | Dementia | | | | |
| Comparator | SRR1568444 | Parkinson's | 1 | 70 | 30 | II |
| | | Disease with | | | | |
| | | Dementia | | | | |
| Comparator | SRR1568479 | Parkinson's | 2 | 84 | 23 | II |
| | | Disease with | | | | |
| | | Dementia | | | | |
| Comparator | SRR1568658 | Parkinson's | 2 | 87 | 0 | II |
| | | Disease with | | | | |
| | | Dementia | | | | |
| Comparator | SRR1568730 | Parkinson's | 2 | 79 | 1 | II |
| | | Disease with | | | | |
| | | Dementia | | | | |
| Comparator | SRR1568365 | Parkinson's | 2 | 73 | 29 | III |
| | | Disease with | | | | |
| | | Dementia | | | | |
| Comparator | SRR1568401 | Parkinson's | 2 | 78 | 16 | III |
| | | Disease with | | | | |
| | | Dementia | | | | |
| Comparator | SRR1568403 | Parkinson's | 2 | 82 | 22 | III |
| | | Disease with | | | | |
| | | Dementia | | | | |
| Comparator | SRR1568427 | Parkinson's | 1 | 78 | 19 | III |
| | | Disease with | | | | |
| | | Dementia | | | | |
| Comparator | SRR1568453 | Parkinson's | 1 | 83 | 7 | III |
| | | Disease with | | | | |
| | | Dementia | | | | |
| Comparator | SRR1568519 | Parkinson's | 2 | 82 | 18 | III |
| | | Disease with | | | | |
| | | Dementia | | | | |
| Comparator | SRR1568549 | Parkinson's | 1 | 75 | 21 | III |
| | | Disease with | | | | |
| | | Dementia | | | | |
| Comparator | SRR1568572 | Parkinson's | 1 | 74 | 17 | III |
| | | Disease with | | | | |
| | | Dementia | | | | |
| Comparator | SRR1568617 | Parkinson's | 2 | 85 | 16 | III |
| | | Disease with | | | | |
| | | Dementia | | | | |
| Comparator | SRR1568629 | Parkinson's | 1 | 83 | 4 | III |
| | | Disease with | | | | |
| | | Dementia | | | | |
| Comparator | SRR1568690 | Parkinson's | 1 | 76 | 2 | III |
| | | Disease with | | | | |
| | | Dementia | | | | |
| Comparator | SRR1568711 | Parkinson's | 1 | 83 | 9 | III |
| | | Disease with | | | | |
| | | Dementia | | | | |
| Comparator | SRR1568754 | Parkinson's | 1 | 85 | 0 | III |
| | | Disease with | | | | |
| | | Dementia | | | | |
| Comparator | SRR1568373 | Parkinson's | 2 | 87 | 18 | IV |
| | | Disease with | | | | |
| | | Dementia | | | | |
| Comparator | SRR1568625 | Parkinson's | 2 | 84 | NA | IV |
| | | Disease with | | | | |
| | | Dementia | | | | |
| AVERAGE | NA | NA | 1.4 ± 0.5 | 80.86 ± 8.2 | 11.98 ± 8.1 | NA |

TABLE 7A
Disease Specific Biomarkers for Alzheimer's Disease Identified in Serum

| Seq. ID | Sequence | Total Reads | Specificity | Sensitivity | p-value |
|---|---|---|---|---|---|
| 255 | CGTGTTCGGACTGGGGTC | 25 | 100% | 19.61% | 1.58E−06 |
| 256 | TGTGATTAGAGGGCTGGAACTTTCACCCCCACCC | 13 | 100% | 17.65% | 6.48E−06 |
| 257 | TCTGTTACGGAACTGTACTCTCTGAGGGCCTCCCACCTGATTC | 21 | 100% | 15.69% | 2.61E−05 |
| 258 | CACCTGTGCGTGTGGGTGCTGCTGCGGGCTGTCAGATGCTGACC | 19 | 100% | 15.69% | 2.61E−05 |
| 259 | CTCAGATCAGACGTGGCG | 17 | 100% | 15.69% | 2.61E−05 |
| 260 | TTTGAGAGGATGATCAGCCACACTGGGACTG | 27 | 100% | 13.73% | 1.03E−04 |
| 261 | CTGTTTCAACCAACGCTTGACTGAGAACTCTTTC | 23 | 100% | 13.73% | 1.03E−04 |
| 262 | TCAGGGTCAGTCTAAGTGAAGACAAAGAGAGGC | 21 | 100% | 13.73% | 1.03E−04 |
| 263 | AGTGCGAGTTTGAGGGCTGTGACCGGCGCT | 19 | 100% | 13.73% | 1.03E−04 |
| 264 | CATGTTGCTTTATTTATCA | 16 | 100% | 13.73% | 1.03E−04 |
| 265 | TGTGGGAGAGTAGGACGCCGCCGGACA | 15 | 100% | 13.73% | 1.03E−04 |
| 266 | TCTGTTACGGAACTGTACTCTCTGAGGGCCTCCCACCTGACTC | 12 | 100% | 13.73% | 1.03E−04 |
| 267 | AGGACTGGTGGAGCGCTTAGAAG | 75 | 100% | 11.76% | 4.01E−04 |
| 268 | GCCCCAGTGGCCTAATGGATAAGGCATTGGCTTAGGGAC | 23 | 100% | 11.76% | 4.01E−04 |
| 269 | CAGGGCACGGTATTTCTTGTTACTTCCCTGCACACGGACTGTG | 23 | 100% | 11.76% | 4.01E−04 |
| 270 | TACAAGGAAGGTCACTACCGTTCTTTCAC | 19 | 100% | 11.76% | 4.01E−04 |
| 271 | CTGCTTTCTTCTTTGGATCGTCGTTCAACT | 19 | 100% | 11.76% | 4.01E−04 |
| 272 | TTAGCAACAACAGGAAGCCCCTTTTATCCT | 19 | 100% | 11.76% | 4.01E−04 |
| 273 | TCTGAATCAACCCTTATTACTCT | 17 | 100% | 11.76% | 4.01E−04 |
| 274 | TCTCATTTGGGCAGAATATGTCAGAGGGAAGATC | 17 | 100% | 11.76% | 4.01E−04 |
| 275 | CCTCCTAAGTATTACACC | 16 | 100% | 11.76% | 4.01E−04 |
| 276 | CCCATCTTGCTGAGATGAGGCC | 16 | 100% | 11.76% | 4.01E−04 |
| 277 | CCTTGTAATAACCTCTAGTCCTTTCC | 15 | 100% | 11.76% | 4.01E−04 |
| 278 | ATTCATGGTGCTTTCAAGTCAGGTTTTCT | 15 | 100% | 11.76% | 4.01E−04 |
| 279 | CATCAGAGACAGTGGCA | 14 | 100% | 11.76% | 4.01E−04 |
| 280 | CCCTGAAGATGTAACTGTCA | 14 | 100% | 11.76% | 4.01E−04 |
| 281 | CCCTGAAGCATACCAAAATGTGTC | 14 | 100% | 11.76% | 4.01E−04 |
| 282 | TGAAAAGGACTTTGAAAAGAGAGTC | 14 | 100% | 11.76% | 4.01E−04 |
| 283 | CTGTCGGGACCCGAAAGATG | 13 | 100% | 11.76% | 4.01E−04 |
| 284 | TCATCTCATCCTGGGGC | 12 | 100% | 11.76% | 4.01E−04 |
| 285 | CTACTCTGAACGATTGAGACC | 12 | 100% | 11.76% | 4.01E−04 |
| 286 | CGGCGGGCTGTCAGATTCTCACC | 12 | 100% | 11.76% | 4.01E−04 |
| 287 | GGGTGATTAGCTCAGCTGGGAGAGCGTCTGCC | 12 | 100% | 11.76% | 4.01E−04 |
| 288 | CCCTAGTCTTCATTTGTTGTTATGTCATTGCCTGCCTT | 12 | 100% | 11.76% | 4.01E−04 |
| 289 | CCCAGGTTCAAGTGATTCTCCTGCCTCAGCCTCCAGAGTACC | 12 | 100% | 11.76% | 4.01E−04 |
| 290 | CTTCACCTGAGAGTGTC | 11 | 100% | 11.76% | 4.01E−04 |
| 291 | CCCCAGAAGCAGGTGTCAAT | 11 | 100% | 11.76% | 4.01E−04 |
| 292 | CCCATATTTCATAATTTCACGCTTCTGTCTTGCATGCTTC | 11 | 100% | 11.76% | 4.01E−04 |
| 293 | CACTTGTGCTTGTGGGTGCTACTGCGGGCGGTCAGATGCTCACC | 11 | 100% | 11.76% | 4.01E−04 |
| 294 | TGGGCAGTGGCTTATGGGAAGATGACCTCTGATTAAATAATTCC | 11 | 100% | 11.76% | 4.01E−04 |
| 295 | CGCGACCTCAGATCCGACGTGGCGACCCGCTGAATTTAAGCC | 39 | 100% | 9.80% | 1.53E−03 |
| 296 | CGTGAGAGAACTCGGGTGAAGGA | 33 | 100% | 9.80% | 1.53E−03 |
| 297 | AAGCACTGAACCGGGCGACTAGTACTAGAGT | 25 | 100% | 9.80% | 1.53E−03 |
| 298 | CTGTCTGGACTACTTCTTTCTCTGATTAATGCCTTGCT | 24 | 100% | 9.80% | 1.53E−03 |
| 299 | CATTTCCTCCATTGTGTCC | 23 | 100% | 9.80% | 1.53E−03 |
| 300 | TATTTGCGTAGAGGTGTTTGTAGTATTCTCTGATGGTAGTA | 23 | 100% | 9.80% | 1.53E−03 |
| 301 | CCTCCTGGAGAGATCTCTTGAGTTCCTGCCTC | 22 | 100% | 9.80% | 1.53E−03 |
| 302 | CGGGAGAGTAGGTCGCGCCAGGTCC | 21 | 100% | 9.80% | 1.53E−03 |
| 303 | CTGTAAGTGTTTGGAGTTGGAATTTAC | 20 | 100% | 9.80% | 1.53E−03 |
| 304 | CCATGCCTGTGGCACACTTCTGTCCTTCACGCTGTCTTCTC | 20 | 100% | 9.80% | 1.53E−03 |
| 305 | CCCTCTCTCAGCATTTTTGCTGTTCGTGAAATGAGGACATAG | 20 | 100% | 9.80% | 1.53E−03 |
| 306 | CCGAGATGGATCTGGCTGGGACCC | 19 | 100% | 9.80% | 1.53E−03 |
| 307 | TCTGTTACGGAAGTGTACTCTCTGAGGGCCTCCCACCTGAGTC | 19 | 100% | 9.80% | 1.53E−03 |
| 308 | AGAAGAAGAAGAGGAAG | 18 | 100% | 9.80% | 1.53E−03 |
| 309 | CCCAGAGTCCATATCAATGG | 18 | 100% | 9.80% | 1.53E−03 |
| 310 | GAGAGGACCGGGTTGGACGA | 18 | 100% | 9.80% | 1.53E−03 |
| 311 | AAAGGGAAGGCTGAACTGCTG | 18 | 100% | 9.80% | 1.53E−03 |
| 312 | ATGGGGTGCAAGCTCTTGATCGAAGCC | 18 | 100% | 9.80% | 1.53E−03 |
| 313 | ACTGTAGTAACTCCTAC | 17 | 100% | 9.80% | 1.53E−03 |
| 314 | TCTTTAGGATCAATTTCCATTC | 17 | 100% | 9.80% | 1.53E−03 |
| 315 | AAGCGAGTCTGAACAGGGCGACTGAGTTTGA | 17 | 100% | 9.80% | 1.53E−03 |
| 316 | CCTTCCTAATTCTTCTTTCAATAGCTATTTA | 17 | 100% | 9.80% | 1.53E−03 |
| 317 | GGCTGGTCCGATGGGAGTGGGTGATCCGAACT | 17 | 100% | 9.80% | 1.53E−03 |
| 318 | GGCTGGTCCGATGGTAGTGGGTTATAGGGATT | 17 | 100% | 9.80% | 1.53E−03 |
| 319 | GAAAAGACATGGAGGGTGTAGAATAAGTGGGAGCTT | 17 | 100% | 9.80% | 1.53E−03 |
| 320 | CCTGCATCAGAGGACAAACCCGCTAATAACTTGATCC | 17 | 100% | 9.80% | 1.53E−03 |
| 321 | CAGGGAGCTGGAGAGGGTTC | 16 | 100% | 9.80% | 1.53E−03 |
| 322 | TGCGAGTGTAGAGGTGAAATTCG | 16 | 100% | 9.80% | 1.53E−03 |
| 323 | CTGTGTCCCCACCCAAATCTCATC | 16 | 100% | 9.80% | 1.53E−03 |
| 324 | GTGTCCATGTTGAAAACTCGCCTG | 16 | 100% | 9.80% | 1.53E−03 |
| 325 | CCCTTCCCATTTTTAATAGTTGTAGC | 16 | 100% | 9.80% | 1.53E−03 |
| 326 | TGCTGCGGGCTGTCAGGATGCTCACC | 16 | 100% | 9.80% | 1.53E−03 |
| 327 | GTTATTTGGATTCTGGGTATGCTCTGG | 16 | 100% | 9.80% | 1.53E−03 |
| 328 | CAGCCCGGGTTCCCTCTTTCTGCCATCTC | 16 | 100% | 9.80% | 1.53E−03 |
| 329 | TAGGTGGATGGTGGATGGGTGGATGATGGA | 16 | 100% | 9.80% | 1.53E−03 |
| 330 | CACCTGTGCGTGTGGGTGATGCTGCGGGCTGTCAGATGCTGACC | 16 | 100% | 9.80% | 1.53E−03 |
| 331 | CCTATCTCAGAATGCCTGAACCAC | 15 | 100% | 9.80% | 1.53E−03 |
| 332 | TTCTGGTAGAATTCAGCTGTGAATCCGTCTTGTCC | 15 | 100% | 9.80% | 1.53E−03 |
| 333 | CCCATTCATTCATTICAATATCCTICAAACATTICTITTC | 15 | 100% | 9.80% | 1.53E−03 |
| 334 | AGGACTGTCCTCGGGAA | 14 | 100% | 9.80% | 1.53E−03 |
| 335 | ATTTGAGAGGGGCTGACCTT | 14 | 100% | 9.80% | 1.53E−03 |
| 336 | CCCCAGAATGATCTTGCCTTC | 14 | 100% | 9.80% | 1.53E−03 |
| 337 | ATACATGAGTTGGGCTTACTGAGTG | 14 | 100% | 9.80% | 1.53E−03 |
| 338 | TAAATGGGTAAGAAGCCCGGCTCGCT | 14 | 100% | 9.80% | 1.53E−03 |
| 339 | CAGAACTGGAACTTGAACCCACATTTC | 14 | 100% | 9.80% | 1.53E−03 |
| 340 | GCATTGGTGGTTCAGTGGTAGAATTCTCGCCTGGTGGA | 14 | 100% | 9.80% | 1.53E−03 |
| 341 | CAAAGGTCAAACAACACAAGTGAGTCTCAAACTCTCAAC | 14 | 100% | 9.80% | 1.53E−03 |
| 342 | CCTCGCGTCGCTTCCTCTTCTCCTTCAGGAGCGTTTTATCCC | 14 | 100% | 9.80% | 1.53E−03 |
| 343 | CAAGTGCAAAGGGAATTCATTTTGAAGAGTTTTATGCAACTGTG | 14 | 100% | 9.80% | 1.53E−03 |
| 344 | AGTTCTACAGTCGGCCGATC | 13 | 100% | 9.80% | 1.53E−03 |
| 345 | AATGGAGGAGTGGTCGGAGGA | 13 | 100% | 9.80% | 1.53E−03 |
| 346 | CAAATGACTATCTCACTGCTC | 13 | 100% | 9.80% | 1.53E−03 |
| 347 | CATATTGTTCTGTGATCTTAACTG | 13 | 100% | 9.80% | 1.53E−03 |
| 348 | GGGACGTTAGCTCAGTTGGTAGAGC | 13 | 100% | 9.80% | 1.53E−03 |
| 349 | TTGATCTCTGGACTGAGGCTTTGTGTGTGCC | 13 | 100% | 9.80% | 1.53E−03 |
| 350 | ACACGATCTCGGCTCACTGCAACCTCTGCCTCC | 13 | 100% | 9.80% | 1.53E−03 |
| 351 | CCCTGGCTCCCTGCTGGGCTTGGGGAGCCTCTTC | 13 | 100% | 9.80% | 1.53E−03 |
| 352 | TGCGAGCGGTCCCGGGTTCACATCCCGGACGAGCCC | 13 | 100% | 9.80% | 1.53E−03 |
| 353 | CCCTCAATCCCTGGTCGAGGGAGAGGGACTTCCTGTC | 13 | 100% | 9.80% | 1.53E−03 |
| 354 | GATTAGGATACAAGGTCTTGCTAGAACTCCCTATCTCCC | 13 | 100% | 9.80% | 1.53E−03 |
| 355 | CTGTGGAACGGGGTGAGATGGGATGGGATGGGACAGGATAGGA | 13 | 100% | 9.80% | 1.53E−03 |
| 356 | CTGGAAGGTTTGACTGT | 12 | 100% | 9.80% | 1.53E−03 |
| 357 | TGCCCTTTGTCATCCCTATGCCT | 12 | 100% | 9.80% | 1.53E−03 |
| 358 | CCCCATGACCCTATTCAAGACTTC | 12 | 100% | 9.80% | 1.53E−03 |
| 359 | CGGTAGCTCGTCAGGCTCATAACC | 12 | 100% | 9.80% | 1.53E−03 |
| 360 | TTCCCTTTGTCATCCTTATGCCTG | 12 | 100% | 9.80% | 1.53E−03 |
| 361 | CTTCAACATCACCTGTAGCCATCAC | 12 | 100% | 9.80% | 1.53E−03 |
| 362 | CCTTCCACCTTGGCCTCCCAAAGTGC | 12 | 100% | 9.80% | 1.53E−03 |
| 363 | AGGGGAATGGAATGGAATGGAATGCAA | 12 | 100% | 9.80% | 1.53E−03 |
| 364 | CGCGGGTGAGTAGGTCGCTGCCAGGTCT | 12 | 100% | 9.80% | 1.53E−03 |
| 365 | AGGGACCCTCTGTGGCGGGTAGTTTGACT | 12 | 100% | 9.80% | 1.53E−03 |
| 366 | TATATGGAAGACATAAAAAGAGAAGCTCC | 12 | 100% | 9.80% | 1.53E−03 |
| 367 | AGGAATTTCGGTCCAGATTGTTTCTTGAGTCACT | 12 | 100% | 9.80% | 1.53E−03 |
| 368 | AAAAAGTCTTTAACTCCACCATTAGCACCCAAAGC | 12 | 100% | 9.80% | 1.53E−03 |
| 369 | CTAAGGGGTCGGGAGTTCGAATCTCTCTGAGCGCAC | 12 | 100% | 9.80% | 1.53E−03 |
| 370 | CGTAGTGTCGGTGGTTCGATTCCGCCCCTGGGCACCA | 12 | 100% | 9.80% | 1.53E−03 |
| 371 | GAGCTGATTGGTACTAATCGGTCGTGAGGCTTGACCT | 12 | 100% | 9.80% | 1.53E−03 |
| 372 | GCTCTAAGTTCGAGTCTCTCTTTCACTTCTTCTCTTGG | 12 | 100% | 9.80% | 1.53E−03 |
| 373 | CCCAGGTTGAGTTTATGGGGGTAGTGCTGTAAGGTCATT | 12 | 100% | 9.80% | 1.53E−03 |
| 374 | AATCGGACTGTTCAACTCACCTGGCAACCACTCCCAGAGCCCC | 12 | 100% | 9.80% | 1.53E−03 |
| 375 | TTTCAAGGACTGTGTTTAATTTCCTTTTGGATTTGTTTATTTTG | 12 | 100% | 9.80% | 1.53E−03 |
| 376 | CGAATAAGCTTTGATCCA | 11 | 100% | 9.80% | 1.53E−03 |
| 377 | CACTGGAATTCTGAGCCCCT | 11 | 100% | 9.80% | 1.53E−03 |
| 378 | CAGGAGTCGGGGGTGGGACG | 11 | 100% | 9.80% | 1.53E−03 |
| 379 | AAAAGAGGACCACCACCAAGA | 11 | 100% | 9.80% | 1.53E−03 |
| 380 | GGTGGTGGCGGCGGTGGTGGC | 11 | 100% | 9.80% | 1.53E−03 |
| 381 | GTCTTACTCTGTTGCTCAGGC | 11 | 100% | 9.80% | 1.53E−03 |
| 382 | CCTCCTCTGGATCACATGGGCTC | 11 | 100% | 9.80% | 1.53E−03 |
| 383 | CCTTCGGGCCTGTCCAGAACCTC | 11 | 100% | 9.80% | 1.53E−03 |
| 384 | TTCGAATCTCACCGCTTCCGCCA | 11 | 100% | 9.80% | 1.53E−03 |
| 385 | CCATCACATAGGGGATTAGATTTCAATGC | 11 | 100% | 9.80% | 1.53E−03 |
| 386 | TGTAAGGGCTGGGTCGGTCGGGCTGGGGC | 11 | 100% | 9.80% | 1.53E−03 |
| 387 | CAGCGCCTTTGCACACGCTATTCTCTCTGCC | 11 | 100% | 9.80% | 1.53E−03 |
| 388 | CGCGGAGCCCAGGGTTCGATTCCCTGTACCG | 11 | 100% | 9.80% | 1.53E−03 |
| 389 | CTGATGGGCTGGGCAGGGCTCCCTGGATGGG | 11 | 100% | 9.80% | 1.53E−03 |
| 390 | CCCCACTTCCGTACTGAGTTTCTCACCTGTTTG | 11 | 100% | 9.80% | 1.53E−03 |
| 391 | AGTACTGTTATTTAGCGTGCTAAATATATTGTCC | 11 | 100% | 9.80% | 1.53E−03 |
| 392 | AGTGCATCGCGCGAAAGTAGGTCGTCGCCGGCTT | 11 | 100% | 9.80% | 1.53E−03 |
| 393 | CCTGATTTTTTTTGCAATTTCTTTGTATTGTTTTTA | 11 | 100% | 9.80% | 1.53E−03 |
| 394 | TGATGGAGTGGCCTGGACTCACATTAAAATAAGTACT | 11 | 100% | 9.80% | 1.53E−03 |
| 395 | CCCCTTACCCATCAAATTTTCCTTAAAAACTCCAATCC | 11 | 100% | 9.80% | 1.53E−03 |
| 396 | CTCTTTGGGGCGGGGTGGGGGAGGGGGAGCCTCGCGTCC | 11 | 100% | 9.80% | 1.53E−03 |
| 397 | CCTGAGCTCTTGTTCGATGTCCAAGGATAATGAGGTGGCA | 11 | 100% | 9.80% | 1.53E−03 |
| 398 | TAAGGAGGAGGAACATTGTGAGCAGGAGAAGGATCTGGGG | 11 | 100% | 9.80% | 1.53E−03 |
| 399 | TCCTGTCCGGTTGAGGCCTTTCTCTTGGGGTCTTGCTGTC | 11 | 100% | 9.80% | 1.53E−03 |
| 400 | CCTTTCATATCTTCTCAAATACTGATTTAATTTTATACTGG | 11 | 100% | 9.80% | 1.53E−03 |
| 401 | CCTAGGTTCAAGTGATCCTCCTGCTTCAGCTTCCTGAGTAGC | 11 | 100% | 9.80% | 1.53E−03 |
| 402 | CCTGGCCTCAAGCAATCCTCCCACCTTGGCCTCCACAAGTAC | 11 | 100% | 9.80% | 1.53E−03 |
| 403 | CATCTCAGCTCCAAACCCACAGGTTGGGTTCAGTTCTTGCATCC | 11 | 100% | 9.80% | 1.53E−03 |

TABLE 7B
Disease Specific Biomarkers for Alzheimer's Disease Identified in Serum

| | Stage | | | | | | | |
|---|---|---|---|---|---|---|---|---|
| | Braak II | Braak II | Braak II | Braak III | Braak III | Braak IV | Braak IV | Braak IV |
| Seq. ID | SRR1568547 | SRR1568553 | SRR1568580 | SRR1568557 | SRR1568686 | SRR1568421 | SRR1568437 | SRR1568534 |
| 255 | | | | | 0.197 | | | |
| 256 | | | | | | 0.92 | | |
| 257 | | | | | | | | |
| 258 | | | | | | | | |
| 259 | | 0.076 | 0.125 | | | | | |
| 260 | | | | | 1.181 | | | |
| 261 | | | | | | 3.678 | | |
| 262 | | | | | | | | |
| 263 | | 0.076 | | | | | | |
| 264 | | | | | | | | |
| 265 | | | 0.125 | | | | 0.301 | |
| 266 | | | | | 0.197 | | | |
| 267 | | | | 0.6 | 11.611 | | | |
| 268 | | | | | 0.787 | | | |
| 269 | | | | | | | | |
| 270 | | | | | | | | |
| 271 | | | | | | 0.92 | | |
| 272 | | | | | | 2.759 | | |
| 273 | | | | | | | | |
| 274 | | | | | 1.574 | | | |
| 275 | | | | | 0.787 | 2.759 | | |
| 276 | | | | | | | | |
| 277 | | | | | | | | |
| 278 | | | | | | | | |
| 279 | | | | | | 3.678 | | |
| 280 | | | | | | | | |
| 281 | | | | | | | | |
| 282 | | 0.229 | | | | | 0.602 | |
| 283 | | 0.305 | | | | 2.759 | 0.602 | |
| 284 | | | | | | | | |
| 285 | | 0.076 | | | | | | |
| 286 | | | | | | | | |
| 287 | 3.825 | 0.153 | | 0.3 | | | 0.301 | |
| 288 | | | | | | 1.839 | | |
| 289 | | | | | | | | |
| 290 | | | | | | | | |
| 291 | | | | | | 0.92 | | |
| 292 | | | | | | | | |
| 293 | | | | | | | | |
| 294 | | | | | | | | |
| 295 | | | | 0.3 | 1.574 | | | |
| 296 | | | | | | | | |
| 297 | | | | | | | | |
| 298 | | | | | | 1.839 | | |
| 299 | | | | | | | | |
| 300 | | | | | | | | |
| 301 | | | | | | | | |
| 302 | | | | | 1.771 | | 2.106 | |
| 303 | | | | | | | | |
| 304 | | | | | | | | |
| 305 | | | | | | | | |
| 306 | | | | | | | 0.301 | |
| 307 | | | | | | | | |
| 308 | | | | | | | | |
| 309 | | | | | | | | |
| 310 | | | | | | | | |
| 311 | | | | | | | | |
| 312 | | | | | 2.362 | | | |
| 313 | | | | | | | | |
| 314 | | | | | | | | |
| 315 | | | | | 1.574 | | | |
| 316 | | | | | | | | |
| 317 | | | | | | | | |
| 318 | | | | | | | | |
| 319 | | | | | | | 0.301 | |
| 320 | | | | | | | | |
| 321 | | | | | | | | |
| 322 | 2.55 | | | 0.9 | 1.771 | | | |
| 323 | | | | | | 5.518 | | |
| 324 | | | | | | 1.839 | | |
| 325 | | | | | | 1.839 | | |
| 326 | | | | | | | | |
| 327 | | | | | | 4.598 | | |
| 328 | | | | | | | | |
| 329 | | | | | | | | |
| 330 | | | | | | | | |
| 331 | | | | | | 0.92 | | |
| 332 | | | | | | 2.759 | | |
| 333 | | | | | | | | |
| 334 | | | 0.25 | | | | | |
| 335 | | | | | | | | |
| 336 | | | | | | | | |
| 337 | | | | | | 0.92 | | |
| 338 | | | | | | | | |
| 339 | | | | | | 1.839 | | |
| 340 | | | 0.25 | | | | | |
| 341 | | | | | | 4.598 | | |
| 342 | | | | | | | | |
| 343 | | | | | | 0.92 | | |
| 344 | | | | | 0.984 | | | |
| 345 | | | | | | | | |
| 346 | | 0.076 | | | | | | |
| 347 | | | | | | | | |
| 348 | | | | | 0.59 | | | |
| 349 | | | | | | | | |
| 350 | | | | | | | | |
| 351 | | | | | | 1.839 | | |
| 352 | | | | | 0.394 | | | |
| 353 | | | | | 0.984 | | | |
| 354 | | | | | | | | |
| 355 | | | | | 0.984 | | | |
| 356 | | | | | | | | |
| 357 | | | | | 0.59 | | | |
| 358 | | | | | | | | |
| 359 | | | 0.125 | | 0.787 | | 0.301 | 0.247 |
| 360 | | | | | 1.378 | | | |
| 361 | | | | | | | | |
| 362 | | | | | | | | |
| 363 | | | | | | 2.759 | | 0.247 |
| 364 | | | | | 1.181 | | 0.602 | 0.247 |
| 365 | | | | | | | | |
| 366 | | | | | | | | |
| 367 | | | | | | 3.678 | | |
| 368 | | | | | | | | 0.494 |
| 369 | | | | | | | | |
| 370 | | | | | 1.378 | | 0.301 | |
| 371 | | | | | | | 0.903 | |
| 372 | | | | | | | | |
| 373 | | | | | | | | |
| 374 | | | | | | 5.518 | | |
| 375 | | | | | | 3.678 | | |
| 376 | | | | | 1.181 | | | |
| 377 | | | | | | | | |
| 378 | | | 0.25 | 0.3 | | | | |
| 379 | | | | | | | | |
| 380 | | 0.076 | | | | | 0.301 | |
| 381 | | 0.076 | | | | | | |
| 382 | | | | | | 0.92 | | |
| 383 | | | | | | | | |
| 384 | | | | | 0.984 | | 0.301 | |
| 385 | | | | | | 1.839 | | |
| 386 | | | | | | | | |
| 387 | | | | | | | | |
| 388 | | 0.076 | 0.125 | | | | | |
| 389 | | | | | 0.59 | | | |
| 390 | | | | | | | | |
| 391 | | | | | | | | |
| 392 | | | | | 0.787 | | | 0.247 |
| 393 | | | | | | 2.759 | | |
| 394 | | | | | | | | |
| 395 | | | | | | | | |
| 396 | | | | | | 2.759 | | |
| 397 | | | | | | | | |
| 398 | | | | | | | | |
| 399 | | | | | | | | |
| 400 | | | | | | | | |
| 401 | | | | | | | | |
| 402 | | | | | | | | |
| 403 | | | | | | | | |
| # Biomarkers | 2 | 10 | 7 | 5 | 26 | 29 | 13 | 5 |
| Per Sample | | | | | | | | |
| % Coverage | 1% | 7% | 5% | 3% | 17% | 19% | 9% | 3% |
| | Stage | | | | | | | |
| | Braak IV | Braak IV | Braak IV | Braak IV | Braak IV | Braak IV | Braak V | Braak V |
| Seq. ID | SRR1568541 | SRR1568586 | SRR1568645 | SRR1568652 | SRR1568734 | SRR1568744 | SRR1568369 | SRR1568371 |
| 255 | | | 9.27 | | | | | |
| 256 | | | | | | | | |
| 257 | | | 7.416 | | | | | 0.067 |
| 258 | | | 1.854 | | | | | |
| 259 | | | | | | | | |
| 260 | | | | | | | | 0.033 |
| 261 | | | | | | | | 0.033 |
| 262 | | | | | | | 0.307 | |
| 263 | | | | | | | | |
| 264 | | | | | | | | 0.033 |
| 265 | | | | | | | | 0.1 |
| 266 | | | 3.708 | | | | | |
| 267 | | | | | | | | |
| 268 | | | | | | | | |
| 269 | | | | | | | | 0.033 |
| 270 | | | | 5.944 | | | | |
| 271 | | | | | | | 0.614 | |
| 272 | | | | | | | | |
| 273 | | | | 1.981 | | | | 0.033 |
| 274 | | | | | | | | |
| 275 | | | | | | | | 0.1 |
| 276 | | | | | | | | |
| 277 | | | | | | | | |
| 278 | | | | | | | 0.307 | |
| 279 | | | | | | | | 0.033 |
| 280 | | | | | | | | 0.033 |
| 281 | | | | | | | | |
| 282 | | | | | | | 0.307 | |
| 283 | | | | | | | | |
| 284 | | | | | | | | |
| 285 | | | | | | | | 0.067 |
| 286 | | | | 0.991 | | | | |
| 287 | | | | | | | | |
| 288 | | | | | | | | |
| 289 | | | | | | | | |
| 290 | | | | | | | | |
| 291 | | | | | | | | |
| 292 | | | | | | | 0.307 | |
| 293 | | | 1.854 | | | | | |
| 294 | | | | | | | | 0.033 |
| 295 | | | | | | | | |
| 296 | | | | 28.73 | | | | 0.033 |
| 297 | | | | | 0.741 | | | 0.134 |
| 298 | | | | | | | | |
| 299 | | | | | | | | |
| 300 | | | 11.124 | | | | | |
| 301 | | | | | | | | |
| 302 | | | | | | | | 0.067 |
| 303 | | | | | | | 0.307 | |
| 304 | | | | | | | | |
| 305 | | | 3.708 | | | | | |
| 306 | | | | | | | | |
| 307 | | | 1.854 | | | | | |
| 308 | | | | | | | | |
| 309 | | | | | | | | |
| 310 | | | | 10.898 | | | | |
| 311 | 0.585 | | | | | | | |
| 312 | | | | | | | 0.307 | |
| 313 | | | | | | | | |
| 314 | | | | 5.944 | | | | 0.067 |
| 315 | | | | | | | | |
| 316 | | | | | | | 0.307 | |
| 317 | | | | | | | | 0.435 |
| 318 | | | | | | | | 0.435 |
| 319 | | | | 7.926 | | | | |
| 320 | | | | 1.981 | | | | |
| 321 | | | | | | | | 0.1 |
| 322 | | | | | | | | 0.033 |
| 323 | | | | | | | | 0.1 |
| 324 | | | | | | | | |
| 325 | | | | | | | | |
| 326 | | | | | | | | |
| 327 | | | 1.854 | | | | | |
| 328 | | | | | | | | |
| 329 | | | | | | | | |
| 330 | | | 1.854 | | | | | |
| 331 | | | | 2.972 | | | | 0.033 |
| 332 | | | | | | | | |
| 333 | | | | | | | | 0.067 |
| 334 | | | | | | | | |
| 335 | | | | 6.935 | | | | |
| 336 | | | | 4.954 | | | | 0.067 |
| 337 | | | | | | | | |
| 338 | | | | | | | | |
| 339 | | | 11.124 | | | | | |
| 340 | | | | | | | | 0.033 |
| 341 | | | | | | | | 0.067 |
| 342 | | | | | | | | 0.1 |
| 343 | | | | | | | | 0.067 |
| 344 | | | 1.854 | | | 1.55 | | |
| 345 | | | | | | | | |
| 346 | | | 1.854 | | | | | |
| 347 | | | | | | | 0.307 | |
| 348 | | | | | | | | 0.167 |
| 349 | | | | | | | | |
| 350 | | | | | | | | 0.134 |
| 351 | | | 7.416 | | | | | |
| 352 | | | | | | | | |
| 353 | | | | | | | | |
| 354 | | | | | | | | |
| 355 | | | | | | | | 0.033 |
| 356 | | | | | | | | |
| 357 | | | | | | | | |
| 358 | | | | 4.954 | | | | 0.1 |
| 359 | | | | | | | | |
| 360 | | | | | | | | |
| 361 | | | | | | | 0.307 | 0.033 |
| 362 | | | | | | | | |
| 363 | | | | | | | | |
| 364 | | | | | | | | |
| 365 | | | | | | | | |
| 366 | | | | | | | | |
| 367 | | | | | | | | |
| 368 | | | | | 2.224 | | | |
| 369 | | | | | | | | |
| 370 | | | | | | | | |
| 371 | | | | | | | 0.307 | 0.033 |
| 372 | | | | | | | | |
| 373 | | | | | | | | 0.201 |
| 374 | | | | | | | | 0.1 |
| 375 | | | | | | | | 0.033 |
| 376 | | | | | | | | |
| 377 | | | | 1.981 | | | | 0.067 |
| 378 | | | | | | | | |
| 379 | | | | | | | | |
| 380 | | | | | | 4.651 | | |
| 381 | | | | | | | | |
| 382 | | | | | | | | |
| 383 | | 2.14 | | | | | | |
| 384 | | | | | | | 0.307 | |
| 385 | | | | | | | | 0.067 |
| 386 | | | | | | | | 0.167 |
| 387 | | | | | | | | |
| 388 | | | | | | | | 0.134 |
| 389 | | | | | | | | 0.033 |
| 390 | | | | | | | | 0.067 |
| 391 | | | 1.854 | | | | | 0.067 |
| 392 | | | | | | | 0.614 | 0.1 |
| 393 | | | | | | | 0.921 | |
| 394 | | | | | | | | 0.033 |
| 395 | | | | 2.972 | | | | 0.1 |
| 396 | | | | | | | | |
| 397 | | | | | | | | |
| 398 | | | | | | | | |
| 399 | | | 5.562 | | | | | 0.067 |
| 400 | | | | | | | | 0.033 |
| 401 | | | | | | | | 0.067 |
| 402 | | | | | | | | |
| 403 | | | 5.562 | 1.981 | | | | 0.1 |
| # Biomarkers | 1 | 1 | 17 | 15 | 2 | 2 | 14 | 51 |
| Per Sample | | | | | | | | |
| % Coverage | 1% | 1% | 11% | 10% | 1% | 1% | 9% | 34% |

| | Stage | | | | | | | |
|---|---|---|---|---|---|---|---|---|
| | Braak V | Braak V | Braak V | Braak V | Braak V | Braak V | Braak V | Braak V |
| Seq. ID | SRR1568407 | SRR1568409 | SRR1568411 | SRR1568446 | SRR1568455 | SRR1568468 | SRR1568475 | SRR1568481 |
| 255 | | | | | | | | |
| 256 | | 0.243 | | | | | | |
| 257 | | | 1.988 | | | | | |
| 258 | | 0.243 | | | | | | |
| 259 | 0.589 | | | | | | | |
| 260 | | | | | | | | |
| 261 | | | | | | | | |
| 262 | | | 7.952 | | | | | 0.199 |
| 263 | 1.325 | | | | | | 1.032 | |
| 264 | | | | | | | | |
| 265 | | | | | | | | |
| 266 | | | | | | | | |
| 267 | 0.442 | | | | | | | |
| 268 | 0.147 | | | | | | | |
| 269 | | 0.487 | | | | | | |
| 270 | | | | 4.457 | | | | |
| 271 | | | | | | | | |
| 272 | | | | 0.743 | | | | |
| 273 | | | | | | | | |
| 274 | | | | 2.228 | | | | |
| 275 | | | | | | | | |
| 276 | | 0.974 | | | | | | |
| 277 | | 0.243 | | | | | | |
| 278 | | | | | | | 1.548 | |
| 279 | | 0.73 | | 2.228 | | | | 0.199 |
| 280 | | 0.73 | | | | | | |
| 281 | | 0.487 | | | | | | |
| 282 | | | | | | | | |
| 283 | | | | | | | | |
| 284 | 0.442 | | | | | | 1.548 | |
| 285 | | | | | | | | |
| 286 | | | 1.988 | | | | | |
| 287 | | | | | | | | |
| 288 | | 0.243 | | | | | | |
| 289 | | 0.243 | | 3.714 | | | | |
| 290 | | 0.487 | | | | | | |
| 291 | | | | | 0.349 | | | |
| 292 | | 0.243 | | | | | | |
| 293 | | 0.487 | 1.988 | | | | | |
| 294 | | 0.243 | 5.964 | | | | | |
| 295 | | | | | | | | |
| 296 | | | | | | | | |
| 297 | | | | | | | | 0.199 |
| 298 | | 0.487 | | | | | | |
| 299 | | | | 5.199 | 0.349 | 16.967 | | |
| 300 | | 0.487 | | | | | | |
| 301 | | | | 3.714 | | | | |
| 302 | | | | | | | | |
| 303 | | | | | | | | |
| 304 | | 0.243 | | | | | | |
| 305 | | | | | | | | |
| 306 | | | | | | | | 0.199 |
| 307 | | | | | | | | |
| 308 | | | | | | | 1.548 | |
| 309 | | 0.73 | | 3.714 | | | | |
| 310 | | | | | | | | |
| 311 | | | | | | | | |
| 312 | | 0.243 | | | | | | |
| 313 | | | | | | | | |
| 314 | | | | 1.486 | | | | |
| 315 | | | | | | | | |
| 316 | | | | | | | | |
| 317 | 0.147 | | | | 0.349 | | | |
| 318 | 0.147 | | | | 0.349 | | | 0.199 |
| 319 | | | | | | | | |
| 320 | | | | | | | | 0.199 |
| 321 | | | 7.952 | | | | | |
| 322 | | | | | | | | |
| 323 | | | | | | | | |
| 324 | | | | | | | | |
| 325 | | 0.243 | | | 0.349 | | | |
| 326 | | | | | | | | |
| 327 | | | 11.928 | | | | | |
| 328 | | | | 4.457 | | | | |
| 329 | | | | | | | | |
| 330 | | | | | | | | |
| 331 | | | | | | | | |
| 332 | | | | 1.486 | | | | |
| 333 | | 0.73 | | | | | | |
| 334 | 0.736 | | | | | | | |
| 335 | | | | | | | | |
| 336 | | | | | | | | |
| 337 | | 0.487 | | | | | | |
| 338 | | | | | | | | |
| 339 | | | | | | | | |
| 340 | 1.178 | | | | | | | |
| 341 | | | | | | | | |
| 342 | | 0.243 | | | | | | |
| 343 | | | 5.964 | | | | | |
| 344 | | | | | | | | |
| 345 | | 0.73 | | | | | | |
| 346 | | | | 3.714 | 1.047 | | | |
| 347 | | 0.487 | | | 0.698 | | | |
| 348 | | | | | | | | |
| 349 | | | | 4.457 | | | 0.516 | |
| 350 | | | | 0.743 | 0.349 | | | |
| 351 | | | | 0.743 | | | | |
| 352 | 0.147 | | | | 0.349 | | | |
| 353 | | | | | | | | |
| 354 | | | | | | | | |
| 355 | | | | | | | | |
| 356 | | | | | | | | |
| 357 | | | | | | | | |
| 358 | | | | | 0.698 | | | |
| 359 | | | | | | | | |
| 360 | | | | | | | | |
| 361 | | | | 0.743 | | | | |
| 362 | | | | | | | | |
| 363 | | | | | | | | |
| 364 | | | | | | | | |
| 365 | | | | | | | | |
| 366 | | 0.243 | | 2.228 | | | | |
| 367 | | | | | | | | |
| 368 | | | | | | | | 0.199 |
| 369 | | | | | | | | |
| 370 | | | | | | | | |
| 371 | | | | | | | | |
| 372 | | | | 3.714 | | | | |
| 373 | | | | | | | | |
| 374 | | | | 0.743 | | | | |
| 375 | | | | | | | | |
| 376 | | | | | | | | |
| 377 | | | | | | | | |
| 378 | | | | | | | | |
| 379 | | | | | | | 0.516 | |
| 380 | | | | | | | | |
| 381 | | | | | | | | |
| 382 | | | | | | | | |
| 383 | | | | | | | | |
| 384 | | | | | | | | |
| 385 | | | | | | | | |
| 386 | | 0.487 | | | | | | |
| 387 | | 0.487 | | | 1.395 | | | |
| 388 | | | | | | | | |
| 389 | | | | | | | | |
| 390 | | | | | | | | |
| 391 | | | | 2.228 | | | | |
| 392 | | | | | | | | |
| 393 | | | | | | | | |
| 394 | | | | | | | | |
| 395 | | | | | | | | |
| 396 | | | | | | | | |
| 397 | | | | | | | | |
| 398 | | | | | | | | |
| 399 | | 0.243 | | | | | | |
| 400 | | 0.243 | | | | | | 0.199 |
| 401 | | 0.487 | | | | | | |
| 402 | | | | | | | | |
| 403 | | | | 1.486 | | | | |
| | 10 | 31 | 8 | 21 | 11 | 1 | 6 | 8 |
| | 7% | 21% | 5% | 14% | 7% | 1% | 4% | 5% |

| Seq. ID | SRR1568515 | SRR1568523 | SRR1568623 | SRR1568639 | SRR1568643 | SRR1568666 | SRR1568669 | SRR1568674 |
|---|---|---|---|---|---|---|---|---|
| 255 | 0.466 | 0.824 | | | | | 0.091 | |
| 256 | 0.466 | 0.412 | | 0.31 | | 0.075 | | |
| 257 | | 1.647 | | 0.155 | | | | |
| 258 | 0.466 | 0.824 | | | | | | |
| 259 | | | | 0.466 | | 0.075 | | 0.628 |
| 260 | | | | | 0.05 | | 0.914 | 1.885 |
| 261 | | | | | | | | |
| 262 | | | | | | 0.151 | | |
| 263 | | | | | | | | |
| 264 | | | | | | 0.151 | | |
| 265 | | | | | | | | |
| 266 | | 0.412 | | | | | | |
| 267 | | | | | 0.202 | | | |
| 268 | 0.466 | | | | 0.101 | | 0.457 | |
| 269 | | | | | | | | |
| 270 | | | | | | 0.075 | | |
| 271 | | | | | | | | |
| 272 | | 3.295 | | | | | | |
| 273 | | 3.295 | | | | | | |
| 274 | | | | | | 0.075 | | |
| 275 | | | | | | | | |
| 276 | | 2.883 | | | | 0.151 | | |
| 277 | | 0.824 | | | | 0.151 | | |
| 278 | | | | | | | | |
| 279 | | | | | | | | |
| 280 | | | | | | 0.151 | | |
| 281 | | | | | | 0.151 | | |
| 282 | | | | | | | | |
| 283 | | | | | 0.101 | | | |
| 284 | | | | | 0.05 | | | |
| 285 | | | | | | | | |
| 286 | | 0.412 | | | | | | |
| 287 | | | | | | | | 0.943 |
| 288 | | | | 0.31 | | | | |
| 289 | | | | | | | | |
| 290 | | 0.824 | | | | | | |
| 291 | | | | | | 0.075 | | |
| 292 | | | | | | 0.226 | | |
| 293 | 0.466 | 0.412 | | | | | | |
| 294 | | | | | | | | |
| 295 | | | | 0.155 | 0.555 | | | |
| 296 | | | | | | 0.075 | 0.091 | |
| 297 | | | | | | | | |
| 298 | | | | | | | | |
| 299 | | | | | | | | |
| 300 | | | | 0.155 | | | | |
| 301 | | | | | | 0.151 | | |
| 302 | | | | | | | | |
| 303 | | | | | | | | |
| 304 | | 2.059 | | | | | | |
| 305 | | 0.412 | | | | | | |
| 306 | | | | | | | 0.091 | |
| 307 | | 3.707 | | 0.155 | | | | |
| 308 | 2.328 | | | | | | | |
| 309 | | | | | | 0.226 | | |
| 310 | | | | | | | 0.091 | 0.314 |
| 311 | 6.054 | | | 0.155 | | 0.151 | | |
| 312 | | | | | | | | |
| 313 | | | | 0.931 | | 0.226 | | |
| 314 | | | | | | | | |
| 315 | | | | | 0.202 | | 0.091 | |
| 316 | | | | | | 0.075 | | |
| 317 | | | | | | | | |
| 318 | | | | | | | | |
| 319 | | | | | | | | |
| 320 | 4.191 | | | | | | | |
| 321 | | | | | | | | |
| 322 | | | | | | | | |
| 323 | | | | | | | | |
| 324 | | | | | | | | |
| 325 | | | | | | | | |
| 326 | 0.466 | 0.824 | | | | | | |
| 327 | | | | | | | | |
| 328 | | 1.647 | | | | | | |
| 329 | | 0.412 | | | | | | |
| 330 | | 0.412 | | | | | | |
| 331 | | | | | | | | |
| 332 | | | | | | | | |
| 333 | | | | | | | | |
| 334 | | | | | | | | |
| 335 | | | | | 0.05 | 0.151 | 0.091 | 0.943 |
| 336 | | | | | | 0.075 | | |
| 337 | | | | | | | | |
| 338 | | | 0.584 | | | | | 3.142 |
| 339 | | | | | | 0.075 | | |
| 340 | | | | | 0.101 | | | |
| 341 | | | | | | | | |
| 342 | | 2.059 | | | | | | 0.314 |
| 343 | | | | | | | | |
| 344 | 0.931 | | | | | | | |
| 345 | | 2.059 | | | | | | |
| 346 | | | | | | | | |
| 347 | | | | | | | | |
| 348 | | | | | 0.05 | | | |
| 349 | | | | | | | | |
| 350 | | | | 0.466 | | | | |
| 351 | | | | | | | | |
| 352 | | | | | | | 0.091 | |
| 353 | | | | | | 0.075 | | |
| 354 | | | | | | 0.075 | | |
| 355 | | | | 0.155 | | | | |
| 356 | | | | | | 0.075 | | |
| 357 | 0.466 | | | 0.776 | | | | |
| 358 | | | | | | 0.075 | | |
| 359 | | | | | | | | |
| 360 | | | | 0.155 | | | | |
| 361 | | | | | | | | |
| 362 | 1.863 | | | | | 0.075 | | |
| 363 | | | | | | 0.075 | | |
| 364 | | | | | | | | |
| 365 | | | | | 0.151 | | 0.183 | |
| 366 | | | | | | | | |
| 367 | | | | | | 0.075 | | |
| 368 | | | | | | | 0.091 | |
| 369 | | | | 0.776 | 0.05 | | | 1.257 |
| 370 | | | | | | | | |
| 371 | | | | | | | | |
| 372 | | | | | | | | |
| 373 | | | | | | 0.075 | | |
| 374 | | | | | | | | |
| 375 | | | | | | | | |
| 376 | | 0.824 | | | 0.05 | | | |
| 377 | | | | 0.466 | | | | |
| 378 | | | | 0.621 | | | | |
| 379 | | | | | | | | |
| 380 | | | | | | | | |
| 381 | | | | | | | | |
| 382 | | | | | | | | |
| 383 | | | | | | 0.075 | | 0.943 |
| 384 | | | | | | | | |
| 385 | | | | | | 0.075 | | |
| 386 | | | | 0.31 | | | | 0.314 |
| 387 | | | | | | 0.075 | | |
| 388 | | | | | | | | 0.314 |
| 389 | | | | | | | | |
| 390 | | | | 0.155 | | 0.075 | | |
| 391 | | | | | | | | |
| 392 | | | | | | | | |
| 393 | | | | | | 0.226 | | |
| 394 | | | | | | | | |
| 395 | | | | | | | | |
| 396 | | | | | | | | |
| 397 | | 1.236 | | | | | | |
| 398 | | | | | | 0.075 | | |
| 399 | | | | | | | | |
| 400 | | | | | | | | |
| 401 | | 1.647 | | | | 0.075 | | |
| 402 | | | | | | | | 0.628 |
| 403 | | | | | 0.05 | | | |
| # Biomarkers | 12 | 24 | 1 | 18 | 14 | 36 | 11 | 12 |
| Per Sample | | | | | | | | |
| % Coverage | 8% | 16% | 1% | 12% | 9% | 24% | 7% | 8% |
| | Stage | | | | | | | |
| | Braak V | Braak V | Braak VI | Braak VI | Braak VI | Braak VI | Braak VI | Braak VI |
| Seq. ID | SRR1568705 | SRR1568719 | SRR1568433 | SRR1568435 | SRR1568490 | SRR1568496 | SRR1568525 | SRR1568530 |
| 255 | | | 0.405 | 1.713 | | | 0.398 | 0.39 |
| 256 | | | | 0.514 | 0.572 | | | |
| 257 | | | | 1.028 | | | | |
| 258 | | | | 1.884 | | | | |
| 259 | | | | 0.685 | 0.572 | | | |
| 260 | | | 0.203 | | | | | |
| 261 | | 0.618 | | | | | 0.796 | |
| 262 | | 0.618 | | 1.884 | | | | |
| 263 | | | 0.608 | | | 0.765 | | |
| 264 | | 2.472 | | 0.343 | | 2.296 | | 0.78 |
| 265 | | | | | | | | |
| 266 | 0.671 | | 0.203 | 0.856 | | | | |
| 267 | | 1.854 | | | | | | |
| 268 | | | | | | | | |
| 269 | | | 0.608 | 2.055 | 1.144 | | | |
| 270 | | | | 0.343 | | 0.765 | | |
| 271 | | 0.618 | | | | 0.765 | | |
| 272 | | | | 0.514 | | 2.296 | | |
| 273 | | | | | 0.572 | | 0.796 | 1.171 |
| 274 | | 0.618 | | | | 0.765 | | |
| 275 | | | | | 1.144 | 0.765 | | |
| 276 | | | | | | | 0.398 | |
| 277 | | | | | | 2.296 | | |
| 278 | | | | 1.028 | | 2.296 | 0.398 | 0.39 |
| 279 | | | | | | | | |
| 280 | | | | 0.856 | | | | |
| 281 | | | 0.203 | 0.685 | | | | 0.39 |
| 282 | | | | 0.514 | | 0.765 | | |
| 283 | | | | | | | | |
| 284 | 2.013 | | | | | | | |
| 285 | | 0.618 | | | 1.144 | | | |
| 286 | | | | 0.171 | 0.572 | | | |
| 287 | | | | | | | | |
| 288 | | | | 0.514 | 1.717 | | | |
| 289 | | | | | 1.144 | 0.765 | | |
| 290 | | 0.618 | | 0.685 | | | | |
| 291 | | | | 0.685 | | 0.765 | | |
| 292 | | | | | | 0.765 | | |
| 293 | | | | 0.856 | | | | |
| 294 | | 0.618 | | | 0.572 | | | |
| 295 | | | | | | | | |
| 296 | | | | | | | | |
| 297 | | | | | | | | |
| 298 | | | | 1.37 | | | | |
| 299 | | | | | | 1.531 | | |
| 300 | | | | 2.227 | | | | |
| 301 | | | | 0.343 | | | | |
| 302 | | | 0.405 | | | | | |
| 303 | | | 0.203 | | | | | |
| 304 | | | | 1.028 | | | | 1.171 |
| 305 | | | | 2.398 | | | | |
| 306 | | | | | | | | |
| 307 | | | | 1.199 | | | | |
| 308 | | | | 0.171 | 1.144 | | | |
| 309 | | | | 0.685 | | 2.296 | | |
| 310 | | | | | 1.717 | | | |
| 311 | | | 0.203 | | | | | |
| 312 | | | | | | | | |
| 313 | | 2.472 | | | | 0.765 | | |
| 314 | | 3.708 | 0.203 | | | | | |
| 315 | | | 0.203 | | 1.717 | | | |
| 316 | | | 0.811 | 0.514 | | | | |
| 317 | | | | | | | | |
| 318 | | | | | | | | |
| 319 | | 1.854 | | | | | | |
| 320 | | | 0.203 | | | | | |
| 321 | | | 0.608 | 0.514 | | | | |
| 322 | | | | | | | | |
| 323 | | 1.236 | | | | | | 0.39 |
| 324 | | | 0.203 | 0.343 | | 0.765 | | |
| 325 | | 3.708 | | 1.028 | | | | |
| 326 | | | 0.608 | 1.199 | | | | |
| 327 | | 1.236 | | | | | | 0.78 |
| 328 | | 1.854 | 0.203 | | | | | |
| 329 | | | | 1.37 | | | | |
| 330 | 0.671 | | 0.203 | 2.055 | | | | |
| 331 | | | | | | | | |
| 332 | | | | 1.199 | | | | |
| 333 | 4.025 | | 0.203 | | | 2.296 | | |
| 334 | 3.355 | | | | 0.572 | | | |
| 335 | | | | | | | | |
| 336 | | | | | | | | |
| 337 | | 2.472 | | 1.028 | | | | |
| 338 | | | | | | | | 0.39 |
| 339 | | | | | 1.717 | 1.531 | | |
| 340 | | | | | | | | |
| 341 | | 2.472 | | 0.343 | 0.572 | | | |
| 342 | | | | | | | | |
| 343 | | 0.618 | | 1.199 | | | | |
| 344 | 2.684 | | | | | | | |
| 345 | | | 0.203 | | | | 0.796 | |
| 346 | | | | | | 2.296 | | |
| 347 | | 1.236 | | 1.028 | | | | |
| 348 | | | | | | | | |
| 349 | | | | 0.685 | | | | |
| 350 | | | | | | | | |
| 351 | | | | | | | | |
| 352 | | | | | | | | |
| 353 | | | | 0.514 | 1.717 | 0.765 | | |
| 354 | | | | | 2.289 | 1.531 | | |
| 355 | | | 0.405 | 0.685 | | | | |
| 356 | | 1.854 | 0.608 | 0.343 | | | | |
| 357 | | | 0.405 | 0.171 | | | | |
| 358 | | 0.618 | | | | | | |
| 359 | | | | | | | | |
| 360 | | | 0.405 | 0.171 | | | | |
| 361 | | | | | | 2.296 | | |
| 362 | | | | 0.514 | | 2.296 | | 0.39 |
| 363 | | | | | | | | 0.78 |
| 364 | | | | | | | | |
| 365 | | | 0.608 | | | | | |
| 366 | | | | | | 1.531 | | |
| 367 | | | | 0.685 | | 1.531 | | |
| 368 | | | | | | | | |
| 369 | | 0.618 | | | | | | |
| 370 | | | 0.203 | | | | | |
| 371 | | | 0.203 | | | | | |
| 372 | | | | 0.171 | | | | 1.561 |
| 373 | | 0.618 | | | | | | 0.78 |
| 374 | | | | 0.171 | | | | |
| 375 | | 0.618 | 0.203 | 0.856 | | | | |
| 376 | | | | | | | | |
| 377 | | | | | 1.144 | 1.531 | | |
| 378 | | | 0.608 | | | | | |
| 379 | | | 0.608 | | | | 1.989 | |
| 380 | 2.684 | | | | | | | |
| 381 | | | | | | | | |
| 382 | | 1.854 | | | | | | 0.39 |
| 383 | | | | 0.856 | | | | |
| 384 | | 0.618 | | | | | | |
| 385 | | | | 0.856 | | | | |
| 386 | | | | | | | | |
| 387 | | | | | | 0.765 | | |
| 388 | | | | | | | | |
| 389 | | 1.854 | | | | | | |
| 390 | | | | 1.028 | | | | |
| 391 | | | | 0.514 | | | | |
| 392 | | | | | | | | 0.39 |
| 393 | | 0.618 | | | | | | |
| 394 | | 0.618 | | 1.028 | | 0.765 | | |
| 395 | | 0.618 | | | | 1.531 | | |
| 396 | | | | 0.572 | | | | |
| 397 | | | 0.203 | 0.685 | | | 0.398 | |
| 398 | | | | 0.856 | 0.572 | | | |
| 399 | | | | | | | | |
| 400 | | | | | 1.144 | | | |
| 401 | | | | | 1.144 | | | |
| 402 | | 0.618 | | 0.856 | | 1.531 | 0.398 | |
| 403 | | | | | | | | |
| # Biomarkers | 7 | 32 | 31 | 59 | 23 | 31 | 9 | 15 |
| Per Sample | | | | | | | | |
| % Coverage | 5% | 21% | 21% | 40% | 15% | 21% | 6% | 10% |
| | Stage | | | | | | | |
| | Braak VI | Braak VI | Braak VI | Braak VI | Braak VI | Braak VI | Braak VI | Braak VI |
| Seq. ID | SRR1568538 | SRR1568562 | SRR1568566 | SRR1568598 | SRR1568600 | SRR1568611 | SRR1568641 | SRR1568648 |
| 255 | | | | | | | 0.674 | |
| 256 | | | | | | | 1.348 | |
| 257 | | 0.227 | | | | | 1.348 | |
| 258 | | 0.227 | | | | | 0.674 | 0.414 |
| 259 | | | | | | | | |
| 260 | | | | | | | | 0.828 |
| 261 | | | | | 3.88 | | 3.37 | |
| 262 | | | | | | | | |
| 263 | | 0.455 | | | | | | |
| 264 | | | | 0.236 | | | | |
| 265 | | 0.455 | 12.099 | | | | 3.37 | |
| 266 | | | | | | | 0.674 | |
| 267 | | | | | | | | |
| 268 | | | | | | | | |
| 269 | | | | | | | 2.022 | |
| 270 | | 0.682 | | | | | | |
| 271 | | | | | 7.76 | | | |
| 272 | | 0.227 | | | | | | |
| 273 | | | | | | | | |
| 274 | | 0.682 | | | | | | |
| 275 | | 0.682 | | | | | | |
| 276 | 0.633 | 0.227 | | | | | | |
| 277 | | 1.137 | | | | | 1.348 | |
| 278 | | | | | | | | |
| 279 | | 0.455 | | | | | | |
| 280 | | 0.455 | | | | | | |
| 281 | | | | | | | 2.696 | |
| 282 | | 0.91 | | | | | | |
| 283 | | | | | | | | |
| 284 | | 0.227 | | | | | | |
| 285 | | 1.137 | | | | | 0.674 | |
| 286 | | | | | | | | 2.899 |
| 287 | | | | | | | | 0.828 |
| 288 | | | | | | | | |
| 289 | | | | 0.236 | | | 0.674 | |
| 290 | | 0.227 | | | | | 0.674 | |
| 291 | | | | | | | 2.022 | |
| 292 | | 0.91 | | | | | | |
| 293 | | | | | | | | |
| 294 | | | | | | | 2.696 | |
| 295 | | | | | | | | |
| 296 | | | | | | | | 0.414 |
| 297 | | | | | | | | 7.04 |
| 298 | | | | 0.236 | | | 6.741 | |
| 299 | | | | | | | | |
| 300 | | | | | | | 0.674 | |
| 301 | | 0.227 | | | | | 8.089 | |
| 302 | | | | | | | | |
| 303 | | | | | 9.053 | | 1.348 | |
| 304 | | | | | | | 3.37 | |
| 305 | | | | 0.118 | | | | |
| 306 | | | | 0.707 | | | 6.741 | |
| 307 | | | | | | | 0.674 | |
| 308 | | | | | 4.526 | | | |
| 309 | | | | | | | | |
| 310 | | | | | | | | 0.828 |
| 311 | | | | | | | | |
| 312 | | | | 0.353 | | | | |
| 313 | | | | | | | 2.022 | |
| 314 | | | | | | | | |
| 315 | | | | | | | | |
| 316 | | | | | | | 5.393 | |
| 317 | | | | | | | | 0.414 |
| 318 | | | | | | 0.265 | | |
| 319 | | | | 0.236 | | | | 1.242 |
| 320 | | | | | | | | 1.656 |
| 321 | | | | | | | 2.022 | |
| 322 | | | | 0.118 | | | | |
| 323 | | | | | | | 2.696 | |
| 324 | | | | | 6.466 | | | |
| 325 | | | | | | | | |
| 326 | | | | | | | 2.022 | |
| 327 | | | | | | | | |
| 328 | | 0.455 | | | | | | |
| 329 | | 0.227 | | | | | 1.348 | |
| 330 | | | | | | | | |
| 331 | | | | | | | 5.393 | |
| 332 | | 0.455 | | | | | | |
| 333 | | | | | | | | |
| 334 | | 0.227 | | | | | | |
| 335 | | | | | | | | |
| 336 | | 0.682 | | | | | 2.022 | |
| 337 | | | | | | | | |
| 338 | | | | | | | | |
| 339 | | | | | | | | |
| 340 | | | | | | | | |
| 341 | | | | | | | | |
| 342 | | 0.91 | | | | | | |
| 343 | | | | | | | | |
| 344 | | | | | | | | |
| 345 | | 0.455 | | | | | | |
| 346 | | | | | | | | |
| 347 | | | | | | | | |
| 348 | | 0.455 | | | | | | |
| 349 | | 0.227 | | | | | | |
| 350 | | | | | 2.587 | | | |
| 351 | | | | | | | 1.348 | 1.656 |
| 352 | | | | | | | | |
| 353 | | | | | | | | |
| 354 | | 0.455 | | | 2.587 | | | |
| 355 | | | | | | | | |
| 356 | | | | | | | 2.022 | |
| 357 | | | | | | | | |
| 358 | | | | | | | | |
| 359 | | | | | | | | |
| 360 | | | | | | | 0.674 | |
| 361 | | | | | | | | |
| 362 | | | | | | | | |
| 363 | | | | | | | | |
| 364 | | | | 0.118 | | | | |
| 365 | | | | | | | | 0.414 |
| 366 | | | | | | | | |
| 367 | | 0.227 | | | | | | |
| 368 | | | | | | | | |
| 369 | | | | | | | | |
| 370 | | 0.227 | | | | | | |
| 371 | | | | | | | | |
| 372 | | 0.227 | | 0.118 | | | | |
| 373 | | 0.455 | | | | | | |
| 374 | | 0.227 | | | | | | |
| 375 | | | | | | | | |
| 376 | | | | 0.118 | | | | |
| 377 | | | | | | | | |
| 378 | | | | | | | | |
| 379 | 0.633 | | | | | | | |
| 380 | | 0.455 | | | | | | |
| 381 | | 0.227 | | 0.118 | | | 4.719 | |
| 382 | | 0.227 | | | | | 3.37 | |
| 383 | | 0.227 | | | | | | |
| 384 | | | | | | | | |
| 385 | | | | 0.118 | | | | |
| 386 | | | | 0.118 | | | | |
| 387 | | | | | | | | |
| 388 | | | | | | | | |
| 389 | | 0.682 | | | | | | |
| 390 | | | | 0.118 | | | | |
| 391 | | | | | | | | 0.828 |
| 392 | | | | | | | | |
| 393 | | 0.227 | | | | | | |
| 394 | | 0.455 | | | | | | |
| 395 | | 0.455 | | | | | | |
| 396 | | 0.455 | | 0.118 | | | 2.696 | |
| 397 | | | | 0.236 | | | | |
| 398 | | 0.455 | | | | | | 0.828 |
| 399 | | 0.227 | | | | | 2.696 | |
| 400 | | | | | 3.88 | | | |
| 401 | | | | | | | | |
| 402 | | | | | | | | |
| 403 | | | | | | | | |
| # Biomarkers | 2 | 44 | 1 | 17 | 8 | 1 | 37 | 14 |
| Per Sample | | | | | | | | |
| % Coverage | 1% | 30% | 1% | 11% | 5% | 1% | 25% | 9% |

| | Stage | | |
|---|---|---|---|
| | Braak VI | Braak VI | Braak VI |
| Seq. ID | SRR1568678 | SRR1568748 | SRR1568756 |
| 255 | | | |
| 256 | | | |
| 257 | | | |
| 258 | | | |
| 259 | | | |
| 260 | | | |
| 261 | | | 0.313 |
| 262 | | 0.244 | |
| 263 | | | 0.078 |
| 264 | | | |
| 265 | | | 0.078 |
| 266 | | | |
| 267 | | | 0.313 |
| 268 | | | 0.782 |
| 269 | | | |
| 270 | | | |
| 271 | | 0.489 | |
| 272 | | | |
| 273 | | | |
| 274 | | | |
| 275 | | | |
| 276 | | | |
| 277 | | | |
| 278 | | | |
| 279 | | | |
| 280 | | 0.244 | |
| 281 | | | |
| 282 | | | |
| 283 | | 0.244 | 0.078 |
| 284 | | | 0.078 |
| 285 | | | |
| 286 | | | |
| 287 | | | |
| 288 | | 0.244 | |
| 289 | | | |
| 290 | | | |
| 291 | | | |
| 292 | | 0.244 | |
| 293 | | | |
| 294 | | | |
| 295 | | | 1.408 |
| 296 | | | |
| 297 | | | 0.156 |
| 298 | | | |
| 299 | | 0.244 | |
| 300 | | | |
| 301 | | | |
| 302 | | | 0.078 |
| 303 | | 0.489 | |
| 304 | | | |
| 305 | | 0.489 | |
| 306 | | | |
| 307 | | | |
| 308 | | | |
| 309 | | | |
| 310 | | | |
| 311 | | | |
| 312 | | | 0.078 |
| 313 | | | |
| 314 | | | |
| 315 | | | |
| 316 | | | |
| 317 | | 0.244 | |
| 318 | | | |
| 319 | | | |
| 320 | | | |
| 321 | | | |
| 322 | | | |
| 323 | | | |
| 324 | | | |
| 325 | | | |
| 326 | | | |
| 327 | | | |
| 328 | | | |
| 329 | | | 0.313 |
| 330 | | | |
| 331 | | 0.489 | |
| 332 | | 0.244 | |
| 333 | | | |
| 334 | | | |
| 335 | | | |
| 336 | | | |
| 337 | | | 0.078 |
| 338 | 0.14 | 0.244 | |
| 339 | | | |
| 340 | | 0.244 | |
| 341 | | | |
| 342 | | | |
| 343 | | | |
| 344 | | | |
| 345 | | | |
| 346 | | | |
| 347 | | | |
| 348 | | | 0.156 |
| 349 | | 0.244 | |
| 350 | | | |
| 351 | | | |
| 352 | | | 0.626 |
| 353 | | | |
| 354 | | | |
| 355 | | | |
| 356 | | | |
| 357 | | | |
| 358 | | | |
| 359 | | | 0.391 |
| 360 | | | |
| 361 | | | 0.469 |
| 362 | | | |
| 363 | | | 0.391 |
| 364 | | | 0.156 |
| 365 | | | 0.235 |
| 366 | | 0.244 | 0.391 |
| 367 | | | |
| 368 | | | 0.391 |
| 369 | | | 0.078 |
| 370 | | | 0.156 |
| 371 | | | 0.469 |
| 372 | | | |
| 373 | | | |
| 374 | | | |
| 375 | | | |
| 376 | | | 0.078 |
| 377 | | | |
| 378 | | | 0.078 |
| 379 | | | 0.078 |
| 380 | | | |
| 381 | | 0.244 | |
| 382 | | | |
| 383 | | | |
| 384 | | | 0.235 |
| 385 | | | |
| 386 | | | |
| 387 | | 0.733 | |
| 388 | | | 0.313 |
| 389 | | 0.244 | |
| 390 | | | |
| 391 | | | |
| 392 | | | |
| 393 | | | |
| 394 | | | |
| 395 | | | |
| 396 | | | |
| 397 | | | |
| 398 | | | |
| 399 | | | |
| 400 | | | |
| 401 | | | |
| 402 | | | |
| 403 | | | |
| # Biomarkers | 1 | 19 | 30 |
| Per Sample | | | |
| % Coverage | 1% | 13% | 20% |

TABLE 8
Identified sRNA biomarkers in serum that have a positive correlation
with Braak Stage in order to monitor Alzheimer's Disease

| | | | | | | | | | | |
|---|---|---|---|---|---|---|---|---|---|---|
| | | | | | Braak | Braak | Braak | Braak | Braak | # |
| Seq. ID | Total Reads | Specificity | Sensitivity | p-value | II Avg | III Avg | IV Avg | V Avg | VI Avg | Hits |

| | | | | | | | | |
|---|---|---|---|---|---|---|---|---|
| 257 | 21 | 100% | 15.69% | 2.61E−05 | 7.416 | 0.964 | 0.868 | 3 |
| 270 | 19 | 100% | 11.76% | 4.01E−04 | 5.944 | 2.266 | 0.597 | 3 |
| 272 | 19 | 100% | 11.76% | 4.01E−04 | 2.759 | 2.019 | 1.012 | 3 |
| 273 | 17 | 100% | 11.76% | 4.01E−04 | 1.981 | 1.664 | 0.846 | 3 |
| 279 | 14 | 100% | 11.76% | 4.01E−04 | 3.678 | 0.798 | 0.455 | 3 |
| 286 | 12 | 100% | 11.76% | 4.01E−04 | 0.991 | 1.200 | 1.214 | 3 |
| 288 | 12 | 100% | 11.76% | 4.01E−04 | 1.839 | 0.277 | 0.825 | 3 |
| 314 | 17 | 100% | 9.80% | 1.53E−03 | 5.944 | 1.754 | 0.203 | 3 |
| 319 | 17 | 100% | 9.80% | 1.53E−03 | 4.114 | 1.854 | 0.739 | 3 |
| 325 | 16 | 100% | 9.80% | 1.53E−03 | 1.839 | 1.433 | 1.028 | 3 |
| 332 | 15 | 100% | 9.80% | 1.53E−03 | 2.759 | 1.486 | 0.633 | 3 |
| 341 | 14 | 100% | 9.80% | 1.53E−03 | 4.598 | 1.270 | 0.458 | 3 |
| 374 | 12 | 100% | 9.80% | 1.53E−03 | 5.518 | 0.422 | 0.199 | 3 |
| 391 | 11 | 100% | 9.80% | 1.53E−03 | 1.854 | 1.148 | 0.671 | 3 |
| 393 | 11 | 100% | 9.80% | 1.53E−03 | 2.759 | 0.588 | 0.227 | 3 |

TABLE 9
Identified sRNA biomarkers in colon epithelium tissue that are associated with Normal individuals.

| SEQ | | | | | | | | | | |
|---|---|---|---|---|---|---|---|---|---|---|
| ID | | | | | | | | | | |
| NO: | Marker | importance | imp_SE | sRNA_name | ref | ext | swaps | chosen | thislbl | otherlbl |
| 405 | GCTGATTGTCACGTTC | 0.61173 | 0.11392 | hsa-mir- | (0:0) | (GC:) | (1: T > C) | 0.9 | 2.305 | 0.767 |
| | TGATT | | | 5701 | | | | | | |
| 406 | GCCCCTGGGCCTATCC | −0.50514 | 0.07172 | hsa-mir- | (0:−1) | (:) | ( ) | 1 | 1.473 | 2.614 |
| | TAGA | | | 331-3p | | | | | | |
| 407 | AGTTCTTCAGTGGCAA | −0.43217 | 0.12976 | hsa-mir- | (0:−3) | (:) | ( ) | 0.7 | −0.639 | 0.822 |
| | GCT | | | 22-5p | | | | | | |
| 408 | ACCCTGTAGAACCGAA | 0.23477 | 0.08481 | hsa-mir- | (1:−1) | (:A) | ( ) | 0.5 | 3.3 | 1.212 |
| | TTTGTA | | | 10b-5p | | | | | | |
| 409 | TAGGTAGTTTCCTGTT | 0.17757 | 0.0569 | hsa-mir- | (0:−1) | (:AT) | (11: A > C) | 0.8 | 0.15 | −0.592 |
| | GTTGGAT | | | 196a-5p | | | | | | |
| 410 | ACCCTGTAGATCTGAA | 0.16483 | 0.10074 | hsa-mir- | (1:−1) | (:) | (10: A > T, | 0.3 | 0.782 | −0.34 |
| | TTTGT | | | 10b-5p | | | 12: C > T) | | | |
| 411 | TGAGATGAAGCTGTAG | 0.16362 | 0.03238 | hsa-mir- | (0:0) | (:C) | (8: C > A, | 0.8 | 0.779 | −0.308 |
| | CTC | | | 4770 | | | 9: A > G) | | | |
| 412 | TACCCTGTAGAACCGA | 0.15816 | 0.04547 | hsa-mir- | (0:−1) | (:) | (19: T > G) | 0.7 | 1.483 | −0.398 |
| | ATTGGT | | | 10b-5p | | | | | | |
| 413 | ACCCTGTAGAACCGAA | 0.1312 | 0.04783 | hsa-mir- | (1:−2) | (:G) | (10: T > A) | 0.5 | 0.875 | −0.605 |
| | TTTGG | | | 10a-5p | | | | | | |
| 414 | TAACAGTCTACAGCCA | −0.12465 | 0.06087 | hsa-mir- | (0:0) | (:) | ( ) | 0.6 | 3.56 | 4.436 |
| | TGGTCG | | | 132-3p | | | | | | |
| 415 | AGTTCTTCAGTGGCAA | −0.11012 | 0.05699 | hsa-mir- | (0:−2) | (:) | ( ) | 0.3 | −0.394 | 1.187 |
| | GCTT | | | 22-5p | | | | | | |
| 416 | TACCCTGTAGAACCGA | 0.09977 | 0.03596 | hsa-mir- | (0:−2) | (:G) | ( ) | 0.5 | 4.121 | 1.664 |
| | ATTTGG | | | 10b-5p | | | | | | |
| 417 | CAGTGCAATGATGAAA | −0.08933 | 0.05037 | hsa-mir- | (0:0) | (:) | (10: T > A, | 0.3 | 0.717 | 2.623 |
| | GGGCAT | | | 130a-3p | | | 12: A > G) | | | |
| 418 | TACCCTGTAGAACCGA | 0.07544 | 0.04788 | hsa-mir- | (0:−3) | (:A) | ( ) | 0.4 | 2.698 | 0.845 |
| | ATTTA | | | 10b-5p | | | | | | |
| 419 | TACAGTTGTTCAACCA | −0.07464 | 0.05019 | hsa-mir- | (1:0) | (:) | ( ) | 0.2 | −0.358 | 0.671 |
| | GTTACT | | | 582-5p | | | | | | |
| 420 | ACCCTGTAGAACCGAA | 0.06375 | 0.06375 | hsa-mir- | (1:0) | (:) | (10: T > A, | 0.1 | 0.747 | −0.188 |
| | TTTGGG | | | 10a-5p | | | 20: T > G) | | | |
| 421 | TACCCTGTAGGACCGA | 0.05883 | 0.03032 | hsa-mir- | (0:−1) | (:) | (10: A > G) | 0.4 | 1.962 | −0.355 |
| | ATTTGT | | | 10b-5p | | | | | | |
| 422 | TGGCAGTGTCTTAGCT | −0.05794 | 0.04762 | hsa-mir- | (0:−2) | (:) | ( ) | 0.2 | −0.482 | 1.044 |
| | GGTT | | | 34a-5p | | | | | | |
| 423 | ACCCTGTAGAACCGAA | 0.04848 | 0.03233 | hsa-mir- | (1:−3) | (:A) | (10: T > A) | 0.2 | 0.32 | −0.63 |
| | TTTA | | | 10a-5p | | | | | | |
| 424 | ACCCTGTAGAACCGAA | 0.04605 | 0.04605 | hsa-mir- | (1:−1) | (:T) | ( ) | 0.1 | 1.076 | −0.146 |
| | TTTGTT | | | 10b-5p | | | | | | |
| 425 | TACCCTGTAGATCCGA | 0.04078 | 0.01861 | hsa-mir- | (0:−1) | (:) | (11: A > T, | 0.4 | 1.192 | −0.283 |
| | TTTTGT | | | 10b-5p | | | 16: A > T) | | | |
| 426 | TACCCTGTAGAACCGA | 0.03972 | 0.03306 | hsa-mir- | (0:−1) | (:) | (16: A > G) | 0.2 | 2.752 | 0.399 |
| | GTTTGT | | | 10b-5p | | | | | | |
| 427 | TTCAAGTAATCCAGGA | 0.03965 | 0.03658 | hsa-mir- | (0:−1) | (:CT) | ( ) | 0.2 | 0.841 | −0.548 |
| | TAGGCCT | | | 26a-5p | | | | | | |
| 428 | TACCCTGTAGAACCGA | 0.03939 | 0.03051 | hsa-mir- | (0:−1) | (:) | (20: G > A) | 0.2 | 1.886 | 0.183 |
| | ATTTAT | | | 10b-5p | | | | | | |
| 429 | TACCCTGTAGAACCGG | 0.03714 | 0.02781 | hsa-mir- | (0:−2) | (:) | (15: A > G) | 0.2 | 0.166 | −0.663 |
| | ATTTG | | | 10b-5p | | | | | | |
| 430 | TATTGCACTTGTCCCG | 0.03206 | 0.03206 | hsa-mir- | (0:2) | (:C) | (22: G > A) | 0.1 | 0.533 | −0.546 |
| | GCCTGTAGC | | | 92a-3p | | | | | | |
| 431 | ACCCTGTAGATCTGAA | 0.02789 | 0.02789 | hsa-mir- | (1:0) | (:A) | (12: C > T) | 0.1 | 0.267 | −0.681 |
| | TTTGTGA | | | 10a-5p | | | | | | |
| 432 | CACTAGATTGTGAGCT | 0.02652 | 0.02652 | hsa-mir- | (0:−3) | (:) | ( ) | 0.1 | 2.028 | 0.439 |
| | CCT | | | 28-3p | | | | | | |
| 433 | TACCCTGTAGTACCGA | 0.02641 | 0.02641 | hsa-mir- | (0:−1) | (:) | (10: A > T) | 0.1 | 1.227 | −0.21 |
| | ATTTGT | | | 10b-5p | | | | | | |
| 434 | CAGTGCAATGTTAAAA | −0.026 | 0.01733 | hsa-mir- | (0:−1) | (:A) | (10: A > T, | 0.2 | −0.212 | 1.183 |
| | GGGCAA | | | 130b-3p | | | 12: G > A) | | | |
| 435 | CTGACCTATGATTTGA | 0.02413 | 0.01324 | hsa-mir- | (0:0) | (:) | (11: A > T) | 0.3 | 1.746 | 0.096 |
| | CAGCC | | | 192-5p | | | | | | |
| 436 | CTGACCTATGAATTGA | 0.02306 | 0.01562 | hsa-mir- | (0:0) | (:CT) | ( ) | 0.2 | 2.004 | 0.427 |
| | CAGCCCT | | | 192-5p | | | | | | |
| 437 | CCACTGCCCCAGGTGC | −0.02248 | 0.02248 | hsa-mir- | (-2:0) | (:) | ( ) | 0.1 | −0.481 | 0.945 |
| | TGCTGG | | | 324-3p | | | | | | |
| 438 | TGAGGTAGTAGGTTGT | 0.02215 | 0.02215 | hsa-let- | (0:0) | (:) | (16: A > G, | 0.1 | 0.975 | −0.325 |
| | GTGGGT | | | 7c-5p | | | 20: T > G) | | | |
| 439 | ACTGTGCGTGTGACAG | −0.02097 | 0.01562 | hsa-mir- | (−1:−2) | (:) | ( ) | 0.2 | −0.666 | 0.215 |
| | CGGCT | | | 210-3p | | | | | | |
| 440 | CTGCGCAAGCTACTGC | −0.0202 | 0.0202 | hsa-let- | (0:−2) | (:) | ( ) | 0.1 | 1.199 | 2.896 |
| | CTTG | | | 7i-3p | | | | | | |
| 441 | CACCCGTAGAACCGAC | −0.02011 | 0.01097 | hsa-mir- | (0:0) | (:A) | ( ) | 0.3 | 3.612 | 4.648 |
| | CTTGCGA | | | 99b-5p | | | | | | |
| 442 | CTGACCTATGTATTGA | 0.01839 | 0.01249 | hsa-mir- | (0:0) | (:) | (10: A > T) | 0.2 | 2.279 | 0.663 |
| | CAGCC | | | 192-5p | | | | | | |
| 443 | TACCCTGTAGAACCGA | 0.01577 | 0.01577 | hsa-mir- | (0:−2) | (:C) | ( ) | 0.1 | 4.555 | 1.079 |
| | ATTTGC | | | 10b-5p | | | | | | |
| 444 | TGAGAACTGAATTCCA | −0.01551 | 0.01551 | hsa-mir- | (0:1) | (:AA) | (17: G > A, | 0.1 | −0.359 | 0.464 |
| | TAGGCTGAA | | | 146a-5p | | | 20: T > C) | | | |
| 445 | TGACCTATGAATTGAC | 0.01402 | 0.01402 | hsa-mir- | (1:3) | (:T) | (18: A > C) | 0.1 | 0.754 | −0.46 |
| | AGCCAATT | | | 215-5p | | | | | | |
| 446 | TACCCTGTAGAACCGA | 0.01382 | 0.01382 | hsa-mir- | (0:−1) | (:A) | ( ) | 0.1 | 5.669 | 4.122 |
| | ATTTGTA | | | 10b-5p | | | | | | |
| 447 | TGAGATGAAGCACTGT | 0.01158 | 0.01158 | hsa-mir- | (0:0) | (:) | (18: C > A) | 0.1 | 2.526 | 1.048 |
| | AGATC | | | 143-3p | | | | | | |
| 448 | TACCCTGTAGAACCGA | 0.0115 | 0.00939 | hsa-mir- | (0:−1) | (:) | (17: T > C) | 0.2 | 1.946 | 0.086 |
| | ACTTGT | | | 10b-5p | | | | | | |
| 449 | CTGACCTATGAACTGA | 0.01068 | 0.0088 | hsa-mir- | (0:0) | (:) | (12: T > C) | 0.2 | 2.713 | 0.568 |
| | CAGCC | | | 192-5p | | | | | | |
| 450 | GATTGTCACGTTCTGA | 0.00994 | 0.00994 | hsa-mir- | (2:0) | (G:) | ( ) | 0.1 | 0.926 | −0.013 |
| | TT | | | 5701 | | | | | | |
| 451 | TTACAGTCTACAGCCA | −0.007 | 0.007 | hsa-mir- | (0:0) | (:) | (1: A > T) | 0.1 | −0.541 | 0.325 |
| | TGGTCG | | | 132-3p | | | | | | |
| 452 | CATTGCACTTGTCTCG | 0.00642 | 0.00642 | hsa-mir- | (0:0) | (:AT) | ( ) | 0.1 | 2.02 | 0.798 |
| | GTCTGAAT | | | 25-3p | | | | | | |
| 453 | TACCCTGTTGAACCGA | 0.00629 | 0.00629 | hsa-mir- | (0:−1) | (:) | (8: A > T) | 0.1 | 0.959 | −0.227 |
| | ATTTGT | | | 10b-5p | | | | | | |
| 454 | CAAAGTGCTGTTCGTG | −0.00623 | 0.00623 | hsa-mir- | (0:−1) | (:) | ( ) | 0.1 | 2.94 | 3.614 |
| | CAGGTA | | | 93-5p | | | | | | |
| 455 | CTCGCTTCTGGCGCCA | −0.00413 | 0.00413 | <NA> | (NA:NA) | (NA:NA) | ( ) | 0.1 | −0.552 | 0.651 |
| | AGCGCCCGGC | | | | | | | | | |
| 456 | AACTGGCCCTCAAAGT | −0.00368 | 0.00368 | hsa-mir- | (0:−2) | (:) | ( ) | 0.1 | 0.083 | 1.702 |
| | CCCG | | | 193b-3p | | | | | | |
| 457 | TGAGAACTGAATTCCA | −0.00364 | 0.00364 | hsa-mir- | (0:−1) | (:AA) | ( ) | 0.1 | 0.256 | 1.187 |
| | TAGGCAA | | | 146b-5p | | | | | | |
| 458 | TGAGGTAGTAGATTGT | 0.00325 | 0.00325 | hsa-let- | (0:2) | (:) | (11: G > A) | 0.1 | 0.75 | −0.212 |
| | ATAGTTTT | | | 7a-5p | | | | | | |
| 459 | ACCCTGTAGATCCGAA | 0.00148 | 0.00148 | hsa-mir- | (1:-5) | (:) | ( ) | 0.1 | 0.215 | −0.459 |
| | T | | | 10a-5p | | | | | | |
| 460 | AGGCTGTGATGCTCTC | 0.00039 | 0.00039 | hsa-mir- | (0:−1) | (:CT) | ( ) | 0.1 | 0.595 | −0.142 |
| | CTGAGCCCT | | | 7974 | | | | | | |
| 461 | TAACACTGTCTGGTAA | 0.00027 | 0.00027 | hsa-mir- | (0:−5) | (:) | ( ) | 0.1 | 1.631 | −0.336 |
| | C | | | 200a-3p | | | | | | |
| 462 | TACCCTGTAGATCCGA | 0.00024 | 0.00024 | hsa-mir- | (0:−1) | (:) | (11: A > T, | 0.1 | 1.832 | −0.081 |
| | ATTCGT | | | 10b-5p | | | 19: T > C) | | | |

TABLE 10
Identified sRNA biomarkers in colon epithelium tissue that are associated with Crohn's disease.

| SEQ | | | | | | | | | | |
|---|---|---|---|---|---|---|---|---|---|---|
| ID | | | | | | | | | | |
| NO: | Marker | importance | imp_SE | sRNA_name | ref | ext | swaps | chosen | thislbl | otherlbl |
| 463 | CCGCCCCACCCCGCGC | 0.74618 | 0.16463 | <NA> | (NA:NA) | (NA:NA) | ( ) | 0.8 | 1.72 | −0.59 |
| | GCGCCGC | | | | | | | | | |
| 464 | CGCTTCTGGCGCCAAG | 0.25545 | 0.08406 | <NA> | (NA:NA) | (NA:NA) | ( ) | 0.7 | 1.39 | −0.62 |
| | CGCCCGGCCGC | | | | | | | | | |
| 465 | AGATTGAGGGTTCGTC | 0.25408 | 0.05563 | <NA> | (NA:NA) | (NA:NA) | ( ) | 0.8 | 2.73 | −0.37 |
| | CCTTCGTGGTCGCC | | | | | | | | | |
| 466 | GGCTTGGTCTAGGGGT | 0.21881 | 0.06902 | <NA> | (NA:NA) | (NA:NA) | ( ) | 0.7 | 2.2 | −0.46 |
| | ATGATTCTCGCTTT | | | | | | | | | |
| 467 | GGCTTTGTCTAGGGGT | 0.18401 | 0.12882 | <NA> | (NA:NA) | (NA:NA) | ( ) | 0.4 | 1.34 | −0.65 |
| | ATGATTCTCGCTT | | | | | | | | | |
| 468 | CCCGCCCCACCCCGCG | 0.15615 | 0.09596 | <NA> | (NA:NA) | (NA:NA) | ( ) | 0.3 | 1.5 | −0.64 |
| | CGCGCCGCT | | | | | | | | | |
| 469 | CGTACGGAAGACCCGC | 0.11296 | 0.05941 | <NA> | (NA:NA) | (NA:NA) | ( ) | 0.3 | 1.26 | −0.61 |
| | TCCCCGGCGCCGCT | | | | | | | | | |
| 470 | GTACGGAAGACCCGCT | 0.10944 | 0.10944 | <NA> | (NA:NA) | (NA:NA) | ( ) | 0.1 | 1.36 | −0.59 |
| | CCCCGGCGCCG | | | | | | | | | |
| 471 | TGGTCTAGCGGTTAGG | 0.09687 | 0.06389 | <NA> | (NA:NA) | (NA:NA) | ( ) | 0.3 | 1.02 | −0.66 |
| | ATTCCTGGTTTT | | | | | | | | | |
| 472 | CGCCCCACCCCGCGCG | 0.09422 | 0.03815 | <NA> | (NA:NA) | (NA:NA) | ( ) | 0.5 | 1.64 | −0.61 |
| | CGCCGC | | | | | | | | | |
| 473 | CCCGCGAGGGGGGCCC | 0.07217 | 0.05546 | <NA> | (NA:NA) | (NA:NA) | ( ) | 0.2 | 1.03 | −0.58 |
| | GGGCAC | | | | | | | | | |
| 474 | GCGCCGCCGCCCCCCC | 0.06871 | 0.04611 | <NA> | (NA:NA) | (NA:NA) | ( ) | 0.2 | 1.64 | −0.67 |
| | CACGCCCGGGGC | | | | | | | | | |
| 475 | GCTCCCCGTCCTCCCC | 0.06762 | 0.06762 | <NA> | (NA:NA) | (NA:NA) | ( ) | 0.1 | 1.58 | −0.67 |
| | CCTCCCC | | | | | | | | | |
| 476 | GCGCAATGAAGGTGAA | 0.06288 | 0.03999 | <NA> | (NA:NA) | (NA:NA) | ( ) | 0.4 | 1.03 | −0.6 |
| | GGCCGGCGC | | | | | | | | | |
| 477 | ACGCTGCCAGTTGAAG | 0.05063 | 0.05063 | hsa-mir- | (0:0) | (:) | (1: A > C) | 0.1 | 0.86 | −0.46 |
| | AACTGT | | | 22-3p | | | | | | |
| 478 | GCCCCTGGGCCTATCC | 0.04958 | 0.03308 | hsa-mir- | (0:0) | (:AA) | ( ) | 0.2 | 0.68 | −0.65 |
| | TAGAAAA | | | 331-3p | | | | | | |
| 479 | GCGGGTCCGGCCGTGT | 0.04831 | 0.04831 | <NA> | (NA:NA) | (NA:NA) | ( ) | 0.1 | 0.65 | −0.67 |
| | CGGCGGC | | | | | | | | | |
| 480 | GGCTTGGTCTAGGGGT | 0.04437 | 0.04437 | <NA> | (NA:NA) | (NA:NA) | ( ) | 0.1 | 3.5 | 0.65 |
| | ATGATTCTCGCT | | | | | | | | | |
| 481 | CCACCTCCCCTGCAAA | 0.03994 | 0.02586 | hsa-mir- | (0:−1) | (:) | ( ) | 0.4 | 0.46 | −0.6 |
| | CGTCC | | | 1306-5p | | | | | | |
| 482 | GGTTAGGATTCCTGGT | 0.03829 | 0.03829 | <NA> | (NA:NA) | (NA:NA) | ( ) | 0.1 | 1.08 | −0.57 |
| | TTT | | | | | | | | | |
| 483 | TCTGGCATGCTAACTA | 0.03622 | 0.03622 | <NA> | (NA:NA) | (NA:NA) | ( ) | 0.1 | 0.84 | −0.67 |
| | GTTACGCGACCCCC | | | | | | | | | |
| 484 | CGCGTCCCCCGAAGAG | 0.03391 | 0.03391 | <NA> | (NA:NA) | (NA:NA) | ( ) | 0.1 | 1.08 | −0.68 |
| | GGGGACGGCGGAGC | | | | | | | | | |
| 485 | GCGGAGCGAGCGCACG | 0.0323 | 0.0323 | <NA> | (NA:NA) | (NA:NA) | ( ) | 0.1 | 0.79 | −0.52 |
| | GGGTCGGCGGCGAC | | | | | | | | | |
| 486 | CCCCCGCCCCACCCCG | 0.02563 | 0.02563 | <NA> | (NA:NA) | (NA:NA) | ( ) | 0.1 | 1.3 | −0.68 |
| | CGCGCGCCGCTCGC | | | | | | | | | |
| 487 | CCGTAGGTGAACCTGC | 0.02433 | 0.01963 | <NA> | (NA:NA) | (NA:NA) | ( ) | 0.2 | 2.36 | −0.5 |
| | GGAAGGATCATTA | | | | | | | | | |
| 488 | GGGCTACGCCTGTCTG | 0.02206 | 0.02206 | <NA> | (NA:NA) | (NA:NA) | ( ) | 0.1 | 2.74 | 0.07 |
| | AGCGTCGCTT | | | | | | | | | |
| 489 | GCTACGCCTGTCTGAG | 0.02103 | 0.02103 | <NA> | (NA:NA) | (NA:NA) | ( ) | 0.1 | 1.48 | −0.46 |
| | CGTCGCTT | | | | | | | | | |
| 490 | CCCCCACAACCGCGCT | 0.0204 | 0.0204 | <NA> | (NA:NA) | (NA:NA) | ( ) | 0.1 | 1.43 | −0.36 |
| | TGACTAGCTT | | | | | | | | | |
| 491 | CCCTACCCCCCCGGCC | 0.01307 | 0.01307 | <NA> | (NA:NA) | (NA:NA) | ( ) | 0.1 | 1.25 | −0.56 |
| | CCGTC | | | | | | | | | |
| 492 | CCCGCCCCACCCCGCG | 0.01108 | 0.01108 | <NA> | (NA:NA) | (NA:NA) | ( ) | 0.1 | 1.7 | −0.59 |
| | CGCGCCGCTCGC | | | | | | | | | |
| 493 | GGGGGTATAGCTCAGT | 0.01022 | 0.01022 | <NA> | (NA:NA) | (NA:NA) | ( ) | 0.1 | 1.12 | −0.58 |
| | GGTAGAGCGTGCTT | | | | | | | | | |
| 494 | GTCGGTCGGGCTGGGG | 0.00996 | 0.00996 | <NA> | (NA:NA) | (NA:NA) | ( ) | 0.1 | 2.53 | −0.51 |
| | CGCGAAGCGGGGCT | | | | | | | | | |
| 495 | TCAGTGGAGAGCATTT | 0.00991 | 0.00991 | <NA> | (NA:NA) | (NA:NA) | ( ) | 0.1 | 0.54 | −0.66 |
| | GACT | | | | | | | | | |
| 496 | CACCCCTAGAACCGAC | 0.0095 | 0.0095 | hsa-mir- | (0:0) | (:) | (5: G > C) | 0.1 | 0.17 | −0.66 |
| | CTTGCG | | | 99b-5p | | | | | | |
| 497 | CCTCACCATCCCTTCT | 0.00892 | 0.00892 | hsa-mir- | (0:1) | (:) | ( ) | 0.1 | 0.2 | −0.65 |
| | GCCTGCA | | | 6511a-3p | | | | | | |
| 498 | GTCAGGATGGCCGAGC | 0.00647 | 0.00647 | <NA> | (NA:NA) | (NA:NA) | ( ) | 0.1 | 2.13 | 0.36 |
| | GGTCT | | | | | | | | | |
| 499 | TCCCTGGTCTAGTGGT | 0.00644 | 0.00644 | <NA> | (NA:NA) | (NA:NA) | ( ) | 0.1 | 1.6 | −0.27 |
| | TAGGATTCGGCGCG | | | | | | | | | |
| 500 | TGAGATGAAGCACTGT | −0.00555 | 0.00555 | hsa-mir- | (0:0) | (:) | (18: C > A) | 0.1 | −0.07 | 1.91 |
| | AGATC | | | 143-3p | | | | | | |
| 501 | GGATCGGCCCCGCCGG | 0.00523 | 0.00523 | <NA> | (NA:NA) | (NA:NA) | ( ) | 0.1 | 1.04 | −0.68 |
| | GGTCGGC | | | | | | | | | |
| 502 | GGAACCTGCGGAAGGA | 0.00215 | 0.00215 | <NA> | (NA:NA) | (NA:NA) | ( ) | 0.1 | 2.24 | −0.33 |
| | TCATTA | | | | | | | | | |
| 503 | TGAGGTAGTAGGTTGT | 0.00179 | 0.00179 | hsa-mir- | (0:1) | (:) | (5: G > T, | 0.1 | 0.92 | −0.53 |
| | ATGGTTG | | | 4510 | | | 12: A > T) | | | |
| 504 | GTCTAGTGGTTAGGAT | 0.00093 | 0.00093 | <NA> | (NA:NA) | (NA:NA) | ( ) | 0.1 | 1.61 | −0.38 |
| | TCGGCGCT | | | | | | | | | |
| 505 | TCCCTGGTCTAGTGGC | 0.00085 | 0.00085 | <NA> | (NA:NA) | (NA:NA) | ( ) | 0.1 | 0.72 | −0.64 |
| | TAGGATTCGGCGCT | | | | | | | | | |
| 506 | GCCGCCCCCCCCACGC | 0.0002 | 0.0002 | <NA> | (NA:NA) | (NA:NA) | ( ) | 0.1 | 0.59 | −0.68 |
| | CCGGGGC | | | | | | | | | |

TABLE 11
Identified sRNA biomarkers in colon epithelium tissue that are associated with Ulcerative colitis.

| SEQ | | | | | | | | | | |
|---|---|---|---|---|---|---|---|---|---|---|
| ID | | | | | | | | | | |
| NO: | Marker | importance | imp_SE | sRNA_name | ref | ext | swaps | chosen | thislbl | otherlbl |
| 507 | TGTCAGTTTGTCAAAT | 0.46706 | 0.1009 | hsa-mir- | (0:2) | (:) | ( ) | 0.9 | 1.892 | 0.1084 |
| | ACCCCAAG | | | 223-3p | | | | | | |
| 508 | CAGCAGCAATTCATGT | 0.29749 | 0.09883 | hsa-mir- | (0:0) | (:T) | ( ) | 0.6 | 0.578 | −0.613 |
| | TTTGAAT | | | 424-5p | | | | | | |
| 509 | GTGGTTGTAGTCCGTG | −0.22154 | 0.09667 | <NA> | (NA:NA) | (NA:NA) | ( ) | 0.5 | −0.373 | 1.2368 |
| | CGAGAATACC | | | | | | | | | |
| 510 | GGATATCATCATATAC | 0.1973 | 0.11602 | hsa-mir- | (0:1) | (:) | ( ) | 0.4 | 2.428 | 0.8535 |
| | TGTAAGT | | | 144-5p | | | | | | |
| 511 | TAACAGTCTCCAGTCA | 0.14329 | 0.07797 | hsa-mir- | (0:−1) | (:) | ( ) | 0.6 | 1.215 | −0.5329 |
| | CGGC | | | 212-3p | | | | | | |
| 512 | TCAGTGCACTACAGAA | 0.13604 | 0.06626 | hsa-mir- | (0:0) | (:T) | (20: G > T) | 0.5 | 0.643 | −0.6209 |
| | CTTTTTT | | | 148a-3p | | | | | | |
| 513 | CCAGTGGGGCTGCTGT | −0.13318 | 0.07284 | hsa-mir- | (0:0) | (:T) | ( ) | 0.3 | 0.857 | 2.7111 |
| | TATCTGT | | | 194-3p | | | | | | |
| 514 | GATAAAGTAGAAAGCA | 0.13252 | 0.06175 | hsa-mir- | (1:0) | (G:) | ( ) | 0.4 | 1.653 | −0.6021 |
| | CTACT | | | 142-5p | | | | | | |
| 515 | TAGGTAGTTTCCTGTT | −0.1183 | 0.04091 | hsa-mir- | (0:−1) | (:AT) | (11: A > C) | 0.6 | −0.676 | −0.1724 |
| | GTTGGAT | | | 196a-5p | | | | | | |
| 516 | ATGCTTATCAGACTGA | 0.11425 | 0.07239 | hsa-mir- | (2:0) | (AT:) | ( ) | 0.3 | 1.241 | −0.512 |
| | TGTTGA | | | 21-5p | | | | | | |
| 517 | TAGTGCAATATTGCTT | 0.10893 | 0.0759 | hsa-mir- | (0:−1) | (:) | ( ) | 0.3 | 0.82 | 0.0483 |
| | ATAGGG | | | 454-3p | | | | | | |
| 518 | CCCATAAAGTAGAAAG | 0.10582 | 0.05342 | hsa-mir- | (−2:0) | (:) | ( ) | 0.5 | 1.414 | −0.294 |
| | CACTACT | | | 142-5p | | | | | | |
| 519 | TACCCATTGCATATCG | 0.097 | 0.07557 | hsa-mir- | (0:−1) | (:) | ( ) | 0.3 | 0.876 | −0.4505 |
| | GAGTT | | | 660-5p | | | | | | |
| 520 | ACTGGACTTGGAGTCA | −0.09333 | 0.05017 | hsa-mir- | (0:3) | (:A) | (13: G > T, | 0.3 | 2.232 | 4.1887 |
| | GAAGGAA | | | 378b | | | 19: A > G | | | |
| 521 | AAGCAGCAATTCATGT | 0.09165 | 0.06219 | hsa-mir- | (1:−1) | (A:) | ( ) | 0.2 | 0.263 | −0.6458 |
| | TTTGA | | | 424-5p | | | | | | |
| 522 | CTGCAGCACGTAAATA | 0.0866 | 0.05794 | hsa-mir- | (2:0) | (CT:) | ( ) | 0.2 | 0.882 | −0.5753 |
| | TTGGCG | | | 16-5p | | | | | | |
| 523 | TGGCAGTGTCTTAGCT | 0.07815 | 0.06409 | hsa-mir- | (0:−2) | (:) | ( ) | 0.3 | 1.71 | −0.1242 |
| | GGTT | | | 34a-5p | | | | | | |
| 524 | ACTGGACTTGGAGTCA | −0.07752 | 0.052 | hsa-mir- | (0:−2) | (:) | (20: A > G, | 0.2 | −0.284 | 1.3769 |
| | GAAGGTT | | | 378c | | | 21: G > T) | | | |
| 525 | TGAGAACTGAATTCCA | 0.07149 | 0.03423 | hsa-mir- | (0:4) | (:) | (24: G > A) | 0.6 | 2.372 | 0.6917 |
| | TAGGCTGTAA | | | 146b-5p | | | | | | |
| 526 | ACTGGACTTGGAGTCA | −0.0679 | 0.04539 | hsa-mir- | (0:−2) | (:) | (20: A > G | 0.2 | 0.289 | 2.0819 |
| | GAAGGAT | | | 378c | | | 21: G > A) | | | |
| 527 | TGAGAACTGAATTCCA | 0.06566 | 0.04343 | hsa-mir- | (0:4) | (:T) | (24: G > A) | 0.3 | 0.687 | −0.4488 |
| | TAGGCTGTAAT | | | 146b-5p | | | | | | |
| 528 | GTTGAGACTCTGAAAT | −0.06461 | 0.05023 | hsa-mir- | (−2:−7) | (G:GATT) | (3: C > G, | 0.2 | −0.649 | 0.1771 |
| | CTGATT | | | 4431 | | | 14: A > A) | | | |
| 529 | TTAATGCTAATCGTGA | 0.06346 | 0.02758 | hsa-mir- | (0:−4) | (:) | ( ) | 0.4 | 2.46 | 0.3365 |
| | TAG | | | 155-5p | | | | | | |
| 530 | TGAGAACTGAATTCCA | 0.06095 | 0.0468 | hsa-mir- | (0:−2) | (:AA) | (17: G > A) | 0.2 | 1.103 | 0.1217 |
| | TAGGAA | | | 146a-5p | | | | | | |
| 531 | CTATACGACCTGCTGC | −0.05799 | 0.05799 | hsa-let- | (0:−1) | (:A) | ( ) | 0.1 | 0.725 | 1.845 |
| | CTTTCA | | | 7d-3p | | | | | | |
| 532 | TACCCTGTAGAACCGA | −0.05773 | 0.04012 | hsa-mir- | (0:0) | (:) | (11: T > A, | 0.2 | −0.445 | 0.5034 |
| | ATTTGCG | | | 10a-5p | | | 21: T > C) | | | |
| 533 | TGGCAGTGTCTTAGCT | 0.05695 | 0.04073 | hsa-mir- | (0:−3) | (:) | ( ) | 0.2 | 0.721 | −0.5822 |
| | GGT | | | 34a-5p | | | | | | |
| 534 | CCAGTGGGGCTGCTGT | −0.05534 | 0.03762 | hsa-mir- | (0:−1) | (:) | ( ) | 0.3 | 1.163 | 2.2638 |
| | TATCT | | | 194-3p | | | | | | |
| 535 | TTGAGAACTGAATTCC | 0.05453 | 0.04544 | hsa-mir- | (−1:0) | (:) | ( ) | 0.2 | 2.563 | 0.8429 |
| | ATGGGTT | | | 146a-5p | | | | | | |
| 536 | TTACAGTCTACAGCCA | 0.04999 | 0.04437 | hsa-mir- | (0:0) | (:) | (1: A > T) | 0.2 | 0.833 | −0.4181 |
| | TGGTCG | | | 132-3p | | | | | | |
| 537 | ACTGGACTTGGAGTCA | −0.04834 | 0.0324 | hsa-mir- | (0:3) | (:) | (19: A > G, | 0.2 | 5.356 | 6.5699 |
| | GAAGGCT | | | 378d | | | 20: A > G) | | | |
| 538 | TGAGAACTGAATTCCA | 0.04829 | 0.0337 | hsa-mir- | (0:2) | (:AG) | ( ) | 0.2 | 0.761 | −0.2346 |
| | TAGGCTGTAG | | | 146b-5p | | | | | | |
| 539 | CCCATAAAGTAGAAAG | 0.04703 | 0.03279 | hsa-mir- | (−2:−1) | (:A) | ( ) | 0.2 | 2.327 | 0.2258 |
| | CACTACA | | | 142-5p | | | | | | |
| 540 | TGAGGTAGTAGTTTGT | 0.04637 | 0.04637 | hsa-let- | (0:−3) | (:) | ( ) | 0.1 | 3.668 | 2.5754 |
| | GCT | | | 7i-5p | | | | | | |
| 541 | CGGCGCAAGCTACTGC | 0.04625 | 0.04625 | hsa-let- | (0:−2) | (:) | (1: T > G) | 0.1 | 0.127 | −0.6692 |
| | CTTG | | | 7i-3p | | | | | | |
| 542 | AGTTCTTCAGTGGCAA | 0.04577 | 0.04577 | hsa-mir- | (0:−3) | (:) | ( ) | 0.1 | 1.084 | −0.0644 |
| | GCT | | | 22-5p | | | | | | |
| 543 | TCCCCTGTAGAACCGA | −0.04267 | 0.02897 | hsa-mir- | (0:−1) | (:) | (1: A > C) | 0.2 | −0.655 | 0.1801 |
| | ATTTGT | | | 10b-5p | | | | | | |
| 544 | ACTGGACTTGGAGTCA | -0.04209 | 0.02716 | hsa-mir- | (0:0) | (:ATT) | (9: A > G, | 0.3 | 1.615 | 3.1346 |
| | GAAGGCATT | | | 422a | | | 11: G > A) | | | |
| 545 | AAGCTCGGTCTGAGGC | −0.04032 | 0.03266 | hsa-mir- | (−1:−2) | (:) | ( ) | 0.2 | 0.598 | 1.7929 |
| | CCCTCA | | | 423-3p | | | | | | |
| 546 | CCAGTGGGGCTGCTGT | −0.03971 | 0.03971 | hsa-mir- | (0:0) | (:A) | ( ) | 0.1 | −0.383 | 1.5327 |
| | TATCTGA | | | 194-3p | | | | | | |
| 547 | TGAGGGAGTAGTTTGT | 0.03743 | 0.02474 | hsa-let- | (0:0) | (:A) | (5: T > G) | 0.3 | 0.516 | −0.5159 |
| | GCTGTTA | | | 7i-5p | | | | | | |
| 548 | AAGAAAGTAGAAAGCA | 0.03726 | 0.03726 | hsa-mir- | (1:0) | (A:) | (1: T > A) | 0.1 | 0.759 | −0.6659 |
| | CTACT | | | 142-5p | | | | | | |
| 549 | CGCTGCCAGTTGAAGA | 0.03671 | 0.03671 | hsa-mir- | (2:0) | (C:) | ( ) | 0.1 | 1.055 | −0.5449 |
| | ACTGT | | | 22-3p | | | | | | |
| 550 | GGCTGGTCCGATGGTA | −0.03534 | 0.03534 | hsa-mir- | (0:−1) | (:) | (8: A > C, | 0.1 | 0.079 | 1.378 |
| | GT | | | 6131 | | | 14: G > T) | | | |
| 551 | CTGGGAGAAGGCTGTT | −0.03467 | 0.03467 | hsa-mir- | (0:0) | (:) | ( ) | 0.1 | 0.783 | 1.6525 |
| | TACTCT | | | 30c-2-3p | | | | | | |
| 552 | AAGCAATTCTCAAAGG | 0.03329 | 0.01693 | hsa-mir- | (−3:−5) | (:) | ( ) | 0.4 | 0.38 | −0.6931 |
| | AGC | | | 5571-5p | | | | | | |
| 553 | CTCGGCGCCCCCTCGA | −0.03132 | 0.02602 | <NA> | (NA:NA) | (NA:NA) | ( ) | 0.2 | −0.37 | 0.6322 |
| | TGCTCT | | | | | | | | | |
| 554 | TGTCTTGCAGGCCGTC | 0.02612 | 0.01998 | hsa-mir- | (0:−1) | (:) | ( ) | 0.2 | 0.613 | −0.6042 |
| | ATGC | | | 431-5p | | | | | | |
| 555 | CGAATCATTATTTGCT | 0.02532 | 0.02532 | hsa-mir- | (0:−3) | (:) | ( ) | 0.1 | 1.521 | −0.0129 |
| | GCT | | | 15b-3p | | | | | | |
| 556 | CAGCAGCAATTCATGT | 0.02138 | 0.02138 | hsa-mir- | (0:0) | (:A) | ( ) | 0.1 | 0.241 | −0.3669 |
| | TTTGAAA | | | 424-5p | | | | | | |
| 557 | ACCAATATTACTGTGC | 0.0205 | 0.01422 | hsa-mir- | (−1:−3) | (:) | ( ) | 0.2 | 3.128 | 1.1757 |
| | TGCT | | | 16-2-3p | | | | | | |
| 558 | TTCAAGTAATCCAGGA | −0.02004 | 0.02004 | hsa-mir- | (0:2) | (:) | (22: G > T) | 0.1 | 3.007 | 4.1471 |
| | TAGGCTTT | | | 26a-5p | | | | | | |
| 559 | TTGAGAACTGAATTCC | 0.01968 | 0.01968 | hsa-mir- | (−1:−1) | (:) | ( ) | 0.1 | 1.968 | 0.5389 |
| | ATGGGT | | | 146a-5p | | | | | | |
| 560 | TATTGCACATTACTAA | 0.01865 | 0.01865 | hsa-mir- | (0:−2) | (:) | ( ) | 0.1 | 3.749 | 1.603 |
| | GTTG | | | 32-5p | | | | | | |
| 561 | TGACCTATGAATTGAC | −0.01793 | 0.01793 | hsa-mir- | (1:2) | (:) | (18: A > C, | 0.1 | −0.659 | 0.189 |
| | AGCCTA | | | 215-5p | | | 20: A > T) | | | |
| 562 | ACTGTAAACGCTTTCT | −0.01783 | 0.01783 | hsa-mir- | (0:0) | (:) | ( ) | 0.1 | 1.014 | 1.2253 |
| | GATG | | | 3607-3p | | | | | | |
| 563 | CATTGCACTTGTCTCG | −0.01738 | 0.01738 | hsa-mir- | (0:0) | (:AT) | ( ) | 0.1 | 0.719 | 1.4522 |
| | GTCTGAAT | | | 25-3p | | | | | | |
| 564 | ATAAAGTAGAAAGCAC | 0.01695 | 0.01695 | hsa-mir- | (1:0) | (:) | ( ) | 0.1 | 2.536 | 0.3764 |
| | TACT | | | 142-5p | | | | | | |
| 565 | AAGTGCAATGATGAAA | 0.01537 | 0.01537 | hsa-mir- | (1:−1) | (A:) | (9: T > G, | 0.1 | 0.631 | −0.6633 |
| | GGGCA | | | 130a-3p | | | 11: A > T) | | | |
| 566 | ACCATAAAGTAGAAAG | 0.01523 | 0.01523 | hsa-mir- | (−1:−2) | (A:) | ( ) | 0.1 | 1.096 | −0.3697 |
| | CACTA | | | 142-5p | | | | | | |
| 567 | CCCCACTGCTAAATTT | −0.01424 | 0.01424 | <NA> | (NA:NA) | (NA:NA) | ( ) | 0.1 | −0.076 | 1.0335 |
| | GACTGGCTTT | | | | | | | | | |
| 568 | TGTCAGTTTGTCAAAT | 0.01423 | 0.01423 | hsa-mir- | (0:2) | (:A) | ( ) | 0.1 | 0.507 | −0.6124 |
| | ACCCCAAGA | | | 223-3p | | | | | | |
| 569 | TACCCAGTAGAACCGA | −0.01326 | 0.01326 | hsa-mir- | (0:−1) | (:) | (5: T > A) | 0.1 | −0.197 | 0.5859 |
| | ATTTGT | | | 10b-5p | | | | | | |
| 570 | TTTGTTCGTTCGGCTC | −0.01282 | 0.01282 | hsa-mir- | (0:0) | (:) | (20: G > A) | 0.1 | −0.245 | 1.5709 |
| | GCGTAA | | | 375 | | | | | | |
| 571 | ATGCTGCCAGTTGAAG | 0.01218 | 0.01218 | hsa-mir- | (0:0) | (:A) | (1: A > T) | 0.1 | 0.462 | −0.555 |
| | AACTGTA | | | 22-3p | | | | | | |
| 572 | TGAGAACCACGTCTGC | 0.01124 | 0.01124 | hsa-mir- | (0:−2) | (:) | ( ) | 0.1 | 0.523 | −0.2778 |
| | TCTG | | | 589-5p | | | | | | |
| 573 | CTGCCAATTCCATAGG | −0.0098 | 0.0098 | hsa-mir- | (0:0) | (:T) | ( ) | 0.1 | 0.349 | 1.5762 |
| | TCACAGT | | | 192-3p | | | | | | |
| 574 | TAGCTTATCAGACTGA | 0.00974 | 0.00974 | hsa-mir- | (0:0) | (:GA) | ( ) | 0.1 | 0.626 | 0.2759 |
| | TGTTGAGA | | | 21-5p | | | | | | |
| 575 | GTAGCTTATCAGACTG | 0.00953 | 0.00953 | hsa-mir- | (−1:2) | (:) | ( ) | 0.1 | 1.628 | 0.0433 |
| | ATGTTGACT | | | 21-5p | | | | | | |
| 576 | TTTGGTCCCCTTCAAC | −0.00945 | 0.00945 | hsa-mir- | (0:0) | (:A) | ( ) | 0.1 | −0.62 | −0.0075 |
| | CAGCTGA | | | 133a-3p | | | | | | |
| 577 | TGTAATAGCAACTCCA | −0.00844 | 0.00844 | hsa-mir- | (0:1) | (:) | (5: C > T) | 0.1 | −0.638 | 0.24 |
| | TGTGGAA | | | 194-5p | | | | | | |
| 578 | GGGACCTATGAATTGA | 0.00774 | 0.00774 | hsa-mir- | (2:0) | (GG:) | (17: C > A) | 0.1 | 0.989 | −0.4886 |
| | CAGAC | | | 192-5p | | | | | | |
| 579 | TAAGGTGCATCTAGTG | 0.00772 | 0.00772 | hsa-mir- | (0:−1) | (:) | (19: T > A) | 0.1 | 2.295 | 0.6414 |
| | CAGATA | | | 18b-5p | | | | | | |
| 580 | GTACTGGAAAGTGCAC | −0.00721 | 0.00721 | <NA> | (NA:NA) | (NA:NA) | ( ) | 0.1 | −0.395 | 1.7345 |
| | TTGGACGAACA | | | | | | | | | |
| 581 | CCCGGGGCTACGCCTG | −0.00713 | 0.00713 | <NA> | (NA:NA) | (NA:NA) | ( ) | 0.1 | 1.856 | 2.7329 |
| | TCTGAGCGTCGCT | | | | | | | | | |
| 582 | AAAGCTGGGTTGAGAG | −0.00655 | 0.00655 | hsa-mir- | (1:2) | (:) | ( ) | 0.1 | 0.172 | 0.9853 |
| | GGCGAAA | | | 320a | | | | | | |
| 583 | CATAAAGTAGAAAGCA | 0.00604 | 0.00537 | hsa-mir- | (0:−2) | (:) | ( ) | 0.2 | 2.95 | 1.1211 |
| | CTA | | | 142-5p | | | | | | |
| 584 | TGTCAGTTTGTCAAAT | 0.00602 | 0.00602 | hsa-mir- | (0:−4) | (:) | ( ) | 0.1 | 2.716 | −0.261 |
| | AC | | | 223-3p | | | | | | |
| 585 | TCCGGTGAGCTCTCGC | 0.00578 | 0.00578 | hsa-mir- | (−1:1) | (T:) | (9: G > C) | 0.1 | 0.207 | −0.4932 |
| | TGGCC | | | 4792 | | | | | | |
| 586 | TATAAAGTAGAAAGCA | 0.00555 | 0.00555 | hsa-mir- | (1:−1) | (T:) | ( ) | 0.1 | 0.13 | −0.6931 |
| | CTAC | | | 142-5p | | | | | | |
| 587 | TGCTGCCAGTTGAAGA | 0.00546 | 0.00546 | hsa-mir- | (2:0) | (T:) | ( ) | 0.1 | 0.158 | −0.6517 |
| | ACTGT | | | 22-3p | | | | | | |
| 588 | AGCTCGGTCTGAGGCC | −0.00518 | 0.00518 | hsa-mir- | (0:2) | (:) | (23: C > T) | 0.1 | 0.091 | 1.3932 |
| | CCTCAGTTT | | | 423-3p | | | | | | |
| 589 | TGTCAGTTTGTCAAAT | 0.00464 | 0.00464 | hsa-mir- | (0:2) | (:) | (22: A > T) | 0.1 | 0.204 | −0.642 |
| | ACCCCATG | | | 223-3p | | | | | | |
| 590 | ATCACAGTGGCTAAGT | 0.00413 | 0.00413 | hsa-mir- | (1:−2) | (A:) | ( ) | 0.1 | 0.487 | −0.6176 |
| | TCC | | | 27a-3p | | | | | | |
| 591 | TGAGAACTGAATTCCA | 0.0039 | 0.0039 | hsa-mir- | (0:−1) | (:AA) | ( ) | 0.1 | 1.542 | 0.5058 |
| | TAGGCAA | | | 146b-5p | | | | | | |
| 592 | TGGGTCTTTGCGGGCG | −0.00383 | 0.00383 | hsa-mir- | (0:0) | (:) | ( ) | 0.1 | 1.582 | 2.1855 |
| | AGATGA | | | 193a-5p | | | | | | |
| 593 | TACCCTGTAGAACCGG | −0.00313 | 0.00313 | hsa-mir- | (0:−2) | (:) | (15: A > G) | 0.1 | −0.657 | −0.2565 |
| | ATTTG | | | 10b-5p | | | | | | |
| 594 | TGAGGGAGTAGATTGT | 0.00301 | 0.00301 | hsa-let- | (0:−1) | (:) | (5: T > G, | 0.1 | 1.916 | 0.1598 |
| | ATAGT | | | 7a-5p | | | 11: G > A) | | | |
| 595 | TACCCTGTTGAACCGA | −0.00297 | 0.00297 | hsa-mir- | (0:−1) | (:) | (8: A > T) | 0.1 | −0.159 | 0.3187 |
| | ATTTGT | | | 10b-5p | | | | | | |
| 596 | TAAGGTGCATCTAGTG | 0.00245 | 0.00245 | hsa-mir- | (0:−2) | (:) | ( ) | 0.1 | 2.559 | 0.72 |
| | CAGAT | | | 18a-5p | | | | | | |
| 597 | GAGAACTGAATTCCAT | 0.0021 | 0.0021 | hsa-mir- | (1:2) | (:) | ( ) | 0.1 | 0.549 | −0.328 |
| | AGGCTGT | | | 146b-5p | | | | | | |
| 598 | TAGCAGCACGCAAATA | 0.00209 | 0.00209 | hsa-mir- | (0:0) | (:) | (10: T > C) | 0.1 | 0.28 | −0.5687 |
| | TTGGCG | | | 16-5p | | | | | | |
| 599 | GGCTCGTTGGTCTAGG | −0.0019 | 0.0019 | hsa-mir- | (0:−2) | (:) | (5: C > G) | 0.1 | −0.534 | 0.0195 |
| | GG | | | 4448 | | | | | | |
| 600 | CAGCAGCAATTCATGT | 0.00173 | 0.00173 | hsa-mir- | (0:−2) | (:) | ( ) | 0.1 | 0.987 | −0.0245 |
| | TTTG | | | 424-5p | | | | | | |
| 601 | AACATTCAACGCTGTC | −0.00169 | 0.00169 | hsa-mir- | (0:−3) | (:) | (8:T > A, | 0.1 | 3.67 | 3.8391 |
| | GGTG | | | 181b-5p | | | 9: T > C) | | | |
| 602 | ATGCAGCACGTAAATA | 0.00169 | 0.00169 | hsa-mir- | (2:0) | (AT:) | ( ) | 0.1 | 0.338 | −0.6428 |
| | TTGGCG | | | 16-5p | | | | | | |
| 603 | TGCCGACGGGCGCTGA | −0.00159 | 0.00159 | <NA> | (NA:NA) | (NA:NA) | ( ) | 0.1 | −0.369 | 0.6898 |
| | CCCCCTT | | | | | | | | | |
| 604 | ATTGGTCGTGGTTGTA | −0.00106 | 0.00106 | <NA> | (NA:NA) | (NA:NA) | ( ) | 0.1 | −0.405 | 0.4592 |
| | GTCCGTGCGAGAA | | | | | | | | | |
| 605 | TGGCAGTGTCTTAGCT | 0.001 | 0.001 | hsa-mir- | (0:−1) | (:) | ( ) | 0.1 | 1.828 | 0.6251 |
| | GGTTG | | | 34a-5p | | | | | | |
| 606 | TGTCAGTTTGTCAAAT | 0.00095 | 0.00095 | hsa-mir- | (0:-5) | (:) | ( ) | 0.1 | 0.047 | −0.6931 |
| | A | | | 223-3p | | | | | | |
| 607 | ACCCTGAGACCCTAAC | 0.00016 | 0.00016 | hsa-mir- | (1:0) | (A:) | ( ) | 0.1 | 0.322 | −0.5771 |
| | TTGTGA | | | 125b-5p | | | | | | |
| 608 | TGGCAGTTTGTCAAAT | 0.00011 | 0.00011 | hsa-mir- | (0:−3) | (:) | (2: T > G) | 0.1 | 1.467 | −0.5979 |
| | ACC | | | 223-3p | | | | | | |

TABLE 12
Identified sRNA biomarkers in colon epithelium tissue that are associated with Diverticular disease.

| SEQ | | | | | | | | | | |
|---|---|---|---|---|---|---|---|---|---|---|
| ID | | | | | | | | | | |
| NO: | Marker | importance | imp_SE | sRNA_name | ref | ext | swaps | chosen | thislbl | otherlbl |
| 609 | ACTGGACTTGGAGTCAG | 1.3057 | 0.12197 | hsa-mir- | (0:0) | (:ATAT) | (9: A > G, | 1 | 1.458 | −0.67 |
| | AAGGCATAT | | | 422a | | | 11: G > A) | | | |
| 610 | TCGACCGGACCTCGACC | 0.23143 | 0.11311 | hsa-mir- | (0:2) | (:A) | (21: C > A) | 0.4 | 1.008 | −0.59 |
| | GGCTAGA | | | 1307-5p | | | | | | |
| 611 | TCAGCACCAGGATATTG | 0.11606 | 0.05936 | hsa-mir- | (0:−1) | (:) | ( ) | 0.4 | 1.535 | −0.58 |
| | TTGGA | | | 3065-3p | | | | | | |
| 612 | TGTAACCGCAACTCCAT | 0.09378 | 0.05427 | hsa-mir- | (0:0) | (:) | (6: A > C) | 0.3 | 1.788 | −0.39 |
| | GTGGA | | | 194-5p | | | | | | |
| 613 | ACTGGACTTGGAGTCAG | 0.08715 | 0.04571 | hsa-mir- | (0:0) | (:ATTA) | (9: A > G, | 0.3 | 1.098 | −0.67 |
| | AAGGCATTA | | | 422a | | | 11: G > A) | | | |
| 614 | AACACTGTCTGGTAAAG | 0.08212 | 0.0662 | hsa-mir- | (1:1) | (:) | ( ) | 0.2 | 1.265 | −0.63 |
| | ATGGC | | | 141-3p | | | | | | |
| 615 | TGTAAACATCCTACACT | 0.08206 | 0.03761 | hsa-mir- | (0:1) | (:TA) | ( ) | 0.5 | 0.138 | −0.69 |
| | CTCAGCTTA | | | 30c-5p | | | | | | |
| 616 | ACTGGACTTTGAGTCAG | 0.06028 | 0.04522 | hsa-mir- | (0:0) | (:A) | (9: A > T, | 0.3 | 0.671 | −0.65 |
| | AAGGCA | | | 422a | | | 11: G > A) | | | |
| 617 | ACTGGACTTGGAGCCAG | 0.05242 | 0.04482 | hsa-mir- | (0:2) | (:AA) | (20: T > G) | 0.2 | 0.921 | −0.65 |
| | AAGGCAA | | | 378f | | | | | | |
| 618 | GTAACAGCAACTCCATG | 0.04186 | 0.02857 | hsa-mir- | (1:1) | (:A) | ( ) | 0.2 | 0.92 | −0.67 |
| | TGGAAA | | | 194-5p | | | | | | |
| 619 | ACTGGACTTGGAGTCAG | 0.03645 | 0.01948 | hsa-mir- | (0:0) | (:AATA) | (9: A > G, | 0.5 | −0.038 | −0.69 |
| | AAGGCAATA | | | 422a | | | 11: G > A) | | | |
| 620 | CTGGACTTGGAGTCAGA | 0.0346 | 0.02916 | hsa-mir- | (1:2) | (:AGA) | (12: C > T, | 0.2 | 0.159 | −0.68 |
| | AGGCAGA | | | 378f | | | 19: T > G) | | | |
| 621 | TGATATGTTTGATATAT | 0.03153 | 0.02537 | hsa-mir- | (0:1) | (:A) | ( ) | 0.2 | 1.842 | −0.53 |
| | TAGGTTA | | | 190a-5p | | | | | | |
| 622 | TGAAATGTTTAGGACCA | 0.02779 | 0.02185 | hsa-mir- | (1:1) | (:AT) | ( ) | 0.2 | 0.309 | −0.68 |
| | CTAGAAT | | | 203a-3p | | | | | | |
| 623 | TGGACTTGGAGTCAGAA | 0.02407 | 0.01645 | hsa-mir- | (2:0) | (:AT) | ( ) | 0.2 | 0.622 | −0.66 |
| | GGCAT | | | 378a-3p | | | | | | |
| 624 | TGTAACAGCAACTCCAT | 0.01862 | 0.01862 | hsa-mir- | (0:2) | (:A) | ( ) | 0.1 | 0.327 | −0.58 |
| | GTGGACTA | | | 194-5p | | | | | | |
| 625 | TCGACCGGACCTCGACC | 0.01749 | 0.01519 | hsa-mir- | (0:0) | (:A) | ( ) | 0.2 | 1.518 | −0.6 |
| | GGCTA | | | 1307-5p | | | | | | |
| 626 | TGAGATGAAGCACTGTA | 0.01455 | 0.01455 | hsa-mir- | (0:1) | (:TA) | ( ) | 0.1 | 0.975 | −0.61 |
| | GCTCATA | | | 143-3p | | | | | | |
| 627 | TTTCAGTCGGATGTTTG | 0.01444 | 0.01444 | hsa-mir- | (1:0) | (:AA) | (16: A > G) | 0.1 | 0.141 | −0.69 |
| | CAGCAA | | | 30e-3p | | | | | | |
| 628 | GACCTATGAATTGACAG | 0.01188 | 0.00963 | hsa-mir- | (2:1) | (:T) | (17: A > C) | 0.2 | 1.014 | −0.58 |
| | CCAT | | | 215-5p | | | | | | |
| 629 | CCACTGCCCCAGGTGCT | 0.01092 | 0.01092 | hsa-mir- | (-2:0) | (:A) | ( ) | 0.1 | 0.692 | −0.6 |
| | GCTGGA | | | 324-3p | | | | | | |
| 630 | CTGACCTATGAATTGAC | 0.0102 | 0.0102 | hsa-mir- | (0:1) | (:TGA) | ( ) | 0.1 | 0.583 | −0.63 |
| | AGCCATGA | | | 192-5p | | | | | | |
| 631 | ACCACAGGGTAGAACCA | 0.00927 | 0.00927 | hsa-mir- | (1:2) | (:GA) | ( ) | 0.1 | 0.682 | −0.58 |
| | CGGACGA | | | 140-3p | | | | | | |
| 632 | TCGACCGGACCTCGACC | 0.00896 | 0.00896 | hsa-mir- | (0:0) | (:GA) | ( ) | 0.1 | −0.463 | −0.68 |
| | GGCTGA | | | 1307-5p | | | | | | |
| 633 | TGGCTCAGTTCAGCAGG | 0.00641 | 0.00641 | hsa-mir- | (0:2) | (:) | ( ) | 0.1 | 0.543 | −0.6 |
| | AACAGGA | | | 24-3p | | | | | | |
| 634 | AGCTTATCAGACTGATG | 0.00487 | 0.00487 | hsa-mir- | (1:0) | (:AA) | ( ) | 0.1 | 0.052 | −0.66 |
| | TTGAAA | | | 21-5p | | | | | | |
| 635 | ATCACATTGCCAGGGAT | 0.00469 | 0.00469 | hsa-mir- | (0:−3) | (:AA) | (13: T > G, | 0.1 | 0.333 | −0.66 |
| | AAAA | | | 23c | | | 17: T > A) | | | |
| 636 | TCAACAAAATCACTGAT | 0.0018 | 0.0018 | hsa-mir- | (0:0) | (:) | ( ) | 0.1 | 0.71 | −0.53 |
| | GCTGGA | | | 3065-5p | | | | | | |
| 637 | ACATTGCCAGGGATTTC | 0.00084 | 0.00084 | hsa-mir- | (3:1) | (:) | ( ) | 0.1 | 1.31 | −0.57 |
| | CA | | | 23a-3p | | | | | | |
| 638 | AACACTGTCTGGTAAAG | 0.00065 | 0.00065 | hsa-mir- | (1:−1) | (:) | ( ) | 0.1 | −0.094 | −0.69 |
| | ATG | | | 141-3p | | | | | | |

1. A method for evaluating Alzheimer's disease in a subject, the method comprising:
providing a biological sample from a subject exhibiting one or more symptoms of Alzheimer's disease, or providing RNA extracted from the sample,
determining the presence or absence of one or more positive sRNA predictors in the sample, wherein the presence of the one or more positive sRNA predictors is indicative of Alzheimer's disease activity.

2. The method of claim 1, wherein the sRNA predictors include one or more sRNA predictors from Table 2A, Table 4A, and/or Table 7A (SEQ ID NOS: 1-403).

3-5. (canceled)

6. The method of claim 2, wherein the positive sRNA predictors include one or more predictors from Table 5 (SEQ ID NOS: 58, 189, 78, 172, 193, 97, 122, 215, 248, 164, 120, 93, 126, 253, 112, 144, 213, 244, 123, 222, 150, 240, 52, 220, 221, 169, 165, and 212).

7. The method of claim 2, wherein the positive sRNA predictors include one or more predictors from Table 8 (SEQ ID NOS: 257, 270, 272, 273, 279, 286, 288, 314, 319, 325, 332, 341, 374, 391, and 393).

8-12. (canceled)

13. The method of claim 1, wherein the sample is a biological fluid.

14. The method of claim 13, wherein the biological fluid is selected from blood, serum, plasma, urine, saliva, or cerebrospinal fluid.

15. (canceled)

16. The method of claim 1, wherein the presence or absence of the sRNAs are determined by a quantitative or qualitative PCR assay.

17-18. (canceled)

19. The method of claim 16, wherein sRNAs are amplified using a stem-loop RT primer.

20-21. (canceled)

22. The method of claim 1, wherein the presence or absence of the sRNAs are determined by nucleic acid sequencing, and sRNAs are identified in the sample by a process that comprises trimming a 3′ sequencing adaptor from individual sRNA sequences.

23-27. (canceled)

28. The method of claim 1, wherein a subject is evaluated at a frequency of at least about once per year, or at least about once every six months, or at least once per month or at least once per week.

29. (canceled)

30. A method for evaluating Alzheimer's disease in a subject, comprising:
providing a biological sample from a subject having one or more mutations correlative with progression to Alzheimer's Disease, or providing RNA extracted from the sample;
determining the presence, absence, or level of one or more positive sRNA predictors as an indication of Alzheimer disease activity and/or progression.

31-54. (canceled)

55. A kit for evaluating samples for Alzheimer's disease, comprising:
sRNA-specific probes and/or primers configured for detecting a plurality of sRNAs listed in Table 2A, Table 4A, or Table 7A (SEQ ID NOS: 1-403).

56-64. (canceled)

65. A method for evaluating a subject for one or more disease conditions, comprising:
providing a biological sample of the subject, and determining the presence or absence of a plurality of sRNAs in an sRNA panel;
classifying the condition of the subject among one or more diseases conditions using a disease classifier;
wherein the disease classifier is trained based on the presence and absence of the sRNAs in the sRNA panel in a set of training samples; the training samples annotated as positive or negative for the one or more disease conditions.

66. The method of claim 65, wherein the presence or absence of the sRNAs in the panel is determined in the training set from sRNA sequence data, and where sRNA sequences are identified in the sRNA sequence data by trimming 5′ and 3′ sequencing adaptors and without consolidating sRNA sequence variants to a reference sequence or genetic locus.

67. The method of claim 66, wherein the presence or absence of sRNAs in the sample is determined by quantitative RT-PCR assays.

68. The method of claim 65, wherein the disease classifier classifies samples among at least three disease conditions, or at least five disease conditions.

69. (canceled)

70. The method of claim 65, wherein the panel contains from about 4 to about 200 sRNAs, or from about 4 to about 100 sRNAs, or from about 4 to about 50 sRNAs.

71-74. (canceled)

75. The method of claim 65, wherein the disease classifier is trained using one or more of supervised, unsupervised, semi-supervised machine learning models such as, Parametric/non-parametric Distance Measures, Logistic Regression, Support Vector Machines, Decision Trees, Random Forests, Neural Networks, Probit Regression, Fisher's Linear Discriminant, Naive Bayes Classifier, Perceptron, Quadratic classifiers, Kernel Estimation, k-Nearest Neighbor, Learning Vector Quantization, and Principal Components Analysis.

76. The method of claim 65, wherein the disease conditions are diseases of the central nervous system.

77-82. (canceled)

83. The method of claim 65, wherein the disease conditions are cancers of different tissue or cell origin.

84-85. (canceled)

86. The method of claim 65, wherein the disease conditions are inflammatory or immunological diseases, and optionally including one or more of Systemic Lupus Erythematosus (SLE), scleroderma, autoimmune vasculitis, diabetes mellitus (type 1 or type 2), Grave's disease, Addison's disease, Sjogren's syndrome, thyroiditis, rheumatoid arthritis, myasthenia gravis, multiple sclerosis, fibromyalgia, psoriasis, Crohn's disease, ulcerative colitis, and celiac disease.

87-89. (canceled)

90. The method of claim 65, wherein at least one, or at least two, or at least five, or at least 10 sRNAs in the panel are positive sRNA predictors, which were identified as present in a plurality of samples annotated as positive for a disease condition in the training set, and absent in all samples annotated as negative for the disease condition in the training set.


SEQUENCE LISTING (638 sequences)

SEQ ID NO: 1 - DNA, 23 bp, Artificial Sequence
  Synthetic polymer.
caggcagttacagatcgaactcc

SEQ ID NO: 2 - DNA, 19 bp, Artificial Sequence
  Synthetic polymer.
ggtcagttacagatcgaac

SEQ ID NO: 3 - DNA, 24 bp, Artificial Sequence
  Synthetic polymer.
ctggctgggttgttcgagacccgc

SEQ ID NO: 4 - DNA, 17 bp, Artificial Sequence
  Synthetic polymer.
ttatgtgatgacttaca

SEQ ID NO: 5 - DNA, 17 bp, Artificial Sequence
  Synthetic polymer.
ttctgtgatgacttaca

SEQ ID NO: 6 - DNA, 23 bp, Artificial Sequence
  Synthetic polymer.
aggttatgggttcgtgtcccacc

SEQ ID NO: 7 - DNA, 18 bp, Artificial Sequence
  Synthetic polymer.
tcttgctccgtccactcc

SEQ ID NO: 8 - DNA, 25 bp, Artificial Sequence
  Synthetic polymer.
ggtagagcatgggactcttaatcgc

SEQ ID NO: 9 - DNA, 19 bp, Artificial Sequence
  Synthetic polymer.
tcgtgctgggcccataacc

SEQ ID NO: 10 - DNA, 23 bp, Artificial Sequence
  Synthetic polymer.
gggttgtgggttcgggtcccacc

SEQ ID NO: 11 - DNA, 17 bp, Artificial Sequence
  Synthetic polymer.
tttatcacgttcgcctc

SEQ ID NO: 12 - DNA, 23 bp, Artificial Sequence
  Synthetic polymer.
aggttccgggctcgggacccggc

SEQ ID NO: 13 - DNA, 21 bp, Artificial Sequence
  Synthetic polymer.
catatgtggtgaatacgtgtt

SEQ ID NO: 14 - DNA, 27 bp, Artificial Sequence
  Synthetic polymer.
gcggtagagcatgggactcttaatccc

SEQ ID NO: 15 - DNA, 27 bp, Artificial Sequence
  Synthetic polymer.
gatccattggggtttccccgcgcaggt

SEQ ID NO: 16 - DNA, 18 bp, Artificial Sequence
  Synthetic polymer.
ccatgggactcttaatcc

SEQ ID NO: 17 - DNA, 21 bp, Artificial Sequence
  Synthetic polymer.
ggtaaacatctccgactggaa

SEQ ID NO: 18 - DNA, 23 bp, Artificial Sequence
  Synthetic polymer.
agggtgtgggttcgaatcccacc

SEQ ID NO: 19 - DNA, 24 bp, Artificial Sequence
  Synthetic polymer.
aaggttccgggttcgtgtcgcggc

SEQ ID NO: 20 - DNA, 24 bp, Artificial Sequence
  Synthetic polymer.
aagtttccgggttcgggccccggc

SEQ ID NO: 21 - DNA, 23 bp, Artificial Sequence
  Synthetic polymer.
aggttgtggattcgtgtcccacc

SEQ ID NO: 22 - DNA, 24 bp, Artificial Sequence
  Synthetic polymer.
gaagttccgggttcgggtcccggc

SEQ ID NO: 23 - DNA, 23 bp, Artificial Sequence
  Synthetic polymer.
aggctgtgggttcgaatcccacc

SEQ ID NO: 24 - DNA, 17 bp, Artificial Sequence
  Synthetic polymer.
gggtgtgatgacttaca

SEQ ID NO: 25 - DNA, 24 bp, Artificial Sequence
  Synthetic polymer.
aagtttccgggttcgggacccggc

SEQ ID NO: 26 - DNA, 24 bp, Artificial Sequence
  Synthetic polymer.
aaggttccgggttcggttcccggc

SEQ ID NO: 27 - DNA, 19 bp, Artificial Sequence
  Synthetic polymer.
actgtggactctgaatcca

SEQ ID NO: 28 - DNA, 24 bp, Artificial Sequence
  Synthetic polymer.
aaggttccgggttcgggtaccggc

SEQ ID NO: 29 - DNA, 19 bp, Artificial Sequence
  Synthetic polymer.
gcacgggactcttaatccc

SEQ ID NO: 30 - DNA, 24 bp, Artificial Sequence
  Synthetic polymer.
aagtttgtgggttcgtatcccacc

SEQ ID NO: 31 - DNA, 23 bp, Artificial Sequence
  Synthetic polymer.
ggagtgtgggttcgtgtcccatc

SEQ ID NO: 32 - DNA, 23 bp, Artificial Sequence
  Synthetic polymer.
aggttgtgggttcgaggcccacc

SEQ ID NO: 33 - DNA, 25 bp, Artificial Sequence
  Synthetic polymer.
agagtttccgggttcgtgtcccggc

SEQ ID NO: 34 - DNA, 18 bp, Artificial Sequence
  Synthetic polymer.
ttgagggtgcgtgtccct

SEQ ID NO: 35 - DNA, 25 bp, Artificial Sequence
  Synthetic polymer.
agaggttccggggtcgggtcccggc

SEQ ID NO: 36 - DNA, 21 bp, Artificial Sequence
  Synthetic polymer.
agtgtgagggttcgtgtccct

SEQ ID NO: 37 - DNA, 22 bp, Artificial Sequence
  Synthetic polymer.
cacccgtagtaccgacctcgcg

SEQ ID NO: 38 - DNA, 25 bp, Artificial Sequence
  Synthetic polymer.
agaggttccgagttcgggtcccggc

SEQ ID NO: 39 - DNA, 33 bp, Artificial Sequence
  Synthetic polymer.
tccccggtggtctagtggttaggattccgcgct

SEQ ID NO: 40 - DNA, 17 bp, Artificial Sequence
  Synthetic polymer.
gacgtcggatcagaaga

SEQ ID NO: 41 - DNA, 17 bp, Artificial Sequence
  Synthetic polymer.
ttttgggatgacttaca

SEQ ID NO: 42 - DNA, 22 bp, Artificial Sequence
  Synthetic polymer.
ttcacgtaatccaggaaaagct

SEQ ID NO: 43 - DNA, 24 bp, Artificial Sequence
  Synthetic polymer.
gaggttacgggttcgtgtcccggc

SEQ ID NO: 44 - DNA, 17 bp, Artificial Sequence
  Synthetic polymer.
atgtgactcttaatctc

SEQ ID NO: 45 - DNA, 21 bp, Artificial Sequence
  Synthetic polymer.
agggtgtgggttcgtcccacc

SEQ ID NO: 46 - DNA, 27 bp, Artificial Sequence
  Synthetic polymer.
tatagcactctggactctgaatccagc

SEQ ID NO: 47 - DNA, 23 bp, Artificial Sequence
  Synthetic polymer.
ccacggactcccaaaagcagctt

SEQ ID NO: 48 - DNA, 23 bp, Artificial Sequence
  Synthetic polymer.
accccgtagatccgaccttgtga

SEQ ID NO: 49 - DNA, 20 bp, Artificial Sequence
  Synthetic polymer.
tcaccgggtgtacatcaagc

SEQ ID NO: 50 - DNA, 22 bp, Artificial Sequence
  Synthetic polymer.
caacggaatctccaaagcagct

SEQ ID NO: 51 - DNA, 23 bp, Artificial Sequence
  Synthetic polymer.
tcttgcactcgtcccggcctcat

SEQ ID NO: 52 - DNA, 17 bp, Artificial Sequence
  Synthetic polymer.
tttcggcactgaggcct

SEQ ID NO: 53 - DNA, 22 bp, Artificial Sequence
  Synthetic polymer.
tcacccgggtgtcaatcagctg

SEQ ID NO: 54 - DNA, 23 bp, Artificial Sequence
  Synthetic polymer.
cccccgtcgaaccgcccttgcga

SEQ ID NO: 55 - DNA, 27 bp, Artificial Sequence
  Synthetic polymer.
gttaaaattcctgaaccgggacgcggc

SEQ ID NO: 56 - DNA, 44 bp, Artificial Sequence
  Synthetic polymer.
ggttcgtgctgacggcctgtatcctaggctacaccctgaggact

SEQ ID NO: 57 - DNA, 20 bp, Artificial Sequence
  Synthetic polymer.
cccccgtcgaaccgaccttg

SEQ ID NO: 58 - DNA, 22 bp, Artificial Sequence
  Synthetic polymer.
ttcacagtggctcagttctgcc

SEQ ID NO: 59 - DNA, 19 bp, Artificial Sequence
  Synthetic polymer.
ttaaactctgtcgtgctgg

SEQ ID NO: 60 - DNA, 21 bp, Artificial Sequence
  Synthetic polymer.
gctaataccggataagaaagc

SEQ ID NO: 61 - DNA, 32 bp, Artificial Sequence
  Synthetic polymer.
tccctggtggtctggtggttaggagtcggcgc

SEQ ID NO: 62 - DNA, 21 bp, Artificial Sequence
  Synthetic polymer.
taaagtgctgaccgtgcagat

SEQ ID NO: 63 - DNA, 25 bp, Artificial Sequence
  Synthetic polymer.
tcctctgtagttcagtcggtagaac

SEQ ID NO: 64 - DNA, 32 bp, Artificial Sequence
  Synthetic polymer.
tccctgtggtctaatggttaggatccggcgct

SEQ ID NO: 65 - DNA, 44 bp, Artificial Sequence
  Synthetic polymer.
ccttggctgggagaacgcctgggaataccgggtgctgtaggctt

SEQ ID NO: 66 - DNA, 23 bp, Artificial Sequence
  Synthetic polymer.
caacatagcgagcccccgtctct

SEQ ID NO: 67 - DNA, 20 bp, Artificial Sequence
  Synthetic polymer.
cagttgccacgttcccgtgg

SEQ ID NO: 68 - DNA, 24 bp, Artificial Sequence
  Synthetic polymer.
tgtaaacctcctggcctggaagct

SEQ ID NO: 69 - DNA, 28 bp, Artificial Sequence
  Synthetic polymer.
cgcattgccgagtagctatgttcggatg

SEQ ID NO: 70 - DNA, 44 bp, Artificial Sequence
  Synthetic polymer.
gacggaaagaccccatgaacctttactgtagctttgtattggac

SEQ ID NO: 71 - DNA, 18 bp, Artificial Sequence
  Synthetic polymer.
ggctaatacctgggactc

SEQ ID NO: 72 - DNA, 25 bp, Artificial Sequence
  Synthetic polymer.
cgcggggtggagcagcctggtagct

SEQ ID NO: 73 - DNA, 30 bp, Artificial Sequence
  Synthetic polymer.
cgggtcgtgggttcgccccacgttgggcgc

SEQ ID NO: 74 - DNA, 30 bp, Artificial Sequence
  Synthetic polymer.
tctacagtccgacgatacgactcttagcgg

SEQ ID NO: 75 - DNA, 32 bp, Artificial Sequence
  Synthetic polymer.
gggcccctacccggccgtcgccggcagtcgag

SEQ ID NO: 76 - DNA, 33 bp, Artificial Sequence
  Synthetic polymer.
tcttccgtagtgtagtggttatgacgttcgcct

SEQ ID NO: 77 - DNA, 18 bp, Artificial Sequence
  Synthetic polymer.
tcaaggctaaaactcaaa

SEQ ID NO: 78 - DNA, 23 bp, Artificial Sequence
  Synthetic polymer.
tacagtactgtgctaactgaaaa

SEQ ID NO: 79 - DNA, 25 bp, Artificial Sequence
  Synthetic polymer.
gccacggtggccgagtggttaaggc

SEQ ID NO: 80 - DNA, 26 bp, Artificial Sequence
  Synthetic polymer.
cccccactgctacatttgactgtctt

SEQ ID NO: 81 - DNA, 27 bp, Artificial Sequence
  Synthetic polymer.
acggataaaaggtacctcggggataac

SEQ ID NO: 82 - DNA, 27 bp, Artificial Sequence
  Synthetic polymer.
cttctagaaatttctgaaaatgctctg

SEQ ID NO: 83 - DNA, 29 bp, Artificial Sequence
  Synthetic polymer.
ccccccactgctaaatttgactggctact

SEQ ID NO: 84 - DNA, 31 bp, Artificial Sequence
  Synthetic polymer.
ggccgcgtgcctaatggataaggcgtctgat

SEQ ID NO: 85 - DNA, 33 bp, Artificial Sequence
  Synthetic polymer.
ctgtgagggtgagcgaatcgctgaaagccggcc

SEQ ID NO: 86 - DNA, 33 bp, Artificial Sequence
  Synthetic polymer.
gcttgcggagtgtagtggttatcacgttcgcct

SEQ ID NO: 87 - DNA, 34 bp, Artificial Sequence
  Synthetic polymer.
caacggataaaaggtactctagggataacaggct

SEQ ID NO: 88 - DNA, 34 bp, Artificial Sequence
  Synthetic polymer.
cattggtggttccgtggtagaattctcgcctgcc

SEQ ID NO: 89 - DNA, 34 bp, Artificial Sequence
  Synthetic polymer.
ggctggtccgatggtagtggggtatcagaacttg

SEQ ID NO: 90 - DNA, 40 bp, Artificial Sequence
  Synthetic polymer.
ttgaccttaccggatggcacaaagagaagtgggcaagttc

SEQ ID NO: 91 - DNA, 30 bp, Artificial Sequence
  Synthetic polymer.
tccctagttcgtttctgggagcggagacca

SEQ ID NO: 92 - DNA, 29 bp, Artificial Sequence
  Synthetic polymer.
tcccatgtggtctagcggttaggattcct

SEQ ID NO: 93 - DNA, 28 bp, Artificial Sequence
  Synthetic polymer.
cgggcctttcggggcctcttccccgggc

SEQ ID NO: 94 - DNA, 18 bp, Artificial Sequence
  Synthetic polymer.
gtggttccggctttggac

SEQ ID NO: 95 - DNA, 25 bp, Artificial Sequence
  Synthetic polymer.
gtgctaatctgcgataagcgtcggt

SEQ ID NO: 96 - DNA, 23 bp, Artificial Sequence
  Synthetic polymer.
tcagtgcatcaccgacctttgtt

SEQ ID NO: 97 - DNA, 23 bp, Artificial Sequence
  Synthetic polymer.
tccctgagaccctttaaacctgt

SEQ ID NO: 98 - DNA, 30 bp, Artificial Sequence
  Synthetic polymer.
ctagtacgagaggaccggagtggacgcatc

SEQ ID NO: 99 - DNA, 20 bp, Artificial Sequence
  Synthetic polymer.
gaggcagcagtagggaatat

SEQ ID NO: 100 - DNA, 22 bp, Artificial Sequence
  Synthetic polymer.
tagcaccatttgcaatcggttg

SEQ ID NO: 101 - DNA, 25 bp, Artificial Sequence
  Synthetic polymer.
ttagacagttcggtccctatctgcc

SEQ ID NO: 102 - DNA, 28 bp, Artificial Sequence
  Synthetic polymer.
tgatgtcggctcatctcatcctggggct

SEQ ID NO: 103 - DNA, 18 bp, Artificial Sequence
  Synthetic polymer.
aatcctggtcggacatca

SEQ ID NO: 104 - DNA, 23 bp, Artificial Sequence
  Synthetic polymer.
tgcaccatggttctctgagcatg

SEQ ID NO: 105 - DNA, 30 bp, Artificial Sequence
  Synthetic polymer.
tggggagttcgagtctctccgcccctgcca

SEQ ID NO: 106 - DNA, 38 bp, Artificial Sequence
  Synthetic polymer.
ccaaggggtcgtgggttcgaatcctgccagccgcacca

SEQ ID NO: 107 - DNA, 18 bp, Artificial Sequence
  Synthetic polymer.
tcgtgatacagttcggtc

SEQ ID NO: 108 - DNA, 29 bp, Artificial Sequence
  Synthetic polymer.
tccggggagcacgcctgttcgagtatcgt

SEQ ID NO: 109 - DNA, 36 bp, Artificial Sequence
  Synthetic polymer.
gccccgttcgtctagcggcctaggacgccggcctct

SEQ ID NO: 110 - DNA, 17 bp, Artificial Sequence
  Synthetic polymer.
cttccacaacgttcccg

SEQ ID NO: 111 - DNA, 18 bp, Artificial Sequence
  Synthetic polymer.
ttcgatcccgtcatcacc

SEQ ID NO: 112 - DNA, 20 bp, Artificial Sequence
  Synthetic polymer.
aaagaggaggagaggagaac

SEQ ID NO: 113 - DNA, 30 bp, Artificial Sequence
  Synthetic polymer.
tccaccacgttcccgtggtaaatcagcttg

SEQ ID NO: 114 - DNA, 41 bp, Artificial Sequence
  Synthetic polymer.
gcaagcaggggtcgtcggttcgatcccgtcatcctccacca

SEQ ID NO: 115 - DNA, 18 bp, Artificial Sequence
  Synthetic polymer.
cccccacgttcccgttgg

SEQ ID NO: 116 - DNA, 19 bp, Artificial Sequence
  Synthetic polymer.
tttggtatctgcgctctgc

SEQ ID NO: 117 - DNA, 22 bp, Artificial Sequence
  Synthetic polymer.
caccttgcgcaatcaggactga

SEQ ID NO: 118 - DNA, 23 bp, Artificial Sequence
  Synthetic polymer.
gggatagtaggtcgttgccaacc

SEQ ID NO: 119 - DNA, 24 bp, Artificial Sequence
  Synthetic polymer.
ggaagaacgggtgctgtaggcttt

SEQ ID NO: 120 - DNA, 28 bp, Artificial Sequence
  Synthetic polymer.
cgagaccaggactttgataggctgggtg

SEQ ID NO: 121 - DNA, 33 bp, Artificial Sequence
  Synthetic polymer.
aagcagcaatgcgacgtatagggtctgacgcct

SEQ ID NO: 122 - DNA, 33 bp, Artificial Sequence
  Synthetic polymer.
tcaaatggtagagcgctcgcttggcttgcgaga

SEQ ID NO: 123 - DNA, 34 bp, Artificial Sequence
  Synthetic polymer.
gacccagttgcctaattggataaggcatcagcct

SEQ ID NO: 124 - DNA, 35 bp, Artificial Sequence
  Synthetic polymer.
tccctggtggtctggtggttaggagtcggcgctct

SEQ ID NO: 125 - DNA, 17 bp, Artificial Sequence
  Synthetic polymer.
atagatcctgaaaccgc

SEQ ID NO: 126 - DNA, 19 bp, Artificial Sequence
  Synthetic polymer.
ctcttcgaggccctgtaat

SEQ ID NO: 127 - DNA, 20 bp, Artificial Sequence
  Synthetic polymer.
aggtcctcaatacgtatttg

SEQ ID NO: 128 - DNA, 20 bp, Artificial Sequence
  Synthetic polymer.
caaggcaaagacgcgtagct

SEQ ID NO: 129 - DNA, 23 bp, Artificial Sequence
  Synthetic polymer.
aactggagagtttgattctggct

SEQ ID NO: 130 - DNA, 23 bp, Artificial Sequence
  Synthetic polymer.
cggtgaatacgttcccgggcctt

SEQ ID NO: 131 - DNA, 23 bp, Artificial Sequence
  Synthetic polymer.
ttccctttttaatcctatgcctg

SEQ ID NO: 132 - DNA, 24 bp, Artificial Sequence
  Synthetic polymer.
agcacgcgcgcacgtgttaggacc

SEQ ID NO: 133 - DNA, 26 bp, Artificial Sequence
  Synthetic polymer.
cagatggcggaattggtagacgcgct

SEQ ID NO: 134 - DNA, 26 bp, Artificial Sequence
  Synthetic polymer.
cgtggttcatttccccctttcgggcg

SEQ ID NO: 135 - DNA, 27 bp, Artificial Sequence
  Synthetic polymer.
ggtcgatgatgattggtaaaaggtctg

SEQ ID NO: 136 - DNA, 31 bp, Artificial Sequence
  Synthetic polymer.
gtcgccggttcaagtccggcagtcggctcca

SEQ ID NO: 137 - DNA, 36 bp, Artificial Sequence
  Synthetic polymer.
aacaccgtggaagttcgagtcttctcctgggcacca

SEQ ID NO: 138 - DNA, 17 bp, Artificial Sequence
  Synthetic polymer.
agggatgtcgctcaacg

SEQ ID NO: 139 - DNA, 17 bp, Artificial Sequence
  Synthetic polymer.
gcctgtagtcgtgcccg

SEQ ID NO: 140 - DNA, 19 bp, Artificial Sequence
  Synthetic polymer.
aatcgatcgagggcttaac

SEQ ID NO: 141 - DNA, 19 bp, Artificial Sequence
  Synthetic polymer.
gcaaccatcctctgctacc

SEQ ID NO: 142 - DNA, 19 bp, Artificial Sequence
  Synthetic polymer.
tcaacttcggaactgcctt

SEQ ID NO: 143 - DNA, 21 bp, Artificial Sequence
  Synthetic polymer.
acattgggactgagccacggc

SEQ ID NO: 144 - DNA, 21 bp, Artificial Sequence
  Synthetic polymer.
ggaggggagtgaaatagaacc

SEQ ID NO: 145 - DNA, 21 bp, Artificial Sequence
  Synthetic polymer.
tgaataccgtgctgtaggctt

SEQ ID NO: 146 - DNA, 22 bp, Artificial Sequence
  Synthetic polymer.
ctaatcgatcgagggcttaacc

SEQ ID NO: 147 - DNA, 22 bp, Artificial Sequence
  Synthetic polymer.
tgaccgggagtcaatcagcttg

SEQ ID NO: 148 - DNA, 22 bp, Artificial Sequence
  Synthetic polymer.
tgaggggcagagcgcgagacta

SEQ ID NO: 149 - DNA, 22 bp, Artificial Sequence
  Synthetic polymer.
tgcggacaaggggaatctgact

SEQ ID NO: 150 - DNA, 22 bp, Artificial Sequence
  Synthetic polymer.
ttatgtagtagattgttatagt

SEQ ID NO: 151 - DNA, 23 bp, Artificial Sequence
  Synthetic polymer.
ccccgtccgccccccgttccccc

SEQ ID NO: 152 - DNA, 23 bp, Artificial Sequence
  Synthetic polymer.
ggaggggcagagagcgagccttt

SEQ ID NO: 153 - DNA, 23 bp, Artificial Sequence
  Synthetic polymer.
taggggtgaaaggctaaacaaac

SEQ ID NO: 154 - DNA, 23 bp, Artificial Sequence
  Synthetic polymer.
tgtctgaacatggggggaccacc

SEQ ID NO: 155 - DNA, 23 bp, Artificial Sequence
  Synthetic polymer.
ttcattcggctgtccgagatgta

SEQ ID NO: 156 - DNA, 24 bp, Artificial Sequence
  Synthetic polymer.
agctagacagcaggacggtggcca

SEQ ID NO: 157 - DNA, 25 bp, Artificial Sequence
  Synthetic polymer.
ttatggccaggctgtctccacccga

SEQ ID NO: 158 - DNA, 26 bp, Artificial Sequence
  Synthetic polymer.
aatagaacctgaaaccggatgcctac

SEQ ID NO: 159 - DNA, 27 bp, Artificial Sequence
  Synthetic polymer.
cgcgctcgccggccgaggtgggatccc

SEQ ID NO: 160 - DNA, 28 bp, Artificial Sequence
  Synthetic polymer.
gcggatgtggctcagctggtagagcatc

SEQ ID NO: 161 - DNA, 32 bp, Artificial Sequence
  Synthetic polymer.
ctcgtaccaaacgagaactttgaaggccgaag

SEQ ID NO: 162 - DNA, 33 bp, Artificial Sequence
  Synthetic polymer.
gcggctgtagtgtagtggtgatcacgttcgccc

SEQ ID NO: 163 - DNA, 34 bp, Artificial Sequence
  Synthetic polymer.
acgtagaggccggaggttcgaatcctctcacccc

SEQ ID NO: 164 - DNA, 35 bp, Artificial Sequence
  Synthetic polymer.
tcattggtggttcagtggtagacttctcgcctgcc

SEQ ID NO: 165 - DNA, 37 bp, Artificial Sequence
  Synthetic polymer.
acgatgtgggattgcattgacaatcaggaggttggct

SEQ ID NO: 166 - DNA, 38 bp, Artificial Sequence
  Synthetic polymer.
aacctatctgtgtaggataggtgggaggctttgaagtc

SEQ ID NO: 167 - DNA, 20 bp, Artificial Sequence
  Synthetic polymer.
ctaaatactcgtacatgacc

SEQ ID NO: 168 - DNA, 22 bp, Artificial Sequence
  Synthetic polymer.
ccctagcttgtgcgctcctgga

SEQ ID NO: 169 - DNA, 22 bp, Artificial Sequence
  Synthetic polymer.
tgcaactcgactccatgaagtc

SEQ ID NO: 170 - DNA, 25 bp, Artificial Sequence
  Synthetic polymer.
tccccgtaatcttcataatccggag

SEQ ID NO: 171 - DNA, 33 bp, Artificial Sequence
  Synthetic polymer.
gcattggtggttcggtggtagaatgctcgcctg

SEQ ID NO: 172 - DNA, 31 bp, Artificial Sequence
  Synthetic polymer.
ttcgagccccgcgggtgcttactgacccttt

SEQ ID NO: 173 - DNA, 43 bp, Artificial Sequence
  Synthetic polymer.
acttggctgggagaccgcctgggaataccgggtgctgtatgct

SEQ ID NO: 174 - DNA, 34 bp, Artificial Sequence
  Synthetic polymer.
ccccatgaagtcggagtcgctagtaatcgcagat

SEQ ID NO: 175 - DNA, 42 bp, Artificial Sequence
  Synthetic polymer.
aattggcatgagtccactttaaatcctttaacgaggatccat

SEQ ID NO: 176 - DNA, 19 bp, Artificial Sequence
  Synthetic polymer.
caaaactcccgtgctgatc

SEQ ID NO: 177 - DNA, 32 bp, Artificial Sequence
  Synthetic polymer.
tgcccgttggtctagggggatgattctcgctt

SEQ ID NO: 178 - DNA, 33 bp, Artificial Sequence
  Synthetic polymer.
tcctcgatagctcagttggtagagcgccggact

SEQ ID NO: 179 - DNA, 21 bp, Artificial Sequence
  Synthetic polymer.
cgagcccaggttggagagcca

SEQ ID NO: 180 - DNA, 21 bp, Artificial Sequence
  Synthetic polymer.
gatcagctaccgtcgtagttc

SEQ ID NO: 181 - DNA, 23 bp, Artificial Sequence
  Synthetic polymer.
gtctttttgtcctcctatgcctg

SEQ ID NO: 182 - DNA, 25 bp, Artificial Sequence
  Synthetic polymer.
atggttcgcactctggactctgaat

SEQ ID NO: 183 - DNA, 34 bp, Artificial Sequence
  Synthetic polymer.
ccacgttcccgtggattccaccacgttcccgggg

SEQ ID NO: 184 - DNA, 35 bp, Artificial Sequence
  Synthetic polymer.
cctaaaaagacggatgttgctgagtgtggacctgg

SEQ ID NO: 185 - DNA, 19 bp, Artificial Sequence
  Synthetic polymer.
tagaaaccgggcggaaaca

SEQ ID NO: 186 - DNA, 35 bp, Artificial Sequence
  Synthetic polymer.
ctggagaccggggttcgatttcccgacggggagcc

SEQ ID NO: 187 - DNA, 35 bp, Artificial Sequence
  Synthetic polymer.
tctgctgaggctaagcccgtgttctaaagatttgt

SEQ ID NO: 188 - DNA, 37 bp, Artificial Sequence
  Synthetic polymer.
ccatgtgtcgtaggttcgaatcctatcggggccgcca

SEQ ID NO: 189 - DNA, 21 bp, Artificial Sequence
  Synthetic polymer.
tcagtgcatgaccgaacttgt

SEQ ID NO: 190 - DNA, 25 bp, Artificial Sequence
  Synthetic polymer.
tagttggttttcggaactgaggcca

SEQ ID NO: 191 - DNA, 40 bp, Artificial Sequence
  Synthetic polymer.
ggacagtgtctggtgggtagtttgactggggcggtctcct

SEQ ID NO: 192 - DNA, 23 bp, Artificial Sequence
  Synthetic polymer.
tgccctttgtcatcctcttcctg

SEQ ID NO: 193 - DNA, 36 bp, Artificial Sequence
  Synthetic polymer.
cgctacctcagatcaggacgtggcgacccgctgaat

SEQ ID NO: 194 - DNA, 34 bp, Artificial Sequence
  Synthetic polymer.
gttgtcgtgggttcgagccccatcagccacccca

SEQ ID NO: 195 - DNA, 28 bp, Artificial Sequence
  Synthetic polymer.
gcggaagtagttcagtggtagaacatca

SEQ ID NO: 196 - DNA, 41 bp, Artificial Sequence
  Synthetic polymer.
cgcgacctcagatcagacgtggcgacccgctgagtgtaagc

SEQ ID NO: 197 - DNA, 25 bp, Artificial Sequence
  Synthetic polymer.
gcaggttcagtcctgccgcggtcgc

SEQ ID NO: 198 - DNA, 27 bp, Artificial Sequence
  Synthetic polymer.
gtgatatagacagcaggacggtggcca

SEQ ID NO: 199 - DNA, 29 bp, Artificial Sequence
  Synthetic polymer.
ccagtgtgaaagtaggttatcttcaggct

SEQ ID NO: 200 - DNA, 21 bp, Artificial Sequence
  Synthetic polymer.
gtaccgggtgtaaatcagctg

SEQ ID NO: 201 - DNA, 28 bp, Artificial Sequence
  Synthetic polymer.
caccgaaatcgcggatatgagcgttcct

SEQ ID NO: 202 - DNA, 31 bp, Artificial Sequence
  Synthetic polymer.
agtctggcacggtgaagagacatgagagggg

SEQ ID NO: 203 - DNA, 33 bp, Artificial Sequence
  Synthetic polymer.
gtaaccggggttcgaatccccgtagggacgcca

SEQ ID NO: 204 - DNA, 17 bp, Artificial Sequence
  Synthetic polymer.
gctgcatggccgtcgtc

SEQ ID NO: 205 - DNA, 18 bp, Artificial Sequence
  Synthetic polymer.
cgggcgctgtaggctttt

SEQ ID NO: 206 - DNA, 18 bp, Artificial Sequence
  Synthetic polymer.
gtcctctcggccgcacca

SEQ ID NO: 207 - DNA, 21 bp, Artificial Sequence
  Synthetic polymer.
cgcagagtcgcgcagcggaag

SEQ ID NO: 208 - DNA, 22 bp, Artificial Sequence
  Synthetic polymer.
cggggtgtagcttagcctggta

SEQ ID NO: 209 - DNA, 23 bp, Artificial Sequence
  Synthetic polymer.
gccggctagctcagtcggtagag

SEQ ID NO: 210 - DNA, 23 bp, Artificial Sequence
  Synthetic polymer.
ttccgtttgtcatcctatggctg

SEQ ID NO: 211 - DNA, 24 bp, Artificial Sequence
  Synthetic polymer.
atcctgtctgaatatggggggacc

SEQ ID NO: 212 - DNA, 25 bp, Artificial Sequence
  Synthetic polymer.
ggctcataacccgaaggtcgtcggt

SEQ ID NO: 213 - DNA, 27 bp, Artificial Sequence
  Synthetic polymer.
tccagggttcagttccctgttcgggcg

SEQ ID NO: 214 - DNA, 29 bp, Artificial Sequence
  Synthetic polymer.
acggataaaaggtacctcggggataacag

SEQ ID NO: 215 - DNA, 32 bp, Artificial Sequence
  Synthetic polymer.
gcatttgtggtgcagtggtagaattctagcct

SEQ ID NO: 216 - DNA, 40 bp, Artificial Sequence
  Synthetic polymer.
cacaacgagatcacctctgggtcgtctgccggtctccacc

SEQ ID NO: 217 - DNA, 41 bp, Artificial Sequence
  Synthetic polymer.
ctgcactacagcctgggcaacatagcgagaccccgtctcta

SEQ ID NO: 218 - DNA, 18 bp, Artificial Sequence
  Synthetic polymer.
attgaccgattgagagct

SEQ ID NO: 219 - DNA, 20 bp, Artificial Sequence
  Synthetic polymer.
ccggggccacgtgcccgtgg

SEQ ID NO: 220 - DNA, 21 bp, Artificial Sequence
  Synthetic polymer.
gttcagatcccggacgagcca

SEQ ID NO: 221 - DNA, 25 bp, Artificial Sequence
  Synthetic polymer.
tcaaacagaactttgaaggccgaag

SEQ ID NO: 222 - DNA, 26 bp, Artificial Sequence
  Synthetic polymer.
cgtgttcaggtgacgtcggggtcacc

SEQ ID NO: 223 - DNA, 26 bp, Artificial Sequence
  Synthetic polymer.
tgtcgggctggggcgcgaagcggggc

SEQ ID NO: 224 - DNA, 33 bp, Artificial Sequence
  Synthetic polymer.
gcccggctagctcagtcggtagatcatgagaca

SEQ ID NO: 225 - DNA, 33 bp, Artificial Sequence
  Synthetic polymer.
tcccacatcgtccagcggttaggattcctggtt

SEQ ID NO: 226 - DNA, 34 bp, Artificial Sequence
  Synthetic polymer.
tccctggtggtctagtgactaggattcggcgctt

SEQ ID NO: 227 - DNA, 17 bp, Artificial Sequence
  Synthetic polymer.
acaaaccggaggaaggt

SEQ ID NO: 228 - DNA, 34 bp, Artificial Sequence
  Synthetic polymer.
ctcgacccttcgaacgcacttgcggccccgggtt

SEQ ID NO: 229 - DNA, 19 bp, Artificial Sequence
  Synthetic polymer.
gtagtaccgccatgtctgt

SEQ ID NO: 230 - DNA, 22 bp, Artificial Sequence
  Synthetic polymer.
cggtggcaccacgttcccgggg

SEQ ID NO: 231 - DNA, 42 bp, Artificial Sequence
  Synthetic polymer.
gccacgatcgactgagattcagcctttgttctgtagatttgt

SEQ ID NO: 232 - DNA, 21 bp, Artificial Sequence
  Synthetic polymer.
tagaggttatcacgtctgctt

SEQ ID NO: 233 - DNA, 26 bp, Artificial Sequence
  Synthetic polymer.
cagatggtagtgggttatcagaactt

SEQ ID NO: 234 - DNA, 33 bp, Artificial Sequence
  Synthetic polymer.
gcttgcgtagggtagtggttatcacgttcgcct

SEQ ID NO: 235 - DNA, 34 bp, Artificial Sequence
  Synthetic polymer.
tagaccgcctgggaataccggttgctgtaggctt

SEQ ID NO: 236 - DNA, 31 bp, Artificial Sequence
  Synthetic polymer.
gggaggctttgaagtgtggacgccagtctgc

SEQ ID NO: 237 - DNA, 22 bp, Artificial Sequence
  Synthetic polymer.
gggatgaaccgaccgccgggtt

SEQ ID NO: 238 - DNA, 31 bp, Artificial Sequence
  Synthetic polymer.
gtcggcagttcaatcctgcccatgggcacca

SEQ ID NO: 239 - DNA, 41 bp, Artificial Sequence
  Synthetic polymer.
atagtgcgtgttcccgtgtgaaagtaggtcatcgtcaggct

SEQ ID NO: 240 - DNA, 19 bp, Artificial Sequence
  Synthetic polymer.
ggtcatctcgggggaacct

SEQ ID NO: 241 - DNA, 39 bp, Artificial Sequence
  Synthetic polymer.
cactccagcctgggcaacatagcgcgaccccgtctctta

SEQ ID NO: 242 - DNA, 20 bp, Artificial Sequence
  Synthetic polymer.
tacgcctgtctgggcgtcgc

SEQ ID NO: 243 - DNA, 21 bp, Artificial Sequence
  Synthetic polymer.
tgaccggggtaaataagcttg

SEQ ID NO: 244 - DNA, 34 bp, Artificial Sequence
  Synthetic polymer.
cagcgatccgaggtcaaatctcggtggaacctcc

SEQ ID NO: 245 - DNA, 35 bp, Artificial Sequence
  Synthetic polymer.
ggctggtccgatgggagggggttatcagaacttat

SEQ ID NO: 246 - DNA, 24 bp, Artificial Sequence
  Synthetic polymer.
cagttcggtccctatctgccgtgg

SEQ ID NO: 247 - DNA, 22 bp, Artificial Sequence
  Synthetic polymer.
tcagtgcactaaagcactttgt

SEQ ID NO: 248 - DNA, 26 bp, Artificial Sequence
  Synthetic polymer.
gacggattgcgtaacttgttcagact

SEQ ID NO: 249 - DNA, 25 bp, Artificial Sequence
  Synthetic polymer.
tgggagagtaggtcgccgccggaca

SEQ ID NO: 250 - DNA, 29 bp, Artificial Sequence
  Synthetic polymer.
gacgaagactgacgctcaggtgcgaaagc

SEQ ID NO: 251 - DNA, 19 bp, Artificial Sequence
  Synthetic polymer.
ggggtagagcactgtttag

SEQ ID NO: 252 - DNA, 27 bp, Artificial Sequence
  Synthetic polymer.
gaagtagaaaagagcacatggtggatg

SEQ ID NO: 253 - DNA, 21 bp, Artificial Sequence
  Synthetic polymer.
tattacactcgtcccggcctc

SEQ ID NO: 254 - DNA, 35 bp, Artificial Sequence
  Synthetic polymer.
tacctggtggtatagtggttaggattcggcgctct

SEQ ID NO: 255 - DNA, 18 bp, Artificial Sequence
  Synthetic polymer.
cgtgttcggactggggtc

SEQ ID NO: 256 - DNA, 34 bp, Artificial Sequence
  Synthetic polymer.
tgtgattagagggctggaactttcacccccaccc

SEQ ID NO: 257 - DNA, 43 bp, Artificial Sequence
  Synthetic polymer.
tctgttacggaactgtactctctgagggcctcccacctgattc

SEQ ID NO: 258 - DNA, 44 bp, Artificial Sequence
  Synthetic polymer.
cacctgtgcgtgtgggtgctgctgcgggctgtcagatgctgacc

SEQ ID NO: 259 - DNA, 18 bp, Artificial Sequence
  Synthetic polymer.
ctcagatcagacgtggcg

SEQ ID NO: 260 - DNA, 31 bp, Artificial Sequence
  Synthetic polymer.
tttgagaggatgatcagccacactgggactg

SEQ ID NO: 261 - DNA, 34 bp, Artificial Sequence
  Synthetic polymer.
ctgtttcaaccaacgcttgactgagaactctttc

SEQ ID NO: 262 - DNA, 33 bp, Artificial Sequence
  Synthetic polymer.
tcagggtcagtctaagtgaagacaaagagaggc

SEQ ID NO: 263 - DNA, 30 bp, Artificial Sequence
  Synthetic polymer.
agtgcgagtttgagggctgtgaccggcgct

SEQ ID NO: 264 - DNA, 19 bp, Artificial Sequence
  Synthetic polymer.
catgttgctttatttatca

SEQ ID NO: 265 - DNA, 27 bp, Artificial Sequence
  Synthetic polymer.
tgtgggagagtaggacgccgccggaca

SEQ ID NO: 266 - DNA, 43 bp, Artificial Sequence
  Synthetic polymer.
tctgttacggaactgtactctctgagggcctcccacctgactc

SEQ ID NO: 267 - DNA, 23 bp, Artificial Sequence
  Synthetic polymer.
aggactggtggagcgcttagaag

SEQ ID NO: 268 - DNA, 39 bp, Artificial Sequence
  Synthetic polymer.
gccccagtggcctaatggataaggcattggcttagggac

SEQ ID NO: 269 - DNA, 43 bp, Artificial Sequence
  Synthetic polymer.
cagggcacggtatttcttgttacttccctgcacacggactgtg

SEQ ID NO: 270 - DNA, 29 bp, Artificial Sequence
  Synthetic polymer.
tacaaggaaggtcactaccgttctttcac

SEQ ID NO: 271 - DNA, 30 bp, Artificial Sequence
  Synthetic polymer.
ctgctttcttctttggatcgtcgttcaact

SEQ ID NO: 272 - DNA, 30 bp, Artificial Sequence
  Synthetic polymer.
ttagcaacaacaggaagccccttttatcct

SEQ ID NO: 273 - DNA, 23 bp, Artificial Sequence
  Synthetic polymer.
tctgaatcaacccttattactct

SEQ ID NO: 274 - DNA, 34 bp, Artificial Sequence
  Synthetic polymer.
tctcatttgggcagaatatgtcagagggaagatc

SEQ ID NO: 275 - DNA, 18 bp, Artificial Sequence
  Synthetic polymer.
cctcctaagtattacacc

SEQ ID NO: 276 - DNA, 22 bp, Artificial Sequence
  Synthetic polymer.
cccatcttgctgagatgaggcc

SEQ ID NO: 277 - DNA, 26 bp, Artificial Sequence
  Synthetic polymer.
ccttgtaataacctctagtcctttcc

SEQ ID NO: 278 - DNA, 29 bp, Artificial Sequence
  Synthetic polymer.
attcatggtgctttcaagtcaggttttct

SEQ ID NO: 279 - DNA, 17 bp, Artificial Sequence
  Synthetic polymer.
catcagagacagtggca

SEQ ID NO: 280 - DNA, 20 bp, Artificial Sequence
  Synthetic polymer.
ccctgaagatgtaactgtca

SEQ ID NO: 281 - DNA, 24 bp, Artificial Sequence
  Synthetic polymer.
ccctgaagcataccaaaatgtgtc

SEQ ID NO: 282 - DNA, 25 bp, Artificial Sequence
  Synthetic polymer.
tgaaaaggactttgaaaagagagtc

SEQ ID NO: 283 - DNA, 20 bp, Artificial Sequence
  Synthetic polymer.
ctgtcgggacccgaaagatg

SEQ ID NO: 284 - DNA, 17 bp, Artificial Sequence
  Synthetic polymer.
tcatctcatcctggggc

SEQ ID NO: 285 - DNA, 21 bp, Artificial Sequence
  Synthetic polymer.
ctactctgaacgattgagacc

SEQ ID NO: 286 - DNA, 23 bp, Artificial Sequence
  Synthetic polymer.
cggcgggctgtcagattctcacc

SEQ ID NO: 287 - DNA, 32 bp, Artificial Sequence
  Synthetic polymer.
gggtgattagctcagctgggagagcgtctgcc

SEQ ID NO: 288 - DNA, 38 bp, Artificial Sequence
  Synthetic polymer.
ccctagtcttcatttgttgttatgtcattgcctgcctt

SEQ ID NO: 289 - DNA, 42 bp, Artificial Sequence
  Synthetic polymer.
cccaggttcaagtgattctcctgcctcagcctccagagtacc

SEQ ID NO: 290 - DNA, 17 bp, Artificial Sequence
  Synthetic polymer.
cttcacctgagagtgtc

SEQ ID NO: 291 - DNA, 20 bp, Artificial Sequence
  Synthetic polymer.
ccccagaagcaggtgtcaat

SEQ ID NO: 292 - DNA, 40 bp, Artificial Sequence
  Synthetic polymer.
cccatatttcataatttcacgcttctgtcttgcatgcttc

SEQ ID NO: 293 - DNA, 44 bp, Artificial Sequence
  Synthetic polymer.
cacttgtgcttgtgggtgctactgcgggcggtcagatgctcacc

SEQ ID NO: 294 - DNA, 44 bp, Artificial Sequence
  Synthetic polymer.
tgggcagtggcttatgggaagatgacctctgattaaataattcc

SEQ ID NO: 295 - DNA, 42 bp, Artificial Sequence
  Synthetic polymer.
cgcgacctcagatccgacgtggcgacccgctgaatttaagcc

SEQ ID NO: 296 - DNA, 23 bp, Artificial Sequence
  Synthetic polymer.
cgtgagagaactcgggtgaagga

SEQ ID NO: 297 - DNA, 31 bp, Artificial Sequence
  Synthetic polymer.
aagcactgaaccgggcgactagtactagagt

SEQ ID NO: 298 - DNA, 38 bp, Artificial Sequence
  Synthetic polymer.
ctgtctggactacttctttctctgattaatgccttgct

SEQ ID NO: 299 - DNA, 19 bp, Artificial Sequence
  Synthetic polymer.
catttcctccattgtgtcc

SEQ ID NO: 300 - DNA, 41 bp, Artificial Sequence
  Synthetic polymer.
tatttgcgtagaggtgtttgtagtattctctgatggtagta

SEQ ID NO: 301 - DNA, 32 bp, Artificial Sequence
  Synthetic polymer.
cctcctggagagatctcttgagttcctgcctc

SEQ ID NO: 302 - DNA, 25 bp, Artificial Sequence
  Synthetic polymer.
cgggagagtaggtcgcgccaggtcc

SEQ ID NO: 303 - DNA, 27 bp, Artificial Sequence
  Synthetic polymer.
ctgtaagtgtttggagttggaatttac

SEQ ID NO: 304 - DNA, 41 bp, Artificial Sequence
  Synthetic polymer.
ccatgcctgtggcacacttctgtccttcacgctgtcttctc

SEQ ID NO: 305 - DNA, 42 bp, Artificial Sequence
  Synthetic polymer.
ccctctctcagcatttttgctgttcgtgaaatgaggacatag

SEQ ID NO: 306 - DNA, 24 bp, Artificial Sequence
  Synthetic polymer.
ccgagatggatctggctgggaccc

SEQ ID NO: 307 - DNA, 43 bp, Artificial Sequence
  Synthetic polymer.
tctgttacggaagtgtactctctgagggcctcccacctgagtc

SEQ ID NO: 308 - DNA, 17 bp, Artificial Sequence
  Synthetic polymer.
agaagaagaagaggaag

SEQ ID NO: 309 - DNA, 20 bp, Artificial Sequence
  Synthetic polymer.
cccagagtccatatcaatgg

SEQ ID NO: 310 - DNA, 20 bp, Artificial Sequence
  Synthetic polymer.
gagaggaccgggttggacga

SEQ ID NO: 311 - DNA, 21 bp, Artificial Sequence
  Synthetic polymer.
aaagggaaggctgaactgctg

SEQ ID NO: 312 - DNA, 27 bp, Artificial Sequence
  Synthetic polymer.
atggggtgcaagctcttgatcgaagcc

SEQ ID NO: 313 - DNA, 17 bp, Artificial Sequence
  Synthetic polymer.
actgtagtaactcctac

SEQ ID NO: 314 - DNA, 22 bp, Artificial Sequence
  Synthetic polymer.
tctttaggatcaatttccattc

SEQ ID NO: 315 - DNA, 31 bp, Artificial Sequence
  Synthetic polymer.
aagcgagtctgaacagggcgactgagtttga

SEQ ID NO: 316 - DNA, 31 bp, Artificial Sequence
  Synthetic polymer.
ccttcctaattcttctttcaatagctattta

SEQ ID NO: 317 - DNA, 32 bp, Artificial Sequence
  Synthetic polymer.
ggctggtccgatgggagtgggtgatccgaact

SEQ ID NO: 318 - DNA, 32 bp, Artificial Sequence
  Synthetic polymer.
ggctggtccgatggtagtgggttatagggatt

SEQ ID NO: 319 - DNA, 36 bp, Artificial Sequence
  Synthetic polymer.
gaaaagacatggagggtgtagaataagtgggagctt

SEQ ID NO: 320 - DNA, 37 bp, Artificial Sequence
  Synthetic polymer.
cctgcatcagaggacaaacccgctaataacttgatcc

SEQ ID NO: 321 - DNA, 20 bp, Artificial Sequence
  Synthetic polymer.
cagggagctggagagggttc

SEQ ID NO: 322 - DNA, 23 bp, Artificial Sequence
  Synthetic polymer.
tgcgagtgtagaggtgaaattcg

SEQ ID NO: 323 - DNA, 24 bp, Artificial Sequence
  Synthetic polymer.
ctgtgtccccacccaaatctcatc

SEQ ID NO: 324 - DNA, 24 bp, Artificial Sequence
  Synthetic polymer.
gtgtccatgttgaaaactcgcctg

SEQ ID NO: 325 - DNA, 26 bp, Artificial Sequence
  Synthetic polymer.
cccttcccatttttaatagttgtagc

SEQ ID NO: 326 - DNA, 26 bp, Artificial Sequence
  Synthetic polymer.
tgctgcgggctgtcaggatgctcacc

SEQ ID NO: 327 - DNA, 27 bp, Artificial Sequence
  Synthetic polymer.
gttatttggattctgggtatgctctgg

SEQ ID NO: 328 - DNA, 29 bp, Artificial Sequence
  Synthetic polymer.
cagcccgggttccctctttctgccatctc

SEQ ID NO: 329 - DNA, 30 bp, Artificial Sequence
  Synthetic polymer.
taggtggatggtggatgggtggatgatgga

SEQ ID NO: 330 - DNA, 44 bp, Artificial Sequence
  Synthetic polymer.
cacctgtgcgtgtgggtgatgctgcgggctgtcagatgctgacc

SEQ ID NO: 331 - DNA, 24 bp, Artificial Sequence
  Synthetic polymer.
cctatctcagaatgcctgaaccac

SEQ ID NO: 332 - DNA, 35 bp, Artificial Sequence
  Synthetic polymer.
ttctggtagaattcagctgtgaatccgtcttgtcc

SEQ ID NO: 333 - DNA, 40 bp, Artificial Sequence
  Synthetic polymer.
cccattcattcatttcaatatccttcaaacatttcttttc

SEQ ID NO: 334 - DNA, 17 bp, Artificial Sequence
  Synthetic polymer.
aggactgtcctcgggaa

SEQ ID NO: 335 - DNA, 20 bp, Artificial Sequence
  Synthetic polymer.
atttgagaggggctgacctt

SEQ ID NO: 336 - DNA, 21 bp, Artificial Sequence
  Synthetic polymer.
ccccagaatgatcttgccttc

SEQ ID NO: 337 - DNA, 25 bp, Artificial Sequence
  Synthetic polymer.
atacatgagttgggcttactgagtg

SEQ ID NO: 338 - DNA, 26 bp, Artificial Sequence
  Synthetic polymer.
taaatgggtaagaagcccggctcgct

SEQ ID NO: 339 - DNA, 27 bp, Artificial Sequence
  Synthetic polymer.
cagaactggaacttgaacccacatttc

SEQ ID NO: 340 - DNA, 38 bp, Artificial Sequence
  Synthetic polymer.
gcattggtggttcagtggtagaattctcgcctggtgga

SEQ ID NO: 341 - DNA, 39 bp, Artificial Sequence
  Synthetic polymer.
caaaggtcaaacaacacaagtgagtctcaaactctcaac

SEQ ID NO: 342 - DNA, 42 bp, Artificial Sequence
  Synthetic polymer.
cctcgcgtcgcttcctcttctccttcaggagcgttttatccc

SEQ ID NO: 343 - DNA, 44 bp, Artificial Sequence
  Synthetic polymer.
caagtgcaaagggaattcattttgaagagttttatgcaactgtg

SEQ ID NO: 344 - DNA, 20 bp, Artificial Sequence
  Synthetic polymer.
agttctacagtcggccgatc

SEQ ID NO: 345 - DNA, 21 bp, Artificial Sequence
  Synthetic polymer.
aatggaggagtggtcggagga

SEQ ID NO: 346 - DNA, 21 bp, Artificial Sequence
  Synthetic polymer.
caaatgactatctcactgctc

SEQ ID NO: 347 - DNA, 24 bp, Artificial Sequence
  Synthetic polymer.
catattgttctgtgatcttaactg

SEQ ID NO: 348 - DNA, 25 bp, Artificial Sequence
  Synthetic polymer.
gggacgttagctcagttggtagagc

SEQ ID NO: 349 - DNA, 31 bp, Artificial Sequence
  Synthetic polymer.
ttgatctctggactgaggctttgtgtgtgcc

SEQ ID NO: 350 - DNA, 33 bp, Artificial Sequence
  Synthetic polymer.
acacgatctcggctcactgcaacctctgcctcc

SEQ ID NO: 351 - DNA, 34 bp, Artificial Sequence
  Synthetic polymer.
ccctggctccctgctgggcttggggagcctcttc

SEQ ID NO: 352 - DNA, 36 bp, Artificial Sequence
  Synthetic polymer.
tgcgagcggtcccgggttcacatcccggacgagccc

SEQ ID NO: 353 - DNA, 37 bp, Artificial Sequence
  Synthetic polymer.
ccctcaatccctggtcgagggagagggacttcctgtc

SEQ ID NO: 354 - DNA, 39 bp, Artificial Sequence
  Synthetic polymer.
gattaggatacaaggtcttgctagaactccctatctccc

SEQ ID NO: 355 - DNA, 43 bp, Artificial Sequence
  Synthetic polymer.
ctgtggaacggggtgagatgggatgggatgggacaggatagga

SEQ ID NO: 356 - DNA, 17 bp, Artificial Sequence
  Synthetic polymer.
ctggaaggtttgactgt

SEQ ID NO: 357 - DNA, 23 bp, Artificial Sequence
  Synthetic polymer.
tgccctttgtcatccctatgcct

SEQ ID NO: 358 - DNA, 24 bp, Artificial Sequence
  Synthetic polymer.
ccccatgaccctattcaagacttc

SEQ ID NO: 359 - DNA, 24 bp, Artificial Sequence
  Synthetic polymer.
cggtagctcgtcaggctcataacc

SEQ ID NO: 360 - DNA, 24 bp, Artificial Sequence
  Synthetic polymer.
ttccctttgtcatccttatgcctg

SEQ ID NO: 361 - DNA, 25 bp, Artificial Sequence
  Synthetic polymer.
cttcaacatcacctgtagccatcac

SEQ ID NO: 362 - DNA, 26 bp, Artificial Sequence
  Synthetic polymer.
ccttccaccttggcctcccaaagtgc

SEQ ID NO: 363 - DNA, 27 bp, Artificial Sequence
  Synthetic polymer.
aggggaatggaatggaatggaatgcaa

SEQ ID NO: 364 - DNA, 28 bp, Artificial Sequence
  Synthetic polymer.
cgcgggtgagtaggtcgctgccaggtct

SEQ ID NO: 365 - DNA, 29 bp, Artificial Sequence
  Synthetic polymer.
agggaccctctgtggcgggtagtttgact

SEQ ID NO: 366 - DNA, 29 bp, Artificial Sequence
  Synthetic polymer.
tatatggaagacataaaaagagaagctcc

SEQ ID NO: 367 - DNA, 34 bp, Artificial Sequence
  Synthetic polymer.
aggaatttcggtccagattgtttcttgagtcact

SEQ ID NO: 368 - DNA, 35 bp, Artificial Sequence
  Synthetic polymer.
aaaaagtctttaactccaccattagcacccaaagc

SEQ ID NO: 369 - DNA, 36 bp, Artificial Sequence
  Synthetic polymer.
ctaaggggtcgggagttcgaatctctctgagcgcac

SEQ ID NO: 370 - DNA, 37 bp, Artificial Sequence
  Synthetic polymer.
cgtagtgtcggtggttcgattccgcccctgggcacca

SEQ ID NO: 371 - DNA, 37 bp, Artificial Sequence
  Synthetic polymer.
gagctgattggtactaatcggtcgtgaggcttgacct

SEQ ID NO: 372 - DNA, 38 bp, Artificial Sequence
  Synthetic polymer.
gctctaagttcgagtctctctttcacttcttctcttgg

SEQ ID NO: 373 - DNA, 39 bp, Artificial Sequence
  Synthetic polymer.
cccaggttgagtttatgggggtagtgctgtaaggtcatt

SEQ ID NO: 374 - DNA, 43 bp, Artificial Sequence
  Synthetic polymer.
aatcggactgttcaactcacctggcaaccactcccagagcccc

SEQ ID NO: 375 - DNA, 44 bp, Artificial Sequence
  Synthetic polymer.
tttcaaggactgtgtttaatttccttttggatttgtttattttg

SEQ ID NO: 376 - DNA, 18 bp, Artificial Sequence
  Synthetic polymer.
cgaataagctttgatcca

SEQ ID NO: 377 - DNA, 20 bp, Artificial Sequence
  Synthetic polymer.
cactggaattctgagcccct

SEQ ID NO: 378 - DNA, 20 bp, Artificial Sequence
  Synthetic polymer.
caggagtcgggggtgggacg

SEQ ID NO: 379 - DNA, 21 bp, Artificial Sequence
  Synthetic polymer.
aaaagaggaccaccaccaaga

SEQ ID NO: 380 - DNA, 21 bp, Artificial Sequence
  Synthetic polymer.
ggtggtggcggcggtggtggc

SEQ ID NO: 381 - DNA, 21 bp, Artificial Sequence
  Synthetic polymer.
gtcttactctgttgctcaggc

SEQ ID NO: 382 - DNA, 23 bp, Artificial Sequence
  Synthetic polymer.
cctcctctggatcacatgggctc

SEQ ID NO: 383 - DNA, 23 bp, Artificial Sequence
  Synthetic polymer.
ccttcgggcctgtccagaacctc

SEQ ID NO: 384 - DNA, 23 bp, Artificial Sequence
  Synthetic polymer.
ttcgaatctcaccgcttccgcca

SEQ ID NO: 385 - DNA, 29 bp, Artificial Sequence
  Synthetic polymer.
ccatcacataggggattagatttcaatgc

SEQ ID NO: 386 - DNA, 29 bp, Artificial Sequence
  Synthetic polymer.
tgtaagggctgggtcggtcgggctggggc

SEQ ID NO: 387 - DNA, 31 bp, Artificial Sequence
  Synthetic polymer.
cagcgcctttgcacacgctattctctctgcc

SEQ ID NO: 388 - DNA, 31 bp, Artificial Sequence
  Synthetic polymer.
cgcggagcccagggttcgattccctgtaccg

SEQ ID NO: 389 - DNA, 31 bp, Artificial Sequence
  Synthetic polymer.
ctgatgggctgggcagggctccctggatggg

SEQ ID NO: 390 - DNA, 33 bp, Artificial Sequence
  Synthetic polymer.
ccccacttccgtactgagtttctcacctgtttg

SEQ ID NO: 391 - DNA, 34 bp, Artificial Sequence
  Synthetic polymer.
agtactgttatttagcgtgctaaatatattgtcc

SEQ ID NO: 392 - DNA, 34 bp, Artificial Sequence
  Synthetic polymer.
agtgcatcgcgcgaaagtaggtcgtcgccggctt

SEQ ID NO: 393 - DNA, 36 bp, Artificial Sequence
  Synthetic polymer.
cctgattttttttgcaatttctttgtattgttttta

SEQ ID NO: 394 - DNA, 37 bp, Artificial Sequence
  Synthetic polymer.
tgatggagtggcctggactcacattaaaataagtact

SEQ ID NO: 395 - DNA, 38 bp, Artificial Sequence
  Synthetic polymer.
ccccttacccatcaaattttccttaaaaactccaatcc

SEQ ID NO: 396 - DNA, 39 bp, Artificial Sequence
  Synthetic polymer.
ctctttggggcggggtgggggagggggagcctcgcgtcc

SEQ ID NO: 397 - DNA, 40 bp, Artificial Sequence
  Synthetic polymer.
cctgagctcttgttcgatgtccaaggataatgaggtggca

SEQ ID NO: 398 - DNA, 40 bp, Artificial Sequence
  Synthetic polymer.
taaggaggaggaacattgtgagcaggagaaggatctgggg

SEQ ID NO: 399 - DNA, 40 bp, Artificial Sequence
  Synthetic polymer.
tcctgtccggttgaggcctttctcttggggtcttgctgtc

SEQ ID NO: 400 - DNA, 41 bp, Artificial Sequence
  Synthetic polymer.
cctttcatatcttctcaaatactgatttaattttatactgg

SEQ ID NO: 401 - DNA, 42 bp, Artificial Sequence
  Synthetic polymer.
cctaggttcaagtgatcctcctgcttcagcttcctgagtagc

SEQ ID NO: 402 - DNA, 42 bp, Artificial Sequence
  Synthetic polymer.
cctggcctcaagcaatcctcccaccttggcctccacaagtac

SEQ ID NO: 403 - DNA, 44 bp, Artificial Sequence
  Synthetic polymer.
catctcagctccaaacccacaggttgggttcagttcttgcatcc

SEQ ID NO: 404 - DNA, 23 bp, Artificial Sequence
  Synthetic polymer.
tggaattctcgggtgccaaggaa

SEQ ID NO: 405 - DNA, 21 bp, Artificial Sequence
  Synthetic polymer.
gctgattgtcacgttctgatt

SEQ ID NO: 406 - DNA, 20 bp, Artificial Sequence
  Synthetic polymer.
gcccctgggcctatcctaga

SEQ ID NO: 407 - DNA, 19 bp, Artificial Sequence
  Synthetic polymer.
agttcttcagtggcaagct

SEQ ID NO: 408 - DNA, 22 bp, Artificial Sequence
  Synthetic polymer.
accctgtagaaccgaatttgta

SEQ ID NO: 409 - DNA, 23 bp, Artificial Sequence
  Synthetic polymer.
taggtagtttcctgttgttggat

SEQ ID NO: 410 - DNA, 21 bp, Artificial Sequence
  Synthetic polymer.
accctgtagatctgaatttgt

SEQ ID NO: 411 - DNA, 19 bp, Artificial Sequence
  Synthetic polymer.
tgagatgaagctgtagctc

SEQ ID NO: 412 - DNA, 22 bp, Artificial Sequence
  Synthetic polymer.
taccctgtagaaccgaattggt

SEQ ID NO: 413 - DNA, 21 bp, Artificial Sequence
  Synthetic polymer.
accctgtagaaccgaatttgg

SEQ ID NO: 414 - DNA, 22 bp, Artificial Sequence
  Synthetic polymer.
taacagtctacagccatggtcg

SEQ ID NO: 415 - DNA, 20 bp, Artificial Sequence
  Synthetic polymer.
agttcttcagtggcaagctt

SEQ ID NO: 416 - DNA, 22 bp, Artificial Sequence
  Synthetic polymer.
taccctgtagaaccgaatttgg

SEQ ID NO: 417 - DNA, 22 bp, Artificial Sequence
  Synthetic polymer.
cagtgcaatgatgaaagggcat

SEQ ID NO: 418 - DNA, 21 bp, Artificial Sequence
  Synthetic polymer.
taccctgtagaaccgaattta

SEQ ID NO: 419 - DNA, 22 bp, Artificial Sequence
  Synthetic polymer.
tacagttgttcaaccagttact

SEQ ID NO: 420 - DNA, 22 bp, Artificial Sequence
  Synthetic polymer.
accctgtagaaccgaatttggg

SEQ ID NO: 421 - DNA, 22 bp, Artificial Sequence
  Synthetic polymer.
taccctgtaggaccgaatttgt

SEQ ID NO: 422 - DNA, 20 bp, Artificial Sequence
  Synthetic polymer.
tggcagtgtcttagctggtt

SEQ ID NO: 423 - DNA, 20 bp, Artificial Sequence
  Synthetic polymer.
accctgtagaaccgaattta

SEQ ID NO: 424 - DNA, 22 bp, Artificial Sequence
  Synthetic polymer.
accctgtagaaccgaatttgtt

SEQ ID NO: 425 - DNA, 22 bp, Artificial Sequence
  Synthetic polymer.
taccctgtagatccgattttgt

SEQ ID NO: 426 - DNA, 22 bp, Artificial Sequence
  Synthetic polymer.
taccctgtagaaccgagtttgt

SEQ ID NO: 427 - DNA, 23 bp, Artificial Sequence
  Synthetic polymer.
ttcaagtaatccaggataggcct

SEQ ID NO: 428 - DNA, 22 bp, Artificial Sequence
  Synthetic polymer.
taccctgtagaaccgaatttat

SEQ ID NO: 429 - DNA, 21 bp, Artificial Sequence
  Synthetic polymer.
taccctgtagaaccggatttg

SEQ ID NO: 430 - DNA, 25 bp, Artificial Sequence
  Synthetic polymer.
tattgcacttgtcccggcctgtagc

SEQ ID NO: 431 - DNA, 23 bp, Artificial Sequence
  Synthetic polymer.
accctgtagatctgaatttgtga

SEQ ID NO: 432 - DNA, 19 bp, Artificial Sequence
  Synthetic polymer.
cactagattgtgagctcct

SEQ ID NO: 433 - DNA, 22 bp, Artificial Sequence
  Synthetic polymer.
taccctgtagtaccgaatttgt

SEQ ID NO: 434 - DNA, 22 bp, Artificial Sequence
  Synthetic polymer.
cagtgcaatgttaaaagggcaa

SEQ ID NO: 435 - DNA, 21 bp, Artificial Sequence
  Synthetic polymer.
ctgacctatgatttgacagcc

SEQ ID NO: 436 - DNA, 23 bp, Artificial Sequence
  Synthetic polymer.
ctgacctatgaattgacagccct

SEQ ID NO: 437 - DNA, 22 bp, Artificial Sequence
  Synthetic polymer.
ccactgccccaggtgctgctgg

SEQ ID NO: 438 - DNA, 22 bp, Artificial Sequence
  Synthetic polymer.
tgaggtagtaggttgtgtgggt

SEQ ID NO: 439 - DNA, 21 bp, Artificial Sequence
  Synthetic polymer.
actgtgcgtgtgacagcggct

SEQ ID NO: 440 - DNA, 20 bp, Artificial Sequence
  Synthetic polymer.
ctgcgcaagctactgccttg

SEQ ID NO: 441 - DNA, 23 bp, Artificial Sequence
  Synthetic polymer.
cacccgtagaaccgaccttgcga

SEQ ID NO: 442 - DNA, 21 bp, Artificial Sequence
  Synthetic polymer.
ctgacctatgtattgacagcc

SEQ ID NO: 443 - DNA, 22 bp, Artificial Sequence
  Synthetic polymer.
taccctgtagaaccgaatttgc

SEQ ID NO: 444 - DNA, 25 bp, Artificial Sequence
  Synthetic polymer.
tgagaactgaattccataggctgaa

SEQ ID NO: 445 - DNA, 24 bp, Artificial Sequence
  Synthetic polymer.
tgacctatgaattgacagccaatt

SEQ ID NO: 446 - DNA, 23 bp, Artificial Sequence
  Synthetic polymer.
taccctgtagaaccgaatttgta

SEQ ID NO: 447 - DNA, 21 bp, Artificial Sequence
  Synthetic polymer.
tgagatgaagcactgtagatc

SEQ ID NO: 448 - DNA, 22 bp, Artificial Sequence
  Synthetic polymer.
taccctgtagaaccgaacttgt

SEQ ID NO: 449 - DNA, 21 bp, Artificial Sequence
  Synthetic polymer.
ctgacctatgaactgacagcc

SEQ ID NO: 450 - DNA, 18 bp, Artificial Sequence
  Synthetic polymer.
gattgtcacgttctgatt

SEQ ID NO: 451 - DNA, 22 bp, Artificial Sequence
  Synthetic polymer.
ttacagtctacagccatggtcg

SEQ ID NO: 452 - DNA, 24 bp, Artificial Sequence
  Synthetic polymer.
cattgcacttgtctcggtctgaat

SEQ ID NO: 453 - DNA, 22 bp, Artificial Sequence
  Synthetic polymer.
taccctgttgaaccgaatttgt

SEQ ID NO: 454 - DNA, 22 bp, Artificial Sequence
  Synthetic polymer.
caaagtgctgttcgtgcaggta

SEQ ID NO: 455 - DNA, 26 bp, Artificial Sequence
  Synthetic polymer.
ctcgcttctggcgccaagcgcccggc

SEQ ID NO: 456 - DNA, 20 bp, Artificial Sequence
  Synthetic polymer.
aactggccctcaaagtcccg

SEQ ID NO: 457 - DNA, 23 bp, Artificial Sequence
  Synthetic polymer.
tgagaactgaattccataggcaa

SEQ ID NO: 458 - DNA, 24 bp, Artificial Sequence
  Synthetic polymer.
tgaggtagtagattgtatagtttt

SEQ ID NO: 459 - DNA, 17 bp, Artificial Sequence
  Synthetic polymer.
accctgtagatccgaat

SEQ ID NO: 460 - DNA, 25 bp, Artificial Sequence
  Synthetic polymer.
aggctgtgatgctctcctgagccct

SEQ ID NO: 461 - DNA, 17 bp, Artificial Sequence
  Synthetic polymer.
taacactgtctggtaac

SEQ ID NO: 462 - DNA, 22 bp, Artificial Sequence
  Synthetic polymer.
taccctgtagatccgaattcgt

SEQ ID NO: 463 - DNA, 23 bp, Artificial Sequence
  Synthetic polymer.
ccgccccaccccgcgcgcgccgc

SEQ ID NO: 464 - DNA, 27 bp, Artificial Sequence
  Synthetic polymer.
cgcttctggcgccaagcgcccggccgc

SEQ ID NO: 465 - DNA, 30 bp, Artificial Sequence
  Synthetic polymer.
agattgagggttcgtcccttcgtggtcgcc

SEQ ID NO: 466 - DNA, 30 bp, Artificial Sequence
  Synthetic polymer.
ggcttggtctaggggtatgattctcgcttt

SEQ ID NO: 467 - DNA, 29 bp, Artificial Sequence
  Synthetic polymer.
ggctttgtctaggggtatgattctcgctt

SEQ ID NO: 468 - DNA, 25 bp, Artificial Sequence
  Synthetic polymer.
cccgccccaccccgcgcgcgccgct

SEQ ID NO: 469 - DNA, 30 bp, Artificial Sequence
  Synthetic polymer.
cgtacggaagacccgctccccggcgccgct

SEQ ID NO: 470 - DNA, 27 bp, Artificial Sequence
  Synthetic polymer.
gtacggaagacccgctccccggcgccg

SEQ ID NO: 471 - DNA, 28 bp, Artificial Sequence
  Synthetic polymer.
tggtctagcggttaggattcctggtttt

SEQ ID NO: 472 - DNA, 22 bp, Artificial Sequence
  Synthetic polymer.
cgccccaccccgcgcgcgccgc

SEQ ID NO: 473 - DNA, 22 bp, Artificial Sequence
  Synthetic polymer.
cccgcgaggggggcccgggcac

SEQ ID NO: 474 - DNA, 28 bp, Artificial Sequence
  Synthetic polymer.
gcgccgccgccccccccacgcccggggc

SEQ ID NO: 475 - DNA, 23 bp, Artificial Sequence
  Synthetic polymer.
gctccccgtcctcccccctcccc

SEQ ID NO: 476 - DNA, 25 bp, Artificial Sequence
  Synthetic polymer.
gcgcaatgaaggtgaaggccggcgc

SEQ ID NO: 477 - DNA, 22 bp, Artificial Sequence
  Synthetic polymer.
acgctgccagttgaagaactgt

SEQ ID NO: 478 - DNA, 23 bp, Artificial Sequence
  Synthetic polymer.
gcccctgggcctatcctagaaaa

SEQ ID NO: 479 - DNA, 23 bp, Artificial Sequence
  Synthetic polymer.
gcgggtccggccgtgtcggcggc

SEQ ID NO: 480 - DNA, 28 bp, Artificial Sequence
  Synthetic polymer.
ggcttggtctaggggtatgattctcgct

SEQ ID NO: 481 - DNA, 21 bp, Artificial Sequence
  Synthetic polymer.
ccacctcccctgcaaacgtcc

SEQ ID NO: 482 - DNA, 19 bp, Artificial Sequence
  Synthetic polymer.
ggttaggattcctggtttt

SEQ ID NO: 483 - DNA, 30 bp, Artificial Sequence
  Synthetic polymer.
tctggcatgctaactagttacgcgaccccc

SEQ ID NO: 484 - DNA, 30 bp, Artificial Sequence
  Synthetic polymer.
cgcgtcccccgaagagggggacggcggagc

SEQ ID NO: 485 - DNA, 30 bp, Artificial Sequence
  Synthetic polymer.
gcggagcgagcgcacggggtcggcggcgac

SEQ ID NO: 486 - DNA, 30 bp, Artificial Sequence
  Synthetic polymer.
cccccgccccaccccgcgcgcgccgctcgc

SEQ ID NO: 487 - DNA, 29 bp, Artificial Sequence
  Synthetic polymer.
ccgtaggtgaacctgcggaaggatcatta

SEQ ID NO: 488 - DNA, 26 bp, Artificial Sequence
  Synthetic polymer.
gggctacgcctgtctgagcgtcgctt

SEQ ID NO: 489 - DNA, 24 bp, Artificial Sequence
  Synthetic polymer.
gctacgcctgtctgagcgtcgctt

SEQ ID NO: 490 - DNA, 26 bp, Artificial Sequence
  Synthetic polymer.
cccccacaaccgcgcttgactagctt

SEQ ID NO: 491 - DNA, 21 bp, Artificial Sequence
  Synthetic polymer.
ccctacccccccggccccgtc

SEQ ID NO: 492 - DNA, 28 bp, Artificial Sequence
  Synthetic polymer.
cccgccccaccccgcgcgcgccgctcgc

SEQ ID NO: 493 - DNA, 30 bp, Artificial Sequence
  Synthetic polymer.
gggggtatagctcagtggtagagcgtgctt

SEQ ID NO: 494 - DNA, 30 bp, Artificial Sequence
  Synthetic polymer.
gtcggtcgggctggggcgcgaagcggggct

SEQ ID NO: 495 - DNA, 20 bp, Artificial Sequence
  Synthetic polymer.
tcagtggagagcatttgact

SEQ ID NO: 496 - DNA, 22 bp, Artificial Sequence
  Synthetic polymer.
cacccctagaaccgaccttgcg

SEQ ID NO: 497 - DNA, 23 bp, Artificial Sequence
  Synthetic polymer.
cctcaccatcccttctgcctgca

SEQ ID NO: 498 - DNA, 21 bp, Artificial Sequence
  Synthetic polymer.
gtcaggatggccgagcggtct

SEQ ID NO: 499 - DNA, 30 bp, Artificial Sequence
  Synthetic polymer.
tccctggtctagtggttaggattcggcgcg

SEQ ID NO: 500 - DNA, 21 bp, Artificial Sequence
  Synthetic polymer.
tgagatgaagcactgtagatc

SEQ ID NO: 501 - DNA, 23 bp, Artificial Sequence
  Synthetic polymer.
ggatcggccccgccggggtcggc

SEQ ID NO: 502 - DNA, 22 bp, Artificial Sequence
  Synthetic polymer.
ggaacctgcggaaggatcatta

SEQ ID NO: 503 - DNA, 23 bp, Artificial Sequence
  Synthetic polymer.
tgaggtagtaggttgtatggttg

SEQ ID NO: 504 - DNA, 24 bp, Artificial Sequence
  Synthetic polymer.
gtctagtggttaggattcggcgct

SEQ ID NO: 505 - DNA, 30 bp, Artificial Sequence
  Synthetic polymer.
tccctggtctagtggctaggattcggcgct

SEQ ID NO: 506 - DNA, 23 bp, Artificial Sequence
  Synthetic polymer.
gccgccccccccacgcccggggc

SEQ ID NO: 507 - DNA, 24 bp, Artificial Sequence
  Synthetic polymer.
tgtcagtttgtcaaataccccaag

SEQ ID NO: 508 - DNA, 23 bp, Artificial Sequence
  Synthetic polymer.
cagcagcaattcatgttttgaat

SEQ ID NO: 509 - DNA, 26 bp, Artificial Sequence
  Synthetic polymer.
gtggttgtagtccgtgcgagaatacc

SEQ ID NO: 510 - DNA, 23 bp, Artificial Sequence
  Synthetic polymer.
ggatatcatcatatactgtaagt

SEQ ID NO: 511 - DNA, 20 bp, Artificial Sequence
  Synthetic polymer.
taacagtctccagtcacggc

SEQ ID NO: 512 - DNA, 23 bp, Artificial Sequence
  Synthetic polymer.
tcagtgcactacagaactttttt

SEQ ID NO: 513 - DNA, 23 bp, Artificial Sequence
  Synthetic polymer.
ccagtggggctgctgttatctgt

SEQ ID NO: 514 - DNA, 21 bp, Artificial Sequence
  Synthetic polymer.
gataaagtagaaagcactact

SEQ ID NO: 515 - DNA, 23 bp, Artificial Sequence
  Synthetic polymer.
taggtagtttcctgttgttggat

SEQ ID NO: 516 - DNA, 22 bp, Artificial Sequence
  Synthetic polymer.
atgcttatcagactgatgttga

SEQ ID NO: 517 - DNA, 22 bp, Artificial Sequence
  Synthetic polymer.
tagtgcaatattgcttataggg

SEQ ID NO: 518 - DNA, 23 bp, Artificial Sequence
  Synthetic polymer.
cccataaagtagaaagcactact

SEQ ID NO: 519 - DNA, 21 bp, Artificial Sequence
  Synthetic polymer.
tacccattgcatatcggagtt

SEQ ID NO: 520 - DNA, 23 bp, Artificial Sequence
  Synthetic polymer.
actggacttggagtcagaaggaa

SEQ ID NO: 521 - DNA, 21 bp, Artificial Sequence
  Synthetic polymer.
aagcagcaattcatgttttga

SEQ ID NO: 522 - DNA, 22 bp, Artificial Sequence
  Synthetic polymer.
ctgcagcacgtaaatattggcg

SEQ ID NO: 523 - DNA, 20 bp, Artificial Sequence
  Synthetic polymer.
tggcagtgtcttagctggtt

SEQ ID NO: 524 - DNA, 23 bp, Artificial Sequence
  Synthetic polymer.
actggacttggagtcagaaggtt

SEQ ID NO: 525 - DNA, 26 bp, Artificial Sequence
  Synthetic polymer.
tgagaactgaattccataggctgtaa

SEQ ID NO: 526 - DNA, 23 bp, Artificial Sequence
  Synthetic polymer.
actggacttggagtcagaaggat

SEQ ID NO: 527 - DNA, 27 bp, Artificial Sequence
  Synthetic polymer.
tgagaactgaattccataggctgtaat

SEQ ID NO: 528 - DNA, 22 bp, Artificial Sequence
  Synthetic polymer.
gttgagactctgaaatctgatt

SEQ ID NO: 529 - DNA, 19 bp, Artificial Sequence
  Synthetic polymer.
ttaatgctaatcgtgatag

SEQ ID NO: 530 - DNA, 22 bp, Artificial Sequence
  Synthetic polymer.
tgagaactgaattccataggaa

SEQ ID NO: 531 - DNA, 22 bp, Artificial Sequence
  Synthetic polymer.
ctatacgacctgctgcctttca

SEQ ID NO: 532 - DNA, 23 bp, Artificial Sequence
  Synthetic polymer.
taccctgtagaaccgaatttgcg

SEQ ID NO: 533 - DNA, 19 bp, Artificial Sequence
  Synthetic polymer.
tggcagtgtcttagctggt

SEQ ID NO: 534 - DNA, 21 bp, Artificial Sequence
  Synthetic polymer.
ccagtggggctgctgttatct

SEQ ID NO: 535 - DNA, 23 bp, Artificial Sequence
  Synthetic polymer.
ttgagaactgaattccatgggtt

SEQ ID NO: 536 - DNA, 22 bp, Artificial Sequence
  Synthetic polymer.
ttacagtctacagccatggtcg

SEQ ID NO: 537 - DNA, 23 bp, Artificial Sequence
  Synthetic polymer.
actggacttggagtcagaaggct

SEQ ID NO: 538 - DNA, 26 bp, Artificial Sequence
  Synthetic polymer.
tgagaactgaattccataggctgtag

SEQ ID NO: 539 - DNA, 23 bp, Artificial Sequence
  Synthetic polymer.
cccataaagtagaaagcactaca

SEQ ID NO: 540 - DNA, 19 bp, Artificial Sequence
  Synthetic polymer.
tgaggtagtagtttgtgct

SEQ ID NO: 541 - DNA, 20 bp, Artificial Sequence
  Synthetic polymer.
cggcgcaagctactgccttg

SEQ ID NO: 542 - DNA, 19 bp, Artificial Sequence
  Synthetic polymer.
agttcttcagtggcaagct

SEQ ID NO: 543 - DNA, 22 bp, Artificial Sequence
  Synthetic polymer.
tcccctgtagaaccgaatttgt

SEQ ID NO: 544 - DNA, 25 bp, Artificial Sequence
  Synthetic polymer.
actggacttggagtcagaaggcatt

SEQ ID NO: 545 - DNA, 22 bp, Artificial Sequence
  Synthetic polymer.
aagctcggtctgaggcccctca

SEQ ID NO: 546 - DNA, 23 bp, Artificial Sequence
  Synthetic polymer.
ccagtggggctgctgttatctga

SEQ ID NO: 547 - DNA, 23 bp, Artificial Sequence
  Synthetic polymer.
tgagggagtagtttgtgctgtta

SEQ ID NO: 548 - DNA, 21 bp, Artificial Sequence
  Synthetic polymer.
aagaaagtagaaagcactact

SEQ ID NO: 549 - DNA, 21 bp, Artificial Sequence
  Synthetic polymer.
cgctgccagttgaagaactgt

SEQ ID NO: 550 - DNA, 18 bp, Artificial Sequence
  Synthetic polymer.
ggctggtccgatggtagt

SEQ ID NO: 551 - DNA, 22 bp, Artificial Sequence
  Synthetic polymer.
ctgggagaaggctgtttactct

SEQ ID NO: 552 - DNA, 19 bp, Artificial Sequence
  Synthetic polymer.
aagcaattctcaaaggagc

SEQ ID NO: 553 - DNA, 22 bp, Artificial Sequence
  Synthetic polymer.
ctcggcgccccctcgatgctct

SEQ ID NO: 554 - DNA, 20 bp, Artificial Sequence
  Synthetic polymer.
tgtcttgcaggccgtcatgc

SEQ ID NO: 555 - DNA, 19 bp, Artificial Sequence
  Synthetic polymer.
cgaatcattatttgctgct

SEQ ID NO: 556 - DNA, 23 bp, Artificial Sequence
  Synthetic polymer.
cagcagcaattcatgttttgaaa

SEQ ID NO: 557 - DNA, 20 bp, Artificial Sequence
  Synthetic polymer.
accaatattactgtgctgct

SEQ ID NO: 558 - DNA, 24 bp, Artificial Sequence
  Synthetic polymer.
ttcaagtaatccaggataggcttt

SEQ ID NO: 559 - DNA, 22 bp, Artificial Sequence
  Synthetic polymer.
ttgagaactgaattccatgggt

SEQ ID NO: 560 - DNA, 20 bp, Artificial Sequence
  Synthetic polymer.
tattgcacattactaagttg

SEQ ID NO: 561 - DNA, 22 bp, Artificial Sequence
  Synthetic polymer.
tgacctatgaattgacagccta

SEQ ID NO: 562 - DNA, 20 bp, Artificial Sequence
  Synthetic polymer.
actgtaaacgctttctgatg

SEQ ID NO: 563 - DNA, 24 bp, Artificial Sequence
  Synthetic polymer.
cattgcacttgtctcggtctgaat

SEQ ID NO: 564 - DNA, 20 bp, Artificial Sequence
  Synthetic polymer.
ataaagtagaaagcactact

SEQ ID NO: 565 - DNA, 21 bp, Artificial Sequence
  Synthetic polymer.
aagtgcaatgatgaaagggca

SEQ ID NO: 566 - DNA, 21 bp, Artificial Sequence
  Synthetic polymer.
accataaagtagaaagcacta

SEQ ID NO: 567 - DNA, 26 bp, Artificial Sequence
  Synthetic polymer.
ccccactgctaaatttgactggcttt

SEQ ID NO: 568 - DNA, 25 bp, Artificial Sequence
  Synthetic polymer.
tgtcagtttgtcaaataccccaaga

SEQ ID NO: 569 - DNA, 22 bp, Artificial Sequence
  Synthetic polymer.
tacccagtagaaccgaatttgt

SEQ ID NO: 570 - DNA, 22 bp, Artificial Sequence
  Synthetic polymer.
tttgttcgttcggctcgcgtaa

SEQ ID NO: 571 - DNA, 23 bp, Artificial Sequence
  Synthetic polymer.
atgctgccagttgaagaactgta

SEQ ID NO: 572 - DNA, 20 bp, Artificial Sequence
  Synthetic polymer.
tgagaaccacgtctgctctg

SEQ ID NO: 573 - DNA, 23 bp, Artificial Sequence
  Synthetic polymer.
ctgccaattccataggtcacagt

SEQ ID NO: 574 - DNA, 24 bp, Artificial Sequence
  Synthetic polymer.
tagcttatcagactgatgttgaga

SEQ ID NO: 575 - DNA, 25 bp, Artificial Sequence
  Synthetic polymer.
gtagcttatcagactgatgttgact

SEQ ID NO: 576 - DNA, 23 bp, Artificial Sequence
  Synthetic polymer.
tttggtccccttcaaccagctga

SEQ ID NO: 577 - DNA, 23 bp, Artificial Sequence
  Synthetic polymer.
tgtaatagcaactccatgtggaa

SEQ ID NO: 578 - DNA, 21 bp, Artificial Sequence
  Synthetic polymer.
gggacctatgaattgacagac

SEQ ID NO: 579 - DNA, 22 bp, Artificial Sequence
  Synthetic polymer.
taaggtgcatctagtgcagata

SEQ ID NO: 580 - DNA, 27 bp, Artificial Sequence
  Synthetic polymer.
gtactggaaagtgcacttggacgaaca

SEQ ID NO: 581 - DNA, 29 bp, Artificial Sequence
  Synthetic polymer.
cccggggctacgcctgtctgagcgtcgct

SEQ ID NO: 582 - DNA, 23 bp, Artificial Sequence
  Synthetic polymer.
aaagctgggttgagagggcgaaa

SEQ ID NO: 583 - DNA, 19 bp, Artificial Sequence
  Synthetic polymer.
cataaagtagaaagcacta

SEQ ID NO: 584 - DNA, 18 bp, Artificial Sequence
  Synthetic polymer.
tgtcagtttgtcaaatac

SEQ ID NO: 585 - DNA, 21 bp, Artificial Sequence
  Synthetic polymer.
tccggtgagctctcgctggcc

SEQ ID NO: 586 - DNA, 20 bp, Artificial Sequence
  Synthetic polymer.
tataaagtagaaagcactac

SEQ ID NO: 587 - DNA, 21 bp, Artificial Sequence
  Synthetic polymer.
tgctgccagttgaagaactgt

SEQ ID NO: 588 - DNA, 25 bp, Artificial Sequence
  Synthetic polymer.
agctcggtctgaggcccctcagttt

SEQ ID NO: 589 - DNA, 24 bp, Artificial Sequence
  Synthetic polymer.
tgtcagtttgtcaaataccccatg

SEQ ID NO: 590 - DNA, 19 bp, Artificial Sequence
  Synthetic polymer.
atcacagtggctaagttcc

SEQ ID NO: 591 - DNA, 23 bp, Artificial Sequence
  Synthetic polymer.
tgagaactgaattccataggcaa

SEQ ID NO: 592 - DNA, 22 bp, Artificial Sequence
  Synthetic polymer.
tgggtctttgcgggcgagatga

SEQ ID NO: 593 - DNA, 21 bp, Artificial Sequence
  Synthetic polymer.
taccctgtagaaccggatttg

SEQ ID NO: 594 - DNA, 21 bp, Artificial Sequence
  Synthetic polymer.
tgagggagtagattgtatagt

SEQ ID NO: 595 - DNA, 22 bp, Artificial Sequence
  Synthetic polymer.
taccctgttgaaccgaatttgt

SEQ ID NO: 596 - DNA, 21 bp, Artificial Sequence
  Synthetic polymer.
taaggtgcatctagtgcagat

SEQ ID NO: 597 - DNA, 23 bp, Artificial Sequence
  Synthetic polymer.
gagaactgaattccataggctgt

SEQ ID NO: 598 - DNA, 22 bp, Artificial Sequence
  Synthetic polymer.
tagcagcacgcaaatattggcg

SEQ ID NO: 599 - DNA, 18 bp, Artificial Sequence
  Synthetic polymer.
ggctcgttggtctagggg

SEQ ID NO: 600 - DNA, 20 bp, Artificial Sequence
  Synthetic polymer.
cagcagcaattcatgttttg

SEQ ID NO: 601 - DNA, 20 bp, Artificial Sequence
  Synthetic polymer.
aacattcaacgctgtcggtg

SEQ ID NO: 602 - DNA, 22 bp, Artificial Sequence
  Synthetic polymer.
atgcagcacgtaaatattggcg

SEQ ID NO: 603 - DNA, 23 bp, Artificial Sequence
  Synthetic polymer.
tgccgacgggcgctgaccccctt

SEQ ID NO: 604 - DNA, 29 bp, Artificial Sequence
  Synthetic polymer.
attggtcgtggttgtagtccgtgcgagaa

SEQ ID NO: 605 - DNA, 21 bp, Artificial Sequence
  Synthetic polymer.
tggcagtgtcttagctggttg

SEQ ID NO: 606 - DNA, 17 bp, Artificial Sequence
  Synthetic polymer.
tgtcagtttgtcaaata

SEQ ID NO: 607 - DNA, 22 bp, Artificial Sequence
  Synthetic polymer.
accctgagaccctaacttgtga

SEQ ID NO: 608 - DNA, 19 bp, Artificial Sequence
  Synthetic polymer.
tggcagtttgtcaaatacc

SEQ ID NO: 609 - DNA, 26 bp, Artificial Sequence
  Synthetic polymer.
actggacttggagtcagaaggcatat

SEQ ID NO: 610 - DNA, 24 bp, Artificial Sequence
  Synthetic polymer.
tcgaccggacctcgaccggctaga

SEQ ID NO: 611 - DNA, 22 bp, Artificial Sequence
  Synthetic polymer.
tcagcaccaggatattgttgga

SEQ ID NO: 612 - DNA, 22 bp, Artificial Sequence
  Synthetic polymer.
tgtaaccgcaactccatgtgga

SEQ ID NO: 613 - DNA, 26 bp, Artificial Sequence
  Synthetic polymer.
actggacttggagtcagaaggcatta

SEQ ID NO: 614 - DNA, 22 bp, Artificial Sequence
  Synthetic polymer.
aacactgtctggtaaagatggc

SEQ ID NO: 615 - DNA, 26 bp, Artificial Sequence
  Synthetic polymer.
tgtaaacatcctacactctcagctta

SEQ ID NO: 616 - DNA, 23 bp, Artificial Sequence
  Synthetic polymer.
actggactttgagtcagaaggca

SEQ ID NO: 617 - DNA, 24 bp, Artificial Sequence
  Synthetic polymer.
actggacttggagccagaaggcaa

SEQ ID NO: 618 - DNA, 23 bp, Artificial Sequence
  Synthetic polymer.
gtaacagcaactccatgtggaaa

SEQ ID NO: 619 - DNA, 26 bp, Artificial Sequence
  Synthetic polymer.
actggacttggagtcagaaggcaata

SEQ ID NO: 620 - DNA, 24 bp, Artificial Sequence
  Synthetic polymer.
ctggacttggagtcagaaggcaga

SEQ ID NO: 621 - DNA, 24 bp, Artificial Sequence
  Synthetic polymer.
tgatatgtttgatatattaggtta

SEQ ID NO: 622 - DNA, 24 bp, Artificial Sequence
  Synthetic polymer.
tgaaatgtttaggaccactagaat

SEQ ID NO: 623 - DNA, 22 bp, Artificial Sequence
  Synthetic polymer.
tggacttggagtcagaaggcat

SEQ ID NO: 624 - DNA, 25 bp, Artificial Sequence
  Synthetic polymer.
tgtaacagcaactccatgtggacta

SEQ ID NO: 625 - DNA, 22 bp, Artificial Sequence
  Synthetic polymer.
tcgaccggacctcgaccggcta

SEQ ID NO: 626 - DNA, 24 bp, Artificial Sequence
  Synthetic polymer.
tgagatgaagcactgtagctcata

SEQ ID NO: 627 - DNA, 23 bp, Artificial Sequence
  Synthetic polymer.
tttcagtcggatgtttgcagcaa

SEQ ID NO: 628 - DNA, 21 bp, Artificial Sequence
  Synthetic polymer.
gacctatgaattgacagccat

SEQ ID NO: 629 - DNA, 23 bp, Artificial Sequence
  Synthetic polymer.
ccactgccccaggtgctgctgga

SEQ ID NO: 630 - DNA, 25 bp, Artificial Sequence
  Synthetic polymer.
ctgacctatgaattgacagccatga

SEQ ID NO: 631 - DNA, 24 bp, Artificial Sequence
  Synthetic polymer.
accacagggtagaaccacggacga

SEQ ID NO: 632 - DNA, 23 bp, Artificial Sequence
  Synthetic polymer.
tcgaccggacctcgaccggctga

SEQ ID NO: 633 - DNA, 24 bp, Artificial Sequence
  Synthetic polymer.
tggctcagttcagcaggaacagga

SEQ ID NO: 634 - DNA, 23 bp, Artificial Sequence
  Synthetic polymer.
agcttatcagactgatgttgaaa

SEQ ID NO: 635 - DNA, 21 bp, Artificial Sequence
  Synthetic polymer.
atcacattgccagggataaaa

SEQ ID NO: 636 - DNA, 23 bp, Artificial Sequence
  Synthetic polymer.
tcaacaaaatcactgatgctgga

SEQ ID NO: 637 - DNA, 19 bp, Artificial Sequence
  Synthetic polymer.
acattgccagggatttcca

SEQ ID NO: 638 - DNA, 20 bp, Artificial Sequence
  Synthetic polymer.
aacactgtctggtaaagatg